(19) United States
(12) Patent Application Publication
(10) Pub. No.: US 2023/0140216 A1
(43) Pub. Date: May 4, 2023

(54) COMBINATION THERAPY FOR TREATING MPS1

(71) Applicant: GAIN THERAPEUTICS SA, Lugano (CH)

(72) Inventors: Ana Maria GARCÍA COLLAZO, Esplugues de Llobregat, Barcelona (ES); Elena CUBERO JORDÀ, Barcelona (ES); Xavier BARRIL ALONSO, Montferri (ES); Roberto MAJ, Saronno (IT); Natalia PEREZ CARMONA, Barcelona (ES)

(73) Assignee: GAIN THERAPEUTICS SA, Lugano (CH)

(21) Appl. No.: 17/792,766

(22) PCT Filed: Feb. 3, 2021
(86) PCT No.: PCT/IB2021/050885

§ 371 Date: Jul. 14, 2022

(30) Foreign Application Priority Data
Feb. 3, 2020 (EP) 20382071.7

(51) Int. Cl. C07D 487/04; A61P 19/08; A61P 3/00; A61K 38/47; A61K 31/519; C07D 495/04; C07D 239/90; A61K 31/517; C07D 401/12; C07D 403/06; C07D 417/12; C07D 403/12

(52) U.S. Cl. CPC C07D 487/04; A61K 31/517; A61K 31/519; A61K 38/47; A61P 3/00; A61P 19/08; C07D 239/90; C07D 401/12; C07D 403/06; C07D 403/12; C07D 417/12; C07D 495/04; C12Y 302/01076

(57) ABSTRACT

The application is directed to compounds of formula (I) and their salts and solvates, wherein B, R¹, R², R³, R^{3’}, R⁴, R^{4’} and R⁵ are as set forth in the specification, as well as to methods for their preparation, pharmaceutical compositions comprising the same, and use thereof for the treatment and/or prevention of, e.g., MPS1, optionally in combination with α-L-iduronidase or an analog or variant thereof, e.g., laronidase.

### CROSS-REFERENCE TO RELATED APPLICATION

This application claims priority to European Pat. Application No. EP20382071.7 filed on Feb. 3, 2020, the entirety of which is incorporated by reference herein.

### FIELD OF THE DISCLOSURE

The present disclosure is related to compounds of formula (I) and (I′), and the use of such compounds, optionally in combination with laronidase, in the treatment and/or prevention of conditions associated with the alteration of the activity of α-L-iduronidase (IDUA) in a patient, such as, for example, mucopolysaccharidosis 1 (MPS1), heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, and a developmental disorder.

### BACKGROUND OF THE INVENTION

### Lysosomal Storage Diseases

Each of the over seventy known lysosomal storage diseases (LSDs) is characterized by a similar pathogenesis, namely, a compromised lysosomal hydrolase. Generally, the activity of a single lysosomal hydrolytic enzyme is reduced or lacking altogether, usually due to inheritance of an autosomal recessive mutation. As a consequence, the substrate of the compromised enzyme accumulates undigested in lysosomes, producing severe disruption of cellular architecture and various disease manifestations.

### Mucopolysaccharidoses

Mucopolysaccharidoses (MPSs) are a group of rare genetic disorders classified as lysosomal storage disorders (LSDs). MPSs are characterized by deficiency of lysosomal enzymes responsible for the normal degradation of glycosaminoglycans (GAGs) or mucopolysaccharides. The enzyme deficiency leads to progressive lysosomal accumulation of GAGs and their excretion in the urine, followed by development of various somatic and neurologic symptoms. (Noh, H. et al., J. Clin. Pharm. Ther. 39:215-224 (2014). GAGs contain long unbranched polysaccharides characterized by a repeating disaccharide unit and are found in the body linked to core proteins to form proteoglycans. Proteoglycans are located primarily in the extracellular matrix and on the surface of cells where they lubricate joints and contribute to structural integrity (see generally Neufeld et al., 1995, The Mucopolysaccharidoses, In: The Metabolic and Molecular Bases of Inherited Diseases, Scriver et al., eds., McGraw-Hill, New York, 7^{th}ed., pages 2465-2494). Dermatan sulfate and heparan sulfate are the two most common GAGs associated with MPSs. Recently, heparan sulfate proteoglycans have been implicated in Alzheimer’s disease and other amyloid-related disorders. (van Horssen, J. et al., Lancet Neurology 2:482-492 (2003); Spillantini, M. G. et al., Acta Neuropathol. 97:585-594 (1999)).

The mucopolysaccharidoses are categorized into seven types (I, II, III, IV, VI, VII and IX) based on the enzyme affected. These are variable in their prevalence, clinical manifestations and degree of severity. Substantial somatic involvement affecting the heart, lungs, bones, joints and gastrointestinal system is seen in most MPS types, along with CNS dysfunction in MPS I, II, III and VII. Noh, H. et al., J. Clin. Pharm. Ther. 39:215-224 (2014); Morishita, K. et al., Rheumatology 50:v19-v25 (2011).

MPS I (also known as MPS1) is caused by a lack of α-L-iduronidase (IDUA) required for the breakdown of GAGs, primarily heparan sulfate and dermatan sulfate. (Parini, R. et al., Orphanet J. Rare Dis. 12:112 (2017)). Clinical manifestations of MPS I include course facies, dysostosis multiplex, hepatosplenomegaly, cardiac disease, and respiratory dysfunction. MPS is further divided into three clinical subtypes: Hurler syndrome (MPS IH, severe), Hurler-Scheie syndrome (MPS IH/S, intermediate) and Scheie syndrome (MPS IS, attenuated; formerly known as MPS V). (Oussoren, E. et al., Mol. Gen. Metab. 109:377-381 (2013)). In each phenotype, considerable heterogeneity and overlap can be found with respect to the symptoms and their severity. Early progressive neurological decline is the hallmark of Hurler syndrome. Premature death is common, if untreated, secondary to cardiac and respiratory failure. Noh, H. et al., J. Clin. Pharm. Ther. 39:215-224 (2014). Early recognition of the phenotype of MPS I patients is essential to timely initiate the most appropriate therapeutic strategy. (Oussoren, E. et al., Mol. Gen. Metab. 109:377-381 (2013)). The clinical diagnosis of MPS I is confirmed on the basis of elevated levels of dermatan sulfate and heparan sulfate in urine, and of deficient IDUA enzyme activity in leukocytes or fibroblasts. (Oussoren, E. et al., Mol. Gen. Metab. 109:377-381 (2013)).

### Therapies for Lysosomal Storage Diseases

Several approaches are being used or pursued for the treatment of LSDs, including haematopoietic stem cell transplantation, gene therapy, and enzyme replacement therapy. Additionally, researchers have identified a number of small molecules for use alone in the management of LSDs. Other, disease-specific approaches are also under consideration.

### Hsct

Haematopoietic stem cell transplantation (HSCT) is possible for MPSs. Although there are major hurdles to overcome, such as finding a compatible donor and reducing the rates of morbidity and mortality associated with the procedure, HSCT can provide a lifelong source of enzymes to rapidly reduce GAG storage and alter the course of the disease in severe MPS I (Hurler syndrome) and MPS VI. The restoration of enzymatic function and subsequent amelioration of disease complications such as improved joint mobility, vision, hearing and cardiopulmonary function can occur through the cross-correction of enzyme deficiency by the engrafted donor cells. Notably, HSCT has been shown to preserve cognition and increase survival in patients with Hurler syndrome if performed before age 2 and before the onset of serious mental involvement. Noh, H. et al., J. Clin. Pharm. Ther. 39:215-224 (2014). Clinical experience with HSCT is very limited for other MPSs.

### Gene Therapy

Replacement of the defective enzyme in a patient with Fabry Disease is considered feasible using a recombinant retrovirus carrying the cDNA encoding α-Gal A to transfect skin fibroblasts obtained from Fabry patients (Medin J A et al., 1996, Correction in Trans for Fabry Disease: Expression, Secretion, and Uptake of α-Galactosidase A in Patient-Derived Cells Driven by a High-Titer Recombinant Retroviral Vector, Proc. Natl. Acad. Sci. USA 93, 7917-7922).

In vitro studies have also suggested that gene therapy may be feasible in Pompe disease. Vectors are being developed from both recombinant retrovirus and recombinant adenovirus (Zaretsky J Z et al., 1997, Retroviral Transfer of Acid α-Glucosidase cDNA to Enzyme-Deficient Myoblasts Results in Phenotypic Spread of the Genotypic Correction by Both Secretion and Fusion, Human Gene Therapy 8, 1555-1563; Pauly D F et al., 1998, Complete Correction of Acid α-Glucosidase Deficiency in Pompe Disease Fibroblasts in Vitro, and Lysosomally Targeted Expression in Neonatal Rat Cardiac and Skeletal Muscle, Gene Therapy 5, 473-480).

Additionally, transfer and expression of the normal α-L-iduronidase gene into autologous bone marrow by retroviral gene transfer has also been demonstrated in non-clinical studies of Hurler Syndrome (Fairbairn et al., 1996, Long-Term in vitro Correction of α-L-Iduronidase Deficiency (Hurler Syndrome) in Human Bone Marrow, Proc. Natl. Acad. Sci. U.S.A. 93, 2025-2030).

### Enzyme Replacement Therapy

Currently, the most effective treatment for MPSs is enzyme replacement thereapy (ERT). Since the advent of ERT, the clinical status and quality of life for many patients have markedly improved. However, major challenges with ERT remain, including high cost, need for frequent administrations, and failure to prevent the adverse skeletal and CNS effects of the disease. Noh, H. et al., J. Clin. Pharm. Ther. 39:215-224 (2014). Approved ERT are available only for MPS I, II, and VI. It is generally accepted that ERT should be offered to all patients with MPS I at the time of diagnosis and continued until severe neuropathy or any other condition develops that may inadvertently affect prognosis.

Laronidase is a recombinant human α-L-iduronidase (rhIDUA) indicated for the treatment of MPS I. In a Phase 3 study, 45 patients (Hurler, 1, Hurler-Scheie, 37, Scheie, 7) with a mean age of 16 years (range 6 to 43 years) were randomly assigned to receive laronidase 0.58 mg/kg or placebo weekly for 26 weeks. By week 4, the mean urinary GAG levels approached the normal range in the active treatment group. After 26 weeks, compared with placebo, the active treatment group showed a significant improvement in the forced vital capacity (FVC) by 5-6% Noh, H. et al., J. Clin. Pharm. Ther. 39:215-224 (2014). Recombinant IDUA (laronidase, Aldurazyme®) was developed as intravenous ERT for the treatment of MPS I. (See U.S. Pat. No. 9,044,473) However, laronidase has no impact on cognitive decline in MPS I, because the enzyme does not cross the blood-brain barrier. (Gugliani, R. et all, Orphanet J. Rare. Dis. 13:110 (2018)). To enable BBB transport, IDUA was reengineered as an IgG-IDUA fusion protein where the IgG domain targets the BBB human insulin receptor to enable transport of the enzyme into the brain. Preliminary results of clinical trials of the fusion protein in MPS I showed the transport into both the CNS and into peripheral organs due to its dual targeting mechanism. (Gugliani, R. et all, Orphanet J. Rare. Dis. 13:110 (2018)).

### Small Molecule Therapy

Recently, a variety of studies have been conducted using several small molecules for storage disease therapy. One class of molecules inhibits upstream generation of lysosomal hydrolase substrate to relieve the input burden to the defective enzyme. This approach has been dubbed “substrate deprivation” therapy. One example of this class of molecules is N-butyldeoxynojirimycin (NB-DNJ), an inhibitor of the ceramide-specific glucosyltransferase (i.e. glucosylceramide synthase) which catalyzes the first step in the synthesis of glycosphingolipids (GSLs). NB-DNJ has been tested in mouse models of Sandhoff disease (Jeyakumar et al., (1999), Proc. Natl. Acad. Sci. USA, (1996), 6388-6393), Tay-Sachs disease (Platt et al., (1997), Science 276, 428-431), as well as in humans with Gaucher’s disease (Cox et al., (2000), Lancet 355, 1481-1485), resulting in an amelioration of symptoms in each of these diseases. A variety of deoxynojirimycin (DNJ) derivatives have also been synthesized as research tools intended for the selective inhibition of the non-lysosomal glucosylceramidase at concentrations in which glucosylceramide synthase and other enzymes are not affected (Overkleeft et al., (1998), J. Biol. Chem. 273, 26522-26527). Certain uses of glucosylceramide synthase inhibitors of the DNJ type either alone (WO 00/62780) or in combination with a glycolipid degrading enzyme (WO 00/62779) have been described.

Another example of the substrate deprivation class of molecules are the amino ceramide-like small molecules which have been developed for glucosylceramide synthase inhibition. Glucosylceramide synthase catalyzes the first glycosylation step in the synthesis of glucosylceramide-based glycosphingolipids. Glucosylceramide itself is the precursor of hundreds of different glycosphingolipids. Amino ceramide-like compounds have been developed for use in Fabry disease (Abe et al., 2000, J. Clin. Invest. 105, 1563-1571; Abe et al., 2000, Kidney Int’l 57, 446-454) and Gaucher’s disease (Shayman et al., 2000, Meth. Enzymol. 31, 373-387; U.S. Pat. Nos. 5,916,911; 5,945,442; 5,952,370; 6,030,995; 6,040,332 and 6,051,598). A variety of amino ceramide-like analogues have been synthesized as improved inhibitors of glucosylceramide synthase (see e.g. Lee et al., 1999, J. Biol. Chem. 274, 14662-14669).

Aminoglycosides such as gentamicin and G418 are small molecules which promote read-through of premature stop-codon mutations. These so-called stop-mutation suppressors have been used in Hurler cells to restore a low level of α-L-iduronidase activity (Keeling et al., 2001, Hum. Molec. Genet. 10, 291-299). They have also been developed for use in treating cystic fibrosis individuals having stop mutations (U.S. Pat. No. 5,840,702).

It has been surprisingly found that compounds of formula (I) are capable of stabilizing the IDUA enzyme against denaturation. Increasing the effective concentration of the IDUA enzyme, laronidase, can overall increase the efficacy of the current treatment of MPS1.

### BRIEF SUMMARY OF THE INVENTION

The present disclosure is related to the discovery that compounds represented by formulae (I) and (I') are capable of binding to wild-type and/or mutated IDUA and are thus useful in the treatment or prevention of conditions associated with the alteration of the activity of α-L-iduronidase in a subject, e.g., MPS1, heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, and a developmental disorder.

In one aspect, the present disclosure provides a method of treating or preventing a condition associated with the alteration of the activity of α-L-iduronidase in a patient in need thereof, comprising administering an effective amount of a compound of formula (I) or formula (I'), or a pharmaceutically acceptable salt or solvate thereof, as described herein, optionally in combination with an effective amount of α-L-iduronidase or an analog or variant thereof, e.g., laronidase. Compounds represented by formulae (I) and (I'), and the pharmaceutically acceptable salts and solvates thereof, are herein collectively referred to as “Compounds of the Disclosure” (each individually referred to as a “Compound of the Disclosure”).

In another aspect, the present disclosure provides a method of treating or preventing MPS1 in a patient in need thereof by administering an effective amount of a Compound of the Disclosure optionally in combination with an effective amount of α-L-iduronidase or an analog or variant thereof, e.g., laronidase.

In another aspect, the present disclosure provides a method of treating or preventing a heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder in a patient in need thereof by administering an effective amount of a Compound of the Disclosure optionally in combination with an effective amount of α-L-iduronidase or an analog or variant thereof, e.g., laronidase.

In one embodiment, the methods described herein do not comprise administering an effective amount of α-L-iduronidase or an analog or variant thereof, e.g., laronidase.

In another embodiment, the methods described herein comprise administering an effective amount of a Compound of the Disclosure in combination with an effective amount of laronidase. In some embodiments, the effective amount of a Compound of the Disclosure and an effective amount of α-L-iduronidase or an analog or variant thereof, e.g., laronidase, are administered simultaneously to the patient. In some embodiments, the effective amount of a Compound of the Disclosure and an effective amount of α-L-iduronidase or an analog or variant thereof, e.g., laronidase, are administered to the patient sequentially. In some embodiments, the effective amount of a Compound of the Disclosure and the effective amount of α-L-iduronidase or an analog or variant thereof, e.g., laronidase, are administered to the patient in separate pharmaceutical compositions. In some embodiments, the effective amount of a Compound of the Disclosure and the effective amount of laronidase are administered to the patient in a single pharmaceutical composition.

In another aspect, the methods described herein further comprise administering to the patient at least one other therapeutic agent. In another aspect, the therapeutic agent is an effective amount of a small molecule chaperone. In another aspect, the small molecule chaperone binds competitively to an enzyme. In another aspect, the small molecule chaperone is selected from the group consisting of iminoalditols, iminosugars, aminosugars, thiophenylglycosides, glycosidase, sulfatase, glycosyl transferase, phosphatase, and peptidase inhibitors. In another aspect, the small molecule chaperone is selected from the group consisting of isofagomine, N-nonyl-1-deoxynojirimycin (NN-DNJ), ambroxol, and miglustat. In another aspect, the small molecule chaperone is selected from the group consisting of isofagomine, N-nonyl-1-deoxynojirimycin (NN-DNJ), and ambroxol. In another aspect, the small molecule chaperone is miglustat.

In another aspect, the therapeutic agent is an effective amount of substrate reduction agent for substrate reduction therapy. In another aspect, the substrate reduction agent is miglustat.

A number of compounds useful in the treatment or prevention of the present disclosure have not been heretofor reported. Thus, one aspect of the present disclosure is directed to the novel compounds of formulae (I) and (I′), and the salts and solvates thereof.

In another aspect, the present disclosure provides compounds of formula (I′), and the salts and solvates thereof.

In another aspect, the present disclosure provides a Compound of the Disclosure, as described herein, for use in the treatment or prevention of a condition associated with the alteration of the activity of α-L-iduronidase in a patient, optionally in a combination with α-L-iduronidase or an analog or variant thereof, e.g., laronidase.

In another aspect, present disclosure provides a Compound of the Disclosure, as described herein, for use in the treatment or prevention MPS1, heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, and a developmental disorder in a patient in need thereof, optionally in a combination with an effective amount of α-L-iduronidase or an analog or variant thereof, e.g., laronidase.

In another aspect, the present disclosure is also directed to the use of a Compound of the Disclosure, as described herein, for the treatment or prevention of a condition associated with the alteration of the activity of α-L-iduronidase in a patient in need thereof, such as MPS1, described herein, optionally in a combination with α-L-iduronidase or an analog or variant thereof, e.g., laronidase.

In another aspect, the present disclosure provides a pharmaceutical composition comprising a Compound of the Disclosure, as described herein, and at least one pharmaceutically acceptable excipient.

In another aspect, the present disclosure provides a Compound of the Disclosure, as described herein, for use as a medicament, optinally in combination with α-L-iduronidase or an analog or variant thereof, e.g., laronidase.

In another aspect, the present disclosure provides use of a Compound of the Disclosure, as described herein, in the preparation of a medicament for the prevention or treatment of a condition associated with the alteration of the activity of α-L-iduronidase in a patient in need thereof, such as MPS1 described herein, said medicament optionally comprising an effective amount of α-L-iduronidase or an analog or variant thereof, e.g., laronidase.

In another aspect, the present disclosure provides a pharmaceutical composition comprising a Compound of the Disclosure, as described herein, and at least one pharmaceutically acceptable excipient, for use in the treatment or prevention of a condition associated with the alteration of the activity of α-L-iduronidase in a patient in need thereof, such as MPS1 described herein. In some aspects, the pharmaceutical composition comprises an effective amount of α-L-iduronidase or an analog or variant thereof, e.g., laronidase.

In another aspect, the present disclosure is directed to a pharmaceutical composition comprising an effective amount of a Compound of the Disclosure and an effective amount of α-L-iduronidase or an analog or variant thereof, e.g., larodinase, and a pharmaceutically acceptable excipient. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to a combination, comprising a Compound of the Disclosure and α-L-iduronidase or an analog or variant thereof, e.g., larodinase. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

Other aspects and advantages of the disclosure will be readily apparent from the following detailed description of the disclosure. The embodiments and advantages of the disclosure will be realized and attained by means of the elements and combinations particularly pointed out in the appended claims.

It is to be understood that both the foregoing summary and the following detailed description are exemplary and explanatory only and are not restrictive of the disclosure as claimed.

### BRIEF DESCRIPTION OF THE DRAWINGS/FIGURES

FIG. 1 is a line graph showing the thermal shift dose response curve from a differential scanning fluorimetry (DSF) assay for Compound A (i.e., the compound of Example 8).

FIG. 2 is a line graph showing the results of Compound A in an IDUA denaturation prevention assay having L-iduronic acid as the reference compound. The results show that Compound A (at 30 µM) prevents IDUA denaturation.

FIG. 3 is a line graph showing the results of Compound A in an IDUA inhibition assay. The results show that Compound A does not inhibit IDUA.

FIGS. 4A-4E show results of cell-based assays in fibroblasts on Compound A. FIG. 4A is graph showing that co-administration of Compound A (at 50 µM) promotes IDUA cell uptake (96h). FIG. 4B is a line graph showing that the effect of Compound A is dose-dependent EC₅₀=16 µM (96h). FIG. 4C is a graph that Compound A is active across a panel of patient-derived fibroblasts (96h). FIG. 4D is a graph showing that the effect of Compound A in fibroblasts increases at longer incubation times. FIG. 4E is a figure showing that Compound A increases the amount of α-L-iduronidase in fibroblasts (96h).

FIG. 5 is a line graph showing that administration of Compound A improves the pharmacokinetic profile of laronidase in mice.

FIGS. 6A and 6B are graphs showing that co-administarion of Compound A with laronidase increases laronidase tissue exposure in mice bone marrow and bone joints cartilage, respectively.

FIG. 7 is a line graph showing the results of compounds of Examples 8, 40, 54, 39, and 72 in an IDUA denaturation prevention assay. The results show that, in addition to the compound of Example 8 (i.e., Compound A), other exemplary Compounds of the Disclosure prevent IDUA denaturation.

### DETAILED DESCRIPTION OF THE INVENTION

Current treatment of MPS1 is based on enzyme replacement therapy (ERT), where the exogenous IDUA enzyme (laronidase) is administered intravenously once per week. As in other ERTs, the short half-life of the enzyme and the desensitisation caused by its immunogenicity are limiting factors to its efficacy. This results in low bioavailability, particularly in tissues with limited or absent blood circulation such as bone and cartilage, where treatment shows scarce efficacy. The inventors have discovered that increasing the effective concentration of the IDUA enzyme, laronidase, can overall increase the efficacy of the current treatment of MPS1. Combination therapy with pharmacological chaperones and ERT can improve tissue uptake and reduce ERT’s immunogenicity by stabilizing the enzyme in its properly folded and active form. Certain Compounds of the Disclosure have been found by the inventors to protect laronidase from pH-dependent denaturalisation and increase the uptake of the enzyme by MPS1 patient-derived fibroblasts. When co-administered with laronidase intravenously to wild-type mice, an exemplary Compound of the Disclosure has been found to increase its plasma activity levels, with a peak at 0.5 h and prolong its enzymatic activity across multiple tissues, including bone and cartilage. These results open new treatment perspectives in diseases with hard-to-treat organs such as bone and cartilaginous tissues for which an unmet medical need exists.

One aspect of the disclosure is based on the use of Compounds of the Disclosure for binding to mutated IDUA. In view of this property, Compounds of the Disclosure are expected to be useful for treating or preventing conditions associated with alteration of the activity of IDUA in a patient, such as MPS1.

Compounds of the Disclosure useful in this aspect of the disclosure are compounds of formula (I):

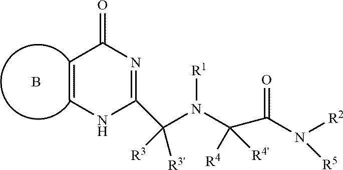

and the pharmaceutically acceptable salts and solvates thereof, wherein
B is a fused benzene ring or a fused 5- or 6-membered heteroaromatic ring, wherein said benzene ring and said 5- or 6-membered heteroaromatic ring is optionally substituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C1-9 heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In another embodiment of this aspect of the disclosure, Compounds of the Disclosure are compounds of formula (I), and the pharmaceutically acceptable salts and solvates thereof, wherein B is a fused, optionally substituted benzene ring. In another embodiment, B is a fused, unsubstituted benzene ring. In another embodiment, B is a fused benzene ring substituted with one or more substituents selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is as defined above.

In another embodiment of this aspect of the disclosure, Compounds of the Disclosure are compounds of formula (I), and the pharmaceutically acceptable salts and solvates thereof, wherein B is a fused, optionally substituted 5-membered heteroaromatic ring. In another embodiment, B is a fused, unsubstituted 5-membered heteroaromatic ring. In another embodiment, B is a fused 5-membered heteroaromatic ring substituted with 1 or 2 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is as defined above. Suitable 5-membered heteroaromatic rings include include thiophene, pyrazole, imidazole, and pyrrole, which can be unsubstituted or substituted as defined above.

In another embodiment of this aspect of the disclosure, Compounds of the Disclosure are compounds of formula (I), and the pharmaceutically acceptable salts and solvates thereof, wherein B is a fused, optionally substituted 6-membered heteroaromatic ring. In another embodiment, B is a fused, unsubstituted 6-membered heteroaromatic ring. In another embodiment, B is a fused 6-membered heteroaromatic ring substituted with 1, 2 or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is as defined above. Suitable 6-membered heteroaromatic rings include include pyridine, pyrazine, pyrimidine, and pyridazine, which can be unsubstituted or substituted as defined above.

In another embodiment of this aspect of the disclosure, Compounds of the Disclosure are compounds of formula (I), and the pharmaceutically acceptable salts and solvates thereof, wherein B is selected from the group consisting of

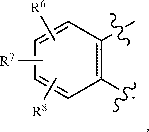

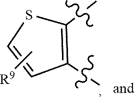

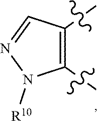

wherein R⁶, R⁷, R⁸, R⁹, and R¹⁰ are each independently selected from the group consisting of hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; wherein Ra is as described herein.

In another embodiment of this aspect of the disclosure, Compounds of the Disclosure are compounds of formula (I), and the pharmaceutically acceptable salts and solvates thereof, wherein B is selected from the group consisting of

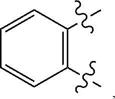

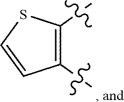

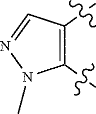

In another embodiment of this aspect of the disclosure, Compounds of the Disclosure are compounds of formula (I), and the pharmaceutically acceptable salts and solvates thereof, wherein B is

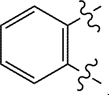

In another embodiment of this aspect of the disclosure, Compounds of the Disclosure are compounds of formula (I), and the pharmaceutically acceptable salts and solvates thereof, wherein B is

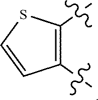

In another embodiment of this aspect of the disclosure, Compounds of the Disclosure are compounds of formula (I), and the pharmaceutically acceptable salts and solvates thereof, wherein B is

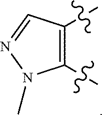

In another embodiment of this aspect of the disclosure, Compounds of the Disclosure are compounds of formula (I), and the pharmaceutically acceptable salts and solvates thereof, wherein B is

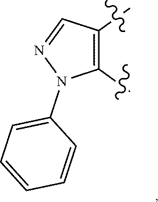

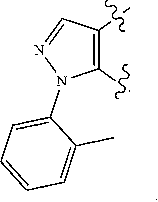

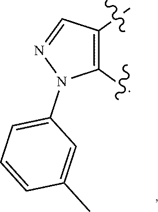

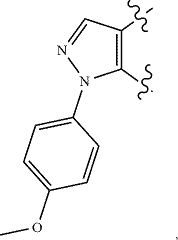

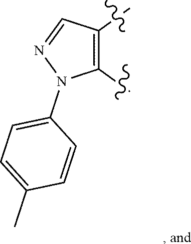

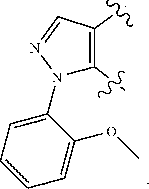

In another embodiment of this aspect of the disclosure, Compounds of the Disclosure are compounds of formula (I), and the pharmaceutically acceptable salts and solvates thereof, wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R³, R^{3'}, R⁴, and R^{4'} are each independently selected from the group consisting of hydrogen, halogen, unsubstituted C₁₋₄ alkyl, and substituted C₁₋₄ alkyl.

In some embodiments, Compounds of the Disclosure are compounds of formula (I), and the pharmaceutically acceptable salts and solvates thereof, wherein R¹ is selected from the group consisting of -C₁₋₄ alkyl, -C₃-₆ cycloalkyl, -C₁₋₄ alkyl-C₃-₆ cycloalkyl, -phenyl, -C₁₋₄ alkyl-phenyl, (5- or 6-membered)-C₁₋₅ heteroaryl, -C₁₋₄ alkyl-(5- or 6-membered)-C₁₋₅ heteroaryl, (5- or 6-membered)-C₂₋₅ heterocyclyl, and -C₁₋₄ alkyl-(5- or 6-membered)-C₂₋₅ heterocyclyl, wherein said alkyl, cycloalkyl, alkylcycloalkyl, phenyl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted phenyl, optionally substituted (5- or 6-membered)-C₁₋₅ heteroaryl, and (5- or 6-membered)-C₂₋₅ heterocyclyl; and wherein said cycloalkyl, alkylcycloalkyl, phenyl, alkylaryl, heteroaryl, alkylheteroaryl, heterocyclyl and alkylheterocyclyl is optionally fused to a further (second) ring.

In some embodiments, Compounds of the Disclosure are compounds of formula (I), and the pharmaceutically acceptable salts and solvates thereof, wherein R¹ is selected from the group consisting of -C₁₋₄ alkyl, -C₃₋₆ cycloalkyl, -C₁₋₄ alkyl-C₃₋₆ cycloalkyl, and -phenyl, wherein said alkyl, cycloalkyl, alkylcycloalkyl, and phenyl groups are optionally substituted with 1, 2 or 3 substituents each independently selected from the group consisting of halogen, hydroxy, -CN, -OH, -O(C₁₋₄ alkyl), -SHalkyl)₂, -S(C₁₋₄ alkyl), -N(C₁₋₄ , and -C₁₋₄ alkyl optionally substituted with 1, 2, or 3 halogen atoms.

In some embodiments, Compounds of the Disclosure are compounds of formula (I), and the pharmaceutically acceptable salts and solvates thereof, wherein R¹ is selected from the group consisting of -C₁₋₄ alkyl and -C₃₋₆ cycloalkyl, wherein said alkyl and cycloalkyl are optionally substituted with 1, 2 or 3 substituents each independently selected from the group consisting of halogen, hydroxy, -CN, -OH, -O(C₁₋₄ alkyl), -SH, -S(C₁₋₄ alkyl), -N(C₁-₄ alkyl)₂, and -C₁₋₄ alkyl optionally substituted with 1, 2, or 3 halogen atoms.

In another embodiment of this aspect of the disclosure, Compounds of the Disclosure are compounds of formula (I), and the pharmaceutically acceptable salts and solvates thereof, wherein R³ and R⁴ are hydrogen, and R^{3'} and R^{4'} are each independently selected from the group consisting of hydrogen, halogen, unsubstituted C₁₋₄ alkyl, and substituted C₁₋₄ alkyl.

In another embodiment of this aspect of the disclosure, Compounds of the Disclosure are compounds of formula (I), and the pharmaceutically acceptable salts and solvates thereof, wherein R³, R^{3'}, R⁴, and R^{4'} are each hydrogen.

In another embodiment of this aspect of the disclosure, Compounds of the Disclosure are compounds of formula (I), and the pharmaceutically acceptable salts and solvates thereof, wherein R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{3'}, R⁴, and R^{4'} are as defined above.

In another embodiment of this aspect of the disclosure, Compounds of the Disclosure are compounds of formula (I), and the pharmaceutically acceptable salts and solvates thereof, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, and R^{4'} are as defined above.

In another embodiment of this aspect of the disclosure, Compounds of the Disclosure are compounds of formula (I), and the pharmaceutically acceptable salts and solvates thereof, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, and R^{4'} are each hydrogen.

In another embodiment of this aspect of the disclosure, Compounds of the Disclosure are compounds of formula (I), and the pharmaceutically acceptable salts and solvates thereof, wherein R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, R⁴, R^{4'}, and R⁵ are as defined above.

In another embodiment of this aspect of the disclosure, Compounds of the Disclosure are compounds of formula (I), and the pharmaceutically acceptable salts and solvates thereof, wherein R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, R⁴, and R^{4'} are each hydrogen and R⁵ is as defined above.

In another embodiment of this aspect of the disclosure, Compounds of the Disclosure are compounds of formula (I), and the pharmaceutically acceptable salts and solvates thereof, wherein R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.

In some embodiments, Compounds of the Disclosure are compounds of formula (I), and the pharmaceutically acceptable salts and solvates thereof, wherein R² is phenyl optionally substituted with 1, 2, or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.

In another embodiment of this aspect of the disclosure, Compounds of the Disclosure are compounds of formula (I), and the pharmaceutically acceptable salts and solvates thereof, wherein R² is selected from the group consisting of

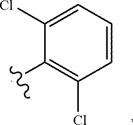

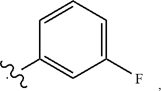

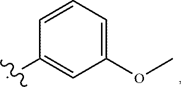

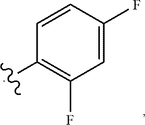

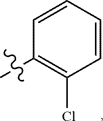

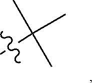

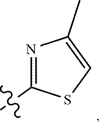

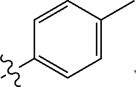

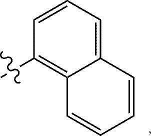

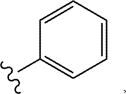

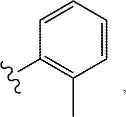

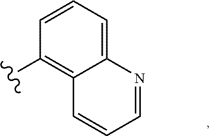

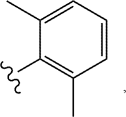

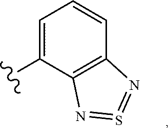

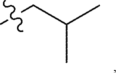

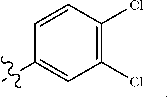

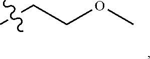

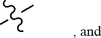

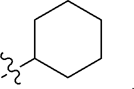

In another embodiment of this aspect of the disclosure, Compounds of the Disclosure are compounds of formula (I), and the pharmaceutically acceptable salts and solvates thereof, wherein R⁵ is unsubstituted C₁₋₆ alkyl. In another embodiment, R⁵ is unsubstituted C₂-₆ alkyl.

In another embodiment of this aspect of the disclosure, Compounds of the Disclosure are compounds of formula (I), and the pharmaceutically acceptable salts and solvates thereof, wherein R² and R⁵ together with the nitrogen atom to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and wherein said heterocyclic ring is optionally fused to a phenyl ring.

In another embodiment of this aspect of the disclosure, Compounds of the Disclosure are compounds of formula (I), and the pharmaceutically acceptable salts and solvates thereof, wherein R² and R⁵ together with the nitrogen atom to which they are attached form a group

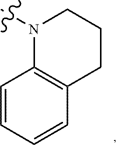

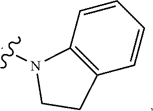

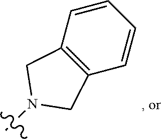

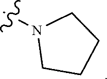

In another embodiment of this aspect of the disclosure, Compounds of the Disclosure are compounds of formula (I), and the pharmaceutically acceptable salts and solvates thereof, wherein Ra is hydrogen or -C₁₋₄ alkyl optionally substituted by 1, 2 or 3 fluorine atoms.

In another embodiment of this aspect of the disclosure, Compounds of the Disclosure are compounds of formula (I), and the pharmaceutically acceptable salts and solvates thereof, wherein Ra -C₃₋₁₀ cycloalkyl or -(5- to 10-membered)-C₂₋₉ heterocyclyl, wherein cycloalkyl or heterocyclyl group is optionally substituted by 1, 2 or 3 fluorine atoms.

In another embodiment, Compounds of the Disclosure are compounds of formula (I′):

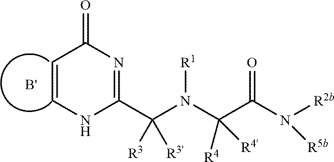

and the pharmaceutically acceptable salts and solvates thereof, wherein
B′ is a fused ring selected from the group consisting of

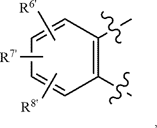

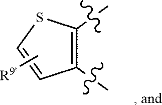

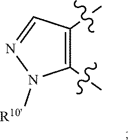

R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring; or
R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆-₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring;
R⁶, R⁷, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, R^{7'}, and R^{8'} is other than hydrogen;
R^{9'} and R^{10'} are each independently selected from the group consisting of halogen, hydroxy, CN, -ORa, -SRa, -N(Ra)₂, (=O),-C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In some embodiments, when B” is B1' or B3' and R^{2b} and R^{5b} together with the nitrogen atom to which they are attached form a 5- or 6-membered heterocyclic ring, wherein 1, 2, or 3 of the carbon atoms of said heterocyclic ring are optionally replaced by a heteroatom selected from the group consisting of N, S, and O, then the heterocyclic ring is:
1) substituted by at least one substituent selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋₁₀ aryl, -(5- to 10-membered)-C₁₋₉ heteroaryl, -(5- to 10-membered)-C₂₋₉ heterocyclyl, and -C₃₋₁₀ cycloalkyl, wherein Ra is as defined above, or
2) fused to a phenyl ring,
   provided that the compound of formula (I′) is not

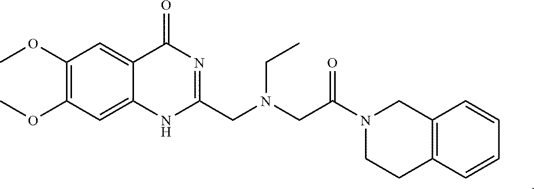

In some embodiments, when R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, then R^{5b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring, provided that the compound of formula (I′) is not

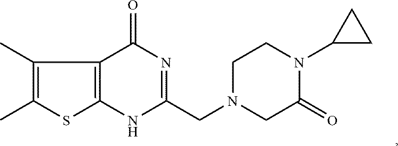

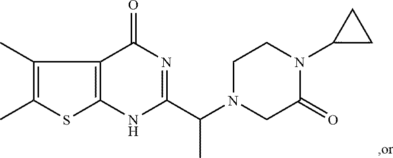

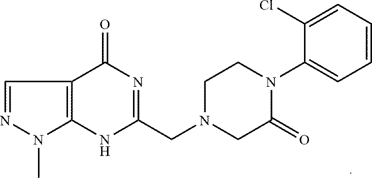

In some embodiments of this aspect of the disclosure, Compounds of the Disclosure are compounds of formula (I′), and the pharmaceutically acceptable salts and solvates thereof, as defined above, wherein B′ and R¹ are as defined above,
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R⁶, R⁷, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

In another embodiment of this aspect of the disclosure, Compounds of the Disclosure are compounds of formula (I′), and the pharmaceutically acceptable salts and solvates thereof, wherein B′ is

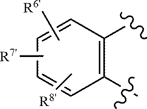

In another embodiment of this aspect of the disclosure, Compounds of the Disclosure are compounds of formula (I′), and the pharmaceutically acceptable salts and solvates thereof, wherein B′ is

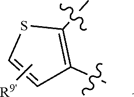

In another embodiment of this aspect of the disclosure, Compounds of the Disclosure are compounds of formula (I′), and the pharmaceutically acceptable salts and solvates thereof, wherein B′ is

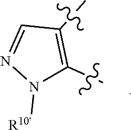

In another embodiment of this aspect of the disclosure, Compounds of the Disclosure are compounds of formula (I′), and the pharmaceutically acceptable salts and solvates thereof, wherein B′ is B3' selected from the group consisting of

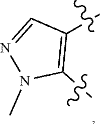

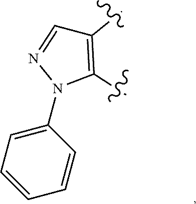

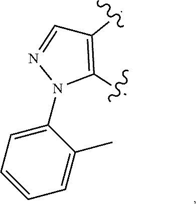

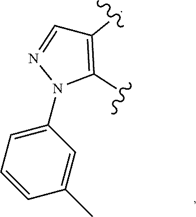

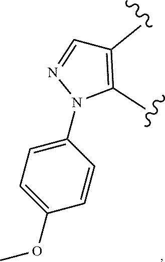

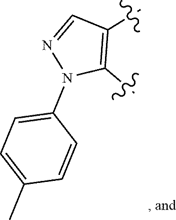

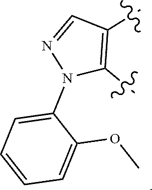

In another embodiment of this aspect of the disclosure, Compounds of the Disclosure are compounds of formula (I′), and the pharmaceutically acceptable salts and solvates thereof, wherein B′ is B3' selected from the group consisting of

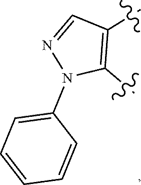

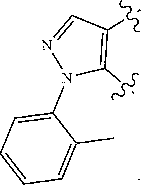

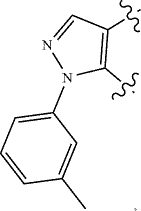

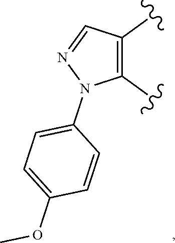

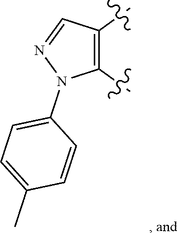

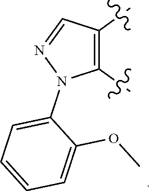

In another embodiment of this aspect of the disclosure, Compounds of the Disclosure are compounds of formula (I′), and the pharmaceutically acceptable salts and solvates thereof, wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and

R³, R^{3'}, R⁴, and R^{4'} are each independently selected from the group consisting of hydrogen, halogen, unsubstituted C₁₋₄ alkyl, and substituted C₁₋₄ alkyl.

In some embodiments, Compounds of the Disclosure are compounds of formula (I′), and the pharmaceutically acceptable salts and solvates thereof, wherein R¹ is selected from the group consisting of -C₁₋₄ alkyl, -C₃-₆ cycloalkyl, -C₁₋₄ alkyl-C₃-₆ cycloalkyl, -phenyl, -C₁₋₄ alkyl-phenyl, (5- or 6-membered)-C₁₋₅ heteroaryl, -C₁₋₄ alkyl-(5- or 6-membered)-C₁₋₅ heteroaryl, (5- or 6-membered)-C₂₋₅ heterocyclyl, and -C₁₋₄ alkyl-(5- or 6-membered)-C₂₋₅ heterocyclyl, wherein said alkyl, cycloalkyl, alkylcycloalkyl, phenyl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted phenyl, optionally substituted (5- or 6-membered)-C₁₋₅ heteroaryl, and (5- or 6-membered)-C₂₋₅ heterocyclyl; and wherein said cycloalkyl, alkylcycloalkyl, phenyl, alkylaryl, heteroaryl, alkylheteroaryl, heterocyclyl and alkylheterocyclyl is optionally fused to a further (second) ring.

In some embodiments, Compounds of the Disclosure are compounds of formula (I′), and the pharmaceutically acceptable salts and solvates thereof, wherein R¹ is selected from the group consisting of -C₁₋₄ alkyl, -C₃-₆ cycloalkyl, -C₁₋₄ alkyl-C₃-₆ cycloalkyl, and -phenyl, wherein said alkyl, cycloalkyl, alkylcycloalkyl, and phenyl groups are optionally substituted with 1, 2 or 3 substituents each independently selected from the group consisting of halogen, hydroxy, -CN, -OH, -O(C₁₋₄ alkyl), -SH, -S(C₁₋₄ alkyl), -N(C₁₋₄ alkyl)₂, and -C₁₋₄ alkyl optionally substituted with 1, 2, or 3 halogen atoms.

In some embodiments, Compounds of the Disclosure are compounds of formula (I′), and the pharmaceutically acceptable salts and solvates thereof, wherein R¹ is selected from the group consisting of -C₁₋₄ alkyl and -C₃-₆ cycloalkyl, wherein said alkyl and cycloalkyl are optionally substituted with 1, 2 or 3 substituents each independently selected from the group consisting of halogen, hydroxy, -CN, -OH, -O(C₁₋₄ alkyl), -SH, -S(C₁₋₄ alkyl), -N(C₁₋₄ alkyl)₂, and -C₁₋₄ alkyl optionally substituted with 1, 2, or 3 halogen atoms.

In another embodiment of this aspect of the disclosure, Compounds of the Disclosure are compounds of formula (I′), and the pharmaceutically acceptable salts and solvates thereof, wherein R³ and R⁴ are hydrogen, and R^{3'} and R^{4'} are each independently selected from the group consisting of hydrogen, halogen, unsubstituted C₁₋₄ alkyl, and substituted C₁₋₄ alkyl.

In another embodiment of this aspect of the disclosure, Compounds of the Disclosure are compounds of formula (I′), and the pharmaceutically acceptable salts and solvates thereof, wherein R³, R^{3'}, R⁴, and R^{4'} are each hydrogen.

In another embodiment of this aspect of the disclosure, Compounds of the Disclosure are compounds of formula (I′), and the pharmaceutically acceptable salts and solvates thereof, wherein R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O.

In some embodiments, Compounds of the Disclosure are compounds of formula (I′), and the pharmaceutically acceptable salts and solvates thereof, wherein R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{5b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring, provided that the compound of formula (I′) is not

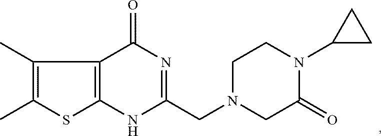

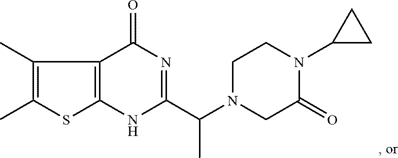

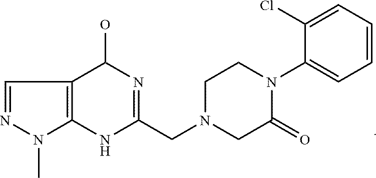

In another embodiment of this aspect of the disclosure, Compounds of the Disclosure are compounds of formula (I′), and the pharmaceutically acceptable salts and solvates thereof, wherein R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{3'}, R⁴, and R^{4'} are as defined above. In some embodiments, R¹ and R³ together with the nitrogen atom to which thet are attached form a 5-membered heterocyclic ring.

In another embodiment of this aspect of the disclosure, Compounds of the Disclosure are compounds of formula (I′), and the pharmaceutically acceptable salts and solvates thereof, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, and R^{4'} are as defined above. In some embodiments, R¹ and R⁴ together with the nitrogen atom to which they are attached form a 5-membered heterocyclic ring.

In another embodiment of this aspect of the disclosure, Compounds of the Disclosure are compounds of formula (I′), and the pharmaceutically acceptable salts and solvates thereof, wherein R¹ and R⁴ together with the nitrogen and carbon atoms to which they are attached form an optionally substituted 5- to 10-membered heterocyclic ring, wherein 1, 2, or 3 of the carbon aroms of said heterocyclic ring are optionally replaced by a heteroatom selected from the group consisting of N, S, and O, and R³, R^{3'}, and R^{4'} are each hydrogen. In some embodiments, R¹ and R⁴ together with the nitrogen atom to which they are attached form a 5-membered heterocyclic ring.

In another embodiment of this aspect of the disclosure, Compounds of the Disclosure are compounds of formula (I′), and the pharmaceutically acceptable salts and solvates thereof, wherein R^{2b} is selected from the group consisting of -C₃₋₅ cycloalkyl, -C₆-₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; and

R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring.

In some embodiments, Compounds of the Disclosure are compounds of formula (I′), and the pharmaceutically acceptable salts and solvates thereof, wherein R^{2b} is phenyl optionally substituted with 1, 2, or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.

In another embodiment of this aspect of the disclosure, Compounds of the Disclosure are compounds of formula (I′), and the pharmaceutically acceptable salts and solvates thereof, wherein R^{2b} is selected from the group consisting of

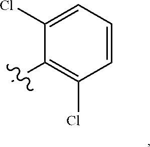

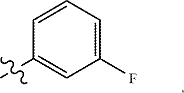

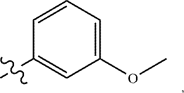

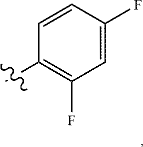

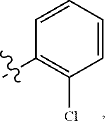

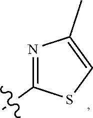

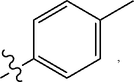

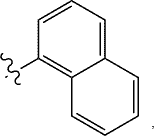

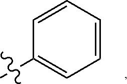

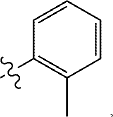

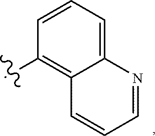

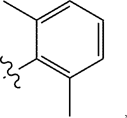

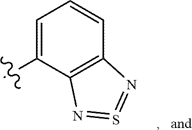

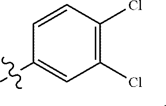

In another embodiment of this aspect of the disclosure, Compounds of the Disclosure are compounds of formula (I′), and the pharmaceutically acceptable salts and solvates thereof, wherein R^{5b} is unsubstituted C₁₋₆ alkyl. In another embodiment, R^{5b} is unsubstituted C₂-₆ alkyl.

In another embodiment of this aspect of the disclosure, Compounds of the Disclosure are compounds of formula (I′), and the pharmaceutically acceptable salts and solvates thereof, wherein R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring; provided that when when B′ is B1′ or B3′ then the heterocyclic ring is:
1) substituted by at least one substituent selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋₁₀ aryl, -(5- to 10-membered)-C₁₋₉ heteroaryl, -(5- to 10-membered)-C₂₋₉ heterocyclyl, and -C₃₋₁₀ cycloalkyl, wherein Ra is as defined above, or
2) fused to a phenyl ring, and
   provided that the compound of formula (I′) is not

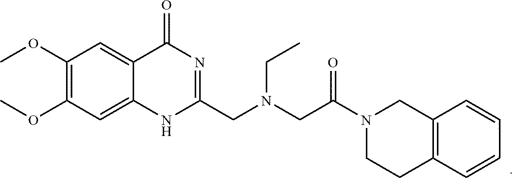

In some embodiments, Compounds of the Disclosure are compounds of formula (I′), and the pharmaceutically acceptable salts and solvates thereof, wherein R^{2b} and R^{5b} together with the nitrogen atom to which they are attached form a group

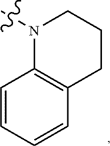

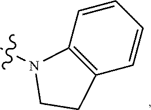

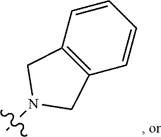

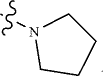

In some embodiments, Compounds of the Disclosure are compounds of formula (I′), and the pharmaceutically acceptable salts and solvates thereof, wherein R^{2b} and R^{5b} together with the nitrogen atom to which they are attached form a group

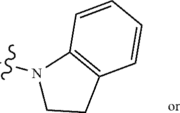

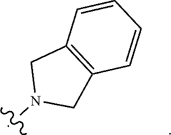

In another embodiment of this aspect of the disclosure, Compounds of the Disclosure are compounds of formula (I′), and the pharmaceutically acceptable salts and solvates thereof, wherein Ra is hydrogen or -C₁₋₄ alkyl optionally substituted by 1, 2 or 3 fluorine atoms.

In another embodiment of this aspect of the disclosure, Compounds of the Disclosure are compounds of formula (I′), and the pharmaceutically acceptable salts and solvates thereof, wherein Ra is -C₃₋₁₀ cycloalkyl or -(5- to 10-membered)-C₂₋₉ heterocyclyl, wherein cycloalkyl or heterocyclyl group is optionally substituted by 1, 2 or 3 fluorine atoms.

In another aspect, Compounds of the Disclosure that can be employed in the methods of the present disclosure include compounds of formula (I) selected from the group consisting of

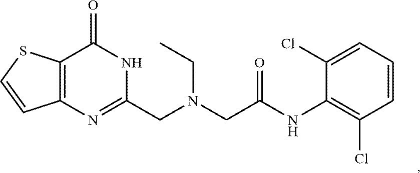

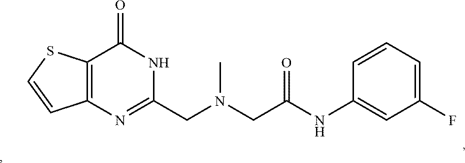

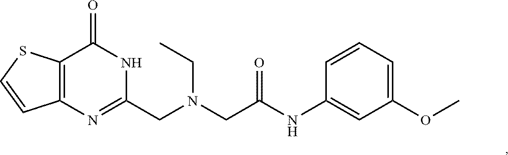

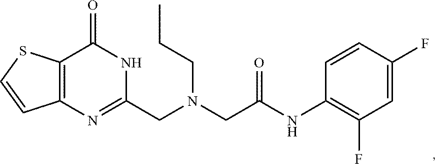

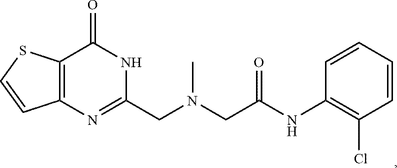

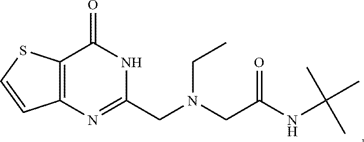

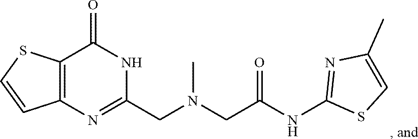

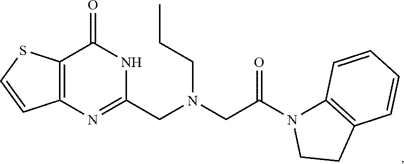

and the pharmaceutically acceptable salts and solvates thereof.

In another aspect, Compounds of the Disclosure that can be employed in the methods of the present disclosure include compounds of formula (I) selected from the group consisting of

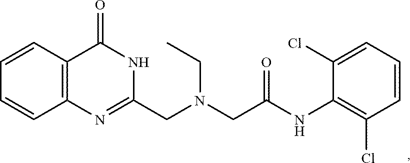

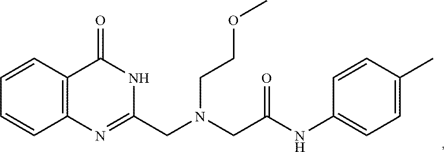

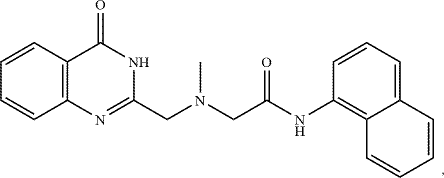

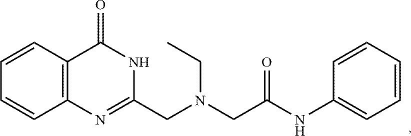

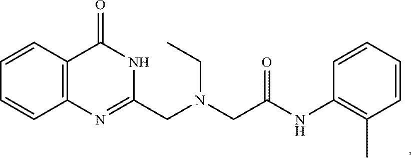

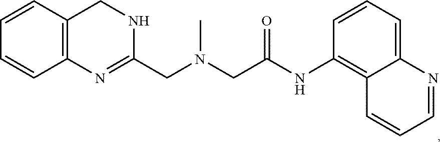

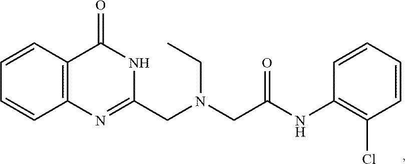

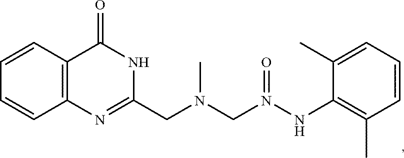

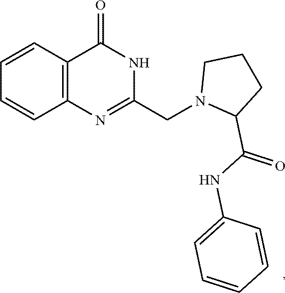

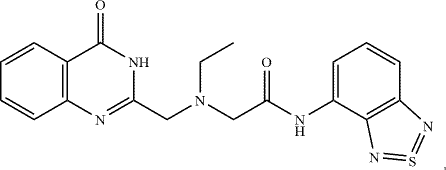

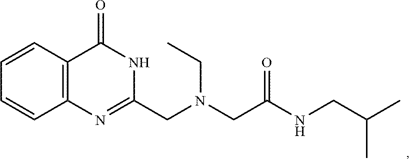

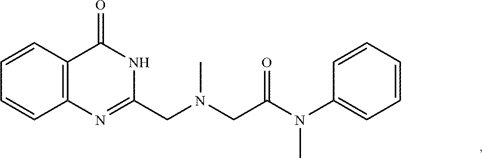

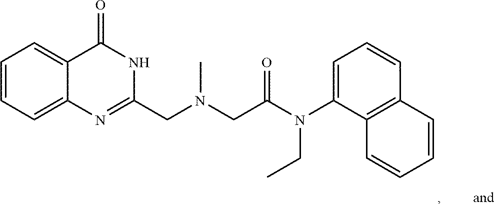

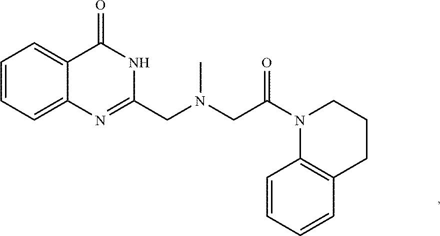

and the pharmaceutically acceptable salts and solvates thereof.

In another aspect, Compounds of the Disclosure that can be employed in the methods of the present disclosure include compounds of formula (I) selected from the group consisting of

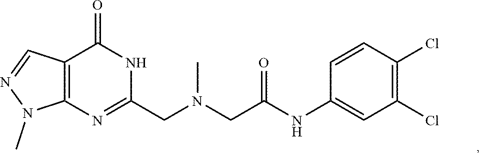

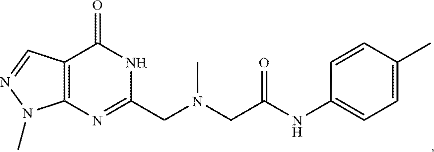

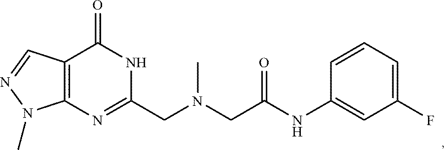

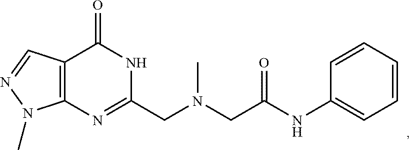

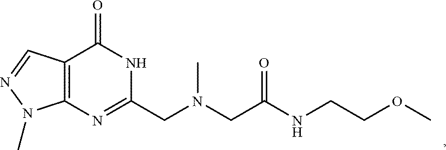

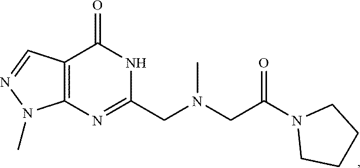

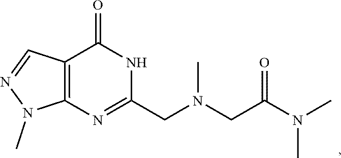

and

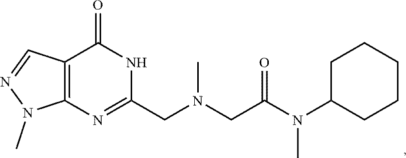

and the pharmaceutically acceptable salts and solvates thereof.

In another aspect, Compounds of the Disclosure include compounds of formula (I′) selected from the group consisting of

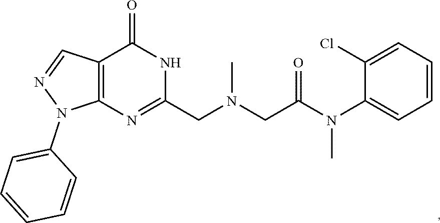

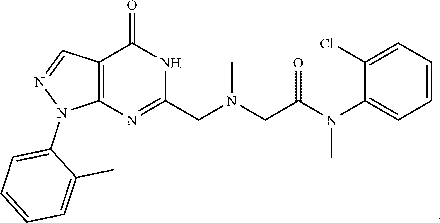

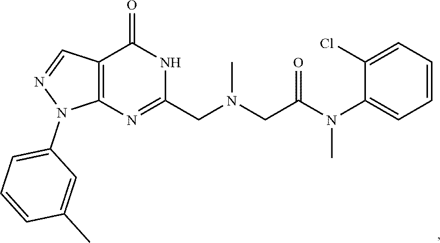

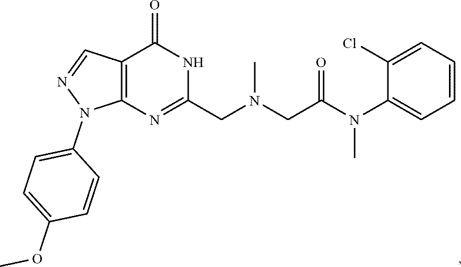

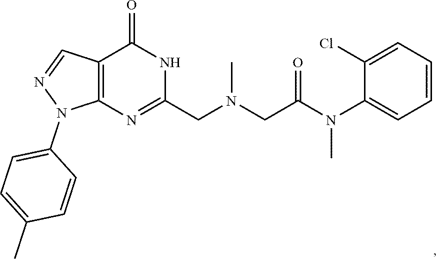

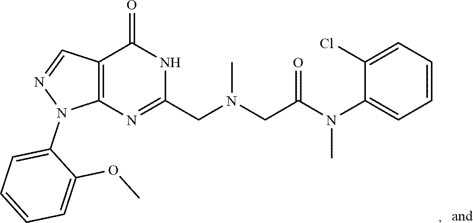

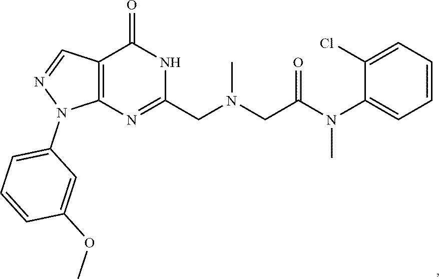

and the pharmaceutically acceptable salts and solvates thereof.

In another aspect, Compounds of the Disclosure include compounds of formula (I′) selected from the group consisting of

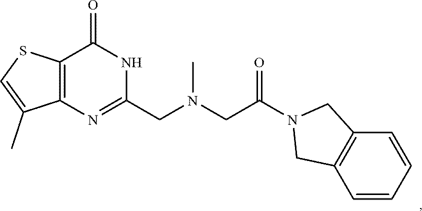

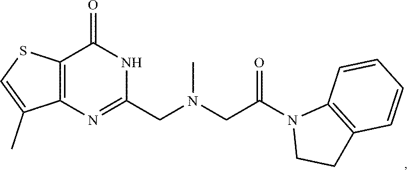

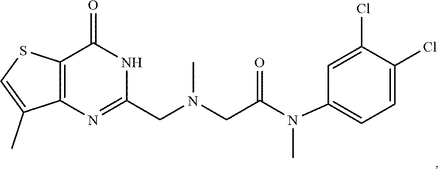

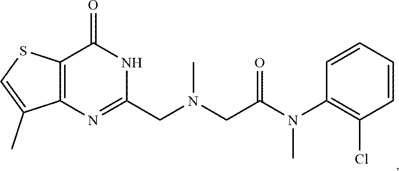

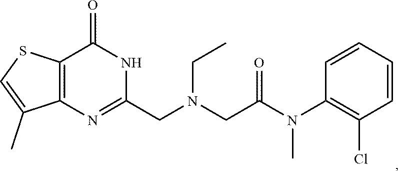

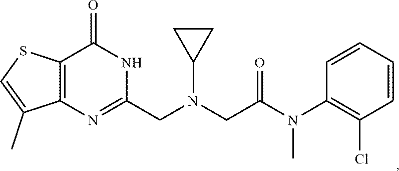

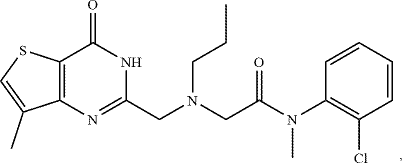

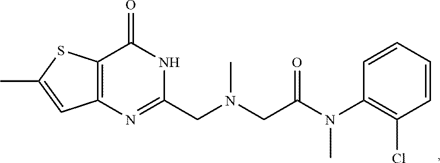

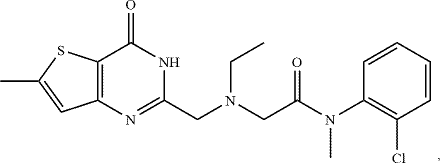

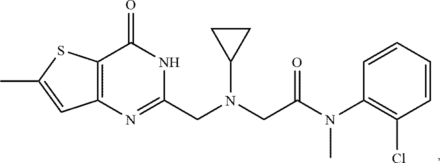

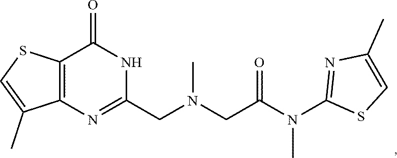

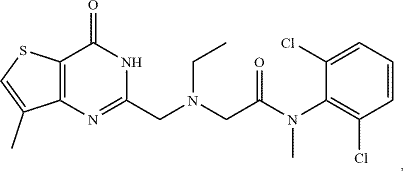

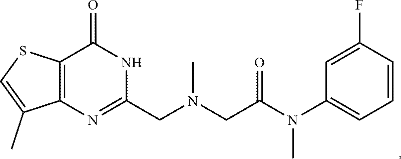

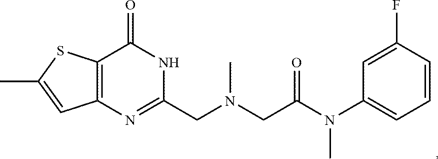

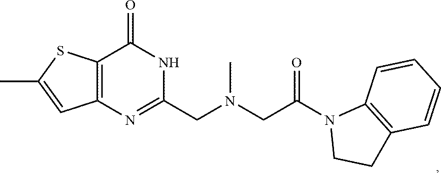

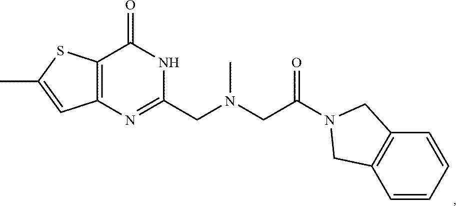

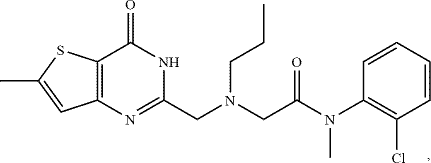

and

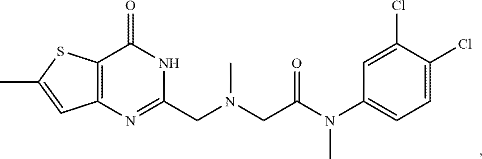

and the pharmaceutically acceptable salts and solvates thereof.

In another aspect, Compounds of the Disclosure include compounds of formula (I′) selected from the group consisting of

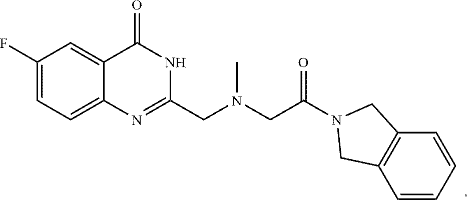

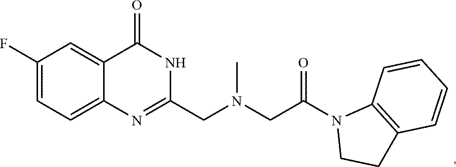

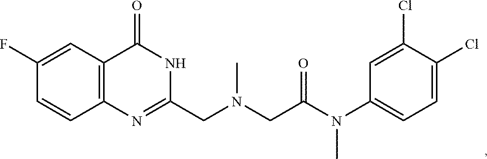

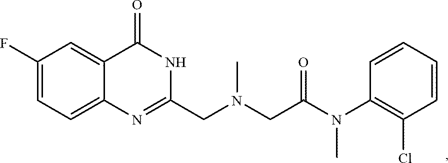

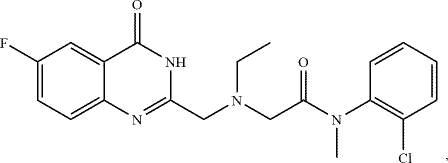

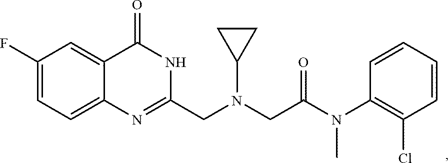

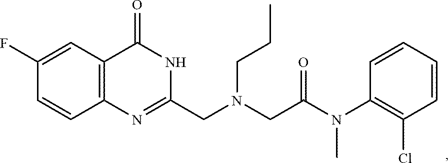

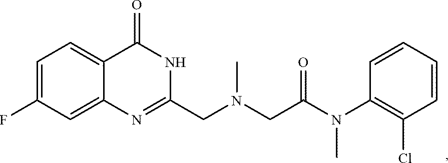

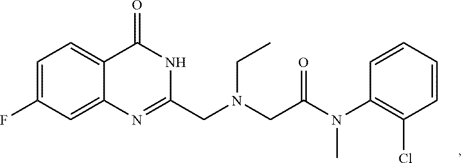

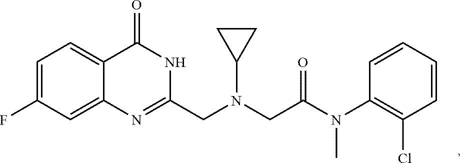

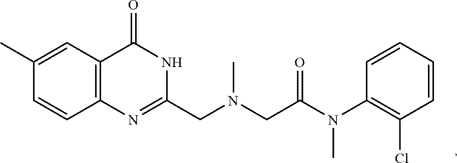

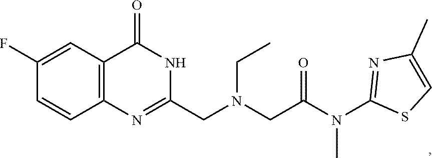

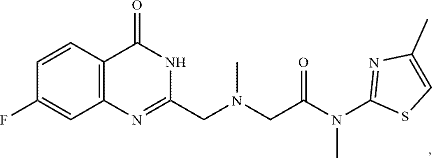

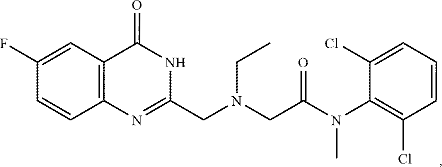

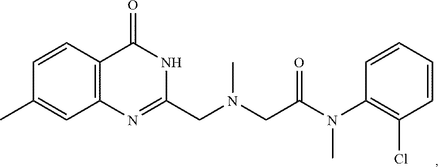

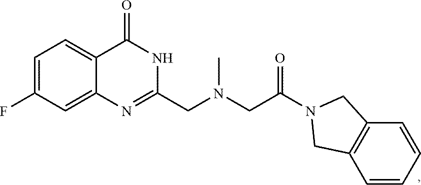

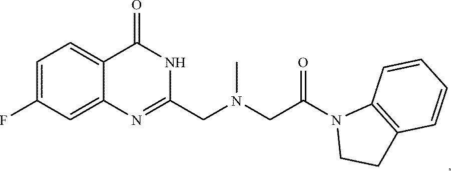

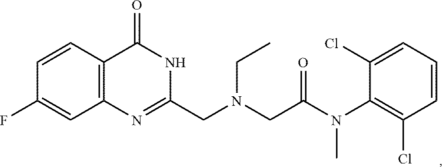

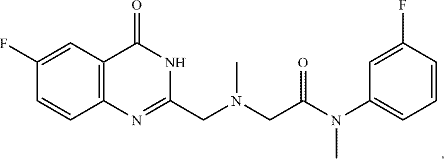

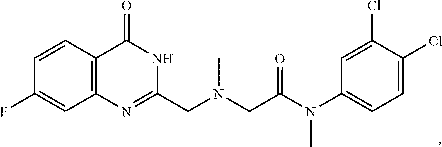

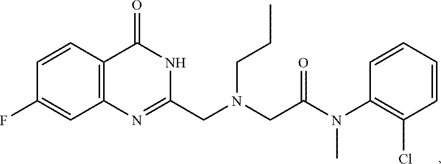

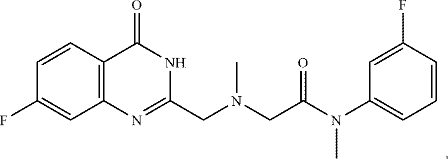

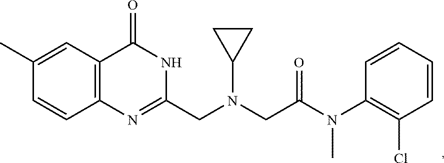

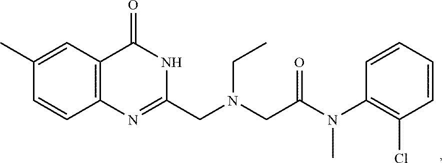

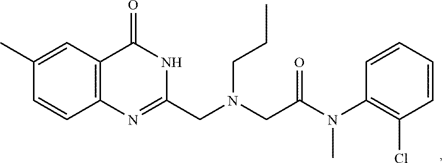

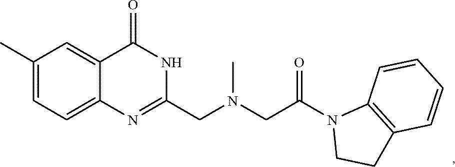

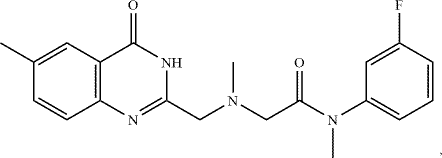

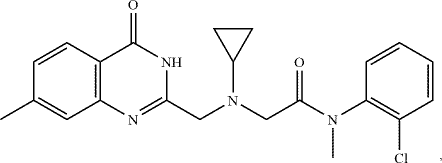

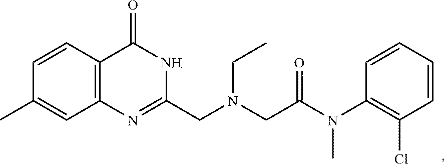

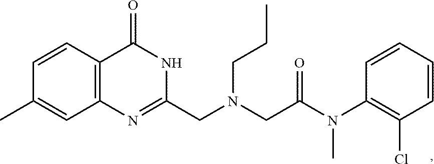

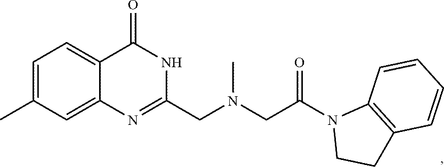

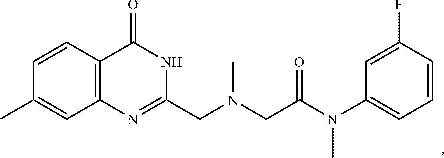

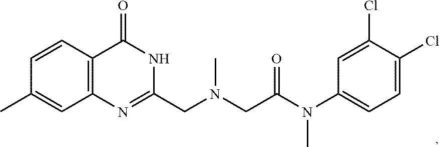

and

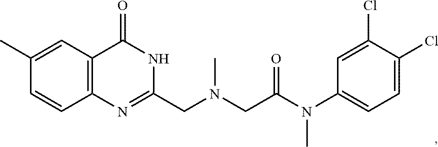

and the pharmaceutically acceptable salts and solvates thereof.

In another aspect, Compounds of the Disclosure include compounds of formula (I′) selected from the group consisting of

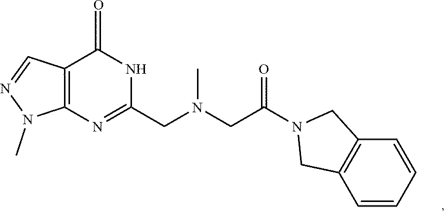

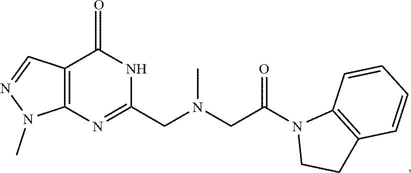

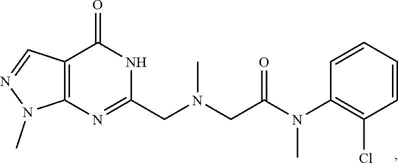

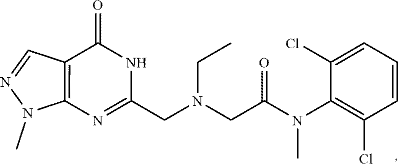

and

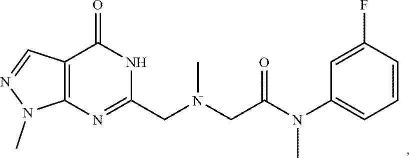

and the pharmaceutically acceptable salts thereof.

In some embodiments, the pharmaceutically acceptable salt of a compound of any one of formulae (I) and (I′) is a hydrochloride salt (a HCl-salt).

As used herein, the terms “halogen” or “halo” refer to -F, -Cl, -Br, or -I.

As used herein, the term “hydroxyl” or “hydroxy” refers to the group -OH.

As used herein, the term “alkyl” refers to a linear or branched hydrocarbon chain radical consisting of carbon and hydrogen atoms, containing no unsaturation, which is attached to the rest of the molecule by a single bond and, unless otherwise specified, an alkyl radical typically has from 1 to 4 carbon atoms, i.e., C₁₋₄ alkyl. Exemplary C₁₋₄ alkyl groups can be methyl, ethyl, n-propyl, i-propyl, n-butyl, tert-butyl, i-butyl and sec-butyl. In another embodiment, the alkyl is C₁₋₂ alkyl (methyl or ethyl).

As used herein, the term “C₁₋₄ alkoxy” refers to oxygen substituted by one of the C₁₋₄ alkyl groups mentioned above (e.g., methoxy, ethoxy, propoxy, iso-propoxy, butoxy, tert-butoxy, iso-butoxy, and sec-butoxy), for example by one of the C₁₋₂ alkyl groups.

As used herein, the term “cycloalkyl” embraces saturated carbocyclic radicals and, unless otherwise specified, a cycloalkyl radical typically has from 3 to 6 carbon atoms. Examples of cycloalkyl groups include cyclopropyl, cyclobutyl, cyclopentyl, cyclohexyl and cycloheptyl. It is, for example, cyclopropyl, cyclopentyl and cyclohexyl. In another embodiment, the cycloalkyl group is C₃₋₁₀ cycloalkyl.

As used herein, the term “alkylcycloalkyl” when employed in the definition of a substituent refers to a cycloalkyl group as defined above which is linked through an alkylene radical, such as C₁₋₄ alkylene, with the core structure which it substitutes. As an example, a cyclopentylethyl substituent is a substituent consisting of a cyclopentyl group linked through an ethylene group to the core structure which it substitutes.

As used herein, the terms “heterocyclyl” or “heterocyclic group” embrace typically a monocyclic or polycyclic, non-aromatic, saturated or unsaturated C₂₋₁₀ carbocyclic ring, such as a 5- to 10-membered radical, in which one or more, for example 1, 2, 3 or 4 of the carbon atoms, for example, 1 or 2 of the carbon atoms are replaced by a heteroatom selected from N, O and S. In one embodiment, the heterocyclyl is a C₃₋₇ heterocyclyl, i.e., a heterocycle having 3-7 carbon atoms and at least one heteroatom. In another embodiment, a heterocyclyl is a (5- to 10-membered)-C₂₋₉ heterocyclyl, i.e., a heterocycle having 5- to 10-members, of which 2-9 members are carbon. In another embodiment, the heteroatom is N. In another embodiment, the heteroatom is O.

In another embodiment, the heterocyclyl radicals are saturated. A heterocyclic radical can be a single ring or two or more fused rings wherein at least one ring contains a heteroatom. When a heterocyclyl radical carries one or more substituents, the substituents can be the same or different.

A said optionally substituted heterocyclyl is typically unsubstituted or substituted with 1, 2 or 3 substituents which can be the same or different. Examples of heterocyclic radicals include piperidyl, pyrrolidyl, pyrrolinyl, piperazinyl, morpholinyl, thiomorpholinyl, pyrazolinyl, pyrazolidinyl, quinuclidinyl, tetrazolyl, cromanyl, isocromanyl, imidazolidinyl, oxiranyl, azaridinyl, 4,5-dihydro-oxazolyl and 3-aza-tetrahydrofuranyl. The substituents are, for example, selected from halogen atoms, for example, fluorine or chlorine atoms, hydroxy groups, alkoxycarbonyl groups in which the alkyl moiety has from 1 to 4 carbon atoms, hydroxycarbonyl groups, carbamoyl groups, nitro groups, cyano groups, C₁₋₄ alkyl groups optionally substituted by one or more halogen atoms, C₁₋₄ alkoxy groups, optionally substituted by one or more halogen atoms and C₁₋₄ hydroxyalkyl groups.

As used herein, the term “alkylheterocyclyl” when employed in the definition of a substituent refers to a heterocyclyl group as defined above which is linked through an alkylene radical with the core structure which it substitutes. In one embodiment, the alkylheterocyclyl is a -C₁₋₄ alkyl-(5- to 10-membered)-C₂₋₉ heterocyclyl.

As used herein, the term “aryl” designates typically a C₆₋₁₀ monocyclic or polycyclic aryl radical such as phenyl and naphthyl. In another embodiment, the aryl is phenyl. A said optionally substituted aryl radical is typically unsubstituted or substituted with 1, 2 or 3 substituents which can be the same or different. The substituents are, for example, selected from halogen atoms, for example, fluorine or chlorine atoms, hydroxy groups, alkoxycarbonyl groups in which the alkyl moiety has from 1 to 4 carbon atoms, hydroxycarbonyl groups, carbamoyl groups, nitro groups, cyano groups, C₁₋₄ alkyl groups optionally substituted by one or more halogen atoms, C₁₋₄ alkoxy groups, optionally substituted by one or more halogen atoms and C₁₋₄ hydroxyalkyl groups. When an aryl radical carries 2 or more substituents, the substituents can be the same or different. Unless otherwise specified, the substituents on an aryl group are typically themselves unsubstituted.

As used herein, the term “alkylaryl” when employed in the definition of a substituent refers to an aryl group as defined above which is linked through an alkylene radical, such as C₁₋₄ alkylene, with the core structure which it substitutes.

As used herein, the term “heteroaryl” designates typically a 5- to 10-membered ring system, comprising at least one heteroaromatic ring and containing at least one heteroatom selected from O, S and N, typically 1, 2, 3, or 4 heteroatoms.

A heteroaryl group can comprise a single ring or two or more fused rings wherein at least one ring contains a heteroatom. A said optionally substituted heteroaryl group is typically unsubstituted or substituted with 1, 2 or 3 substituents which can be the same or different. The substituents are, for example, selected from halogen atoms, for example, fluorine, chlorine or bromine atoms, alkoxycarbonyl groups in which the alkyl moiety has from 1 to 4 carbon atoms, carbamoyl groups, nitro groups, hydroxy groups, C₁₋₄ alkyl groups, optionally substituted by one or more halogen atoms and C₁₋₄ alkoxy groups, optionally substituted by one or more halogen atoms. When a heteroaryl radical carries 2 or more substituents, the substituents can be the same or different. Unless otherwise specified, the substituents on a heteroaryl radical are typically themselves unsubstituted.

Examples of heteroaryl groups include pyridyl, pyrazinyl, pyrimidinyl, pyridazinyl, furyl, tetrazolyl, benzofuranyl, oxadiazolyl, oxazolyl, isoxazolyl, benzoxazolyl, imidazolyl, benzimidazolyl, thiazolyl, thiadiazolyl, thienyl, pyrrolyl, pyridinyl, benzothiazolyl, indolyl, indazolyl, purinyl, quinolyl, isoquinolyl, phthalazinyl, naphthyridinyl, quinoxalinyl, quinazolinyl, quinolizinyl, cinnolinyl, triazolyl, indolizinyl, indolinyl, isoindolinyl, isoindolyl, imidazolidinyl, pteridinyl, thianthrenyl, pyrazolyl, 2H-pyrazolo[3,4-d]pyrimidinyl, 1H-pyrazolo[3,4-d]pyrimidinyl, thieno[2,3-d]pyrimidinyl, and the various pyrrolopyridyl radicals.

In another embodiment, the heteroaryl is a (5- to 10-membered)-C₂₋₉ heteroaryl. In another embodiment, the heteroaryl is optionally substituted with 1, 2, or 3 groups independently selected from the group consisting of halogen, hydroxy, -CN, -ORb, -SRb, -N(Rb)₂, -C₁₋₄alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; said cycloalkyl, alkylcycloalkyl, aryl, alkylaryl, heteroaryl, alkylheteroaryl, heterocyclyl, and alkylheterocyclyl is optionally fused to a further (second) ring.

The mention of optionally substituted heteroaryl radicals or rests within the present disclosure is intended to cover the N-oxides obtainable from these radicals when they comprise N-atoms.

As used herein, the term “alkylheteroaryl” when employed in the definition of a substituent refers to an heteroaryl group as defined above which is linked through an alkylene radical with the core structure which it substitutes. In another embodiment, the alkylheteroaryl is a -C₁₋₄ alkyl-(5- to 10-membered)-C₁₋₉ heteroaryl.

As used herein, the term “alkenylheteroaryl” when employed in the definition of a substituent refers to an heteroaryl group as defined above which is linked through an alkenylene radical with the core structure which it substitutes. In another embodiment, the alkenylheteroaryl is a -C₂₋₄ alkenyl-(5- to 10-membered)-C₁₋₉ heteroaryl.

The term “pharmaceutically acceptable” refers to compositions and molecular entities that are physiologically tolerable and do not typically produce an allergic reaction or a similar unfavorable reaction, such as gastric disorders, dizziness and suchlike, when administered to a human or animal. For example, the term “pharmaceutically acceptable” means it is approved by a regulatory agency of a state or federal government or is included in the U.S. Pharmacopoeia or other generally recognized pharmacopoeia for use in animals, and more particularly in humans.

The term “treatment” or “treating” refers to administering a therapy in an amount, manner or mode effective to improve a condition, symptom, or parameter associated with a condition or to prevent progression of a condition, to either a statistically significant degree or to a degree detectable to one skilled in the art. An effective amount, manner, or mode can vary depending on the subject and can be tailored to the patient.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “prevention” or “to prevent” refers to the reduction in the risk of acquiring or developing a given disease or disorder, or the reduction or inhibition of the recurrence or a disease or disorder.

The term “patient” as used herein refers to a human. In some embodiments, the patient is an adult. In some embodiments, the patient is a geriatric patient. In some embodiments, the patient is a child. In some embodiments, the patient is an infant. In some embodiments, the patient is a toddler. In some embodiments, the patient is a preadolescent. In some embodiments, the patient is an adolescent.

As used herein, the term “child” is a human being between the stages of birth and puberty.

The term “puberty” is the process of physical changes through which a child’s body matures into an adult body capable of sexual reproduction. On average, girls begin puberty around ages 10-11 and end puberty around 15-17; boys begin around ages 11-12 and end around 16-17.

As used herein, the term “infant” is the synonym for “baby,” the very young offspring of a human. The term “infant” is typically applied to young children under one year of age.

As used herein, the term “toddler” refers to a child of 12 to 36 months old.

As used herein, the term “preadolescent” refers to a person of 10-13 years old.

As used herein, the term “adolescent” refers to a person between ages 10 and 19.

The term “about”, as used herein in connection with a measured quantity, refers to the normal variations in that measured quantity, as expected by the skilled artisan making the measurement and exercising a level of care commensurate with the objective of measurement and precision of the measuring equipment. Typically, the term “about” includes the recited number ± 10%. Thus, “about 10” means 9 to 11.

As used herein, the term “optionally substituted” refers to a group that can be unsubstituted or substituted.

The term “solvate” means any form of the active compound of the disclosure which has another molecule (for example a polar solvent such as water or ethanol, a cyclodextrin or a dendrimer) attached to it through noncovalent bonds. Methods of solvation are known within the art.

The disclosure also provides salts of the Compounds of the Disclosure. Nonlimiting examples are sulphates; hydrohalide salts; phosphates; lower alkane sulphonates; arylsulphonates; salts of C₁₋₂₀ aliphatic mono-, di- or tribasic acids which can contain one or more double bonds, an aryl nucleus or other functional groups such as hydroxy, amino, or keto; salts of aromatic acids in which the aromatic nuclei may or may not be substituted with groups such as hydroxyl, lower alkoxyl, amino, mono- or di- lower alkylamino sulphonamido. Also included within the scope of the disclosure are quaternary salts of the tertiary nitrogen atom with lower alkyl halides or sulphates, and oxygenated derivatives of the tertiary nitrogen atom, such as the N-oxides. In preparing dosage formulations, those skilled in the art will select the pharmaceutically acceptable salts.

Solvates and salts can be prepared by methods known in the state of the art. Note that the non-pharmaceutically acceptable solvates also fall within the scope of the disclosure because they can be useful in preparing pharmaceutically acceptable salts and solvates.

The Compounds of the Disclosure also seek to include compounds that differ only in the presence of one or more isotopically enriched atoms. For example, compounds having the present structures except for the replacement of a hydrogen by a deuterium or tritium, or the replacement of a carbon by a carbon enriched in ¹¹C, ¹³C or ¹⁴C or the replacement of a nitrogen by a ¹⁵N enriched nitrogen are within the scope of this disclosure.

Some of the compounds disclosed herein can contain one or more asymmetric centers and can thus give rise to enantiomers, diastereomers, and other stereoisomeric forms, such as epimers. The present disclosure is meant to encompass the uses of all such possible forms, as well as their racemic and resolved forms and mixtures thereof. The individual enantiomers can be separated according to methods known to those of ordinary skill in the art in view of the present disclosure. When the compounds described herein contain olefinic double bonds or other centers of geometric asymmetry, and unless specified otherwise, it is intended that they include both E and Z geometric isomers. All tautomers are intended to be encompassed by the present disclosure as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The term “epimer” refers to diastereomers that have opposite configuration at only one of two or more tetrahedral streogenic centers present in the respective molecular entities.

The term “stereogenic center” is an atom, bearing groups such that an interchanging of any two groups leads to a stereoisomer.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The terms “a” and “an” refer to one or more.

Some reactions for preparing Compounds of the Disclosure involve employing amino protecting groups. As used herein, an “amine protecting group” or “amino protecting group” refers to a group that blocks (i.e., protects) the amine functionality while reactions are carried out on other functional groups or parts of the molecule. Those skilled in the art will be familiar with the selection, attachment, and cleavage of amine protecting groups and will appreciate that many different protective groups are known in the art, the suitability of one protective group or another being dependent on the particular synthetic scheme planned. Treatises on the subject are available for consultation, such as Wuts, P. G. M. & Greene, T. W., Greene’s Protective Groups in Organic Synthesis, 4rd Ed. (J. Wiley & Sons, 2007), herein incorporated by reference in its entirety. Suitable amine protecting groups include methyl carbamate, tert-butyloxycarbonyl (tert-butyl carbamate; BOC), 9-fluorenylmethyl carbamate, benzyl carbamate, 2-(trimethylsilyl)ethyl carbamate, trifluoroacetamide, benzylamine, allylamine, tritylamine, trichloroacetyl, trifluoroacetyl, p-toluenesulfonyl, and allyl carbamate. In another embodiment, the protected amino group can be a phthalimide-protected amino group (NPhth).

“Concurrent administration,” “administered in combination,” “simultaneous administration,” and similar phrases mean that a two or more agents are administered concurrently to the subject being treated. By “concurrently,” it is meant that each agent is administered either simultaneously or sequentially in any order at different points in time. However, if not administered simultaneously, it is meant that they are administered to an individual in a sequence and sufficiently close in time so as to provide the desired therapeutic effect and can act in concert. For example, a Compound of the Disclosure and IDUA (such as laronidase) can be administered at the same time or sequentially in any order at different points in time. The Compound of the Disclosure and IDUA can be administered separately, in any appropriate form and by any suitable route, e.g., by SC and by IV injection, respectively. When the Compound of the Disclosure and IDUA are not administered concurrently, it is understood that they can be administered in any order to a subject in need thereof. For example, the Compound of the Disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, or more before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, or more after) IDUA. In various embodiments, the Compound of the Disclosure and IDUA are administered 1 minute apart, 10 minutes apart, 30 minutes apart, less than 1 hour apart, about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart, no more than 48 hours apart, no more than 3 days apart, or no more than 1 week apart. In one embodiment, the Compound of the Disclosure is administered 1-14 days prior to the day IDUA is administered. In one embodiment, the Compound of the Disclosure is administered 1-7 days prior to the day IDUA is administered. In another embodiment, IDUA is administered on the day the Compound of the Disclosure is administered.

As used herein, the term “enzyme replacement therapy” or “ERT” refers to administering an exogenously-produced natural or recombinant enzyme or analog thereof to a patient in need thereof. In the case of a lysosomal storage disease, e.g. MPS1, for example, the patient accumulates harmful levels of a substrate (i.e., material stored) in lysosomes due to a deficiency or defect in an enzyme responsible for metabolizing the substrate, or due to a deficiency in an enzymatic activator required for proper enzymatic function. Enzyme replacement therapy is provided to the patient to reduce the levels of (i.e., debulk) accumulated substrate in affected tissues. Enzyme replacement therapies for treating MPS1 are known in the art. In accordance with a combination therapy of the disclosure, a lysosomal enzyme, e.g., α-L-iduronidase (IDUA, laronidase) or an analog or variant thereof, can be used for enzyme replacement therapy to reduce the levels of corresponding substrate, α-L-iduronic acid, in a patient having MPS1.

Iduronidase (EC 3.2.1.76, L-iduronidase, alpha-L-iduronidase, laronidase), sold as ALDURAZYME®, is an enzyme with the systematic name glycosaminoglycan alpha-L-iduronohydrolase. This enzyme catalyses the hydrolysis of unsulfated alpha-L-iduronosidic linkages in dermatan sulfate. It is a glycoprotein enzyme found in the lysosomes of cells. It is involved in the degeneration of glycosaminoglycans such as dermatan sulfate and heparan sulfate. The enzyme acts by hydrolyzing the terminal alpha-L-iduronic acid residues of these molecules, degrading them. The protein is reported as having a mass of approximately 83 kilodaltons. ALDURAZYME® can be administered at a dose of 0.58 mg/kg of body weight once weekly as an intravenous infusion.

As used herein, the term “substrate reduction therapy” or “SRT” is a therapeutic approach used to treat certain metabolic disorders, e.g., lysosomal storage disorders, in which substrate, e.g., glycolipid, accumulation is counteracted not by replacing the deficient enzyme but by reducing the substrate level to better balance residual activity of the deficient enzyme. See, e.g., Coutinho et al., Int. J. Mol. Sci. 17:1065 (2016). Substrate reduction therapy and enzyme replacement therapy (see above) can have unique, independent, and potentially complementary mechanisms of action in the treatment of lysosomal storage disease and other diseases.

The general principle of SRT is that a substrate reduction agent is administered to a patient to partially inhibit the biosynthesis of the substrate, which accumulates in the absence of a specific lysosomal enzyme. As used herein, the term “substrate reduction agent” is a small molecule that reduces the number of substrate molecules requiring catabolism within the lysosome, thus contributing to balance the rate of synthesis with the impaired rate of catabolism. Substrate reduction agents are known in the art.

As used herein, an “effective amount” of an enzyme, when administered to a subject in a combination therapy of the disclosure, is an amount sufficient to improve the clinical course of a lysosomal storage disease, e.g., MPS1, where clinical improvement is measured by any of the variety of defined parameters well known to the skilled artisan.

As used herein the term “small molecule chaperone” refers to a compound, other than a Compound of the Disclosure, that is capable of binding allosterically or competitively to a mutated enzyme, e.g., α-L-iduronidase, thereby stabilizing the enzyme against degradation. In some embodiments, the small molecule chaperone facilitates proper folding and transport of an enzyme to its site of action. Small molecule chaperones for the treatment of lysosomal storage diseases are known in the art. See, e.g., US 2016/0207933 A1 and WO 2011/049737 A1.

As used herein, the term “solid cancers” refer solid cancers that are due to aberrant cell signalling of HS-dependent growth factors or morphogens that increase tumor growth and associated angiogenesis.

As used herein, the term “infectious diseases” refer to, e.g., diseases selected from the group consisting HIV, herpes simplex, and human papilloma virus. Various viral-docking proteins utilize HS proteoglycans on host cells as receptors or coreceptors including pseudorabies virus gC protein, herpes simplex gC and gD proteins, P. falciparum erythrocyte membrane protein 1 (PfEMP1), human papilloma virus L1 capsid protein and HIV-1 transactivating factor Tat.

As used heresin, “inflammatory disorders” refer to disorders or conditions where HS-protein interactions support several steps of the inflammatory process. Vascular endothelial HS acts as a ligand for L-selectin during neutrophil rolling, supports chemokine transcytosis and presents chemokines at the lumenal surface of the endothelium.

### Synthesis of Compounds of the Disclosure

Another aspect of the disclosure refers to procedures to obtain compounds of formula (I) and formula (I′). The following methods describe the procedures for obtaining compounds of general formulae (I) and (I′), or solvates or salts thereof.

Various synthetic routes for synthesizing compounds of formula (I) and formula (I′) are summarized in the schemes below.

Scheme 1 illustrates the synthetic path to obtain compounds of formulae (I) and (I′).

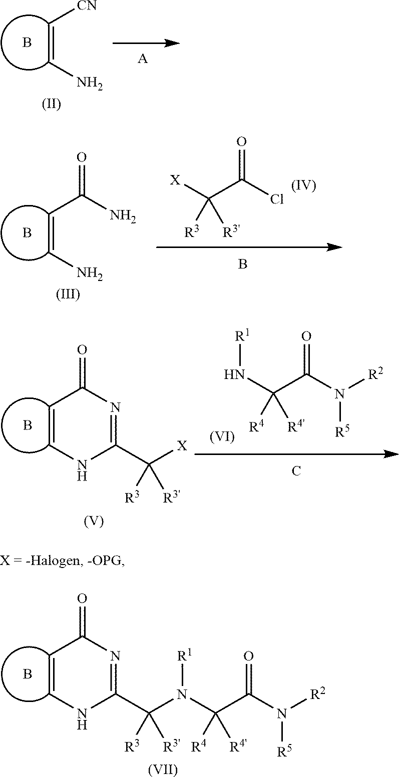

where B, R¹, R², R³, R^{3'}, R⁴, R^{4'} and R⁵ are as defined above for formula (I).

### Method 1

### Step 1 (Reaction A)

In a first method, according to the disclosure, a compound of formula (II), wherein B is as defined above can be transformed in a compound of formula (III) according to the disclosure as illustrated in reaction A of the scheme above (Scheme 1).

The cyano group of the compound of formula (II) is subsequently reduced in the appropriate aldehyde group under standard reductive conditions in the presence of a suitable reducing agent or catalyst (e.g., diisobutylaluminum hydride, sodium hypophosphite, lithium aluminum hydride, nickel, aluminum oxide, platinum oxide), an appropriate solvent (e.g., dichloromethane, tetrahydrofuran, ether, methanol, ethanol, water or mixture thereof) and for example, at around -78° C., room temperature, reflux or microwave irradiation reaction conditions. The reaction can also be carried out in the presence of an acid, such as acetic acid or base (e.g., pyridine) or under hydrogen atmosphere.

### Step 2 (Reaction B)

In a first method, according to the disclosure, a compound of formula (IV), wherein X can be -OPG, or halogen, where PG is a protecting group and R³ is defined above, is reacted with a (Z)-3-aminoacrylamide source to yield a compound of formula (V) according to the disclosure as illustrated in reaction B of the scheme above (Scheme 1).

Reaction B is carried out under standard condensation conditions, for example in the presence of an activating agent (e.g., 1-ethyl-3-(3-dimethylaminopropyl)carbodiimide hydrochloride (EDC.HCl) and hydroxybenzotriazole (HOBt), or pentafluorophenol (PFP) and N,N′-di-isopropylcarbodiimide (DIC) in the presence of diethylaminopyridine (DMAP), or (benzotriazole-1-yloxy)tris(dimethylamino)phosphonium hexafluorophosphate (BOP), preferably in the presence of a base, such as N-methylmorpholine (NMM) or N,N′-diidopropylethylamine (DIEA), in an appropriate solvent (e.g., dichloromethane, chloroform, dimethylformamide or mixture thereof) and for example at around room temperature or reflux temperature.

The compound of formula (IV) is commercially available or can be obtained by procedures described in the literature as is known by the person skilled in the art.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

### Step 3 (Reaction C)

In a first method, according to the disclosure, a compound of formula (V), wherein B and R³ are as defined above and X can be different leaving group or leaving group precursors, where PG is a protecting group, is reacted with an amine (VI), wherein R¹, R², R⁴, and R⁵ are as defined above, to yield a compound of formula (I) according to the disclosure as illustrated in reaction C of the scheme above (Scheme 1).

The X group of the compound of formula (V) is converted into a leaving group by standard methods, for instance the hydroxyl group can be deprotected by standard methods and subsequently transformed into a leaving group such as halogen, triflate, tosylate or a mesylate group.

The leaving group of the compound of formula (V) is converted by reaction with an amine (VI) to a corresponding amine group to yield the compound of formula (I) according to the disclosure as illustrated in reaction C of the schemes above (Scheme 1). Reaction C is carried out under standard nucleophilic substitution conditions, for example in the presence a suitable base (e.g., N,N-diisopropylethylamine, 4-dimethylaminopyridine, 2,6-lutidine, triethylamine, pyridine, ammonium chloride, sodium hydride, potassium carbonate, sodium carbonate, sodium hydrogen carbonate, sodium hydroxide, sodium acetate or sodium nitrite) and an appropriate solvent (e.g., acetonitrile, dichloromethane, tetrahydrofuran, benzene, diethyl ether, toluene, dimethylformamide, water, ethanol or mixture thereof). Such reactions can be used a base or acid in a further step such as, acetic acid, hydrogen chloride or sodium hydroxide.

The reaction mixture is stirred at a low temperature or room temperature or heated until the starting materials have been consumed. The reaction can be carried out with protecting groups present and those protecting groups can be removed after reaction. Suitable protecting groups are known to the person skilled in the art (see T. W. Greene, “Protective Groups in Organic Synthesis,” 3rd Edition, New York, 1999).

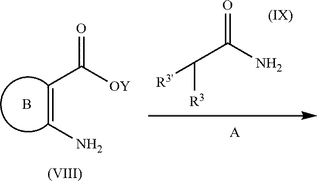

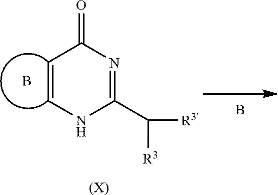

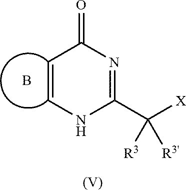

X = -Halogen, -OPG,
B and R³, and R^{3′} are as defined above for formula (I).

### Method 2

### Step 5 (Reaction A)

In a second method, according to the disclosure, a compound of formula (IX), wherein R³ is defined above, is reacted with a (Z)-3-aminoacrylic acid source where Y is a small alkyl group (VIII), to yield a compound of formula (X) according to the disclosure as illustrated in reaction A of the scheme above (Scheme 2).

The ester group in compound (VIII) can be hydrolyzed to the carboxylic acid group following standard methods and then the acid can be converted to the amide under standard condensation or amide coupling conditions, for example in the presence of a suitable coupling agent (e.g., 1,1’-carbonyldiimidazole, N,N′-cyclohexylcarbodiimide, 1-(3-dimethylaminopropyl)-3-ethylcarbodiimide (or hydrochloride thereof), N,N′-disuccinimidyl carbonate, benzotriazol-1-yloxytris(dimethylamino)phosphonium hexafluoro-phosphate, 2-(1H-benzotriazol-1-yl)-1,1,3,3-tetramethyluronium hexafluorophosphate (i.e. O-(1H-benzotriazol-1-yl)-N,N,N′,N′-tetramethyluronium hexafluorophosphate), benzotriazol-1-yloxytris-pyrrolidinophosphonium hexafluorophosphate, bromo-tris-pyrrolidinophosphonium hexafluorophosphate, 2-(1H-benzotriazol-1-yl)-1,1,3,3-tetramethyluronium tetra-fluorocarbonate, 1-cyclohexylcarbodiimide-3-propyloxymethyl polystyrene, O-(7-azabenzotriazol-1-yl)-N,N,N′,N′-tetramethyluronium hexafluorophosphate, or O-benzotriazol-1-yl-N,N,N′,N′-tetramethyluronium hexfluoroborate), optionally in the presence of a suitable base (e.g., sodium hydride, sodium bicarbonate, potassium carbonate, pyridine, triethylamine, dimethylaminopyridine, diisopropylamine, sodium hydroxide, potassium tert-butoxide, and/or lithium diisopropylamide (or variants thereof) and an appropriate solvent (e.g., tetrahydrofuran, pyridine, toluene, dichloromethane, chloroform, acetonitrile, dimethylformamide, trifluoromethylbenzene, dioxane, or triethylamine). Such reactions can be performed in the presence of a further additive, such as 1-hydroxybenzotriazole hydrate.

Reaction A is carried out under standard condensation conditions, for example in the presence of an activating agent (e.g., 1-ethyl-3-(3-dimethylaminopropyl)carbodiimide hydrochloride (EDC.HCl) and hydroxybenzotriazole (HOBt), or pentafluorophenol (PFP) and N,N′-di-isopropylcarbodiimide (DIC) in the presence of diethylaminopyridine (DMAP), or (benzotriazole-1-yloxy)tris(dimethylamino)phosphonium hexafluorophosphate (BOP), preferably in the presence of a base, such as N-methylmorpholine (NMM) or N,N′-diidopropylethylamine (DIEA), in an appropriate solvent (e.g., dichloromethane, chloroform, dimethylformamide or mixture thereof) and for example at around room temperature or reflux temperature.The compound of formula (VIII) is commercially available or can be obtained by procedures described in the literature as is known by the person skilled in the art.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

### Step 6 (Reaction B)

In a second method, according to the disclosure, a compound of formula (X), wherein R³ is defined above, can be transformed in a compound of formula (V) according to the disclosure as illustrated in reaction B of the scheme above (Scheme 2).

Reaction B is carried out under standard radical halogenation conditions, for example radical bromination conditions by treatment with Br₂ or a source of bromine (ex; NBS) in the presence of UV light or an acid as glycial acetic acid, and for example at 40° C. or reflux temperature.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

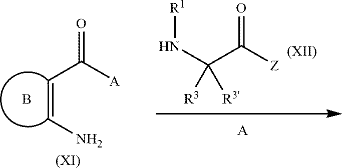

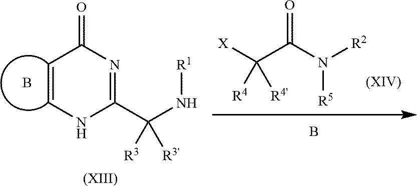

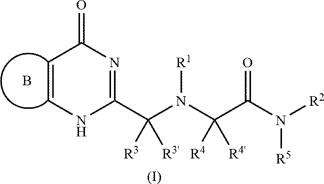

X = -Halogen, -OPG
A = -OH, -NH₂,
Z = -Cl, -NH₂, -OPG
wherein B, R¹, R², R³, R^{3'}, R⁴, R^{4′}, and R⁵ are as defined above for formula (I).

### Method 3

### Step 7 (Reaction A)

In a third method, according to the disclosure, a compound of formula (XII), wherein Z can be -Cl, -OPG or -NH₂, and R³ and R¹ are defined above, is reacted with a (Z)-3-aminoacrylamide (A=-NH₂) source or a (Z)-3-aminoacrylic acid source (A=-OH) (XI), to yield a compound of formula (XIII) according to the disclosure as illustrated in reaction A of the scheme above (Scheme 3).

Reaction A is carried out under standard condensation conditions, for example in the presence of an activating agent (e.g., 1-ethyl-3-(3-dimethylaminopropyl)carbodiimide hydrochloride (EDC.HCl) and hydroxybenzotriazole (HOBt), or pentafluorophenol (PFP) and N,N′-di-isopropylcarbodiimide (DIC) in the presence of diethylaminopyridine (DMAP), or (benzotriazole-1-yloxy)tris(dimethylamino)phosphonium hexafluorophosphate (BOP), preferably in the presence of a base, such as N-methylmorpholine (NMM) or N,N′-diidopropylethylamine (DIEA), in an appropriate solvent (e.g., dichloromethane, chloroform, dimethylformamide or mixture thereof) and for example at around room temperature or reflux temperature. The compound of formula (XII) is commercially available or can be obtained by procedures described in the literature as is known by the person skilled in the art.

The reaction can be carried out with protecting groups present and those protecting groups can be removed after the reaction. Suitable protecting groups are known to the person skilled in the art (see T. W. Greene, “Protective Groups in Organic Synthesis,” 3rd Edition, New York, 1999).

### Step 8 (Reaction B)

In a third method, according to the disclosure, a compound of formula (XIII), wherein B, R¹ and R³ are as defined above is reacted with a compound (XIV), wherein R² and R⁵ are as defined above and X can be different leaving group or leaving group precursors, where PG is a protecting group, to yield a compound of formula (I) according to the disclosure as illustrated in reaction B of the scheme above (Scheme 3).

The X group of the compound of formula (XIV) is converted into a leaving group by standard methods, for instance the hydroxyl group can be deprotected by standard methods and subsequently transformed into a leaving group such as halogen, triflate, tosylate or a mesylate group.

The leaving group of the compound of formula (XIV) is converted by reaction with an amine (XIII) to a corresponding amine group to yield the compound of formula (I) according to the disclosure as illustrated in reaction B of the schemes above (Scheme 3). Reaction B is carried out under standard nucleophilic substitution conditions, for example in the presence a suitable base (e.g., N,N-diisopropylethylamine, 4-dimethylaminopyridine, 2,6-lutidine, triethylamine, pyridine, ammonium chloride, sodium hydride, potassium carbonate, sodium carbonate, sodium hydrogen carbonate, sodium hydroxide, sodium acetate or sodium nitrite) and an appropriate solvent (e.g., acetonitrile, dichloromethane, tetrahydrofuran, benzene, diethyl ether, toluene, dimethylformamide, water, ethanol or mixture thereof). Such reactions can be used a base or acid in a further step such as, acetic acid, hydrogen chloride or sodium hydroxide.

The reaction mixture is stirred at a low temperature or room temperature or heated until the starting materials have been consumed. The reaction can be carried out with protecting groups present and those protecting groups can be removed after reaction. Suitable protecting groups are known to the person skilled in the art (see T. W. Greene, “Protective Groups in Organic Synthesis,” 3rd Edition, New York, 1999).

### Use of the Compounds of the Disclosure

Compounds of the Disclosure have the ability to stabilize α-L-iduronidase (IDUA), and thus increase this enzyme. Therefore, Compounds of the Disclosure can be used/administered to treat and/or prevent conditions associated with alteration of the activity of IDUA in a patient, such as for example mucopolysaccharidosis 1 (MPS1), heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, and a developmental disorder. See, e.g., Parini, R., et al., Oprhanet Journal of Rare Diseases 12:112 (1027); Morishita, K. and Petty, R. E., Rheumatology 50:v19-v25 (2011); Van Horssen, J., et al., Lancet Neurology 2:482-492 (2003); Spillatini, M. G., et al., Acta Neuropathol 97:585-594 (1999); Snow, A. D., et al., American Journal of Pathology 137(5):1253-1270 (1990); Tran-Lundmark, K., et al., Circulation Research 44:43-52 (2008); and Lindahl, U. and Kjellen, L., J. Intern. Med. 273:555-571 (2013). In one embodiment, the condition associated with alteration of the activity of IDUA is MPS1. In another embodiment, the condition associated with alteration of the activity of IDUA is selected from the group consisting of a heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), and inflammatory arthritis. In another embodiment, the condition associated with alteration of the activity of IDUA is selected from the group consisting of an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, and a developmental disorder.

In some embodiment, MPS1 is Hurler disease, Hurler-Scheie syndrome, or Scheie syndrome.

In another aspect, the present disclosure is directed to a method of treating or preventing a condition associated with the associated with alteration of the activity of IDUA in a patient in need thereof, comprising administering to the patient in need thereof an effective amount of a Compound of the Disclosure, optionally in combination with an effective amount of α-L-iduronidase or an analog or variant thereof, e.g., laronidase. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein. In some embodiments, the method comprises administering an effective amount of the Compound of the Disclosure. In some embodiments, the method comprises administering an effective amount of the Compound of the Disclosure in combination with an effective amount of α-L-iduronidase or an analog or variant thereof, e.g., laronidase. In some embodiments, the method comprises administering an effective amount of the Compound of the Disclosure and an effective amount of α-L-iduronidase or an analog or variant thereof, e.g., laronidase, simultaneously to the patient. In some embodiments, the method comprises administering an effective amount of the Compound of the Disclosure and an effective amount of α-L-iduronidase or an analog or variant thereof, e.g., laronidase, to the patient sequentiallty. In some embodiments, the administration of the Compound of the Disclosure in combination with with an effective amount of α-L-iduronidase or an analog or variant thereof, e.g., laronidase, to the patient, comprises administering the Compound of the Disclosure and α-L-iduronidase or an analog or variant thereof, e.g., larodinase, to the patient in separate pharmaceutical compositions. In some embodiments, the administration of the Compound of the Disclosure in combination with laronidase to the patient, comprises administering an effective amount of the Compound of the Disclosure and an effective amount of α-L-iduronidase or an analog or variant thereof, e.g., larodinase, together in a single pharmaceutical composition.

In another aspect, the present disclosure is directed to a method of treating or preventing MPS1 in a patient in need thereof, comprising administering an effective amount of a Compound of the Disclosure. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein. In some embodiments, the method comprises administering an effective amount of the Compound of the Disclosure concurrently with an effective amount of α-L-iduronidase or an analog or variant thereof, e.g., laronidase. In some embodiments, the method comprises administering an effective amount of the Compound of the Disclosure simultaneously with an effective amount of α-L-iduronidase or an analog or variant thereof, e.g., laronidase.

In another aspect, the present disclosure is directed to a method of treating or preventing heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder in a patient in need thereof, comprising administering an effective amount of a Compound of the Disclosure. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein. In some embodiments, the method comprises administering an effective amount of the Compound of the Disclosure concurrently with an effective amount of α-L-iduronidase or an analog or variant thereof, e.g., laronidase. In some embodiments, the method comprises administering an effective amount of the Compound of the Disclosure simultaneously with an effective amount of α-L-iduronidase or an analog or variant thereof, e.g., laronidase.

In another aspect, any method described herein can further comprise administering to the patient at least one other therapeutic agent. In another aspect, the therapeutic agent is an effective amount of a small molecule chaperone. In another aspect, the small molecule chaperone binds competitively to an enzyme. In another aspect, the small molecule chaperone is selected from the group consisting of iminoalditols, iminosugars, aminosugars, thiophenylglycosides, glycosidase, sulfatase, glycosyl transferase, phosphatase, and peptidase inhibitors. In another aspect, the small molecule chaperone is selected from the group consisting of isofagomine, N-nonyl-1-deoxynojirimycin (NN-DNJ), ambroxol, and miglustat. In another aspect, the small molecule chaperone is selected from the group consisting of isofagomine, N-nonyl-1-deoxynojirimycin (NN-DNJ), and ambroxol. In another aspect, the small molecule chaperone is miglustat. In another aspect, the therapeutic agent is an effective amount of substrate reduction agent for substrate reduction therapy. In another aspect, the substrate reduction agent is miglustat.

In another aspect, the present disclosure is directed to a Compound of the Disclosure, as described herein, for use in the prevention or treatment of a condition associated with the alteration of the activity of IDUA in a patient in need thereof. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In some embodiments, the present disclosure is directed to a Compound of the Disclosure, as described herein, for use in the prevention or treatment of a condition associated with the alteration of the activity of IDUA in a patient in need thereof in combination with an effective amount of α-L-iduronidase or an analog or variant thereof, e.g., laronidase. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to a Compound of the Disclosure, as described herein, for use in the prevention or treatment of MPS1 alone or in combination with an effective amount of α-L-iduronidase or an analog or variant thereof, e.g., laronidase. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to a Compound of the Disclosure, as described herein, for use in the prevention or treatment of heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder, alone or in combination with an effective amount of α-L-iduronidase or an analog or variant thereof, e.g., laronidase. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to a Compound of the Disclosure, as described herein, for use as a medicament. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to use of a Compound of the Disclosure, as described herein, in the preparation of a medicament for the prevention or treatment of a condition associated with the alteration of the activity of IDUA in a patient in need thereof, such as MPS1, heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder described herein. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

In another aspect, the present disclosure is directed to a pharmaceutical composition comprising a Compound of the Disclosure, as described herein, and at least one pharmaceutically acceptable excipient, for use in the treatment or prevention of a condition associated with the alteration of the activity of IDUA in a patient in need thereof, such as MPS1, heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder described herein. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

### Pharmaceutical Compositions

The present disclosure is also directed to pharmaceutical compositions, comprising an effective amount of a Compound of the Disclosure and at least one pharmaceutically acceptable excipient. In some embodiments, the composition comprises an effective amount of a compound of formula (I) or formula (I′), or a pharmaceutically acceptable salt or solvate thereof, as described herein, and at least one pharmaceutically acceptable excipient.

Due to their activity, Compounds of the Disclosure can be used in human medicine. As described above, Compounds of the Disclosure are useful, e.g., for treating or preventing MPS1, heart valve disease, dysostosis multiplex, an eye disease (such as, e.g., glaucoma, corneal clouding, and retinal degeneration), an ear disease (e.g., hearing loss), respiratory obstructions or insufficiency, nerve compression (e.g., carpal tunnel syndrome), inflammatory arthritis, an amyloid related disorder (e.g., AA amyloidosis, Alzheimer’s disease, TTR amyloidosis, type a diabetes, Parkinson’s disease, amyotrophic lateral sclerosis (ALS), prion disease, and AL amyloidosis), disease conditions associated with lipoprotein metabolism (such as, e.g., atherosclerosis and Alzheimer’s disease), solid cancers, infectious disease, an inflammatory disorder, or a developmental disorder. Compounds of the Disclosure can be administered to any patient suffering any of said conditions. The term “patient” as used herein refers to any human that can experience the beneficial effects of a Compound of the Disclosure.

When administered to a patient, a Compound of the Disclosure can be administered as a component of a composition that comprises a pharmaceutically acceptable excipient or carrier.

Compounds of the Disclosure can be administered in combination with at least one other therapeutic agent. Administration of Compounds of the Disclosure with at least one other therapeutic agent can be sequential or concurrent. In another aspect, the Compound of the Invention and the at least one other therapeutic agent are administered in separate dosage forms. In another aspect, the Compound of the Invention and the at least one other therapeutic agent are administered concurrently in the same dosage form.

The term “excipient” refers to a vehicle, diluent, or adjuvant that is administered with the active ingredient. Such pharmaceutical excipients can be sterile liquids, such as water and oils, including those of petroleum, animal, vegetable, or synthetic origin, such as peanut oil, soybean oil, mineral oil, sesame oil, and similar. Water or saline aqueous solutions and aqueous dextrose and glycerol solutions, for example, for injectable solutions, can be used as vehicles. Suitable pharmaceutical vehicles are described in “Remington’s Pharmaceutical Sciences” by E.W. Martin, 21^{st} Edition, 2005; or “Handbook of Pharmaceutical Excipients,” Rowe C.R.; Paul J. S.; Marian E.Q., sixth Edition, incorporated herein by reference.

Examples of pharmaceutical compositions include any solid composition (tablets, pills, capsules, granules, etc.) or liquid compositions (solutions, suspensions, or emulsions) for oral, topical, or parenteral administration.

In another embodiment, the pharmaceutical compositions are in an oral delivery form. Pharmaceutical forms suitable for oral administration can be tablets and capsules, and can contain conventional excipients known in the art, such as binders, for example syrup, gum Arabic, gelatin, sorbitol, tragacanth, or polyvinylpyrrolidone; fillers, for example lactose, sugar, cornstarch, calcium phosphate, sorbitol, or glycine; lubricants for the preparation of tablets, for example magnesium stearate; disintegrants, for example starch, polyvinylpyrrolidone, sodium starch glycolate, or microcrystalline cellulose; or pharmaceutically acceptable wetting agents, such as sodium lauryl sulphate.

Solid oral compositions can be prepared by conventional methods of blending, filling, or preparation of tablets. Repeated blending operations can be used to distribute the active ingredient in all the compositions that use large amounts of fillers. Such operations are conventional in the art. The tablets can be prepared, for example, by dry or wet granulation and optionally can be coated by well-known methods in normal pharmaceutical practice, in particular using enteric coating.

Pharmaceutical compositions can also be adapted for parenteral administration, such as sterile solutions, suspensions, or lyophilized products in the appropriate unit dosage form. Suitable excipients, such as fillers, buffering agents, or surfactants can be used.

The mentioned formulations can be prepared using standard methods, such as those described or referred to in the Spanish and U.S. Pharmacopoeias and similar reference texts.

In general, the effective amount of a Compound of the Disclosure to be administered depends on the relative efficacy of the compound chosen, the severity of the condition or disorder being treated, and the patient’s weight. The active compound can be administered one or more times a day, for example 1, 2, 3, or 4 times daily, with typical total daily doses in the range from about 0.01 mg/kg of body weight/day to about 1000 mg/kg of body weight/day. In another embodiment, the effective dosage amount of a Compound of the Disclosure is about 500 mg/kg of body weight/day or less. In another embodiment, the effective dosage amount of a Compound of the Disclosure is about 100 mg/kg of body weight/day or less. In another embodiment, the effective dosage amount ranges from about 0.01 mg/kg of body weight/day to about 100 mg/kg of body weight/day of a Compound of the Disclosure; in another embodiment, from about 0.02 mg/kg of body weight/day to about 50 mg/kg of body weight/day of a Compound of the Disclosure; and in another embodiment, from about 0.025 mg/kg of body weight/day to about 20 mg/kg of body weight/day of a Compound of the Disclosure.

A composition of the disclosure can be prepared by a method comprising admixing a Compound of the Disclosure with a pharmaceutically acceptable excipient or carrier. Admixing can be accomplished using methods known for admixing a compound and a pharmaceutically acceptable excipient or carrier. In another embodiment, the Compound of the Disclosure is present in the composition in an effective amount.

In another aspect, the present disclosure is directed to a pharmaceutical composition comprising an effective amount of a Compound of the Disclosure and an effective amount of larodinase, and a pharmaceutically acceptable excipient. In some embodiments, the Compound of the Disclosure is a compound of formula (I), or a pharmaceutically acceptable salt or solvate thereof, as described herein. In some embodiments, the Compound of the Disclosure is a compound of formula (I′), or a pharmaceutically acceptable salt or solvate thereof, as described herein.

The following examples are illustrative, but not limiting, of the compounds, compositions and methods of the present disclosure. Suitable modifications and adaptations of the variety of conditions and parameters normally encountered in clinical therapy and which are obvious to those skilled in the art in view of this disclosure are within the spirit and scope of the disclosure.

### EXAMPLES

Hereinafter, the term “h” means hours, “eq” means equivalents, “min” means minutes, “HPLC” means high-performance liquid chromatography, “TLC” means thin layer chromatography, “LC-MS” or “HPLC-MS” means Liquid chromatography-mass spectrometry, “CDCl₃” means deuterated chloroform, “DMSO-d₆” means deuterated dimethyl sulfoxide, “EtOAc” means ethyl acetate, “THF” means tetrahydrofurane, “DMF” means dimethylformamide, “DCM” means dichloromethane, and “DMEDA” means 1,2-dimethylethylenediamine.

¹H NMR spectra were recorded on a Bruker (400 MHz and 500 MHz).

HPLC spectra were recorded on Waters 2695 and Waters UPLC - H class.

LC-MS analysis of the compounds was conducted as per one of the following methods.

Method-A: Aquity UPLC BEH C18 (50 mm × 2.1 mm, 1.7 µm); wavelength: 215 nm; flow: 0.6 mL/min; run time: 4.2 min; Mobile phase A: 0.1% of formic acid in water and B: 0.1% formic acid in acetonitrile; Time and mobile phase-gradient (time in min/%A): 0/95, 0.3/95, 2.0/5, 3.5/5, 3.6/95, MASS: Waters Acquity UPLC with SQD(ESI/APCI).

Method-B: Acquity UPLC BEH C18 (50 mm × 2.1 mm, 1.7 µm); wavelength: MaxPlot; flow: 0.6 mL/min; run time: 10 min; Mobile phase A: 10 Mm Ammonium Acetate in water and B: Acetonitrile; Time and mobile phase-gradient (time in min/%B): 0/3, 1/3, 7.5/100, 9.0/3, 10.0/3; Instrument: Waters Acquity UPLC, Mass: SQ Detector 2 (ESI/APCI).

Method-C: X-BRIDGE C18 (4.6 mm × 75 mm 3.5 µm); wavelength: 215 nm; flow: 2 mL/min; run time: 5.0 min; Mobile phase A: 10 mM of acetonitrile in water and B: 100% acetonitrile; Time and mobile phase-gradient (time in min/%B): 0.0/10, 0.2/10, 2.5/75, 3.0/100, 4.8/100, 5.0/10; MASS: Agilent 1200 SERIES, Mass:6130SQD (ESI/APCI).

Method-D: Aquity UPLC BEH C18 (50 mm × 2.1 mm, 1.7 µm); wavelength: 215 nm; flow: 0.8 mL/min; run time: 3.2 min; Mobile phase A: 0.1% of formic acid in water and B: 1.0% formic acid in acetonitrile; Time and mobile phase-gradient (time in min/%B): 0.0/2, 0.2/2, 1.5/98, 2.6/98, 2.61/2, 3.2/2; MASS: Agilent 1290 infinity, Mass:6150 SQD (ESI/APCI).

Method-E: Aquity UPLC BEH C18 (50 mm × 2.1 mm, 1.7 µm); wavelength: 215 nm; flow: 0.6 mL/min; run time: 6 min; Mobile phase A: 0.1% of formic acid in water and B: 0.1% formic acid in acetonitrile; Time and mobile phase-gradient (time in min/%B): 0.0/5, 0.3/5, 2/95, 0.6/98, 3.7/95, 4.2/5,5.7/5; MASS: Agilent 1290 infinity, Mass:6150 SQD (ESI/APCI).

Method-F: Aquity UPLC BEH C18 (50 mm × 2.1 mm, 1.7 µm); wavelength: 215 nm; flow: 0.5 mL/min; run time: 6 min; Mobile phase A: 0.1% of formic acid in water and B: 0.1% formic acid in acetonitrile; Time and mobile phase-gradient (time in min/%B): 0.0/5, 0.3/5, 2/95, 0.6/98, 3.7/95, 4.2/5,5.7/5; MASS: Agilent 1290 infinity, Mass:6150 SQD (ESI/APCI).

Method-G SunFire C18 (50 mm × 2.1 mm, 5 µm); wavelength: PDA MaxPlot 210.0 - 400 nm; flow: 0.30 mL/min; column temperature: 35° C.; run time: 9 min; mobile phase A: ACN/MeOH (50:50), B: 100 mM ammonium acetate solution, C: water; gradient: A:B:C 0.5 min in 10:5:85 + from 10:5:85 to 95:5:0 in 4 min + 4.5 min in 95:5:0; chromatographic system: Waters Alliance HT 2795 and PDA 2996; mass spectrometer: Micromass ZQ2000 single quadrupole (ESI).

Method-H: SunFire C18 (100 mm × 2.1 mm, 3.5 µm); wavelength: PDA MaxPlot 210.0 - 400 nm; flow: 0.30 mL/min; column temperature: 35° C.; run time: 30 min; mobile phase A: ACN/MeOH (50:50), B: 100 mM ammonium acetate solution, C: water; gradient: A:B:C 5 min in 10:5:85 + from 10:5:85 to 95:5:0 in 15 min + 10 min in 95:5:0; chromatographic system: Waters Alliance HT 2795 and PDA 2996; mass spectrometer: Micromass ZQ2000 single quadrupole (ESI).

Method-I: Sunfire C18 (150 mm × 19 mm, 10 µm); wavelength: the wavelength is selected taking into consideration the UV maximum absorption of the target; flow: 10 mL/min; column temperature: 30° C.; run time: 30 min; mobile phase, A: ACNB: ammonium bicarbonate solution 10 mM (pH 7); gradient: A:B 1 min in 25:75 + from 25:75 to 85:15 in 17 min + 22 min in 85:15; chromatographic system: Dionex 3000 (PLCP001) equipped with a foxy R1 fraction collector.

Method-J: BEH Phenyl (100 mm × 2.1 mm, 1.7 µm); wavelength: PDA MaxPlot 215.0 - 400 nm; flow: 0.40 mL/min; column temperature: 35° C.; run time: 9 min; mobile phase A: water adjusted at pH 3 with formic acid, B: MeOH + 0.1% HCOOH; gradient: A:B 0.5 min in 90:10 + from 90:10 to 15:85 in 4.5 min + 4 min in 15:85; chromatographic system: Acquity H Class UPLC; mass spectrometer: Acquity QDa.

### Synthesis of Examples 1 to 7

### Synthesis of Intermediate 1

Synthetic scheme:

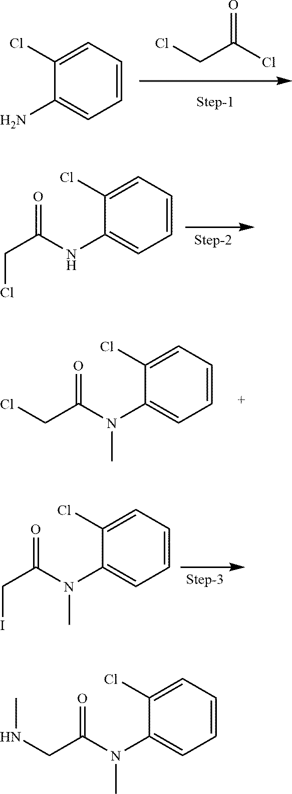

(2-chloro-N-(2-chlorophenyl)acetamide):

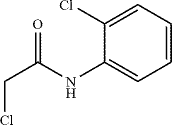 [SMILES: O=C(CCl)Nc1ccccc1Cl]

Chloroacetyl chloride (21.1 g, 188.9 mmol, 1.2 eq) was added dropwise to a stirred solution of 2-chloroaniline (20.0 g,157.4 mmol, leq) and trimethylamine (25.4 g, 251.9 mmol, 1.6 eq) in DCM (300 mL) at 0° C. The reaction mixture was stirred for 30 minutes at 0-5° C. and then at RT for 3 h. The reaction mixture was quenched with minimum amount of aqueous NaHCO₃ solution, the organic product was extracted with DCM (3X500 mL). The combined organic extracts were dried over anhydrous Na₂SO₄. Solvent was distilled under reduced pressure to give the crude compound.

Yield: (21.0 g, 15%).

ES-MS [M+H] ⁺: 204.00; Rt =1.86 min (Method-A).

N-(2-chlorophenyl)-2-iodo-N-methylacetamide:

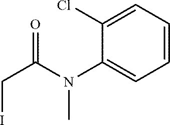

2-chloro-N-(2-chlorophenyl)acetamide (21.0 g, 103.4 mmol, 1.0 eq) in THF (150 mL) was added to a stirred solution of NaH (60% dispersion in mineral oil) (4.9 g, 206.9 mmol, 2.0 eq) in THF (50 mL) at 0° C. and stirred at RT for 30 minutes. MeI (29.3 g, 206.9 mmol, 2.0 eq) was added dropwise in to reaction mixture at 0° C. The reaction mixture was stirred at room temperature for 6 h. After consumption of starting materials. The reaction mixture was cooled and quenched with ice water; the organic product was extracted with EtOAc (3X400 mL). The combined organic extracts were dried over anhydrous Na₂SO₄. Solvent was distilled under reduced pressure to give the crude compound. The crude product was purified by column chromatography (silica gel 230-400 mesh; 10-25% EtOAc in Pet. Ether as eluent) to afford 6.4 g of N-(2-chlorophenyl)-2-iodo-N-methylacetamide.

Yield: (6.4 g, 20%).

ES-MS [M-H]⁺: 309.87; Rt = 1.81 min (Method-A).

¹H NMR (400 MHz, CDCl₃): δ 7.54-7.44 (m, 2 H), 7.39-7.37(m, 2 H), 3.65-3.62 (d, J=10.4 Hz, 1 H), 3.39-3.35 (d, J=10 Hz, 1 H), 3.23 (s, 3 H).

Intermediate 1: N-(2-chlorophenyl)-N-methyl-2-(methylamino)acetamide

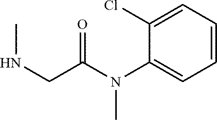

N-(2-chlorophenyl)-2-iodo-N-methylacetamide (6.4 g, 20.7 mmol, 1.0 eq) in THF (40 mL) was added to a stirred solution of NaH (60% dispersion in mineral oil) (0.994 g, 41.4 mmol, 2.0 eq) in at 0° C. Reaction mixture then stirred at RT for 30 minutes and added MeNH₂ (2.0 mol solution in THF, 31 mL, 62.1 mmol, 3.0 eq) dropwise in THF (20 mL) at 0° C. The reaction mixture was stirred at room temperature for 6h. After consumption of starting materials (reaction was monitored by TLC), the reaction mixture was cooled and quenched with ice water, the organic product was extracted with 10% MeOH in DCM (3X250 mL). The combined organic extracts were dried over anhydrous Na₂SO₄. Solvent was distilled under reduced pressure to give the crude compound. The crude product was purified by column chromatography (silica gel 230-400 mesh; 10-15% MeOH in DCM as eluent) to afford 1.5 g of N-(2-chlorophenyl)-N-methyl-2-(methylamino)acetamide.

Yield: (1.5 g, 34%).

ES-MS [M-H]⁺: 213.01; Rt = 1.15 min (Method-A).

### Synthesis of Example 1

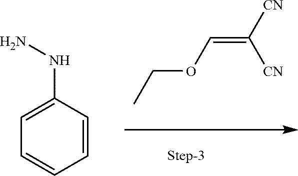

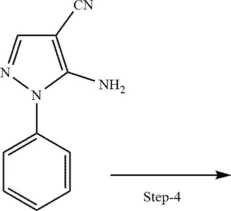

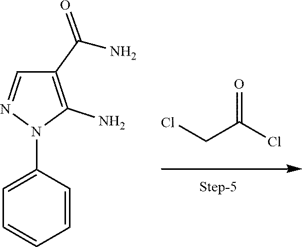

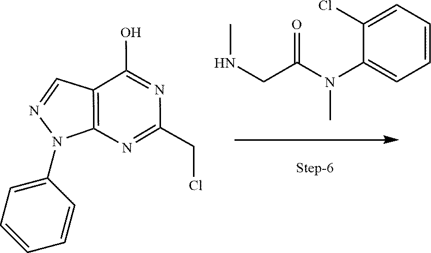

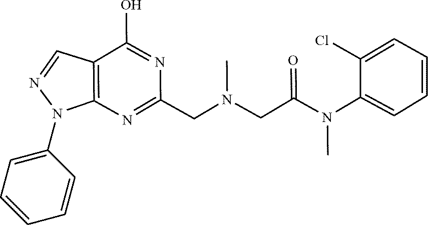 [SMILES: CN(CC(=O)N(C)c1ccccc1Cl)Cc1nc(O)c2cnn(-c3ccccc3)c2n1]

### Intermediate 2: (5-amino-1-phenyl-1H-pyrazole-4-carbonitrile)

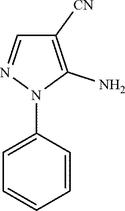 [SMILES: N#Cc1cnn(-c2ccccc2)c1N]

(Etoxymethylene)malononitrile (1.1 g, 9.2 mmol, 1 eq) was added to a suspension of compound phenylhydrazine (1.0 g, 9.2 mmol, 1 eq) in EtOH (15 mL) at 0° C. The reaction mixture was heated to 100° C. for 3 h. The reaction was monitored by TLC. The reaction mixture was concentrated with vacuo, the product was extracted with EtOAc (3X50 mL). The combined organic extracts were dried over anhydrous Na₂SO₄. Solvent was distilled under reduced pressure to give the crude compound.

Yield: (1.0 g).

ES-MS [M-H]⁺: 184.92; Rt = 1.56 min (Method-A).

Intermediate 3: (5-amino-1-phenyl-1H-pyrazole-4-carboxamide)

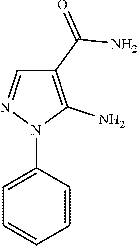 [SMILES: NC(=O)c1cnn(-c2ccccc2)c1N]

Potassium hydroxide (1.5 g, 27.1 mmol, 5 eq) was added to a stirred solution of compound (5-amino-1-phenyl-1H-pyrazole-4-carbonitrile) (1.0 g, 5.43 mmol, 1 eq) in DMSO:water (10 mL:4.0 mL) at 0° C. The reaction mixture was stirred at same temperature for 15 minutes then 30% solution of hydrogen peroxide in water (3 mL, 5 eq) was added drop wise at same temperature and then stirred at RT for 30 min. The reaction mixture was diluted with water, extracted with EtOAc (3X100 mL). The combined organic layer was dried over anhydrous Na₂SO₄, filtered and concentrated under reduced pressure to get crude compound (5-amino-1-phenyl-1H-pyrazole-4-carboxamide).

Yield: (0.8 g).

ES-MS [M+H] ⁺: 202.99; Rt = 1.34 min (Method-A).

Intermediate 4: (6-(chloromethyl)-1-phenyl-1H-pyrazolo[3,4-d]pyrimidin-4-ol):

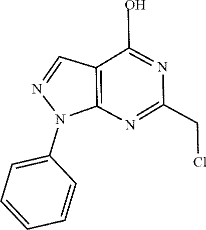

Chloroacetyl chloride (0.532 g, 4.75 mmol, 1.2 eq) was added to a stirred solution of compound (5-amino-1-phenyl-1H-pyrazole-4-carboxamide) (0.8 g, 3.96 mmol, 1 eq) in DMF:Toluene (16 mL:1.6 mL) at 0° C. The reaction mixture was stirred at RT for 3 h then refluxed for 5 h. The reaction mixture was cooled, concentrated under reduced pressure and then quenched with minimum amount of aqueous NaHCO₃ solution, the organic product was extracted with EtOAc (3X25 mL). The combined organic extracts were dried over anhydrous Na₂SO₄. Solvent was distilled under reduced pressure to give the crude compound. The crude product was purified by column chromatography (silica gel 230-400 mesh; 10-20% EtOAc in Pet. Ether as eluent) to afford 180 mg of (6-(chloromethyl)-1-phenyl-1H-pyrazolo[3,4-d]pyrimidin-4-ol).

Yield: (0.18 g, 18%).

ES-MS [M+H] ⁺: 261.02; Rt =1.84 min (Method-A).

¹H NMR (400 MHz, DMSO-d₆): δ 10.94(s, 1 H), 8.31(s, 1 H), 7.58-7.50(m, 5 H), 4.32(s, 2 H).

Example 1: (N-(2-chlorophenyl)-2-(((4-hydroxy-1-phenyl-1H-pyrazolo[3,4-d]pyrimidin-6-yl)methyl)(methyl)amino)-N-methylacetamide):

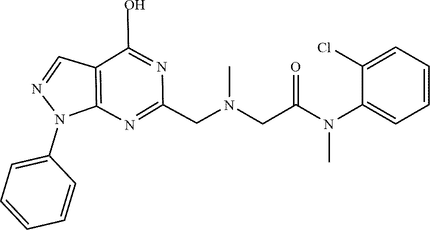

To a solution of N-(2-chlorophenyl)-N-methyl-2-(methylamino)acetamide (0.14 g, 0.692 mmol, 1.0 eq) in THF (5 mL) was added NaH at 0° C. and stirred at same temperature for 15 minutes. To the reaction mixture was added a solution of (6-(chloromethyl)-1-phenyl-1H-pyrazolo[3,4-d]pyrimidin-4-ol) (0.18 g,0.692 mmol,1.0 eq) in THF(5 mL) and stirred at 70° C. for 5 h. The reaction mixture was quenched with ice-water, the organic product was extracted with EtOAc (3X50 mL). The combined organic extracts were dried over anhydrous Na₂SO₄. Solvent was distilled under reduced pressure to give the crude compound. The crude product was purified by reverse phase prep HPLC to afford 50 mg of H- (N-(2-chlorophenyl)-2-(((4-hydroxy-1-phenyl-1H-pyrazolo[3,4-d]pyrimidin-6-yl)methyl)(methyl)amino)-N-methylacetamide) as white solid.

Yield: (0.05 g, 17%).

ES-MS [M-H] ⁻: 435.17; Rt = 1.81 min (Method-A).

¹H NMR (400 MHz, CDCl₃): δ 10.69 (s, 1 H), 7.92 (s, 1 H), 7.58-7.43 (m, 6 H), 7.38-7.26 (m, 3 H) 3.20-3.18 (m, 5 H), 3.04-2.92 (q, J=16.8 Hz, 2 H), 2.32 (s, 3 H).

Synthesis of Example 2:

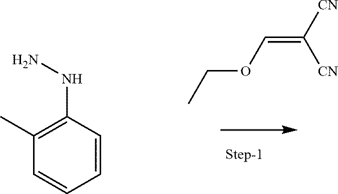

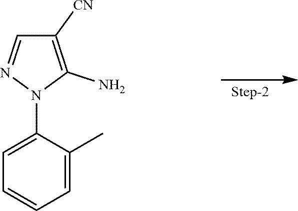

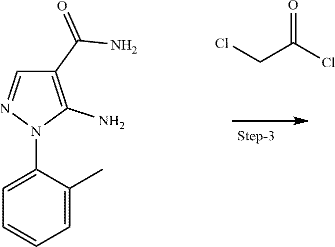

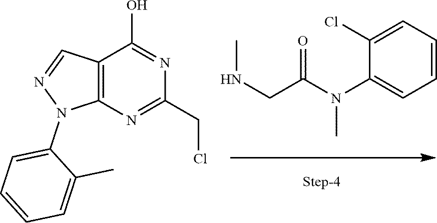

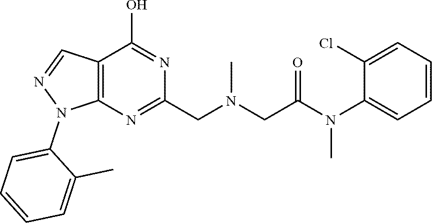 [SMILES: Cc1ccccc1-n1ncc2c(O)nc(CN(C)CC(=O)N(C)c3ccccc3Cl)nc21]

Intermediate 5: (5-amino-1-o-tolyl-1H-pyrazole-4-carbonitrile):

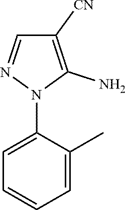

Prepared following procedure described for Intermediate 2.

Yield: (1.5 g).

ES-MS [M-H]⁺: 198.98; Rt = 1.63 min (Method-A).

Intermediate 6: (5-amino-1-o-tolyl-1H-pyrazole-4-carboxamide):

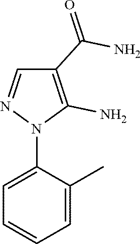 [SMILES: Cc1ccccc1-n1ncc(C(N)=O)c1N]

Prepared following procedure described for Intermediate 3.

Yield: (1.50 g, crude).

ES-MS [M+H] ⁺: 216.94; Rt = 1.38 min (Method-A).

Intermediate 7: (6-(chloromethyl)-1-o-tolyl-1H-pyrazolo[3,4-d]pyrimidin-4-ol):

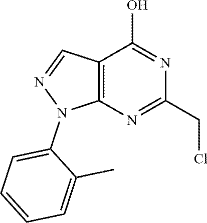 [SMILES: Cc1ccccc1-n1ncc2c(O)nc(CCl)nc21]

Prepared following procedure described for Intermediate 4.

Yield: (0.3 g, 16%).

ES-MS [M-H] ⁺: 273.04; Rt = 1.78 min (Method-A).

¹H NMR (400 MHz, DMSO-d₆): δ 10.77(s, 1 H), 8.28 (s, 1 H), 7.46-7.34(m, 3 H), 7.25-7.23(m, 1 H), 4.20(s, 2 H), 2.01(s, 3 H).

Example 2: (N-(2-chlorophenyl)-2-(((4-hydroxy-1-o-tolyl-1H-pyrazolo[3,4-d]pyrimidin-6-yl)methyl)(methyl)amino)-N-methylacetamide):

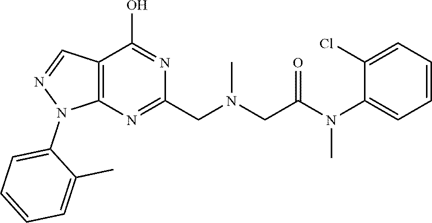 [SMILES: Cc1ccccc1-n1ncc2c(O)nc(CN(C)CC(=O)N(C)c3ccccc3Cl)nc21]

To a solution of N-(2-chlorophenyl)-N-methyl-2-(methylamino)acetamide (0.232 g, 1.09 mmol, 1 eq) in THF (8 mL) was added NaH (60% in mineral oil, 0.35 g, 8.64 mmol, 8 eq) at 0° C. and stirred at same temperature for 15 minutes then a solution of (6-(chloromethyl)-1-o-tolyl-1H-pyrazolo[3,4-d]pyrimidin-4-ol) (0.3 g, 1.09 mmol,1.0 eq) in THF(8 mL) was added and stirred at 70° C. for 5 h. The reaction mixture was quenched with ice-water, the organic product was extracted with EtOAc (3X50 mL). The combined organic extracts were dried over anhydrous Na₂SO₄. Solvent was distilled under reduced pressure to give the crude compound. The crude product was purified by reverse phase prep HPLC to afford 49 mg of H- (N-(2-chlorophenyl)-2-(((4-hydroxy-1-o-tolyl-1H-pyrazolo[3,4-d]pyrimidin-6-yl)methyl)(methyl)amino)-N-methylacetamide) as pale yellow solid.

Yield: (0.08 g, 16%).

ES-MS [M-H] ⁻: 449.27; Rt = 1.87 min (Method-A).

¹H NMR (400 MHz, CDCl₃): δ 10.46(s, 1 H), 7.94 (s, 1 H), 7.52-7.50 (m, 1 H), 7.38-7.22 (m, 7 H), 3.14-3.12 (m, 5 H), 2.92-2.80 (q, J=16.4 Hz, 2 H), 2.17 (s, 3 H), 2.15 (s, 3 H).

Synthesis of Example 3:

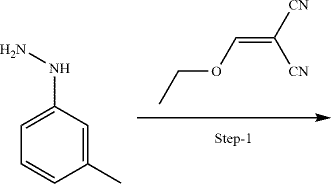

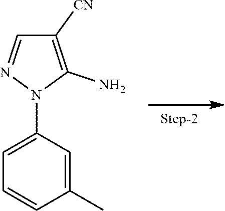

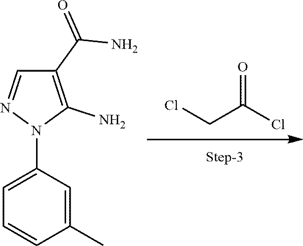

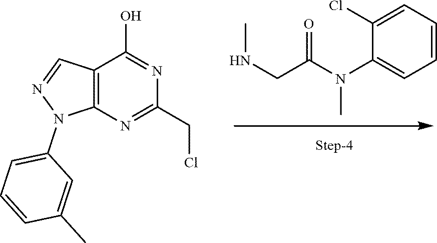

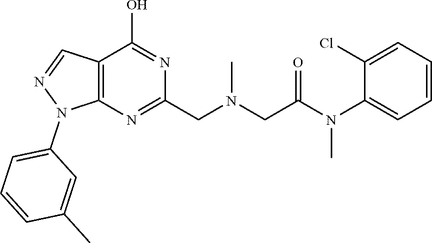 [SMILES: Cc1cccc(-n2ncc3c(O)nc(CN(C)CC(=O)N(C)c4ccccc4Cl)nc32)c1]

Intermediate 8: (5-amino-1-m-tolyl-1H-pyrazole-4-carbonitrile):

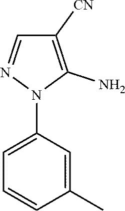

Prepared following procedure described for Intermediate 2.

Yield: (1.0 g, crude).

ES-MS [M-H]⁺: 198.98; Rt = 1.73 min (Method-A).

Intermediate 9: (5-amino-1-m-tolyl-1H-pyrazole-4-carboxamide):

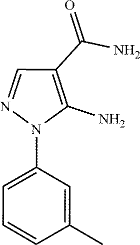 [SMILES: Cc1cccc(-n2ncc(C(N)=O)c2N)c1]

Prepared following procedure described for Intermediate 3.

Yield: (1.1 g).

ES-MS [M+H] ⁺: 216.91; Rt = 1.50 min (Method-A).

Intermediate 10: (6-(chloromethyl)-1-m-tolyl-1H-pyrazolo[3,4-d]pyrimidin-4-ol):

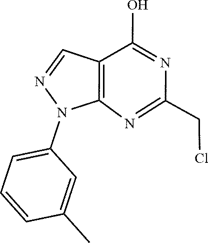 [SMILES: Cc1cccc(-n2ncc3c(O)nc(CCl)nc32)c1]

Prepared following procedure described for Intermediate 4.

Yield: (0.18 g, 13%).

ES-MS [M+H] ⁺: 275.05; Rt = 1.92 min (Method-A).

¹H NMR (400 MHz, DMSO-d₆): δ 12.73 (s, 1 H), 8.33 (s, 1 H), 7.85-7.83 (m, 2 H), 7.47-7.43 (t, J=7.6 Hz, 1 H), 7.25-7.23 (d, J=7.2 Hz, 1 H), 4.62 (s, 2 H), 2.41 (s, 3 H).

Example 3: (N-(2-chlorophenyl)-2-(((4-hydroxy-1-m-tolyl-1H-pyrazolo[3,4-d]pyrimidin-6-yl)methyl)(methyl)amino)-N-methylacetamide):

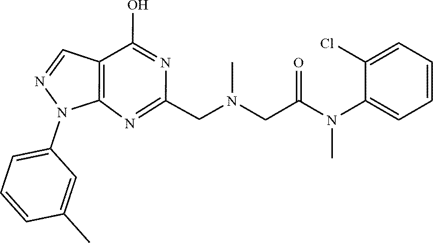 [SMILES: Cc1cccc(-n2ncc3c(O)nc(CN(C)CC(=O)N(C)c4ccccc4Cl)nc32)c1]

To a solution of N-(2-chlorophenyl)-N-methyl-2-(methylamino)acetamide (14.0 g, 6.56 mmol, 1 eq) in THF (5 mL) was added NaH (60% mineral oil, 2.09 g, 52.48 mmol, 8 eq) at 0° C. and stirred at same temperature for 15 minutes then a solution of (6-(chloromethyl)-1-m-tolyl-1H-pyrazolo[3,4-d]pyrimidin-4-ol) (0.18 g,6.56 mmol, 1 eq) in THF (5 mL) was added and stirred at 70° C. for 5 h. The reaction mixture was quenched with ice water, the organic product was extracted with EtOAc (3X25 mL). The combined organic extracts were dried over anhydrous Na₂SO₄. Solvent was distilled under reduced pressure to give the crude compound. The crude product was purified by reverse phase prep HPLC to afford 93 mg of (N-(2-chlorophenyl)-2-(((4-hydroxy-1-m-tolyl-1H-pyrazolo[3,4-d]pyrimidin-6-yl)methyl)(methyl)amino)-N-methylacetamide) as white solid.

Yield: (0.09 g, 15%).

ES-MS [M-H] ⁻:449.23; Rt =1.87 min (Method-A).

¹H NMR (400 MHz, CDCl₃): δ 11.09 (s, 1 H), 8.22 (s, 1 H), 7.86-7.83 (m,2 H), 7.56-7.53 (m, 1 H), 7.40-7.34 (m, 3 H), 7.29-7.27 (m, 1 H), 7.16-7.14 (m,1 H), 3.79-3.67 (q, J =16.4 Hz, 2 H), 3.24 (s, 2 H), 3.16-3.05 (q, J=16.8 Hz, 2 H), 2.43 (s, 3H), 2.40 (s, 3 H).

Synthesis of Example 4:

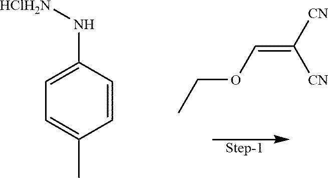

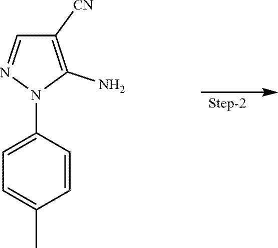

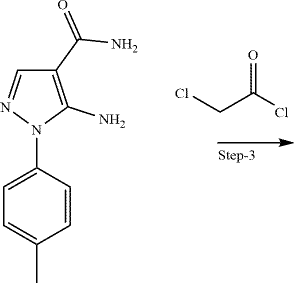

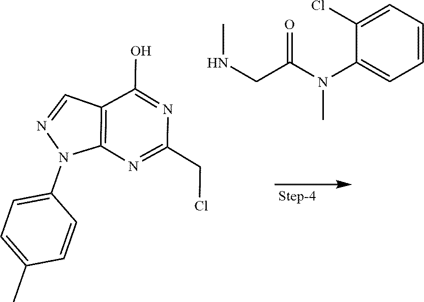

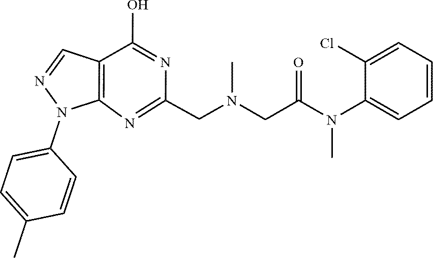 [SMILES: Cc1ccc(-n2ncc3c(O)nc(CN(C)CC(=O)N(C)c4ccccc4Cl)nc32)cc1]

Intermediate 11: (5-amino-1-p-tolyl-1H-pyrazole-4-carbonitrile):

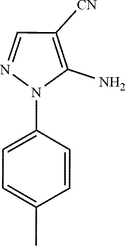

Prepared following procedure described for Intermediate 2.

Yield: (1.5 g, crude).

ES-MS [M+H]⁺: 198.98; Rt = 1.72 min (Method-A).

Intermediate 12: (5-amino-1-p-tolyl-1H-pyrazole-4-carboxamide):

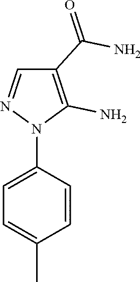 [SMILES: Cc1ccc(-n2ncc(C(N)=O)c2N)cc1]

Prepared following procedure described for Intermediate 3.

Yield: (0.35 g, 18%).

ES-MS [M+H] ⁺: 217.01; Rt = 1.50 min (Method-A).

Intermediate 13: (6-(chloromethyl)-1-p-tolyl-1H-pyrazolo[3,4-d]pyrimidin-4-ol):

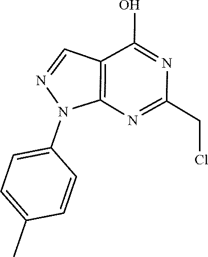 [SMILES: Cc1ccc(-n2ncc3c(O)nc(CCl)nc32)cc1]

Prepared following procedure described for Intermediate 4.

Yield: (0.25 g, 71%).

ES-MS [M+H] ⁺: 275.02; Rt = 1.86 min (Method-A).

Example 4: (N-(2-chlorophenyl)-2-(((4-hydroxy-1-p-tolyl-1H-pyrazolo[3,4-d]pyrimidin-6-yl)methyl)(methyl)amino)-N-methylacetamide):

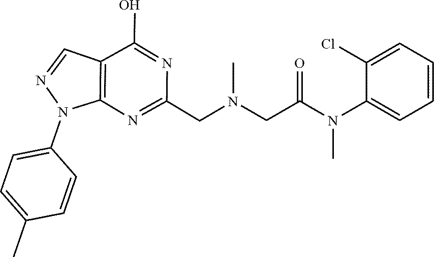 [SMILES: Cc1ccc(-n2ncc3c(O)nc(CN(C)CC(=O)N(C)c4ccccc4Cl)nc32)cc1]

To a solution of N-(2-chlorophenyl)-N-methyl-2-(methylamino)acetamide (0.19 g, 0.91 mmol, 1 eq) in THF (5 mL) was added NaH (60% in mineral oil, 0.05 g, 1.36 mmol, 1.5 eq) at 0° C. and stirred at same temperature for 15 minutes then solution of (6-(chloromethyl)-1-p-tolyl-1H-pyrazolo[3,4-d]pyrimidin-4-ol) (0.2 g, 0.91 mmol, 1 eq) in THF was added and stirred at 70° C. for 5 h. The reaction mixture was quenched with ice water, the organic product was extracted with EtOAc (3X25 mL). The combined organic extracts were dried over anhydrous Na₂SO₄. Solvent was distilled under reduced pressure to give the crude compound. The crude product was purified by reverse phase prep HPLC to afford 0.015 g of (N-(2-chlorophenyl)-2-(((4-hydroxy-1-p-tolyl-1H-pyrazolo[3,4-d]pyrimidin-6-yl)methyl)(methyl)amino)-N-methylacetamide) as off white solid.

Yield: (0.015 g, 3%).

ES-MS [M-H] ⁺: 449.17; Rt = 1.94 min (Method-A).

¹H NMR (400 MHz, DMSO-d₆): δ 10.61(s, 1 H), 7.89 (s, 1 H), 7.53-7.51 (m, 1 H), 7.45-7.43 (m, 2 H), 7.38-7.36 (m, 2 H), 7.26 (s, 3 H), 3.18 (s, 5 H), 3.03-2.91 (m, 2 H), 2.40(s, 3 H), 2.32 (s, 3 H).

Synthesis of Example 5:

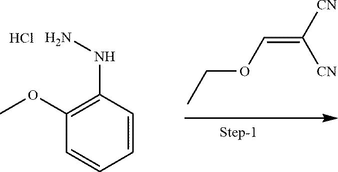

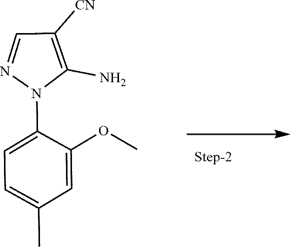

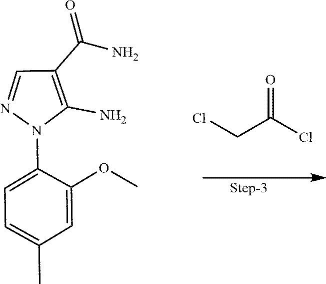

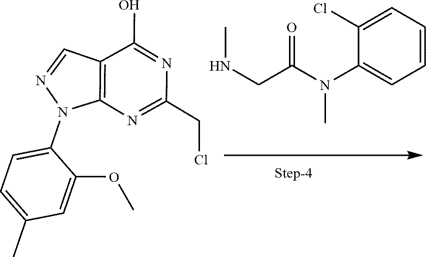

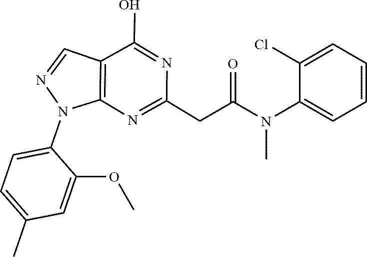 [SMILES: COc1cc(C)ccc1-n1ncc2c(O)nc(CC(=O)N(C)c3ccccc3Cl)nc21]

Intermediate 14: (5-amino-1-(2-methoxyphenyl)-1H-pyrazole-4-carbonitrile):

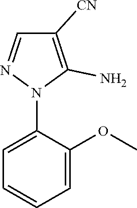 [SMILES: COc1ccccc1-n1ncc(C#N)c1N]

Prepared following procedure described for Intermediate 2.

Yield: (1.5 g, crude).

ES-MS [M+H]⁺: 215.03; Rt = 1.54 min (Method-A).

Intermediate 15: (5-amino-1-(2-methoxyphenyl)-1H-pyrazole-4-carboxamide):

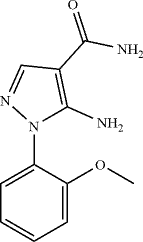 [SMILES: COc1ccccc1-n1ncc(C(N)=O)c1N]

Prepared following procedure described for Intermediate 3.

Yield: (0.35 g, 21%).

ES-MS [M+H] ⁺: 233.16; Rt = 1.91 min (Method-E).

Intermediate 16: (6-(chloromethyl)-1-(2-methoxyphenyl)-1H-pyrazolo[3,4-d]pyrimidin-4-ol):

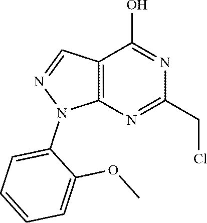 [SMILES: COc1ccccc1-n1ncc2c(O)nc(CCl)nc21]

Prepared following procedure described for Intermediate 4.

Yield: (0.25 g, 57%).

ES-MS [M+H] ⁺: 291.08; Rt = 1.72 min (Method-A).

Example 5: (N-(2-chlorophenyl)-2-(((4-hydroxy-1-(2-methoxyphenyl)-1H-pyrazolo[3,4-d]pyrimidin-6-yl)methyl)(methyl)amino)-N-methylacetamide):

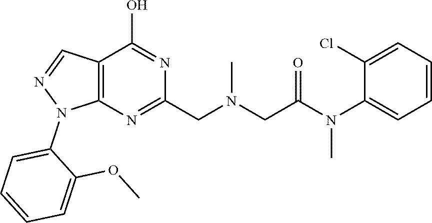 [SMILES: COc1ccccc1-n1ncc2c(O)nc(CN(C)CC(=O)N(C)c3ccccc3Cl)nc21]

To a solution of N-(2-chlorophenyl)-N-methyl-2-(methylamino)acetamide (0.14 g, 0.68 mmol, 1 eq)in THF (5 mL) was added NaH (60% in mineral oil, 0.04 g, 1.03 mmol, 1.5 eq) at 0° C. and stirred at same temperature for 15 minutes, added a solution of (6-(chloromethyl)-1-(2-methoxyphenyl)-1H-pyrazolo[3,4-d]pyrimidin-4-ol (0.2 g, 0.68 mmol, 1 eq) in THF and stirred at 70° C. for 5 h. The reaction mixture was quenched with ice water, the organic product was extracted with EtOAc (3X25 mL). The combined organic extracts were dried over anhydrous Na₂SO₄. Solvent was distilled under reduced pressure to give the crude compound. The crude product was purified by reverse phase prep HPLC to afford 0.015 g of (N-(2-chlorophenyl)-2-(((4-hydroxy-1-(2-methoxyphenyl)-1H-pyrazolo[3,4-d]pyrimidin-6-yl)methyl)(methyl)amino)-N-methylacetamide) as off white solid.

Yield: (0.015 g, 4%).

ES-MS [M-H] ⁺: 465.14; Rt = 1.84 min (Method-A).

¹H NMR (400 MHz, CDCl₃): δ 10.12 (s, 1 H), 7.92 (s, 1 H), 7.52-7.50 (m, 1 H), 7.46-7.42 (m, 2 H), 7.37-7.33 (m, 2 H), 7.19-7.21 (br s, 1 H), 7.10-7.05 (m, 2 H), 3.92 (s, 3 H), 3.15 (s, 5 H), 2.94-2.79 (m, 2 H), 2.21 (s, 3 H).

Synthesis of Example 6:

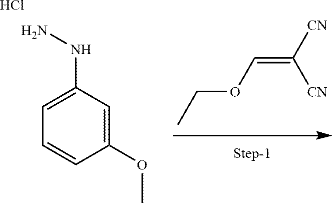

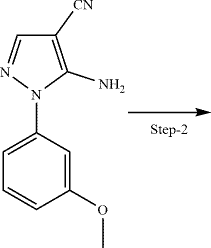

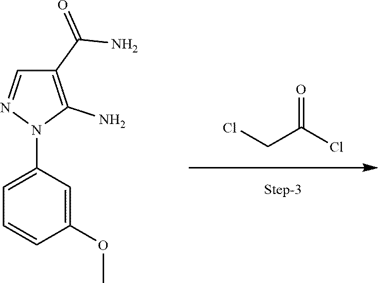

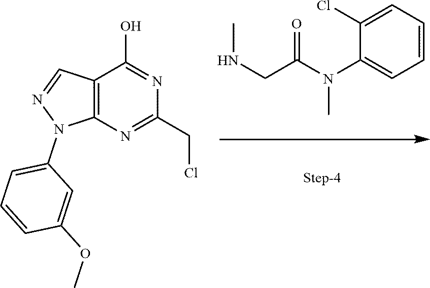

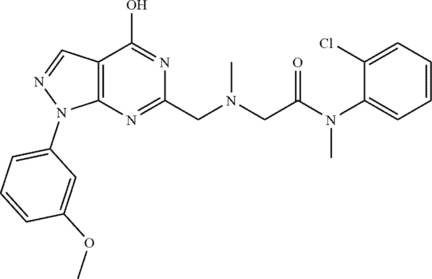 [SMILES: COc1cccc(-n2ncc3c(O)nc(CN(C)CC(=O)N(C)c4ccccc4Cl)nc32)c1]

Intermediate 17: (5-amino-1-(3-methoxyphenyl)-1H-pyrazole-4-carbonitrile):

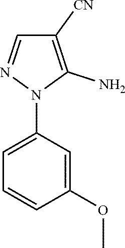 [SMILES: COc1cccc(-n2ncc(C#N)c2N)c1]

Prepared following procedure described for Intermediate 2.

Yield: (1.3 g, crude).

ES-MS [M+H]⁺: 215.14; Rt = 3.06 min (Method-F).

Intermediate 18: (5-amino-1-(3-methoxyphenyl)-1H-pyrazole-4-carboxamide):

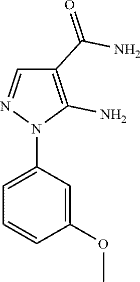 [SMILES: COc1cccc(-n2ncc(C(N)=O)c2N)c1]

Prepared following procedure described for Intermediate 3.

Yield: (0.7 g, 50%).

ES-MS [M+H] ⁺: 233.02; Rt = 1.43 min (Method-A).

Intermediate 19: (6-(chloromethyl)-1-(3-methoxyphenyl)-1H-pyrazolo[3,4-d]pyrimidin-4-ol):

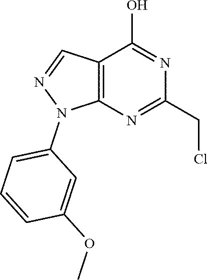 [SMILES: COc1cccc(-n2ncc3c(O)nc(CCl)nc32)c1]

Prepared following procedure described for Intermediate 4.

Yield: (0.25 g, 28%).

ES-MS [M+H] ⁺: 291.00; Rt = 1.76 min (Method-A).

Example 6: (N-(2-chlorophenyl)-2-(((4-hydroxy-1-(3-methoxyphenyl)-1H-pyrazolo[3,4-d]pyrimidin-6-yl)methyl)(methyl)amino)-N-methylacetamide):

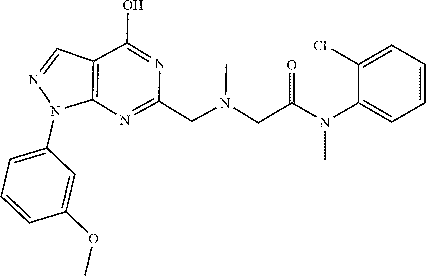 [SMILES: COc1cccc(-n2ncc3c(O)nc(CN(C)CC(=O)N(C)c4ccccc4Cl)nc32)c1]

To a solution of N-(2-chlorophenyl)-N-methyl-2-(methylamino)acetamide (0.14 g, 0.68 mmol, 1 eq) in THF (5 mL) was added NaH (60% in mineral oil, 0.04 g, 1.02 mmol, 1.5 eq) at 0° C. and stirred at same temperature for 15 minutes, added a solution of (6-(chloromethyl)-1-(3-methoxyphenyl)-1H-pyrazolo[3,4-d]pyrimidin-4-ol) (0.2 g, 0.68 mmol, 1 eq) in THF and stirred at 70° C. for 5 h. The reaction mixture was quenched with ice water, the organic product was extracted with EtOAc (3X25 mL). The combined organic extracts were dried over anhydrous Na₂SO₄. Solvent was distilled under reduced pressure to give the crude compound. The crude product was purified by reverse phase prep HPLC to afford 0.015 g of (N-(2-chlorophenyl)-2-(((4-hydroxy-1-(3-methoxyphenyl)-1H-pyrazolo[3,4-d]pyrimidin-6-yl)methyl)(methyl)amino)-N-methylacetamide) as off white solid.

Yield: (0.050 g, 12%).

ES-MS [M-H] ⁺: 465.28; Rt = 1.86 min (Method-A).

¹H NMR (400 MHz, CDCl₃): δ 10.71 (s, 1 H), 7.91 (s, 1 H), 7.54-7.51 (m, 1 H), 7.38-7.34 (m, 3 H), 7.31-7.27 (m, 1 H), 7.15-7.11 (m, 2 H), 6.99-6.96 (s, 1 H), 3.84 (s, 3 H), 3.21-3.18 (m, 5 H), 3.04-2.92 (q, J=16.8 Hz, 2 H), 2.32 (s, 3 H).

### Synthesis of Example 7:

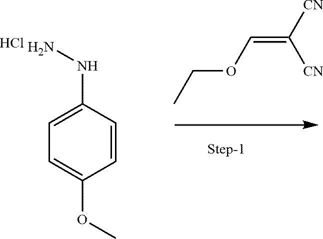

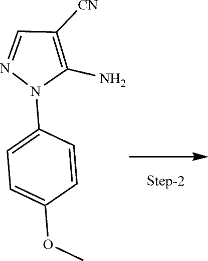

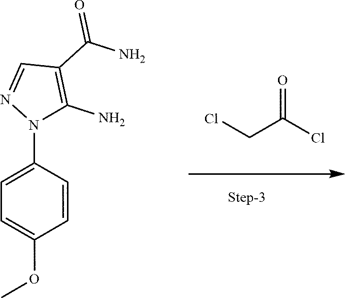

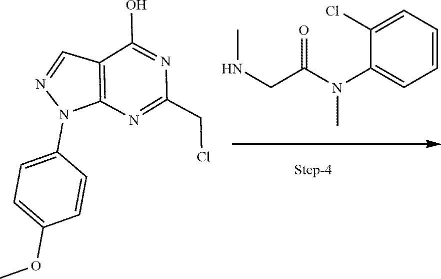

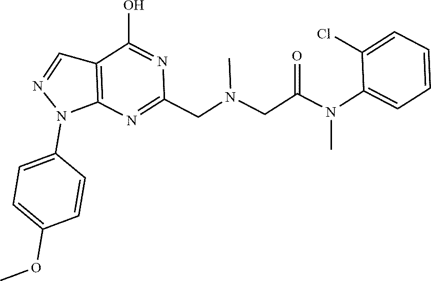 [SMILES: COc1ccc(-n2ncc3c(O)nc(CN(C)CC(=O)N(C)c4ccccc4Cl)nc32)cc1]

Intermediate 20: (5-amino-1-(4-methoxyphenyl)-1H-pyrazole-4-carbonitrile):

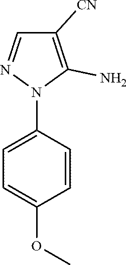 [SMILES: COc1ccc(-n2ncc(C#N)c2N)cc1]

Prepared following procedure described for Intermediate 2.

Yield: (1.3 g, crude).

ES-MS [M+H]⁺: 214.93; Rt = 1.61 min (Method-A).

Intermediate 21: (5-amino-1-(4-methoxyphenyl)-1H-pyrazole-4-carboxamide):

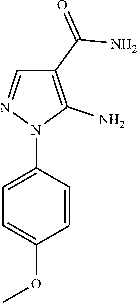 [SMILES: COc1ccc(-n2ncc(C(N)=O)c2N)cc1]

Prepared following procedure described for Intermediate 3.

Yield: (0.7 g, crude).

ES-MS [M+H] ⁺: 232.99; Rt = 1.39 min (Method-A).

Intermediate 22: (6-(chloromethyl)-1-(4-methoxyphenyl)-1H-pyrazolo[3,4-d]pyrimidin-4-ol):

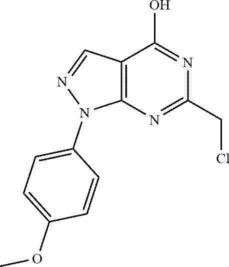 [SMILES: COc1ccc(-n2ncc3c(O)nc(CCl)nc32)cc1]

Prepared following procedure described for Intermediate 4.

Yield: (0.25 g, 28%).

ES-MS [M+H] ⁺: 289.02; Rt = 1.74 min (Method-A).

Example 7: (N-(2-chlorophenyl)-2-(((4-hydroxy-1-(4-methoxyphenyl)-1H-pyrazolo[3,4-d]pyrimidin-6-yl)methyl)(methyl)amino)-N-methylacetamide):

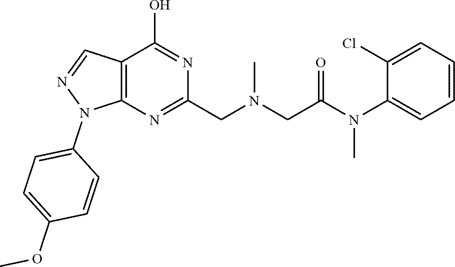 [SMILES: COc1ccc(-n2ncc3c(O)nc(CN(C)CC(=O)N(C)c4ccccc4Cl)nc32)cc1]

To a solution of N-(2-chlorophenyl)-N-methyl-2-(methylamino)acetamide (0.14 g, 0.68 mmol, 1 eq) in THF (5ml) was added NaH (60% in mineral oil, 0.04 g, 1.02 mmol, 1.5 eq) at 0° C. and stirred at same temperature for 15 minutes, added a solution of (6-(chloromethyl)-1-(4-methoxyphenyl)-1H-pyrazolo[3,4-d]pyrimidin-4-ol) (0.2 g, 0.68 mmol, 1 eq) in THF and stirred at 70° C. for 5 h. The reaction mixture was quenched with ice water, the organic product was extracted with EtOAc (3X25 mL). The combined organic extracts were dried over anhydrous Na₂SO₄. Solvent was distilled under reduced pressure to give the crude compound. The crude product was purified by reverse phase prep HPLC to afford 0.018 g of (N-(2-chlorophenyl)-2-(((4-hydroxy-1-(4-methoxyphenyl)-1H-pyrazolo[3,4-d]pyrimidin-6-yl)methyl)(methyl)amino)-N-methylacetamide) as off white solid.

Yield: (0.018 g, 5%).

ES-MS [M-H] ⁺: 465.14; Rt = 1.83 min (Method-A).

¹H NMR (400 MHz, CDCl₃): δ 10.59(s, 1 H), 7.89(s, 1 H), 7.53-7.51 (m, 1 H), 7.48-7.45(m, 2 H), 7.38-7.35(m, 2 H), 7.29-7.27(m, 1 H), 6.99-6.96 (m, 2 H), 3.85(s, 3 H), 3.20-3.18(m, 5 H), 3-02-2.90(m, 2 H), 2.32(s, 3 H).

### Examples 8-37

The following Examples 8-37 were purchased and tested in the differential scanning fluorimetry (DSF) assay as described below. The test results are provided in Table 2 below.

### Example 8

N-(2,6-dichlorophenyl)-2-(ethyl((4-oxo-3,4-dihydrothieno[3,2-d]pyrimidin-2-yl)methyl)amino)acetamide

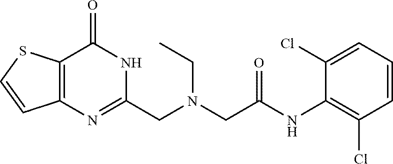 [SMILES: CCN(CC(=O)Nc1c(Cl)cccc1Cl)Cc1nc2ccsc2c(=O)[nH]1]

### Example 9

N-(3-fluorophenyl)-2-(methyl((4-oxo-3,4-dihydrothieno[3,2-d]pyrimidin-2-yl)methyl)amino)acetamide

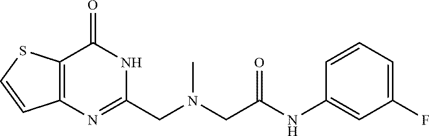 [SMILES: CN(CC(=O)Nc1cccc(F)c1)Cc1nc2ccsc2c(=O)[nH]1]

### Example 10

2-(ethyl((4-oxo-3,4-dihydrothieno[3,2-d]pyrimidin-2-yl)methyl)amino)-N-(3-methoxyphenyl)acetamide

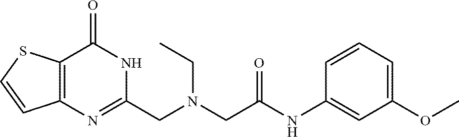 [SMILES: CCN(CC(=O)Nc1cccc(OC)c1)Cc1nc2ccsc2c(=O)[nH]1]

### Example 11

N-(2,4-difluorophenyl)-2-(((4-oxo-3,4-dihydrothieno[3,2-d]pyrimidin-2-yl)methyl)(propyl)amino)acetamide

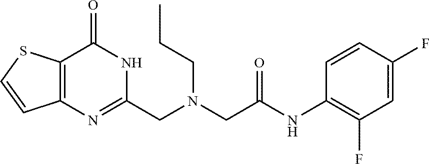 [SMILES: CCCN(CC(=O)Nc1ccc(F)cc1F)Cc1nc2ccsc2c(=O)[nH]1]

### Example 12

N-(2-chlorophenyl)-2-(methyl((4-oxo-3,4-dihydrothieno[3,2-d]pyrimidin-2-yl)methyl)amino)acetamide

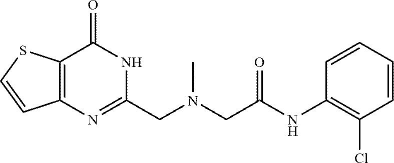 [SMILES: CN(CC(=O)Nc1ccccc1Cl)Cc1nc2ccsc2c(=O)[nH]1]

### Example 13

N-(tert-butyl)-2-(ethyl((4-oxo-3,4-dihydrothieno[3,2-d]pyrimidin-2-yl)methyl)amino)acetamide

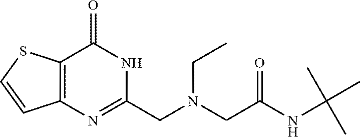 [SMILES: CCN(CC(=O)NC(C)(C)C)Cc1nc2ccsc2c(=O)[nH]1]

### Example 14

2-(methyl((4-oxo-3,4-dihydrothieno[3,2-d]pyrimidin-2-yl)methyl)amino)-N-(4-methylthiazol-2-yl)acetamide

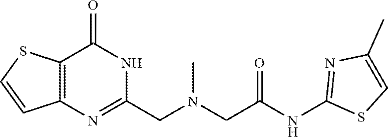 [SMILES: Cc1csc(NC(=O)CN(C)Cc2nc3ccsc3c(=O)[nH]2)n1]

### Example 15

2-(((2-(indolin-1-yl)-2-oxoethyl)(propyl)amino)methyl)thieno[3,2-d]pyrimidin-4(3H)-one

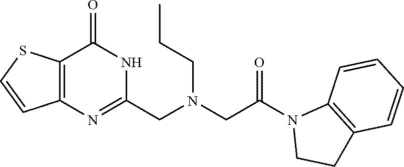 [SMILES: CCCN(CC(=O)N1CCc2ccccc21)Cc1nc2ccsc2c(=O)[nH]1]

### Example 16

N-(2,6-dichlorophenyl)-2-(ethyl((4-oxo-3,4-dihydroquinazolin-2-yl)methyl)amino)acetamide

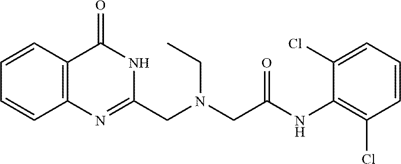 [SMILES: CCN(CC(=O)Nc1c(Cl)cccc1Cl)Cc1nc2ccccc2c(=O)[nH]1]

### Example 17

2-((2-methoxyethyl)((4-oxo-3,4-dihydroquinazolin-2-yl)methyl)amino)-N-(p-tolyl)acetamide

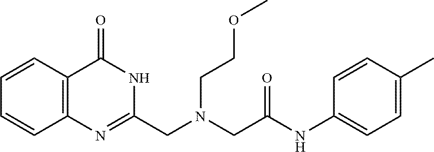 [SMILES: COCCN(CC(=O)Nc1ccc(C)cc1)Cc1nc2ccccc2c(=O)[nH]1]

### Example 18

2-(methyl((4-oxo-3,4-dihydroquinazolin-2-yl)methyl)amino)-N-(naphthalen-1-yl)acetamide

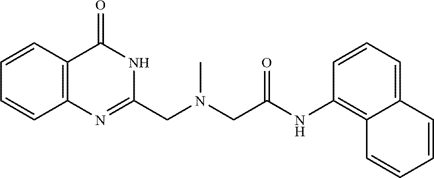 [SMILES: CN(CC(=O)Nc1cccc2ccccc12)Cc1nc2ccccc2c(=O)[nH]1]

### Example 19

2-(ethyl((4-oxo-3,4-dihydroquinazolin-2-yl)methyl)amino)-N-phenylacetamide

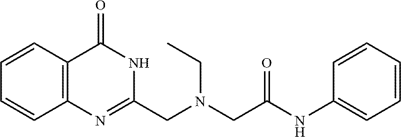 [SMILES: CCN(CC(=O)Nc1ccccc1)Cc1nc2ccccc2c(=O)[nH]1]

### Example 20

2-(ethyl((4-oxo-3,4-dihydroquinazolin-2-yl)methyl)amino)-N-(o-tolyl)acetamide

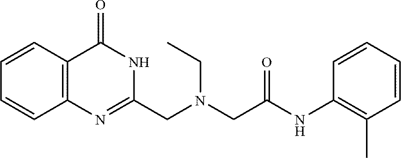 [SMILES: CCN(CC(=O)Nc1ccccc1C)Cc1nc2ccccc2c(=O)[nH]1]

### Example 21

2-(methyl((4-oxo-3,4-dihydroquinazolin-2-yl)methyl)amino)-N-(quinolin-5-yl)acetamide

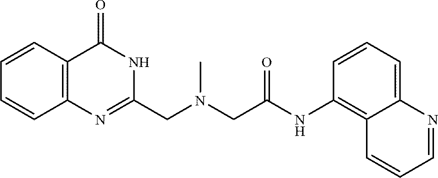 [SMILES: CN(CC(=O)Nc1cccc2ncccc12)Cc1nc2ccccc2c(=O)[nH]1]

### Example 22

N-(2-chlorophenyl)-2-(ethyl((4-oxo-3,4-dihydroquinazolin-2-yl)methyl)amino)acetamide

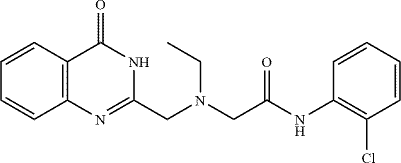 [SMILES: CCN(CC(=O)Nc1ccccc1Cl)Cc1nc2ccccc2c(=O)[nH]1]

### Example 23

N-(2,6-dimethylphenyl)-2-(methyl((4-oxo-3,4-dihydroquinazolin-2-yl)methyl)amino)acetamide

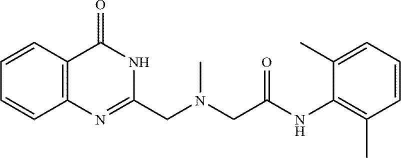 [SMILES: Cc1cccc(C)c1NC(=O)CN(C)Cc1nc2ccccc2c(=O)[nH]1]

### Example 24

1-((4-oxo-3,4-dihydroquinazolin-2-yl)methyl)-N-phenylpyrrolidine-2-carboxamide

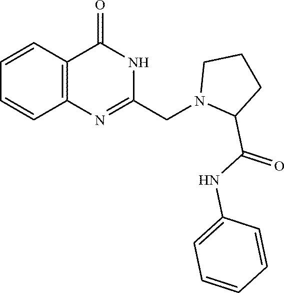 [SMILES: O=C(Nc1ccccc1)C1CCCN1Cc1nc2ccccc2c(=O)[nH]1]

### Example 25

N-(benzo[c][1,2,5]thiadiazol-4-yl)-2-(ethyl((4-oxo-3,4-dihydroquinazolin-2-yl)methyl)amino)acetamide

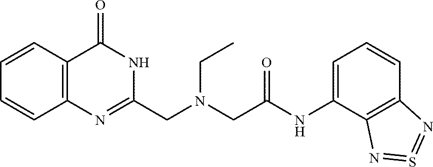 [SMILES: CCN(CC(=O)Nc1cccc2c1N=S=N2)Cc1nc2ccccc2c(=O)[nH]1]

### Example 26

2-(ethyl((4-oxo-3,4-dihydroquinazolin-2-yl)methyl)amino)-N-isobutylacetamide

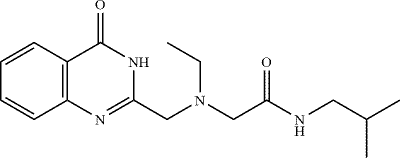 [SMILES: CCN(CC(=O)NCC(C)C)Cc1nc2ccccc2c(=O)[nH]1]

### Example 27

N-methyl-2-(methyl((4-oxo-3,4-dihydroquinazolin-2-yl)methyl)amino)-N-phenylacetamide

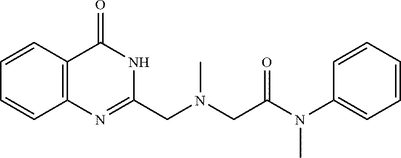 [SMILES: CN(CC(=O)N(C)c1ccccc1)Cc1nc2ccccc2c(=O)[nH]1]

### Example 28

N-ethyl-2-(methyl((4-oxo-3,4-dihydroquinazolin-2-yl)methyl)amino)-N-(naphthalen-1-yl)acetamide

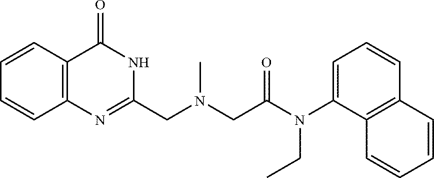 [SMILES: CCN(C(=O)CN(C)Cc1nc2ccccc2c(=O)[nH]1)c1cccc2ccccc12]

### Example 29

2-(((2-(3,4-dihydroquinolin-1(2H)-yl)-2-oxoethyl)(methyl)amino)methyl)quinazolin-4(3H)-one

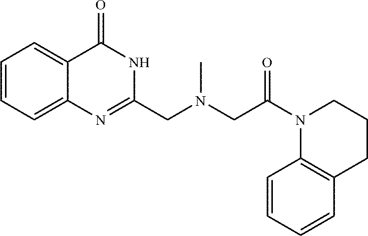 [SMILES: CN(CC(=O)N1CCCc2ccccc21)Cc1nc2ccccc2c(=O)[nH]1]

### Example 30

N-(3,4-dichlorophenyl)-2-(methyl((1-methyl-4-oxo-4,5-dihydro-1H-pyrazolo[3,4-d]pyrimidin-6-yl)methyl)amino)acetamide

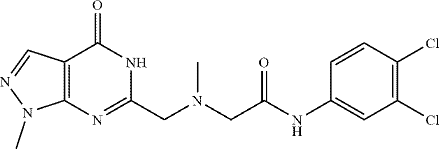 [SMILES: CN(CC(=O)Nc1ccc(Cl)c(Cl)c1)Cc1nc2c(cnn2C)c(=O)[nH]1]

### Example 31

2-(methyl((1-methyl-4-oxo-4,5-dihydro-1H-pyrazolo[3,4-d]pyrimidin-6-yl)methyl)amino)-N-(p-tolyl)acetamide

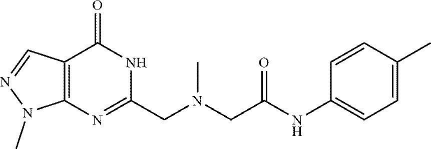 [SMILES: Cc1ccc(NC(=O)CN(C)Cc2nc3c(cnn3C)c(=O)[nH]2)cc1]

### Example 32

N-(3-fluorophenyl)-2-(methyl((1-methyl-4-oxo-4,5-dihydro-1H-pyrazolo[3,4-d]pyrimidin-6-yl)methyl)amino)acetamide

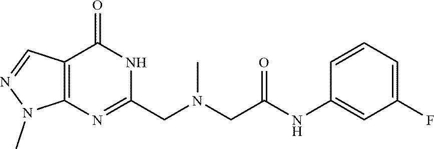 [SMILES: CN(CC(=O)Nc1cccc(F)c1)Cc1nc2c(cnn2C)c(=O)[nH]1]

### Example 33

2-(methyl((1-methyl-4-oxo-4,5-dihydro-1H-pyrazolo[3,4-d]pyrimidin-6-yl)methyl)amino)-N-phenylacetamide

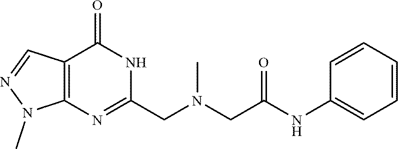 [SMILES: CN(CC(=O)Nc1ccccc1)Cc1nc2c(cnn2C)c(=O)[nH]1]

### Example 34

N-(2-methoxyethyl)-2-(methyl((1-methyl-4-oxo-4,5-dihydro-1H-pyrazolo[3,4-d]pyrimidin-6-yl)methyl)amino)acetamide

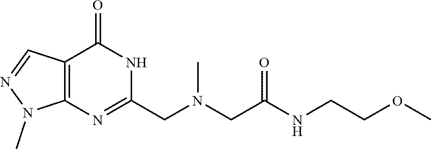 [SMILES: COCCNC(=O)CN(C)Cc1nc2c(cnn2C)c(=O)[nH]1]

### Example 35

1-methyl-6-((methyl(2-oxo-2-(pyrrolidin-1-yl)ethyl)amino)methyl)-1,5-dihydro-4H-pyrazolo[3,4-d]pyrimidin-4-one

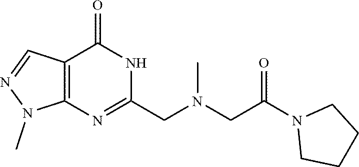 [SMILES: CN(CC(=O)N1CCCC1)Cc1nc2c(cnn2C)c(=O)[nH]1]

### Example 36

N,N-dimethyl-2-(methyl((1-methyl-4-oxo-4,5-dihydro-1H-pyrazolo[3,4-d]pyrimidin-6-yl)methyl)amino)acetamide

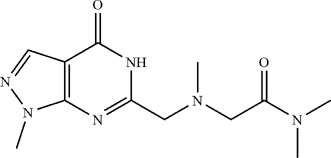 [SMILES: CN(CC(=O)N(C)C)Cc1nc2c(cnn2C)c(=O)[nH]1]

### Example 37

N-cyclohexyl-N-methyl-2-(methyl((1-methyl-4-oxo-4,5-dihydro-1H-pyrazolo[3,4-d]pyrimidin-6-yl)methyl)amino)acetamide

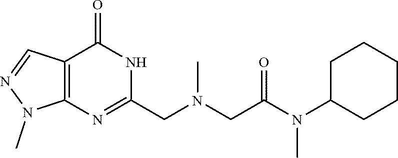 [SMILES: CN(CC(=O)N(C)C1CCCCC1)Cc1nc2c(cnn2C)c(=O)[nH]1]

### Synthesis of Examples 38-94

General Procedure A

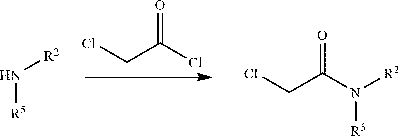

A mixture of the appropriate amine (1.0 eq.) and potassium carbonate (2 eq. or 1 eq.) in acetone (0.33M) was cooled down to 0° C. and stirred for 30 min. To the cold mixture, chloroacetyl chloride (1.5 eq. or 1 eq.) was added dropwise over vigorous stirring. The resulting mixture was warmed up and stirred at room temperature for 3 h or overnight. The reaction was quenched with water (70 mL) and the resulting suspension was cooled down to 0-5° C. and stirred for 1 h. The suspended solid was isolated by vacuum filtration and vacuum dried at 60° C. for 4 h to obtain the desired acetamide compound.

In same cases the reaction was quenched with water (75 mL) and the pH was adjusted to 7.0-8.0 with NaOH 3N. It was extracted with EtOAc (100 mL). The organic phase was dried over anhydrous sodium sulfate, filtered and concentrated to dryness. The reaction was quenched with water and the resulting suspended solid was stirred at room temperature for 1 h. The solid was isolated by vacuum filtration and vacuum dried at 60° C. overnight to obtain the corresponding 2-chloro acetamide as a solid which may be subsequently recrystallized in EtOAc/Hex.

Intermediate 23: 2-chloro-1-(isoindolin-2-yl)ethan-1-one:

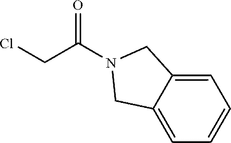 [SMILES: O=C(CCl)N1Cc2ccccc2C1]

Basification with NaOH 1N until pH = 4.5 - 5.0 was required to precipitate the acetamide. Appearance: dark brown solid.

Yield: (0.35 g, 36%).

ES-MS [M+H] ⁺: 196.0 / 198.1, Rt = 5.362 min (Method-G).

Intermediate 24: 2-chloro-1-(indolin-1-yl)ethan-1-one:

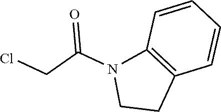 [SMILES: O=C(CCl)N1CCc2ccccc21]

Appearance: beige solid.

Yield: (0.92 g, 93%).

ES-MS [M+H] ⁺: 196.0 / 198.0, Rt = 5.618 min (Method-G).

Intermediate 25: 2-chloro-N-(4-methylthiazol-2-yl)acetamide:

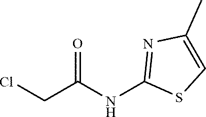

Isolated as a pale yellow solid.

Yield: (0.86 g, 51%).

ES-MS [M+H] ⁺: 191.0 / 192.9, Rt = 4.878 min (Method-G).

Intermediate 26: 2-chloro-N-(2,6-dichlorophenyl)acetamide:

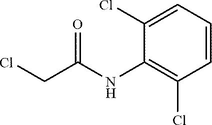 [SMILES: O=C(CCl)Nc1c(Cl)cccc1Cl]

Isolated as a white solid Yield: (0.45 g, 78%).

ES-MS [M+H] ⁺: 238.0 / 239.9, Rt = 5.260 min (Method-G).

Intermediate 27: 2-chloro-N-(2,6-dichlorophenyl)acetamide:

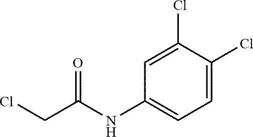 [SMILES: O=C(CCl)Nc1ccc(Cl)c(Cl)c1]

Isolated as an off-white solid.

Yield: (4.43 g, 97%).

ES-MS [M+H] ⁺: 236.2 / 238.2 / 240.1, Rt = 6.239 min (Method-G).

Intermediate 28: 2-chloro-N-(2-fluorophenyl)acetamide:

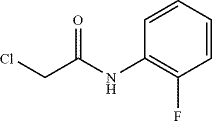 [SMILES: O=C(CCl)Nc1ccccc1F]

Isolated as an off-white solid.

Yield: (6.92 g, 82%).

ES-MS [M+H] ⁺: 188.0, Rt = 5.333 min (Method-G).

Intermediate 29: 2-chloro-N-methyl-N-(4-methylthiazol-2-yl)acetamide and 2-iodo-N-methyl-N-(4-methylthiazol-2-yl)acetamide:

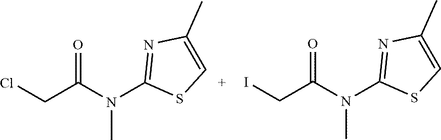

To an anhydrified solution of 2-chloro-N-(4-methylthiazol-2-yl)acetamide (3.0 g, 16 mmol, 1.0 eq.) in anhydrous THF (46 mL) NaH was added (0.48 g, 24 mmol, 1.25 eq.) and it was stirred at room temperature for 30 min. Then, iodomethane (1.27 mL, 20 mmol, 1.25 eq.) was added dropwise. The reaction mixture was stirred for 5 h. Since no conversion was observed, a 1 M solution of tBuOK in THF (8 mL, 8 mmol, 0.5 eq.) and iodomethane (1.27 mL, 20 mmol, 1.25 eq.) were added and stirred overnight. The reaction was quenched with water (40 mL) and the aqueous layer was extracted with ethyl acetate (3×). The organic layers were combined, dried over anhydrous sodium sulfate, filtered and distilled to dryness. The resultant crude was purified by flash column chromatography (EtOAc/Hex 20-60%) to obtain a ca. 3:1 mixture of 2-chloro-N-methyl-N-(4-methylthiazol-2-yl)acetamide and 2-iodo-N-methyl-N-(4-methylthiazol-2-yl)acetamide as a yellow solid.

Yield: (0.67 g, 18%).

ES-MS [M+H] ⁺: 205.0 / 207.0, Rt = 4.769 min (Method-G); chloride compound.

ES-MS [M+H] ⁺: 296.9, Rt = 5.163 min (Method-G); iodide compound.

Intermediate 30: 2-chloro-N-(2,6-dichlorophenyl)-N-methylacetamide and 2-iodo-N-(2,6-dichlorophenyl)-N-methylacetamide:

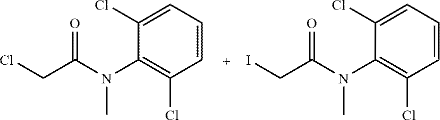

To an anhydrified solution of 2-chloro-N-(2,6-dichlorophenyl)acetamide (0.39 g, 1.63 mmol, 1 eq.) in THF (10 mL), NaH (0.04 g, 1.79 mmol, 1.25 eq.) was charged in portions. The mixture was cooled down to -78° C. and stirred for 30 minutes. To the cold reaction mixture, iodomethane (0.11 mL, 1.79 mmol, 1.25 eq.) was added drop-wise. The mixture was stirred for 5 h. The reaction was quenched with water (25 mL) and the aqueous layer was extracted with ethyl acetate (3×). The organic layers were combined, dried over anhydrous sodium sulphate, filtered and distilled to dryness to obtain a mixture of 2-chloro-N-(2,6-dichlorophenyl)-N-methylacetamide and 2-iodo-N-(2,6-dichlorophenyl)-N-methylacetamide isolated as a yellow oil.

Yield: (0.49 g).

ES-MS [M+H] ⁺: 251.9 / 253.9 / 256.0, Rt = 5.996 min (Method-G); chloride compound.

ES-MS [M+H] ⁺: 343.9 / 345.9, Rt = 6.197 min (Method-G); iodide compound.

Intermediate 31: 2-chloro-N-(3,4-dichlorophenyl)-N-methylacetamide:

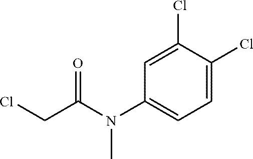

A 3 neck round bottom flask was charged with 2-chloro-N-(2,6-dichlorophenyl)acetamide (1.74 g, 7.30 mmol, 1 eq.) and it was dissolved in anhydrous DMF (73 mL) under N₂ atmosphere. The solution was cooled down to 0° C. and NaH (0.35 g, 8.75 mmol, 1.2 eq.) was added in portions. The resulting mixture was stirred at this temperature for 20 min and MeI (3.14 g, 21.89 mmol, 3 eq.) was added dropwise. The reaction mixture was stirred at 0° C. for 2 h. After this time, a saturated NH₄Cl aqueous solution was added carefully and the aqueous phase was extracted with EtOAc (3×). The organic layers were combined and washed twice with water, dried over anhydrous sodium sulfate, filtered and concentrated to dryness. The resultant crude was purified by flash column chromatography (Hexanes/EtOAc, 25%-100%) to obtain 2-chloro-N-(3,4-dichlorophenyl)-N-methylacetamide isolated as a yellow solid.

Yield: (1.46 g, 75% yield).

ES-MS [M+H] ⁺: 251.9 / 253.9, Rt = 6.251 min (Method-G).

Intermediate 32: 2-chloro-N-(2-fluorophenyl)-N-methylacetamide and 2-iodo-N-(2-fluorophenyl)-N-methylacetamide:

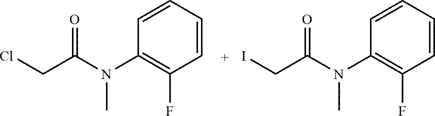

A 100 mL round bottom flask, provided with a magnetic stirrer and a condenser was charged with 2-chloro-N-(2-fluorophenyl)acetamide (4.50 g, 23.99 mmol, 1.0 eq.) and K₂CO₃ (9.95 g, 71.96 mmol, 3 eq.). A THF:DMF 9/1 mixture was added (68 mL), followed by addition of iodomethane (4.6 mL, 71.96 mmol, 3 eq.). The mixture was stirred at 45° C. overnight. The reaction was allowed to warm to room temperature. It was subsequently quenched with water (250 mL) and acidified with 3 N hydrochloric acid aqueous solution until pH = 7.0 - 7.2. The organic solvents were removed by distillation under reduced pressure and the aqueous layer was extracted with ethyl acetate (3×). The organic layers were combined, dried over anhydrous sodium sulfate, filtered and distilled to dryness. The resultant crude was purified by flash column chromatography (EtOAc/ Hexanes 20-50%) to obtain a ca. 1:3 mixture of 2-chloro-N-(2-fluorophenyl)-N-methylacetamide and 2-iodo-N-(2-fluorophenyl)-N-methylacetamide isolated as a yellow oil.

Yield: (1.27 g, 20%).

ES-MS [M+H] ⁺: 202.0, Rt = 5.441 min (Method-G); chloride compound.

ES-MS [M+H] ⁺: 293.9, Rt = 5.702 min (Method-G); iodide compound.

General procedure B

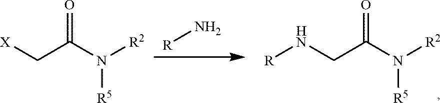

wherein X is Cl or I, and R is methyl or ethyl.

The corresponding amine solution 2 M in THF (methylamine and ethylamine, 10-20 eq.) or cyclopropylamine (10 eq.) dissolved in THF or propylamine (10 eq.) dissolved in THF was added over the mixture of 2-chloro-N-(2-chlorophenyl)-N-methylacetamide and 2-iodo-N-(2-chlorophenyl)-N-methylacetamide (1.0 eq.). The resulting mixture was stirred at room temperature for 16 h. The reaction mixture was concentrated to dryness and the crude was treated with ethyl acetate (50 mL). The organic layer was washed twice with water (25 mL), dried over anhydrous sodium sulphate, filtered and concentrated to dryness. The resultant crude was purified by flash column chromatography (EtOAc/Hexanes, 20%-50%) or (DCM/DCM:MeOH (90:10) (sometimes with NH₃ 2%), 0%-50%) to obtain the corresponding acetamides.

Intermediate 33: 1-(isoindolin-2-yl)-2-(methylamino)ethan-1-one:

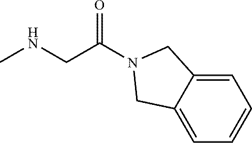

Appearance: dark brown solid.

Yield: (0.25 g, 73%).

ES-MS [M+H] ⁺: 191.0, Rt = 4.950 min (Method-G).

¹H NMR (400 MHz, Chloroform-d) δ 7.32 - 7.29 (m, 2 H), 7.28 - 7.23 (m, 2 H), 4.83 (s, 2 H), 4.78 (s, 2 H), 3.44 (s, 2 H), 2.50 (s, 3 H).

Intermediate 34: 1-(indolin-1-yl)-2-(methylamino)ethan-1-one:

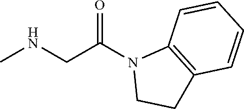

Appearance: pale brown solid.

Yield: (0.38 g, 77%).

ES-MS [M+H] ⁺: 191.1, Rt = 5.057 min (Method-G).

¹H NMR (400 MHz, Chloroform-d) δ 8.23 (d, J = 8.0 Hz, 1 H), 7.20 (ddd, J = 10.0, 7.5, 1.7 Hz, 2 H), 7.02 (td, J = 7.5, 1.1 Hz, 1 H), 4.00 (t, J = 8.5 Hz, 2 H), 3.46 (s, 2 H), 3.21 (t, J = 8.4 Hz, 2 H), 2.51 (s, 3 H)

Intermediate 35: N-(2-chlorophenyl)-N-methyl-2-(methylamino)acetamide:

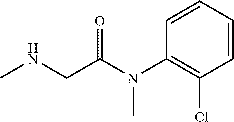

Isolated as a yellow oil.

Yield: (0.33 g, 52%).

ES-MS [M+H] ⁺: 213.0 / 215.0, Rt = 5.438 min (Method-G).

Intermediate 36: N-(2-chlorophenyl)-N-methyl-2-(ethylamino)acetamide:

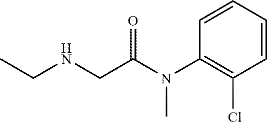

Isolated as a yellow oil.

Yield: (0.54 g, 73%).

ES-MS [M+H] ⁺: 227.1 / 229.0, Rt = 5.560 min (Method-G).

Intermediate 37: N-(2-chlorophenyl)-N-methyl-2-(cyclopropylamino)acetamide:

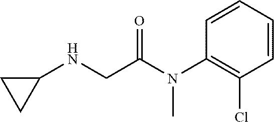 [SMILES: CN(C(=O)CNC1CC1)c1ccccc1Cl]

Isolated as a yellow oil.

Yield: (0.64 g, 93%).

ES-MS [M+H] ⁺: 239.1 / 241.1, Rt = 5.707 min (Method-G).

¹H NMR (400 MHz, DMSO-d₆) δ 7.69 - 7.63 (m, 1 H), 7.57 - 7.52 (m, 1 H), 7.50 -7.44 (m, 2 H), 3.10 (s, 3 H), 2.99 (d, J = 16.2 Hz, 1 H), 2.81 (d, J = 16.2 Hz, 1 H), 2.06 (qt, J = 6.7, 3.6 Hz, 1 H), 0.28 - 0.23 (m, 2 H), 0.09 - 0.05 (m, 2 H).

Intermediate 38: N-(2-chlorophenyl)-N-methyl-2-(propylamino)acetamide:

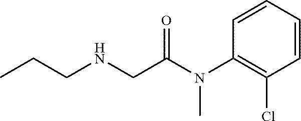 [SMILES: CCCNCC(=O)N(C)c1ccccc1Cl]

Isolated as a yellow oil.

Yield: (0.45 g, 58%).

ES-MS [M+H] ⁺: 241.1 / 243.1, Rt = 5.560 min (Method-G).

¹H NMR (400 MHz, DMSO-d₆) δ 7.68 - 7.63 (m, 1 H), 7.56 - 7.51 (m, 1 H), 7.49 -7.43 (m, 2 H), 3.10 (s, 3 H), 2.95 (d, J = 16.2 Hz, 1 H), 2.75 (d, J = 16.2 Hz, 1 H), 2.39 - 2.25 (m, 2 H), 1.30 (q, J = 7.3 Hz, 2 H), 0.79 (t, J = 7.4 Hz, 3 H).

Intermediate 39: N-(2,6-dichlorophenyl)-2-(ethylamino)-N-methylacetamide:

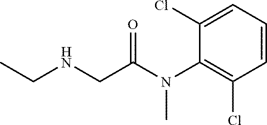 [SMILES: CCNCC(=O)N(C)c1c(Cl)cccc1Cl]

Isolated as a yellow oil.

Yield: (0.20 g, 40%).

ES-MS [M+H] ⁺: 261.1 / 263.0 / 265.0, Rt = 5.706 min (Method-G).

¹H NMR (400 MHz, DMSO-d₆) δ 7.68 (d, J = 8.2 Hz, 2 H), 7.53 - 7.49 (m, 1 H), 3.06 (s, 3 H), 2.84 (s, 2 H), 2.41 (q, J = 7.1 Hz, 2 H), 0.91 (t, J = 7.1 Hz, 3 H).

Intermediate 40: N-(3,4-dichlorophenyl)-N-methyl-2-(methylamino)acetamide:

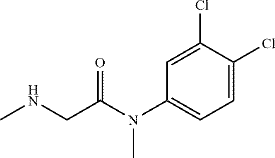

Yield: (0.66 g, 84%).

ES-MS [M+H] ⁺: 247.0 / 248.8, Rt = 5.838 min (Method-G).

¹H NMR (400 MHz, Chloroform-d) δ 7.50 (d, J = 8.5 Hz, 1 H), 7.35 (d, J = 2.4 Hz, 1 H), 7.09 (dd, J = 8.5, 2.5 Hz, 1 H), 3.26 (s, 3 H), 3.13 (s, 2 H), 2.39 (s, 3 H).

Intermediate 41: N-(2-fluorophenyl)-N-methyl-2-(methylamino)acetamide:

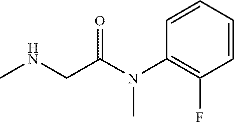

Isolated as a yellow oil.

Yield: (0.38 g, 45%).

ES-MS [M+H] ⁺: 197.0, Rt = 5.188 min (Method-G).

Intermediate 42: N-methyl-2-(methylamino)-N-(4-methylthiazol-2-yl)acetamide:

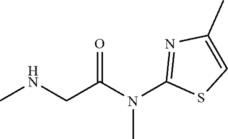

Methylamine solution 2M in THF (17 mL, 33.94 mmol, 15 eq.) was added over the mixture of 2-chloro-N-methyl-N-(4-methylthiazol-2-yl)acetamide and 2-iodo-N-methyl-N-(4-methylthiazol-2-yl)acetamide (0.67 g, 2.26 mmol, 1.0 eq.). The resulting mixture was stirred at room temperature for 5 h. The reaction mixture was concentrated to dryness and the crude was treated with ethyl acetate (50 mL). The organic layer was washed with 1M NaOH (25 mL) and water (25 mL), dried over anhydrous sodium sulfate, filtered and concentrated to dryness. The resultant crude was purified by flash column chromatography (DCM/DCM:MeOH (90:10) with NH₃ 2%, 0%-50%) to obtain N-methyl-2-(methylamino)-N-(4-methylthiazol-2-yl)acetamide as a yellow oil.

Yield: (0.115 g, 25%).

ES-MS [M+H] ⁺: 200.0, Rt = 4.837 min (Method-G).

Intermediate 43: N-methyl-2-(ethylamino)-N-(4-methylthiazol-2-yl)acetamide:

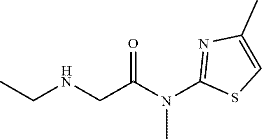

Ethylamine solution 2M in THF (3.2 mL, 31.06 mmol, 10 eq.) was added over the mixture of 2-chloro-N-methyl-N-(4-methylthiazol-2-yl)acetamide and 2-iodo-N-methyl-N-(4-methylthiazol-2-yl)acetamide (0.19 g, 0.64 mmol, 1.0 eq.) and it was stirred at room temperature for 16 h. The reaction mixture was concentrated to dryness and the crude was treated with DCM (50 mL). The organic layer was washed with water (15 mL), dried over anhydrous sodium sulfate, filtered and concentrated to dryness. The resultant crude was purified by flash column chromatography (DCM/DCM:MeOH (90:10) with NH₃ 2%, 0%-50%) to obtain N-methyl-2-(ethylamino)-N-(4-methylthiazol-2-yl)acetamide as a yellow solid.

Yield: (0.05 g, 36%).

ES-MS [M+H] ⁺: 214.1, Rt = 5.060 min (Method-G).

General Procedure C

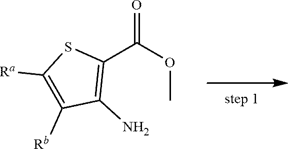

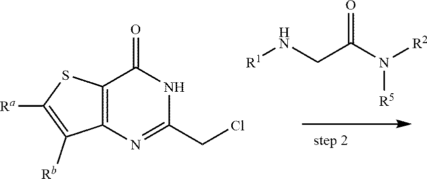

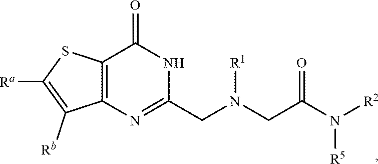

where R^{a} is methyl and R^{b} is H or R^{a} is H and R^{b} is methyl, and R¹, R², and R⁵ are as defined above.

### Step 1

To a stirred solution of methyl 3-amino-4-methylthiophene-2-carboxylate (1.0 eq.) or methyl 3-amino-5-methylthiophene-2-carboxylate (1.0 eq.) in hydrochloric acid 4 M in dioxane (6.4 eq.), chloroacetonitrile (1.3 eq.) was added. The resulting mixture was stirred for 15 h. The reaction mixture was concentrated to dryness and the crude was treated with water (15 mL) and basified with sodium bicarbonate until pH = 8.0. The resulting suspended solid was collected by vacuum filtration, washed with water and vacuum dried at 60° C. overnight to obtain 2-(chloromethyl)-7-methylthieno[3,2-d]pyrimidin-4(3H)-one or 2-(chloromethyl)-6-methylthieno[3,2-d]pyrimidin-4(3H)-one as a solid.

### Step 2

To a stirred mixture of 2-(chloromethyl)-7-methylthieno[3,2-d]pyrimidin-4(3H)-one or 2-(chloromethyl)-6-methylthieno[3,2-d]pyrimidin-4(3H)-one (1.0 eq.) and any of the intermediates 33-43 (1.0 eq.) in ethanol (1.5 mL), DIPEA (1.0 eq.) was added. The mixture was heated up to 90° C. and allowed to stir for 16 h. The reaction was quenched with water (10 mL). The aqueous layer was extracted with DCM (x3). The organic layers were combined, dried over anhydrous sodium sulphate, filtered and concentrated to dryness. The resulting crude was purified by flash column chromatography (DCM/DCM:MeOH (90:10), 0%-50%) to obtain the corresponding product as a solid.

Intermediate 44: 2-(chloromethyl)-7-methylthieno[3,2-d]pyrimidin-4(3H)-one:

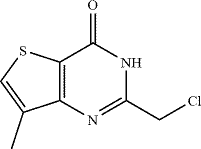

Isolated a grey solid.

Yield: (3.02 g, quantitative).

ES-MS [M+H] ⁺: 215.0 / 216.9, Rt = 4.820 min (Method-G).

¹H NMR (400 MHz, DMSO-d₆) δ 7.70 (d, J = 1.2 Hz, 1 H), 4.55 (s, 2 H), 2.29 (d, J = 1.2 Hz, 3 H).

Intermediate 45: 2-(chloromethyl)-7-methylthieno[3,2-d]pyrimidin-4(3H)-one:

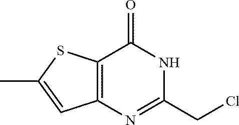

The reaction was performed at 60° C. Isolated as grey solid.

Yield: (1.10 g, 92%).

ES-MS [M+H] ⁺:215.0 / 216.9, Rt = 4.562 min (Method-G).

¹H NMR (400 MHz, DMSO-d₆) δ 12.73 (s, 1 H), 7.14 (q, J = 1.0 Hz, 1 H), 4.55 (s, 2 H), 2.59 (d, J = 1.2 Hz, 3 H).

### Example 38

2-(((isoindolin-2-yl)-2-oxoethyl)(methyl)amino)methyl)-7-methylthieno[3,2-d]pyrimidin-4(3H)-one

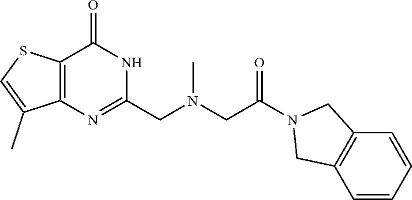 [SMILES: Cc1csc2c(=O)[nH]c(CN(C)CC(=O)N3Cc4ccccc4C3)nc12]

The product precipitated after treating the reaction mixture with water (2.5 mL). The suspended solid was isolated by vacuum filtration and vacuum dried at 60° C. overnight.

Yield: (0.058 g, 56%).

ES-MS [M+H] ⁺: 369.2 / 370.1, Rt = 17.100 min (Method-H).

¹H NMR (400 MHz, DMSO-d₆) δ 12.35 (s, 1 H), 7.80 (d, J = 1.3 Hz, 1 H), 7.40 -7.34 (m, 1 H), 7.31 (d, J = 2.8 Hz, 3 H), 4.83 (s, 2 H), 4.68 (s, 2 H), 3.68 (s, 2 H), 3.58 (s, 2 H), 2.44 (s, 3 H), 2.28 (d, J = 1.1 Hz, 3 H).

### Example 39

2-(((indolin-1-yl)-2-oxoethyl)(methyl)amino)methyl)-7-methylthieno[3,2-d]pyrimidin-4(3H)-one

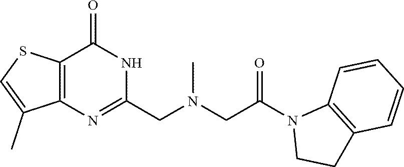 [SMILES: Cc1csc2c(=O)[nH]c(CN(C)CC(=O)N3CCc4ccccc43)nc12]

Yield: (0.023 g, 65%).

ES-MS [M+H] ⁺: 369.2, Rt = 17.760 min (Method-H).

¹H NMR (400 MHz, DMSO-d₆) δ 12.16 (s, 1 H), 8.09 (d, J = 8.0 Hz, 1 H), 7.80 (d, J = 1.2 Hz, 1 H), 7.33 - 7.21 (m, 1 H), 7.16 (td, J = 7.7, 1.4 Hz, 1 H), 7.00 (td, J = 7.4, 1.1 Hz, 1 H), 4.09 (t, J = 8.4 Hz, 2 H), 3.75 (s, 2 H), 3.64 (s, 2 H), 3.14 (t, J = 8.4 Hz, 2 H), 2.46 (s, 3 H), 2.29 (d, J = 1.1 Hz, 3 H).

### Example 40

2-(N-((3,4-dihydro-7-methyl-4-oxothieno[3,2-d]pyrimidin-2-yl)methyl)-N-methylamino)-N-(3,4-dichlorophenyl)-N-methylacetamide

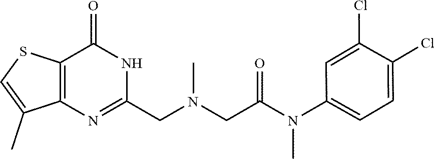 [SMILES: Cc1csc2c(=O)[nH]c(CN(C)CC(=O)N(C)c3ccc(Cl)c(Cl)c3)nc12]

Yield: (0.079 g, 80%).

ES-MS [M+H] ⁺: 425.1 / 426.9, Rt = 19.307 min (Method-H).

¹H NMR (400 MHz, DMSO-d₆) δ 12.06 (s, 1 H), 7.80 (d, J= 1.2 Hz, 1 H), 7.74 (s, 1 H), 7.66 (d, J= 8.6 Hz, 1 H), 7.39 (dd, J= 8.6, 2.5 Hz, 1 H), 3.64 (s, 2 H), 3.19 (s, 3 H), 2.50 (p, J= 1.9 Hz, 2 H), 2.36 (s, 3 H), 2.26 (d, J= 1.2 Hz, 3 H).

### Example 41

N-chlorophenyl)-N-methyl-2-(methyl((7-methyl-4-oxo-3,4-dihydrothieno[3,2-d]pyrimidin-2-yl)methyl)amino)acetamide

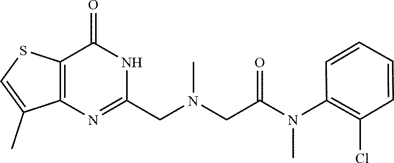 [SMILES: Cc1csc2c(=O)[nH]c(CN(C)CC(=O)N(C)c3ccccc3Cl)nc12]

The purification by flash column chromatography was performed using (DCM/EtOAc, 0%-50%) to obtain the corresponding product as a solid.

Yield: (0.041 g, 39%).

ES-MS [M+H] ⁺: 391.3 / 392.9, Rt = 17.978 min (Method-H).

¹H NMR (400 MHz, DMSO-d₆) δ 12.00 (s, 1 H), 7.79 (d, J = 1.4 Hz, 1 H), 7.61 -7.48 (m, 2 H), 7.39 (dd, J = 6.0, 3.5 Hz, 2 H), 3.58 (d, J = 1.5 Hz, 2 H), 3.27 - 3.15 (m, 1 H), 3.09 (s, 3 H), 3.01 (d, J = 16.4 Hz, 1 H), 2.33 (s, 3 H), 2.24 (d, J = 1.2 Hz, 3 H).

### Example 42

N-chlorophenyl)-2-(ethyl((7-methyl-4-oxo-3,4-dihydrothieno[3,2-d]pyrimidin-2-yl)methyl)amino)-N-methylacetamide

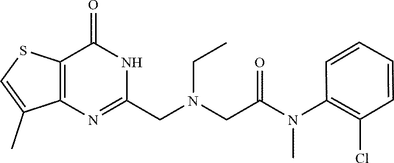 [SMILES: CCN(CC(=O)N(C)c1ccccc1Cl)Cc1nc2c(C)csc2c(=O)[nH]1]

Yield: (0.044 g, 47%).

ES-MS [M+H] ⁺: 405.1 / 407.1, Rt = 18.887 min (Method-H).

¹H NMR (400 MHz, DMSO-d₆) δ 12.11 (s, 1 H), 7.80 (q, J = 1.1 Hz, 1 H), 7.69 -7.60 (m, 1 H), 7.60 - 7.51 (m, 1 H), 7.50 - 7.38 (m, 2 H), 3.75 - 3.57 (m, 2 H), 3.29 - 3.21 (m, 1 H), 3.11 (s, 4 H), 2.63 (q, J = 7.2 Hz, 2 H), 2.26 (d, J = 1.2 Hz, 3 H), 0.85 (t, J = 7.1 Hz, 3 H).

### Example 43

N-chlorophenyl)-2-(cyclopropyl((7-methyl-4-oxo-3,4-dihydrothieno[3,2-d]pyrimidin-2-yl)methyl)amino)-N-methylacetamide

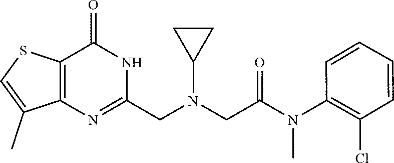 [SMILES: Cc1csc2c(=O)[nH]c(CN(CC(=O)N(C)c3ccccc3Cl)C3CC3)nc12]

Yield: (0.026 g, 33%).

ES-MS [M+H] ⁺: 417.1, Rt = 19.063 min (Method-H).

¹H NMR (400 MHz, DMSO-d₆) δ 12.03 (s, 1 H), 7.80 (d, J = 1.3 Hz, 1 H), 7.68 -7.57 (m, 1 H), 7.58 - 7.48 (m, 1 H), 7.49 - 7.31 (m, 2 H), 3.86 (d, J = 4.0 Hz, 2 H), 3.25 -3.13 (m, 2 H), 3.10 (s, 3 H), 2.40 (dq, J = 6.5, 3.4 Hz, 1 H), 2.26 (d, J = 1.1 Hz, 3 H), 0.30 (dd, J = 6.6, 2.4 H_{z}, 2 H), 0.20 - 0.15 (m, 2 H).

### Example 44

N-chlorophenyl)-N-methyl-2-(((7-methyl-4-oxo-3,4-dihydrothieno[3,2-d]pyrimidin-2-yl)methyl)(propyl)amino)acetamide

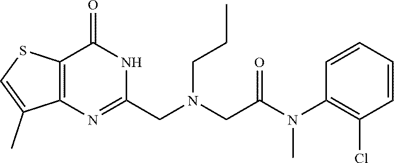 [SMILES: CCCN(CC(=O)N(C)c1ccccc1Cl)Cc1nc2c(C)csc2c(=O)[nH]1]

Yield: (0.042 g, 43%).

ES-MS [M+H] ⁺: 419.1 / 421.0, Rt = 19.756 min (Method-H).

¹H NMR (400 MHz, DMSO-d₆) δ 12.13 (s, 1 H), 7.80 (t, J = 1.2 Hz, 1 H), 7.70 -7.61 (m, 1 H), 7.60 - 7.53 (m, 1 H), 7.45 - 7.45 (m, 2 H), 3.79 - 3.58 (m, 2 H), 3.27 (d, J = 7.2 Hz, 1 H), 3.12 (s, 3H), 3.07 (d, J= 16.9 Hz, 1 H), 2.62 - 2.51 (m, 2 H), 2.26 (d, J = 1.1 Hz, 3 H), 1.24 (h, J= 7.3 Hz, 2 H), 0.70 (t, J= 7.3 Hz, 3 H).

### Example 45

N-chlorophenyl)-N-methyl-2-(methyl((6-methyl-4-oxo-3,4-dihydrothieno[3,2-d]pyrimidin-2-yl)methyl)amino)acetamide

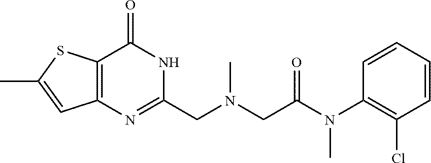 [SMILES: Cc1cc2nc(CN(C)CC(=O)N(C)c3ccccc3Cl)[nH]c(=O)c2s1]

Yield: (0.059, 64%).

ES-MS [M+H] ⁺: 391.1 / 392.9, Rt = 17.404 min (Method-H).

¹H NMR (400 MHz, DMSO-d₆) δ 11.73 (s, 1 H), 7.67 - 7.56 (m, 1 H), 7.53 (dd, J = 6.0, 3.5 Hz, 1 H), 7.41 (dd, J = 6.0, 3.6 Hz, 2 H), 7.05 (d, J = 1.1 Hz, 1 H), 3.54 (s, 2 H), 3.18 (d, J = 16.4 Hz, 1 H), 3.09 (s, 3 H), 2.97 (d, J = 16.5 Hz, 1 H), 2.57 (d, J = 1.1 Hz, 3 H), 2.31 (s, 3 H).

### Example 46

N-chlorophenyl)-2-(ethyl((6-methyl-4-oxo-3,4-dihydrothieno[3,2-d]pyrimidin-2-yl)methyl)amino)-N-methylacetamide

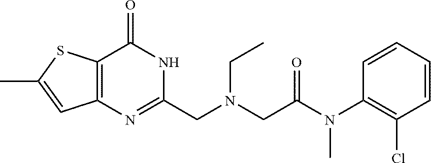 [SMILES: CCN(CC(=O)N(C)c1ccccc1Cl)Cc1nc2cc(C)sc2c(=O)[nH]1]

Yield: (0.040, 42%).

ES-MS [M+H] ⁺: 405.3 / 407.0, Rt = 18.331 min (Method-H).

¹H NMR (400 MHz, DMSO-d₆) δ 12.01 (s, 1 H), 7.63 (dd, J= 5.9, 3.6 Hz, 1 H), 7.57 (dd, J = 6.1, 3.4 Hz, 1 H), 7.44 (dd, J = 6.0, 3.6 Hz, 2 H), 7.05 (d, J = 1.2 Hz, 1 H), 3.74 -3.55 (m, 2 H), 3.31- 3.17 (m, 1 H), 3.11 (s, 3 H), 3.03 (d, J= 16.8 Hz, 1 H), 2.72 - 2.58 (m, 2 H), 2.57 (d, J = 1.1 Hz, 3 H), 0.82 (t, J = 7.2 Hz, 3 H).

### Example 47

N-chlorophenyl)-2-(cyclopropyl((6-methyl-4-oxo-3,4-dihydrothieno[3,2-d]pyrimidin-2-yl)methyl)amino)-N-methylacetamide

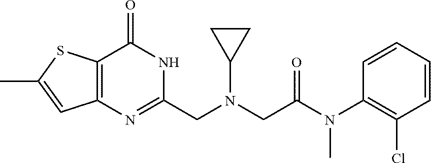 [SMILES: Cc1cc2nc(CN(CC(=O)N(C)c3ccccc3Cl)C3CC3)[nH]c(=O)c2s1]

Yield: (0.070 g, 72%).

ES-MS [M+H] ⁺: 417.2, Rt = 18.917 min (Method-H).

¹H NMR (400 MHz, DMSO-d₆) δ 11.95 (s, 1 H), 7.67 - 7.60 (m, 1 H), 7.58 - 7.51 (m, 1 H), 7.49 - 7.39 (m, 2 H), 7.07 (d, J = 1.1 Hz, 1 H), 3.90 - 3.72 (m, 2 H), 3.28 (d, J = 6.9 Hz, 1 H), 3.13 (d, J = 16.7 Hz, 1 H), 3.10 (s, 3 H), 2.58 (d, J = 1.1 Hz, 3 H), 2.38 (tt, J = 6.7, 3.6 Hz, 1 H), 0.33 - 0.22 (m, 2 H), 0.22 - 0.11 (m, 2 H).

### Example 48

N-methyl-2-(methyl((7-methyl-4-oxo-3,4-dihydrothieno[3,2-d]pyrimidin-2-yl)methyl)amino)-N-(4-methylthiazol-2-yl)acetamide

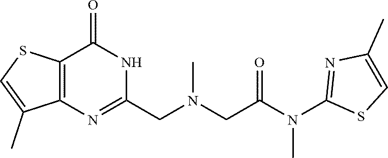 [SMILES: Cc1csc(N(C)C(=O)CN(C)Cc2nc3c(C)csc3c(=O)[nH]2)n1]

Yield: (0.046 g, 54%).

ES-MS [M+H] ⁺: 378.1 / 379.0 / 380.0, Rt = 15.695 min (Method-H).

¹H NMR (400 MHz, DMSO-d₆) δ 11.95 (bs, 1 H), 7.80 (s, 1 H), 6.65 (s, 1 H), 3.74 (s, 2 H), 3.63 (s, 3 H), 3.54 (s, 2 H), 2.43 (s, 3 H), 2.29 (s, 6 H).

### Example 49

N-(2,6-dichlorophenyl)-2-(ethyl((7-methyl-4-oxo-3,4-dihydrothieno[3,2-d]pyrimidin-2-yl)methyl)amino)-N-methylacetamide

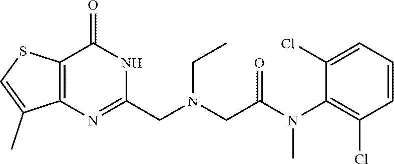 [SMILES: CCN(CC(=O)N(C)c1c(Cl)cccc1Cl)Cc1nc2c(C)csc2c(=O)[nH]1]

Yield: (0.042 g, 48%).

ES-MS [M+H] ⁺: 439.2 / 441.2, Rt = 5.801 min (Method-J).

¹H NMR (400 MHz, DMSO-d₆) δ 12.02 (s, 1 H), 7.80 (d, J = 1.2 Hz, 1 H), 7.64 -7.55 (m, 2 H), 7.44 (dd, J = 8.7, 7.6 Hz, 1 H), 3.67 (s, 2 H), 3.19 (s, 2 H), 3.06 (s, 3 H), 2.68 (q, J = 7.2 Hz, 2 H), 2.25 (d, J = 1.1 Hz, 3 H), 0.88 (t, J = 7.1 Hz, 3 H).

### Example 50

N-fluorophenyl)-N-methyl-2-(methyl((7-methyl-4-oxo-3,4-dihydrothieno[3,2-d]pyrimidin-2-yl)methyl)amino)acetamide

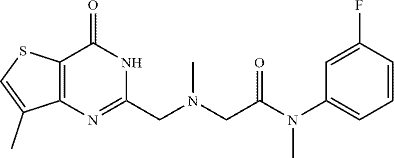 [SMILES: Cc1csc2c(=O)[nH]c(CN(C)CC(=O)N(C)c3cccc(F)c3)nc12]

Yield: (0.061 g, 66%).

ES-MS [M+H] ⁺: 375.1 / 376.1, Rt = 17.272 min (Method-H).

¹H NMR (400 MHz, DMSO-d₆) δ 12.04 (s, 1 H), 7.80 (t, J = 1.2 Hz, 1 H), 7.44 (td, J = 8.2, 6.6 Hz, 1 H), 7.31 (d, J = 10.2 Hz, 1 H), 7.20 (ddd, J = 10.0, 5.5, 1.8 Hz, 2 H), 3.62 (s, 2 H), 3.32 (s, 2 H), 3.19 (s, 3 H), 2.34 (s, 3 H), 2.26 (d, J = 1.1 Hz, 3 H).

### Example 51

N-fluorophenyl)-N-methyl-2-(methyl((6-methyl-4-oxo-3,4-dihydrothieno[3,2-d]pyrimidin-2-yl)methyl)amino)acetamide

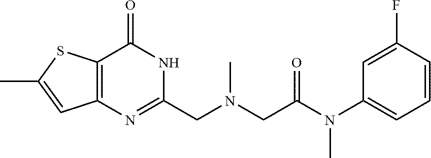 [SMILES: Cc1cc2nc(CN(C)CC(=O)N(C)c3cccc(F)c3)[nH]c(=O)c2s1]

Yield: (0.063 g, 72%).

ES-MS [M+H] ⁺: 375.1 Rt = 16.763 min (Method-H).

¹H NMR (400 MHz, DMSO-d₆) δ 11.96 (s, 1 H), 7.45 (td, J = 8.2, 6.6 Hz, 1 H), 7.31 (d, J = 10.2 Hz, 1 H), 7.24 - 7.13 (m, 2 H), 7.07 (d, J = 1.2 Hz, 1 H), 3.57 (s, 2 H), 3.28 (s, 2 H), 3.19 (s, 3 H), 2.57 (d, J = 1.1 Hz, 3 H), 2.32 (s, 3 H).

### Example 52

2-(((indolin-1-yl)-2-oxoethyl)(methyl)amino)methyl)-6-methylthieno[3,2-d]pyrimidin-4(3H)-one

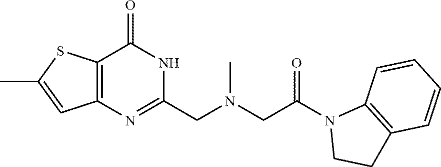 [SMILES: Cc1cc2nc(CN(C)CC(=O)N3CCc4ccccc43)[nH]c(=O)c2s1]

Yield: (0.048 g, 50%).

ES-MS [M+H] ⁺: 369.2, Rt = 16.151 min (Method-H).

¹H NMR (400 MHz, DMSO-d₆) δ 12.09 (s, 1 H), 8.08 (d, J = 8.0 Hz, 1 H), 7.24 (d, J = 7.3 Hz, 1 H), 7.20 - 7.12 (m, 1 H), 7.08 (t, J = 1.1 Hz, 1 H), 7.00 (td, J = 7.4, 1.1 Hz, 1 H), 4.07 (t, J = 8.5 Hz, 2 H), 3.69 (s, 2 H), 3.62 (s, 2 H), 3.14 (t, J = 8.4 Hz, 2 H), 2.57 (d, J = 1.1 Hz, 3 H), 2.44 (s, 3 H).

### Example 53

2-(((isoindolin-2-yl)-2-oxoethyl)(methyl)amino)methyl)-6-methylthieno[3,2-d]pyrimidin-4(3H)-one

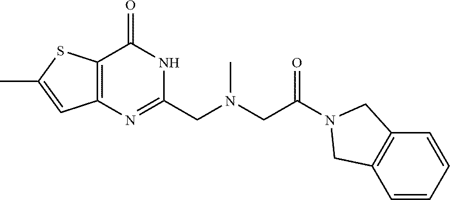 [SMILES: Cc1cc2nc(CN(C)CC(=O)N3Cc4ccccc4C3)[nH]c(=O)c2s1]

Yield: (0.035 g, 50%).

ES-MS [M+H] ⁺: 369.2, Rt = 16.538 min (Method-H).

¹H NMR (400 MHz, DMSO-d₆) δ 12.26 (s, 1 H), 7.50 - 7.23 (m, 4 H), 7.07 (s, 1 H), 4.82 (s, 2 H), 4.67 (s, 2 H), 3.63 (s, 2 H), 3.55 (s, 2 H), 2.57 (s, 3 H), 2.41 (s, 3 H).

### Example 54

N-chlorophenyl)-N-methyl-2-(((6-methyl-4-oxo-3,4-dihydrothieno[3,2-d]pyrimidin-2-yl)methyl)(propyl)amino)acetamide

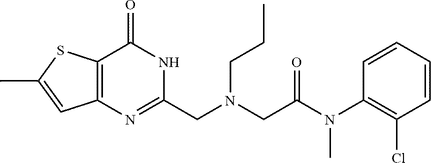 [SMILES: CCCN(CC(=O)N(C)c1ccccc1Cl)Cc1nc2cc(C)sc2c(=O)[nH]1]

Yield: (0.038 g, 33%).

ES-MS [M+H] ⁺: 419.1 / 421.0, Rt = 19.207 min (Method-H).

¹H NMR (400 MHz, DMSO-d₆) δ 12.06 (s, 1 H), 7.68 - 7.62 (m, 1 H), 7.61 - 7.54 (m, 1 H), 7.47 (m, 02 H), 7.06 (d, J = 1.2 Hz, 1 H), 3.76 - 3.54 (m, 2 H), 3.29 - 3.22 (d, J = 16.9 Hz, 1 H), 3.12 (s, 3 H), 3.04 (d, J = 16.9 Hz, 1 H), 2.57 (d, J = 1.1 Hz, 3 H), 2.53 (m, 2 H), 1.22 (m, 2 H), 0.69 (t, J = 7.3 Hz, 3 H).

### Example 55

N-(3,4-dichlorophenyl)-N-methyl-2-(methyl((6-methyl-4-oxo-3,4-dihydrothieno[3,2-d]pyrimidin-2-yl)methyl)amino)acetamide

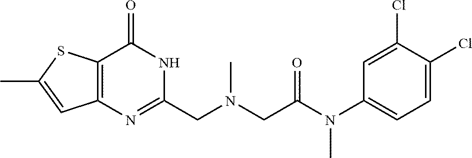 [SMILES: Cc1cc2nc(CN(C)CC(=O)N(C)c3ccc(Cl)c(Cl)c3)[nH]c(=O)c2s1]

Yield: (0.069 g, 69%).

ES-MS [M+H] ⁺: 425.1 / 426.9. Rt = 18.791 min (Method-H).

¹H NMR (400 MHz, DMSO-d₆) δ 11.96 (s, 1 H), 7.73 (s, 1 H), 7.67 (d, J = 8.6 Hz, 1 H), 7.38 (dd, J= 8.6, 2.5 Hz, 1 H), 7.06 (d, J = 1.3 Hz, 1 H), 3.57 (s, 2 H), 3.33 (s, 2 H), 3.18 (s, 3 H), 2.57 (d, J = 1.1 Hz, 3 H), 2.33 (s, 3 H).

General Procedure D

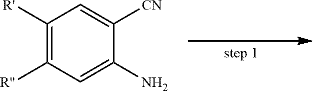

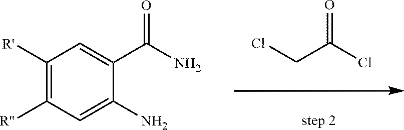

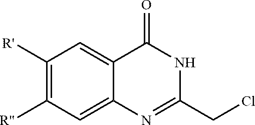

### Step 1

The corresponding benzonitrile (1.0 eq.) was dissolved in sulphuric acid (1.2 M). The reaction mixture was heated up to 50° C. and stirred for 2 h. The reaction was quenched by slowly pouring the mixture into cold water (100 mL) followed by a basification with NaOH 25% w/w until precipitation of solid was observed. The suspension was stirred at 0° C. for 1 h and the product was isolated by vacuum filtration and vacuum dried at 60° C. overnight, to obtain the corresponding amide as a solid.

### Step 2

To a mixture of the corresponding benzamide (1.0 eq.) in acetic acid (1 M), chloroacetyl chloride (2.0 eq.) was added. The mixture was heated up to reflux and stirred for 2 h. The reaction mixture was cooled down to room temperature and slowly poured into cold water (50 mL). The suspended solid was stirred at 0° C. for 30 minutes, isolated by vacuum filtration and vacuum dried at 60° C. overnight, to obtain the corresponding chloride as a solid.

Intermediate 46: 2-amino-5-fluorobenzamide:

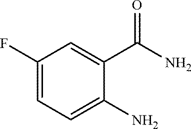 [SMILES: NC(=O)c1cc(F)ccc1N]

Precipitation of the amide observed at pH = 2.8 - 3.2. White solid.

Yield: (1.03 g, 61%).

ES-MS [M+H] ⁺: 138.0 / 155.1, Rt = 3.252 min (Method-G).

Intermediate 47: 2-amino-4-fluorobenzamide:

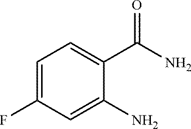 [SMILES: NC(=O)c1ccc(F)cc1N]

Precipitation of the amide observed at pH = 1.8 - 2.0. Beige solid.

Yield: (0.30 g, 52%).

ES-MS [M+H] ⁺: 138.0 / 155.0, Rt = 3.818 min (Method-G).

Intermediate 48: 2-amino-5-methylbenzamide:

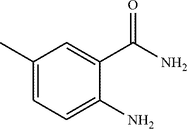

The reaction was performed at room temperature.

Yield: (0.50 g, 88%).

ES-MS [M+H] ⁺: 134.0 / 151.1, Rt = 4.229 min (Method-G).

Intermediate 49: 2-amino-4-methylbenzamide:

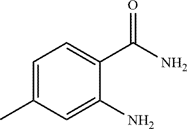

The reaction was performed at room temperature.

Yield: (1.09 g, 99%).

ES-MS [M+H] ⁺: 134.0 / 151.1, Rt = 4.190 min (Method-G).

Intermediate 50: 2-(chloromethyl)-6-fluoroquinazolin-4(3H)-one:

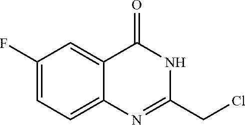 [SMILES: O=c1[nH]c(CCl)nc2ccc(F)cc12]

Yield: (1.50 g, 97%).

ES-MS [M+H] ⁺: 213.0 / 215.0, Rt = 4.900 min (Method-G).

¹H NMR (400 MHz, DMSO-d₆) δ 12.72 (s, 1 H), 7.85 - 7.68 (m, 3 H), 4.55 (s, 2 H).

Intermediate 51: 2-(chloromethyl)-7-fluoroquinazolin-4(3H)-one:

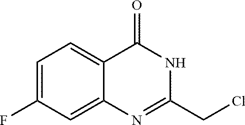 [SMILES: O=c1[nH]c(CCl)nc2cc(F)ccc12]

The reaction was maintained at reflux for 3 h.

Yield: (0.26 g, 70%).

ES-MS [M+H] ⁺: 213.0 / 215.0, Rt = 5.011 min (Method-G).

¹H NMR (400 MHz, DMSO-d₆) δ 12.68 (s, 1 H), 8.18 (dd, J = 8.8, 6.3 Hz, 1 H), 7.49 (dd, J = 10.1, 2.5 Hz, 1H), 7.42 (td, J = 8.7, 2.6 Hz, 1 H), 4.54 (s, 2 H).

Intermediate 52: 2-(chloromethyl)-6-methylquinazolin-4(3H)-one:

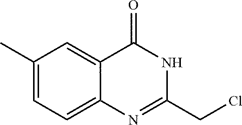

The solid was treated with methanol (10 mL) at room temperature for 30 minutes. The suspended solid was isolated by vacuum filtration and vacuum dried at 60° C. for 4 h, to obtain the product as an off-white solid.

Yield: (0.34 g, 50%).

ES-MS [M+H] ⁺: 209.1 / 211.0, Rt = 5.211 min (Method-G).

¹H NMR (400 MHz, DMSO-d₆) δ 12.47 (s, 1 H), 8.00 (d, J = 8.1 Hz, 1 H), 7.49 (s, 1 H), 7.37 (dd, J = 8.1, 1.7 Hz, 1 H), 4.53 (s, 2 H), 2.46 (s, 3 H).

Intermediate 53: 2-(chloromethyl)-7-methylquinazolin-4(3H)-one:

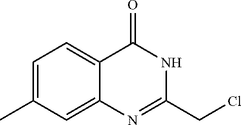

The solid was triturated in MeOH. The suspended solid was isolated by vacuum filtration and vacuum dried at 60° C. for 4 h, to obtain the product as a beige solid.

Yield: (0.88 g, 58%).

ES-MS [M+H] ⁺: 209.1 / 211.0, Rt = 5.211 min (Method-G).

¹H NMR (400 MHz, DMSO-d₆) δ 12.48 (s, 1 H), 7.92 (d, J = 2.1 Hz, 1 H), 7.66 (dd, J = 8.3, 2.1 Hz, 1 H), 7.58 (d, J = 8.2 Hz, 1 H), 4.53 (s, 2 H), 2.45 (s, 3 H).

General procedure E

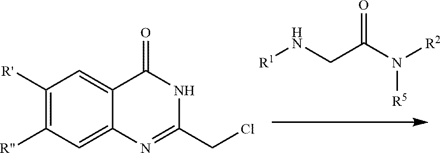

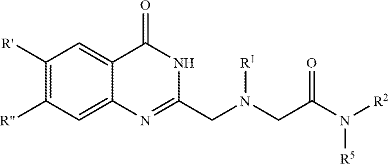

where R¹, R² and R⁵ are as defined above and R′ and R″ are as defined for R⁶, R⁷, and R⁸.

To a stirred mixture of the corresponding chloride (1.0 eq.) and any of the intermediates 33-43 (1.0 eq.) in ethanol (1.5 mL), DIPEA (1.0 eq.) was added. The mixture was heated up to 90° C. and allowed to stir for 16 h. The reaction was quenched with water (10 mL). The aqueous layer was extracted with DCM (x3). The organic layers were combined, dried over anhydrous sodium sulphate, filtered and concentrated to dryness. The resulting crude was purified by flash column chromatography (DCM/DCM:MeOH (90:10), 0%-50%) to obtain the corresponding product as a solid.

### Example 56

6-fluoro-2-(((2-(isoindolin-2-yl)-2-oxoethyl)(methyl)amino)methyl)quinazolin-4(3H)-one

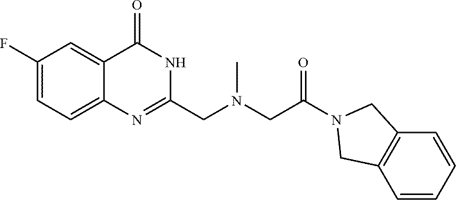 [SMILES: CN(CC(=O)N1Cc2ccccc2C1)Cc1nc2ccc(F)cc2c(=O)[nH]1]

The product was extracted using EtOAc (x3) instead of DCM.

Yield: (0.026 g, 29%).

ES-MS [M+H] ⁺: 367.2, Rt = 17.281 min (Method-H).

¹H NMR (400 MHz, DMSO-d₆) δ 12.28 (s, 1 H), 7.79 (ddd, J = 8.7, 2.8, 0.7 Hz, 1 H), 7.75 - 7.57 (m, 2 H), 7.37 (td, J = 4.3, 3.9, 1.1 Hz, 1 H), 7.34 - 7.18 (m, 3 H), 4.83 (s, 2 H), 4.68 (s, 2 H), 3.64 (s, 2 H), 3.58 (s, 2 H), 2.44 (s, 3 H).

### Example 57

6-fluoro-2-(((2-(indolin-1-yl)-2-oxoethyl)(methyl)amino)methyl)quinazolin-4(3H)-one

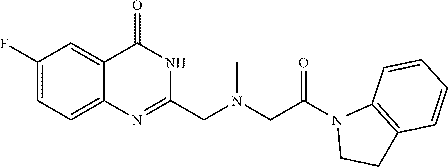 [SMILES: CN(CC(=O)N1CCc2ccccc21)Cc1nc2ccc(F)cc2c(=O)[nH]1]

Yield: (0.050 g, 57%).

ES-MS [M+H] ⁺: 367.2, Rt = 17.814 min (Method-H).

¹H NMR (400 MHz, DMSO-d₆) δ 12.12 (s, 1 H), 8.08 (d, J = 8.1 Hz, 1 H), 7.82 -7.75 (m, 1 H), 7.75 - 7.62 (m, 2 H), 7.24 (d, J = 7.4 Hz, 1 H), 7.16 (t, J = 8.1 Hz, 1 H), 7.00 (td, J = 7.4, 1.1 Hz, 1 H), 4.08 (t, J = 8.5 Hz, 2 H), 3.70 (s, 2 H), 3.64 (s, 2 H), 3.14 (t, J = 8.6 Hz, 2 H), 2.47 (s, 3 H).

### Example 58

N-(3,4-dichlorophenyl)-2-(((6-fluoro-4-oxo-3,4-dihydroquinazolin-2-yl)methyl)(methyl)amino)-N-methylacetamide

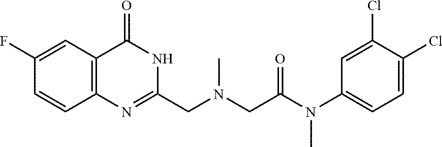 [SMILES: CN(CC(=O)N(C)c1ccc(Cl)c(Cl)c1)Cc1nc2ccc(F)cc2c(=O)[nH]1]

Yield: (0.074 g, 75%).

ES-MS [M+H] ⁺: 423.1 / 424.8, Rt = 19.182 min (Method-H).

¹H NMR (400 MHz, DMSO-d₆) δ 12.00 (s, 1 H), 7.80 - 7.75 (m, 1 H), 7.74 (s, 1 H), 7.71 - 7.66 (m, 2 H), 7.65 (s, 1 H), 7.39 (dd, J = 8.7, 2.5 Hz, 1 H), 3.57 (s, 2 H), 3.18 (s, 3 H), 2.50 (p, J = 1.9 Hz, 2 H), 2.35 (s, 3 H).

### Example 59

N-chlorophenyl)-2-(((6-fluoro-4-oxo-3,4-dihydroquinazolin-2-yl)methyl)(methyl)amino)-N-methylacetamide

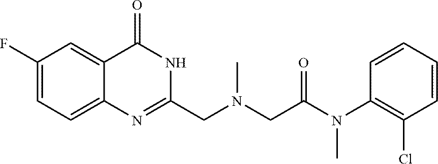 [SMILES: CN(CC(=O)N(C)c1ccccc1Cl)Cc1nc2ccc(F)cc2c(=O)[nH]1]

Yield: (0.080 g, 61%).

ES-MS [M+H] ⁺: 389.1 / 391.1, Rt = 17.984 min (Method-H).

¹H NMR (400 MHz, DMSO-d₆) δ 11.95 (s, 1 H), 7.77 (ddd, J = 8.6, 2.4, 1.2 Hz, 1 H), 7.71 - 7.63 (m, 2 H), 7.62 - 7.49 (m, 2 H), 7.46 - 7.31 (m, 2 H), 3.55 (d, J = 1.0 Hz, 2 H), 3.21 (d, J = 16.5 Hz, 1 H), 3.09 (s, 3 H), 3.00 (d, J = 16.4 Hz, 1 H), 2.33 (s, 3 H).

### Example 60

N-chlorophenyl)-2-(ethyl((6-fluoro-4-oxo-3,4-dihydroquinazolin-2-yl)methyl)amino)-N-methylacetamide

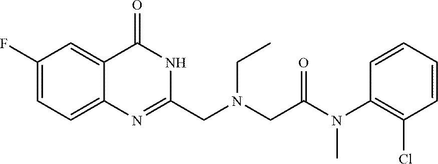 [SMILES: CCN(CC(=O)N(C)c1ccccc1Cl)Cc1nc2ccc(F)cc2c(=O)[nH]1]

The crude required from a second purification by flash column chromatography (EtOAc/Hex, 20%-100%).

Yield: (0.043 g, 43%).

ES-MS [M+H] ⁺: 403.2 / 405.1, Rt = 18.883 min (Method-H).

¹H NMR (400 MHz, DMSO-d₆) δ 12.06 (s, 1 H), 7.80 - 7.72 (m, 1 H), 7.70 - 7.63 (m, 3 H), 7.63 - 7.52 (m, 2 H), 7.47 - 7.33 (m, 2 H), 3.62 (d, J = 7.9 Hz, 2 H), 3.27 (d, J = 26.4 Hz, 1 H), 3.09 (s, 3 H), 3.03 (d, J = 16.9 Hz, 1 H), 2.62 (q, J = 7.1 Hz, 2 H), 0.82 (t, J = 7.1 Hz, 3 H).

### Example 61

N-chlorophenyl)-2-(cyclopropyl((6-fluoro-4-oxo-3,4-dihydroquinazolin-2-yl)methyl)amino)-N-methylacetamide

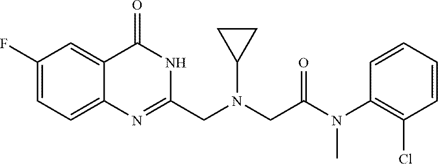 [SMILES: CN(C(=O)CN(Cc1nc2ccc(F)cc2c(=O)[nH]1)C1CC1)c1ccccc1Cl]

The product was extracted using EtOAc (x3) instead of DCM. The purification was performed using EtOAc/Hex, 10%-65%.

Yield: (0.041 g, 35%).

ES-MS [M+H] ⁺: 415.1 / 417.1, Rt = 19.227 min (Method-H).

¹H NMR (400 MHz, DMSO-d₆) δ 11.83 (s, 1 H), 7.66 - 7.56 (m, 1 H), 7.53 (d, J = 1.8 Hz, 1 H), 7.52 -7.50 (m, 1 H), 7.47 - 7.43 (m, 1 H), 7.42 - 7.35 (m, 1 H), 7.34 - 7.18 (m, 2 H), 3.66 (d, J = 4.4 Hz, 2 H), 3.15 - 3.13 (m, 1 H), 3.00 (d, J = 16.7 Hz, 1 H), 2.94 (s, 3 H), 2.25 (tt, J = 6.7, 3.6 Hz, 1 H), 0.18 - 0.09 (m, 2 H), 0.02 (q, J = 3.3 Hz, 2 H).

### Example 62

N-chlorophenyl)-2-(((6-fluoro-4-oxo-3,4-dihydroquinazolin-2-yl)methyl)(propyl)amino)-N-methylacetamide

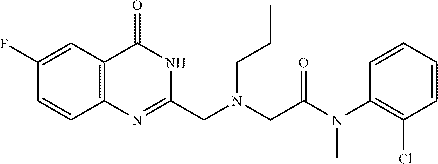 [SMILES: CCCN(CC(=O)N(C)c1ccccc1Cl)Cc1nc2ccc(F)cc2c(=O)[nH]1]

Yield: (0.066 g, 67%).

ES-MS [M+H] ⁺: 417.1 / 419.1, Rt = 19.719 min (Method-H).

¹H NMR (400 MHz, DMSO-d₆) δ 12.12 (s, 1 H), 7.89 - 7.72 (m, 1 H), 7.68 (dd, J = 6.6, 1.6 Hz, 2 H), 7.66 - 7.61 (m, 1 H), 7.61 - 7.54 (m, 1 H), 7.51 - 7.40 (m, 2 H), 3.80 - 3.53 (m, 2 H), 3.26 (d, J = 6.3 Hz, 1 H), 3.12 (s, 3 H), 3.06 (d, J = 17.0 Hz, 1 H), 2.64 - 2.52 (m, 2 H), 1.37 - 1.14 (m, 2 H), 0.69 (t, J = 7.3 Hz, 3 H).

### Example 63

N-chlorophenyl)-2-(((7-fluoro-4-oxo-3,4-dihydroquinazolin-2-yl)methyl)(methyl)amino)-N-methylacetamide

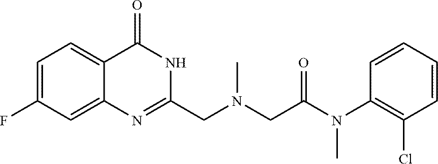 [SMILES: CN(CC(=O)N(C)c1ccccc1Cl)Cc1nc2cc(F)ccc2c(=O)[nH]1]

The product was extracted using EtOAc (x3) instead of DCM.

Yield: (0.025 g, 24%).

ES-MS [M+H] ⁺: 389.1 / 391.1, Rt = 18.140 min (Method-H).

¹H NMR (400 MHz, DMSO-d₆) δ 11.88 (s, 1 H), 8.14 (ddt, J = 8.1, 6.3, 0.9 Hz, 1 H), 7.69 - 7.46 (m, 2 H), 7.46 - 7.14 (m, 4 H), 3.54 (s, 2 H), 3.25 (s, 1 H), 3.07 (d, J = 0.9 Hz, 3 H), 2.98 (d, J = 16.5 Hz, 1 H), 2.32 (d, J = 0.9 Hz, 3 H).

### Example 64

N-chlorophenyl)-2-(ethyl((7-fluoro-4-oxo-3,4-dihydroquinazolin-2-yl)methyl)amino)-N-methylacetamide hydrochloride

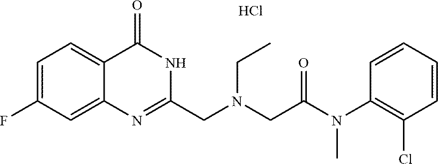 [SMILES: CCN(CC(=O)N(C)c1ccccc1Cl)Cc1nc2cc(F)ccc2c(=O)[nH]1 ; Cl]

The compound was delivered as a hydrochloride salt since the free base was a yellow oil. The salt formation was performed by adding HCl 4M solution in dioxane (1.2 eq.) to the free base of N-(2-chlorophenyl)-2-(ethyl((7-fluoro-4-oxo-3,4-dihydroquinazolin-2-yl)methyl)amino)-N-methylacetamide (1.0 eq.). The mixture was allowed to stir at room temperature for 6 h. The mixture was concentrated to dryness and the resulting crude was coevaporated with diethyl ether, to obtain the hydrochloride salt as a pale brown solid.

Yield: (0.031 g, 30%).

ES-MS [M+H] ⁺: 403.2 / 405.0, Rt = 19.030 min (Method-H).

¹H NMR (400 MHz, Methanol-d4) δ 7.63 - 7.59 (m, 1 H), 7.54 - 7.46 (m, 1 H), 8.28 (dd, J = 9.5, 6.1 Hz, 3 H), 7.42 - 7.22 (m, 2 H), 4.89 (s, 1 H), 4.79 (s, 1 H), 4.27 (s, 2 H), 4.00 - 3.68 (m, 2 H), 3.29 (s, 3 H), 1.27 (t, J = 7.4 Hz, 3 H).

### Example 65

N-chlorophenyl)-2-(cyclopropyl((6-fluoro-4-oxo-3,4-dihydroquinazolin-2-yl)methyl)amino)-N-methylacetamide

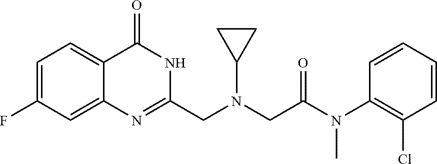 [SMILES: CN(C(=O)CN(Cc1nc2cc(F)ccc2c(=O)[nH]1)C1CC1)c1ccccc1Cl]

The product was extracted using EtOAc (x3) instead of DCM.

Yield: (0.057 g, 49%).

ES-MS [M+H] ⁺: 415.1 / 417.1, Rt = 19.298 min (Method-H).

¹H NMR (400 MHz, DMSO-d₆) δ 11.95 (s, 1 H), 8.15 (dd, J = 8.8, 6.4 Hz, 1 H), 7.69 - 7.59 (m, 1 H), 7.59 - 7.50 (m, 1 H), 7.49 - 7.24 (m, 4 H), 3.84 (d, J = 4.6 Hz, 2 H), 3.32 (s, 1 H), 3.20 (d, J= 17.8 Hz, 1 H), 3.10 (s, 3 H), 2.41 (td, J = 6.6, 3.3 Hz, 1 H), 0.30 (dd, J = 6.5, 2.4 Hz, 2 H), 0.18 (p, J = 3.8 Hz, 2 H).

### Example 66

N-chlorophenyl)-N-methyl-2-(methyl((6-methyl-4-oxo-3,4-dihydroquinazolin-2-yl)methyl)amino)acetamide

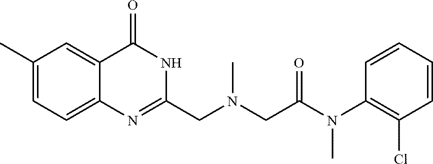 [SMILES: Cc1ccc2nc(CN(C)CC(=O)N(C)c3ccccc3Cl)[nH]c(=O)c2c1]

Yield: (0.036 g, 40%).

ES-MS [M+H] ⁺: 385.2 / 387.0, Rt = 18.060 min (Method-H).

¹H NMR (400 MHz, DMSO-d₆) δ 11.73 - 11.57 (m, 1 H), 7.98 (d, J = 8.0 Hz, 1 H), 7.67 - 7.57 (m, 1 H), 7.57 - 7.49 (m, 1 H), 7.47 - 7.34 (m, 3 H), 7.31 (ddd, J = 8.1, 1.7, 0.7 Hz, 1 H), 3.53 (s, 2 H), 3.29 - 3.17 (m, 1 H), 3.10 (s, 3 H), 3.00 (d, J = 16.4 Hz, 1 H), 2.44 (s, 3 H), 2.32 (s, 3 H).

### Example 67

2-(ethyl((6-fluoro-4-oxo-3,4-dihydroquinazolin-2-yl)methyl)amino)-N-methyl-N-(4-methylthiazol-2-yl)acetamide

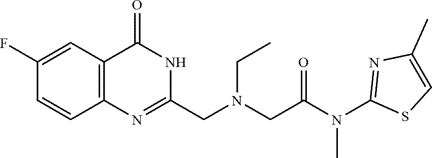 [SMILES: CCN(CC(=O)N(C)c1nc(C)cs1)Cc1nc2ccc(F)cc2c(=O)[nH]1]

Yield: (0.016 g, 17%).

ES-MS [M+H] ⁺: 390.1 / 391.1, Rt = 17.056 min (Method-H).

¹H NMR (400 MHz, DMSO-d₆) δ 12.05 (s, 1 H), 7.78 (ddd, J = 8.6, 2.6, 0.9 Hz, 1 H), 7.72 - 7.65 (m, 2 H), 6.67 (q, J = 1.1 Hz, 1 H), 3.74 (s, 2 H), 3.65 (s, 3 H), 3.60 (s, 2 H), 2.74 (q, J = 7.1 Hz, 2 H), 2.30 (d, J = 1.3 Hz, 3 H), 0.98 (t, J = 7.2 Hz, 3 H).

### Example 68

2-((fluoro-4-oxo-3,4-dihydroquinazolin-2-yl)methyl)(methyl)amino)-N-methyl-N-(4-methylthiazol-2-yl)acetamide

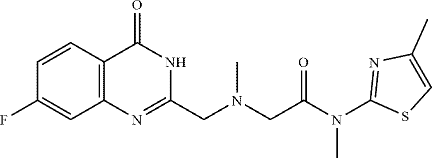 [SMILES: Cc1csc(N(C)C(=O)CN(C)Cc2nc3cc(F)ccc3c(=O)[nH]2)n1]

Yield: (0.019 g, 21%).

ES-MS [M+H] ⁺: 376.2, Rt = 16.055 min (Method-H).

¹H NMR (400 MHz, DMSO-d₆) δ 11.89 (s, 1 H), 8.16 (dd, J = 8.8, 6.4 Hz, 1 H), 7.41 (dd, J = 10.2, 2.6 Hz, 1 H), 7.35 (td, J = 8.7, 2.6 Hz, 1 H), 6.65 (d, J = 1.4 Hz, 1 H), 3.71 (s, 2 H), 3.63 (s, 3 H), 3.55 (s, 2 H), 2.44 (s, 3 H), 2.29 (d, J = 1.2 Hz, 3 H).

### Example 69

2-(N-ethyl-N-((6-fluoro-3,4-dihydro-4-oxoquinazolin-2-yl)methyl)amino)-N-(2,6-dichlorophenyl)-N- methylacetamide

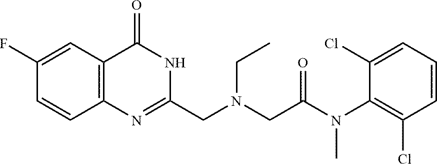 [SMILES: CCN(CC(=O)N(C)c1c(Cl)cccc1Cl)Cc1nc2ccc(F)cc2c(=O)[nH]1]

The purification was performed using EtOAc/Hex, 10%-100%.

Yield: (0.038 g, 38%).

ES-MS [M+H] ⁺: 437.3, Rt = 5.947 min (Method-J).

¹H NMR (400 MHz, DMSO-d₆) δ 11.97 (s, 1 H), 7.77 (ddd, J = 8.6, 2.6, 0.9 Hz, 1 H), 7.70 - 7.65 (m, 2 H), 7.66 - 7.58 (m, 2 H), 7.46 (dd, J = 8.7, 7.7 Hz, 1 H), 3.64 (s, 2 H), 3.17 (s, 2 H), 3.07 (s, 3 H), 2.69 (q, J = 7.1 Hz, 2 H), 0.87 (t, J = 7.2 Hz, 3 H).

### Example 70

2-(N-((3,4-dihydro-7-methyl-4-oxoquinazolin-2-yl)methyl)-N-methylamino)-N-(2-chlorophenyl)-N-methylacetamide

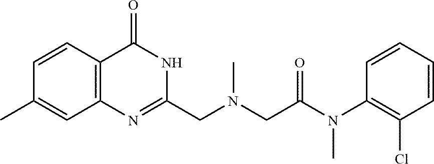 [SMILES: Cc1ccc2c(=O)[nH]c(CN(C)CC(=O)N(C)c3ccccc3Cl)nc2c1]

Yield: (0.062 g, 69%).

ES-MS [M+H] ⁺: 385.2, Rt = 18.161 min (Method-H).

¹H NMR (400 MHz, DMSO-d₆) δ 11.71 (s, 1 H), 7.89 (td, J = 1.5, 0.7 Hz, 1 H), 7.65 - 7.57 (m, 2 H), 7.57 - 7.52 (m, 1 H), 7.49 (d, J = 8.3 Hz, 1 H), 7.41 (dd, J = 5.9, 3.5 Hz, 2 H), 3.53 (d, J = 1.2 Hz, 2 H), 3.21 (d, J = 16.5 Hz, 1 H), 3.09 (s, 3 H), 2.99 (d, J = 16.5 Hz, 1 H), 2.43 (s, 3 H), 2.32 (s, 3 H).

### Example 71

7-fluoro-2-(((2-(isoindolin-2-yl)-2-oxoethyl)(methyl)amino)methyl)quinazolin-4(3H)-one

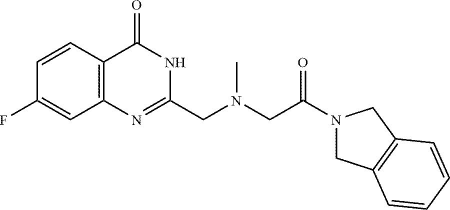 [SMILES: CN(CC(=O)N1Cc2ccccc2C1)Cc1nc2cc(F)ccc2c(=O)[nH]1]

The product precipitated after treating the reaction mixture with water (2.5 mL). The suspended solid was isolated by vacuum filtration and vacuum dried at 60° C. overnight.

Yield: (0.067 g, 65%).

ES-MS [M+H] ⁺: 367.2 / 368.1, Rt = 17.288 min (Method-H).

¹H NMR (400 MHz, DMSO-d₆) δ 12.25 (s, 1 H), 8.18 (dd, J = 8.8, 6.3 Hz, 1 H), 7.54 - 7.16 (m, 6 H), 4.83 (s, 2 H), 4.68 (s, 2 H), 3.65 (s, 2 H), 3.59 (s, 2 H), 2.44 (s, 3 H).

### Example 72

7-fluoro-2-(((2-(indolin-1-yl)-2-oxoethyl)(methyl)amino)methyl)quinazolin-4(3H)-one

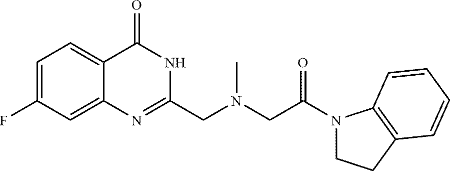 [SMILES: CN(CC(=O)N1CCc2ccccc21)Cc1nc2cc(F)ccc2c(=O)[nH]1]

The crude was treated with water (10 mL) and the suspended solid was stirred at room temperature for 30 minutes. The solid was isolated by vacuum filtration and vacuum dried at 60° C. overnight, to obtain the product as a pale brown solid.

Yield: (0.067 g, 78%).

ES-MS [M+H] ⁺: 367.2, Rt = 17.930 min (Method-H).

¹H NMR (400 MHz, DMSO-d₆) δ 12.07 (s, 1 H), 8.17 (dd, J = 8.8, 6.3 Hz, 1 H), 8.08 (d, J = 8.1 Hz, 1 H), 7.41 (dd, J = 10.2, 2.5 Hz, 1 H), 7.35 (td, J = 8.7, 2.6 Hz, 1 H), 7.24 (d, J = 7.4 Hz, 1 H), 7.15 (t, J = 7.8 Hz, 1 H), 7.00 (td, J = 7.4, 1.1 Hz, 1 H), 4.08 (t, J = 8.5 Hz, 2 H), 3.71 (s, 2 H), 3.64 (s, 2 H), 3.14 (t, J = 8.5 Hz, 2 H), 2.47 (s, 3 H).

### Example 73

2-(N-ethyl-N-((7-fluoro-3,4-dihydro-4-oxoquinazolin-2-yl)methyl)amino)-N-(2,6-dichlorophenyl)-N- methylacetamide

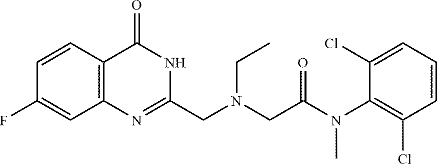 [SMILES: CCN(CC(=O)N(C)c1c(Cl)cccc1Cl)Cc1nc2cc(F)ccc2c(=O)[nH]1]

The purification was performed using EtOAc/Hex, 10%-100%.

Yield: (0.047 g, 69%).

ES-MS [M+H] ⁺: 437.2, Rt = 5.949 min (Method-H).

¹H NMR (400 MHz, DMSO-d₆) δ 11.92 (s, 1 H), 8.28 - 8.07 (m, 1 H), 7.75 - 7.59 (m, 2 H), 7.46 (dd, J = 8.6, 7.7 Hz, 1 H), 7.36 (d, J = 9.4 Hz, 2 H), 3.65 (s, 2 H), 3.17 (s, 2 H), 3.07 (s, 3 H), 2.68 (q, J = 7.1 Hz, 2 H), 0.87 (t, J = 7.2 Hz, 3 H).

### Example 74

2-((fluoro-4-oxo-3,4-dihydroquinazolin-2-yl)methyl)(methyl)amino)-N-(3-fluorophenyl)-N-methylacetamide

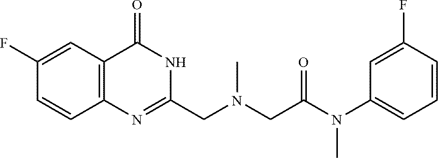 [SMILES: CN(CC(=O)N(C)c1cccc(F)c1)Cc1nc2ccc(F)cc2c(=O)[nH]1]

Yield: (0.060 g, 64%).

ES-MS [M+H] ⁺: 373.1, Rt = 17.300 min (Method-H).

¹H NMR (400 MHz, DMSO-d₆) δ 12.01 (s, 1 H), 7.81 - 7.73 (m, 1 H), 7.73 - 7.63 (m, 2 H), 7.44 (td, J = 8.2, 6.6 Hz, 1 H), 7.30 (d, J = 10.2 Hz, 1 H), 7.24 - 7.13 (m, 2 H), 3.57 (s, 2 H), 3.33 (s, 2 H), 3.19 (s, 3 H), 2.34 (s, 3 H).

### Example 75

2-(N-(fluoro-3,4-dihydro-4-oxoquinazolin-2-yl)methyl)-N-methylamino)-N-(3,4-dichlorophenyl)-N-methylacetamide

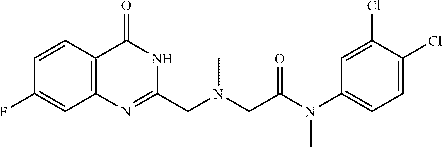 [SMILES: CN(CC(=O)N(C)c1ccc(Cl)c(Cl)c1)Cc1nc2cc(F)ccc2c(=O)[nH]1]

Yield: (0.078 g, 78%).

ES-MS [M+H] ⁺: 423.1 / 424.9, Rt = 19.370 min (Method-H).

¹H NMR (400 MHz, DMSO-d₆) δ 11.13 (s, 1 H), 7.34 (dd, J = 8.8, 6.3 Hz, 1 H), 6.92 (s, 1 H), 6.84 (d, J = 8.6 Hz, 1 H), 6.68 - 6.43 (m, 3 H), 2.76 (s, 2 H), 2.37 (s, 3 H), 1.68 (p, J = 1.8 Hz, 2 H), 1.54 (s, 3 H).

### Example 76

N-chlorophenyl)-2-(((7-fluoro-4-oxo-3,4-dihydroquinazolin-2-yl)methyl)(propyl)amino)-N-methylacetamide

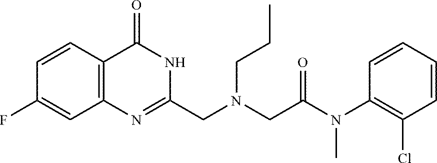 [SMILES: CCCN(CC(=O)N(C)c1ccccc1Cl)Cc1nc2cc(F)ccc2c(=O)[nH]1]

Yield: (0.063 g, 64%).

ES-MS [M+H] ⁺: 417.0 / 419.0, Rt = 19.876 min (Method-H).

¹H NMR (400 MHz, DMSO-d₆) δ 12.07 (s, 1 H), 8.16 (dd, J = 8.7, 6.4 Hz, 1 H), 7.67 - 7.62 (m, 1 H), 7.62 - 7.56 (m, 1 H), 7.50 - 7.43 (m, 2 H), 7.42 - 7.29 (m, 2 H), 3.74 - 3.59 (m, 2 H), 3.26 (d, J = 7.8 Hz, 1 H), 3.12 (s, 3 H), 3.06 (d, J = 17.0 Hz, 1 H), 2.57 - 2.51 (m, 2 H), 1.35 - 1.13 (m, 2 H), 0.69 (t, J = 7.3 Hz, 3 H).

### Example 77

2-(N-(fluoro-3,4-dihydro-4-oxoquinazolin-2-yl)methyl)-N-methylamino)-N-(3-fluorophenyl)-N-methylacetamide

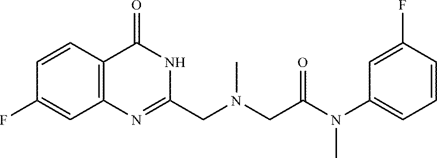 [SMILES: CN(CC(=O)N(C)c1cccc(F)c1)Cc1nc2cc(F)ccc2c(=O)[nH]1]

Yield: (0.066 g, 75%).

ES-MS [M+H] ⁺: 373.1, Rt = 17.479 min (Method-H).

¹H NMR (400 MHz, DMSO-d₆) δ 11.96 (s, 1 H), 8.16 (dd, J = 8.8, 6.3 Hz, 1 H), 7.45 (td, J = 8.2, 6.6 Hz, 1 H), 7.41 - 7.27 (m, 3 H), 7.25 - 7.13 (m, 2 H), 3.58 (s, 2 H), 3.32 (s, 2 H), 3.19 (s, 3 H), 2.35 (s, 3 H).

### Example 78

2-(N-cyclopropyl-N-((3,4-dihydro-6-methyl-4-oxoquinazolin-2-yl)methyl)amino)-N-(2-chlorophenyl)-N- methylacetamide

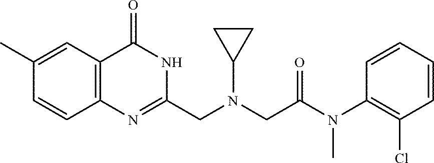 [SMILES: Cc1ccc2nc(CN(CC(=O)N(C)c3ccccc3Cl)C3CC3)[nH]c(=O)c2c1]

Yield: (0.022 g, 23%).

ES-MS [M+H] ⁺: 410.8 / 413.1, Rt = 19.226 min (Method-H).

¹H NMR (400 MHz, DMSO-d₆) δ 11.74 (s, 1 H), 7.97 (d, J = 8.1 Hz, 1 H), 7.66 -7.58 (m, 1 H), 7.58 - 7.51 (m, 1 H), 7.49 - 7.34 (m, 3 H), 7.31 (dd, J = 8.3, 1.7 Hz, 1 H), 3.87 - 3.74 (m, 2 H), 3.35 (s, 1 H), 3.20 (d, J = 17.9 Hz, 1 H), 3.11 (s, 3 H), 2.45 (s, 3 H), 2.39 (dq, J = 6.7, 3.4 Hz, 1 H), 0.43 - 0.22 (m, 2 H), 0.24 - 0.08 (m, 2 H).

### Example 79

N-chlorophenyl)-2-(ethyl((6-methyl-4-oxo-3,4-dihydroquinazolin-2-yl)methyl)amino)-N-methylacetamide

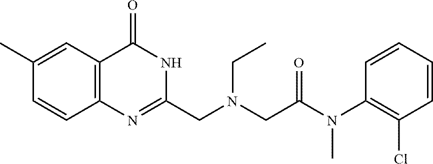 [SMILES: CCN(CC(=O)N(C)c1ccccc1Cl)Cc1nc2ccc(C)cc2c(=O)[nH]1]

Yield: (0.077 g, 80%).

ES-MS [M+H] ⁺: 399.3, Rt = 18.959 min (Method-H).

¹H NMR (400 MHz, DMSO-d₆) δ 11.82 (s, 1 H), 7.98 (d, J = 8.0 Hz, 1 H), 7.63 (dd, J = 6.1, 3.4 Hz, 1 H), 7.58 (dd, J = 6.0, 3.5 Hz, 1 H), 7.44 (dd, J = 5.9, 3.6 Hz, 2 H), 7.40 (dt, J = 1.6, 0.7 Hz, 1 H), 7.34 - 7.27 (m, 1 H), 3.72 - 3.49 (m, 2 H), 3.30 - 3.19 (m, 1 H), 3.11 (s, 3 H), 3.05 (d, J = 16.8 Hz, 1 H), 2.62 (q, J = 7.1 Hz, 2 H), 2.44 (s, 3 H), 0.83 (t, J = 7.1 Hz, 3 H).

### Example 80

N-chlorophenyl)-N-methyl-2-(((6-methyl-4-oxo-3,4-dihydroquinazolin-2-yl)methyl)(propyl)amino)acetamide

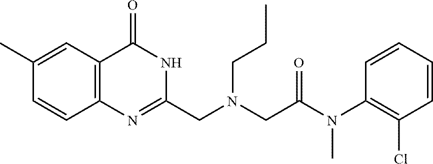 [SMILES: CCCN(CC(=O)N(C)c1ccccc1Cl)Cc1nc2ccc(C)cc2c(=O)[nH]1]

Yield: (0.073 g, 73%).

ES-MS [M+H] ⁺: 413.1 / 415.0, Rt = 19.804 min (Method-H).

¹H NMR (400 MHz, DMSO-d₆) δ 11.86 (s, 1 H), 7.99 (dd, J = 8.1, 1.8 Hz, 1 H), 7.70 - 7.62 (m, 1 H), 7.59 (dd, J = 6.2, 3.2 Hz, 1 H), 7.51 - 7.43 (m, 2 H), 7.42 - 7.36 (m, 1 H), 7.31 (dd, J = 8.1, 1.7 Hz, 1 H), 3.74 - 3.47 (m, 2 H), 3.33 - 3.19 (m, 1 H), 3.12 (d, J = 1.8 Hz, 3 H), 3.05 (d, J = 16.9 Hz, 1 H), 2.51 (dd, J = 4.9, 3.0 Hz, 2 H), 2.44 (s, 3 H), 1.37 - 1.14 (m, 2 H), 0.68 (t, J = 7.3 Hz, 3 H).

### Example 81

2-(((indolin-1-yl)-2-oxoethyl)(methyl)amino)methyl)-6-methylquinazolin-4(3H)-one

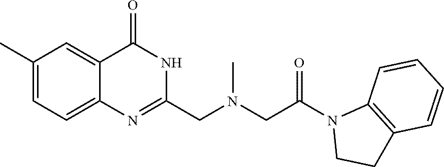 [SMILES: Cc1ccc2nc(CN(C)CC(=O)N3CCc4ccccc43)[nH]c(=O)c2c1]

Yield: (0.064 g, 54%).

ES-MS [M+H] ⁺: 363.1 / 364.1, Rt = 17.869 min (Method-H).

¹H NMR (400 MHz, DMSO-d₆) δ 11.86 (s, 1 H), 8.09 (d, J = 8.0 Hz, 1 H), 7.99 (d, J = 8.0 Hz, 1 H), 7.46 - 7.41 (m, 1 H), 7.31 (dd, J = 8.1, 1.7 Hz, 1 H), 7.24 (d, J = 7.3 Hz, 1 H), 7.20 - 7.12 (m, 1 H), 7.00 (td, J = 7.4, 1.1 Hz, 1 H), 4.08 (t, J = 8.4 Hz, 2 H), 3.68 (s, 2 H), 3.63 (s, 2 H), 3.14 (t, J = 8.5 Hz, 2 H), 2.65 (s, 3 H), 2.44 (s, 3 H)

### Example 82

N-fluorophenyl)-N-methyl-2-(methyl((6-methyl-4-oxo-3,4-dihydroquinazolin-2-yl)methyl)amino)acetamide

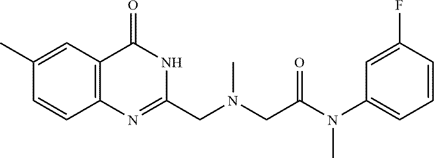 [SMILES: Cc1ccc2nc(CN(C)CC(=O)N(C)c3cccc(F)c3)[nH]c(=O)c2c1]

Yield: (0.055 g, 60%).

ES-MS [M+H] ⁺: 369.2, Rt = 17.482 min (Method-H).

¹H NMR (400 MHz, DMSO-d₆) δ 11.75 (s, 1 H), 7.98 (d, J = 8.0 Hz, 1 H), 7.45 (td, J = 8.1, 6.6 Hz, 1 H), 7.44 - 7.37 (m, 1 H), 7.37 - 7.26 (m, 2 H), 7.26 - 7.11 (m, 2 H), 3.55 (s, 2 H), 3.32 (s, 2 H), 3.19 (s, 3 H), 2.44 (s, 3 H), 2.34 (s, 3 H).

### Example 83

N-chlorophenyl)-2-(cyclopropyl((7-methyl-4-oxo-3,4-dihydroquinazolin-2-yl)methyl)amino)-N-methylacetamide

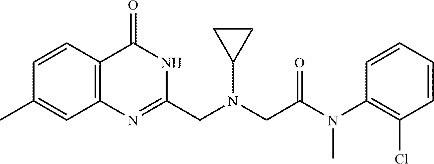 [SMILES: Cc1ccc2c(=O)[nH]c(CN(CC(=O)N(C)c3ccccc3Cl)C3CC3)nc2c1]

Yield: (0.068 g, 68%).

ES-MS [M+H] ⁺: 411.3, Rt = 19.243 min (Method-H).

¹H NMR (400 MHz, DMSO-d₆) δ 11.76 (s, 1 H), 7.89 (dt, J = 2.4, 0.7 Hz, 1 H), 7.61 (ddd, J = 8.3, 4.2, 2.1 Hz, 2 H), 7.58 - 7.53 (m, 1 H), 7.51 (d, J = 8.3 Hz, 1 H), 7.46 - 7.38 (m, 2 H), 3.89 - 3.72 (m, 2 H), 3.29 (d, J = 8.0 Hz, 1 H), 3.15 (d, J = 16.7 Hz, 1 H), 3.10 (s, 3 H), 2.43 (s, 3 H), 2.39 (dt, J = 6.5, 3.1 Hz, 1H), 0.35 - 0.24 (m, 2 H), 0.24 - 0.13 (m, 2 H).

### Example 84

N-chlorophenyl)-2-(ethyl((7-methyl-4-oxo-3,4-dihydroquinazolin-2-yl)methyl)amino)-N-methylacetamide

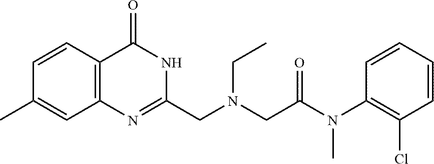 [SMILES: CCN(CC(=O)N(C)c1ccccc1Cl)Cc1nc2cc(C)ccc2c(=O)[nH]1]

Yield: (0.077 g, 80%).

ES-MS [M+H] ⁺: 399.1 / 401.0, Rt = 18.998 min (Method-H).

¹H NMR (400 MHz, DMSO-d₆) δ 11.85 (s, 1 H), 7.90 (d, J = 1.8 Hz, 1 H), 7.68 -7.61 (m, 1 H), 7.61 - 7.55 (m, 2 H), 7.49 (d, J = 8.5 Hz, 1 H), 7.44 (dd, J = 5.9, 3.6 Hz, 2 H), 3.74 - 3.48 (m, 2 H), 3.24 (d, J = 3.2 Hz, 1 H), 3.11 (s, 3 H), 3.04 (d, J = 16.8 Hz, 1 H), 2.62 (q, J = 7.1 Hz, 2 H), 2.43 (s, 3 H), 0.83 (t, J = 7.1 Hz, 3H).

### Example 85

N-chlorophenyl)-N-methyl-2-(((7-methyl-4-oxo-3,4-dihydroquinazolin-2-yl)methyl)(propyl)amino)acetamide

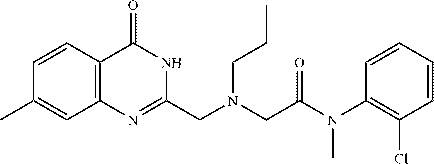 [SMILES: CCCN(CC(=O)N(C)c1ccccc1Cl)Cc1nc2cc(C)ccc2c(=O)[nH]1]

The crude required from a second purification by flash column chromatography (Hex /EtOAc, 25%-75%).

Yield: (0.030 g, 30%).

ES-MS [M+H] ⁺: 413.2, Rt = 19.888 min (Method-H).

¹H NMR (400 MHz, DMSO-d₆) δ 11.89 (s, 1 H), 8.03 - 7.81 (m, 1 H), 7.70 - 7.63 (m, 1 H), 7.63 - 7.55 (m, 2 H), 7.50 (d, J = 8.3 Hz, 1 H), 7.48 - 7.42 (m, 2 H), 3.79 - 3.50 (m, 2 H), 3.31 - 3.19 (m, 2 H), 3.12 (s, 3 H), 3.05 (d, J = 17.0 Hz, 1 H), 2.60 - 2.50 (m, 1 H), 2.43 (s, 3 H), 1.30 - 1.17 (m, 2 H), 0.68 (t, J = 7.3 Hz, 3 H).

### Example 86

2-(((indolin-1-yl)-2-oxoethyl)(methyl)amino)methyl)-7-methylquinazolin-4(3H)-one

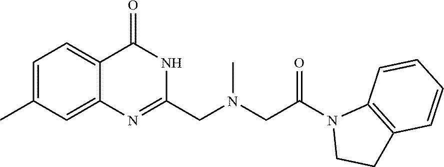 [SMILES: Cc1ccc2c(=O)[nH]c(CN(C)CC(=O)N3CCc4ccccc43)nc2c1]

Yield: (0.072 g, 75%).

ES-MS [M+H] ⁺: 363.1, Rt = 17.939 min (Method-H).

¹H NMR (400 MHz, DMSO-d₆) δ 11.89 (s, 1 H), 8.10 (d, J = 8.0 Hz, 1 H), 7.99 -7.82 (m, 1 H), 7.61 (ddd, J = 8.3, 2.1, 0.6 Hz, 1 H), 7.54 (d, J = 8.3 Hz, 1 H), 7.28 - 7.21 (m, 1 H), 7.16 (ddd, J = 8.6, 7.5, 1.3 Hz, 1 H), 7.00 (td, J = 7.4, 1.1 Hz, 1 H), 4.08 (t, J = 8.5 Hz, 2 H), 3.67 (s, 2 H), 3.63 (s, 2 H), 3.14 (t, J = 8.5 Hz, 2 H), 2.45 (s, 3 H), 2.44 (s, 3 H).

### Example 87

2-(N-((3,4-dihydro-7-methyl-4-oxoquinazolin-2-yl)methyl)-N-methylamino)-N-(3-fluorophenyl)-N-methylacetamide

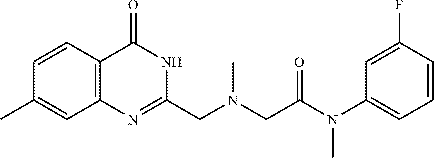 [SMILES: Cc1ccc2c(=O)[nH]c(CN(C)CC(=O)N(C)c3cccc(F)c3)nc2c1]

Yield: (0.068 g, 77%).

ES-MS [M+H] ⁺: 369.2, Rt = 17.508 min (Method-H).

¹H NMR (400 MHz, DMSO-d₆) δ 11.78 (s, 1 H), 7.98 - 7.80 (m, 1 H), 7.61 (ddd, J = 8.3, 2.1, 0.6 Hz, 1 H), 7.51 (d, J = 8.3 Hz, 1 H), 7.48 - 7.38 (m, 1 H), 7.31 (d, J = 10.1 Hz, 1 H), 7.25 - 7.14 (m, 2 H), 3.55 (s, 2 H), 3.33 (s, 2 H), 3.19 (s, 3 H), 2.43 (s, 3 H), 2.33 (s, 3 H).

### Example 88

2-(N-((3,4-dihydro-7-methyl-4-oxoquinazolin-2-yl)methyl)-N-methylamino)-N-(3,4-dichlorophenyl)-N- methylacetamide

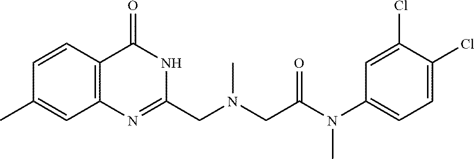 [SMILES: Cc1ccc2c(=O)[nH]c(CN(C)CC(=O)N(C)c3ccc(Cl)c(Cl)c3)nc2c1]

Yield: (0.086 g, 85%).

ES-MS [M+H] ⁺: 419.2 / 421.0, Rt = 19.400 min (Method-H).

¹H NMR (400 MHz, DMSO-d₆) δ 11.77 (s, 1 H), 7.90 (dt, J = 2.2, 0.7 Hz, 1 H), 7.74 (s, 1 H), 7.66 (d, J = 8.6 Hz, 1 H), 7.61 (ddd, J = 8.3, 2.1, 0.7 Hz, 1 H), 7.51 (d, J = 8.3 Hz, 1 H), 7.39 (dd, J = 8.6, 2.5 Hz, 1 H), 3.55 (s, 2 H), 3.32 (s, 2 H), 3.18 (s, 3 H), 2.43 (s, 3 H), 2.34 (s, 3 H).

### Example 89

2-(N-((3,4-dihydro-6-methyl-4-oxoquinazolin-2-yl)methyl)-N-methylamino)-N-(3,4-dichlorophenyl)-N- methylacetamide

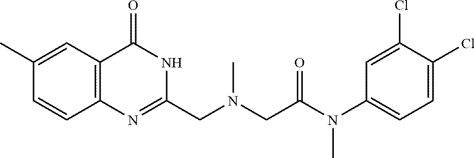 [SMILES: Cc1ccc2nc(CN(C)CC(=O)N(C)c3ccc(Cl)c(Cl)c3)[nH]c(=O)c2c1]

Yield: (0.020 g, 46%).

ES-MS [M+H] ⁺: 419.2 / 421.0, Rt = 19.381 min (Method-H).

¹H NMR (400 MHz, DMSO-d₆) δ 11.69 (s, 1 H), 7.98 (d, J = 8.0 Hz, 1 H), 7.74 (s, 1 H), 7.66 (d, J = 8.6 Hz, 1 H), 7.51 - 7.38 (m, 1 H), 7.38 (d, J = 2.4 Hz, 1 H), 7.31 (ddd, J = 8.1, 1.7, 0.6 Hz, 1H), 3.56 (s, 2 H), 3.32 (s, 2 H), 3.19 (d, J = 4.3 Hz, 3 H), 2.45 (s, 3 H), 2.35 (s, 3 H).

### Synthesis of Intermediate 54

6-(chloromethyl)-1-methyl-1,5-dihydro-4H-pyrazolo[3,4-d]pyrimidin-4-one

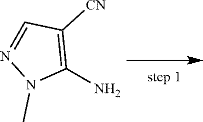

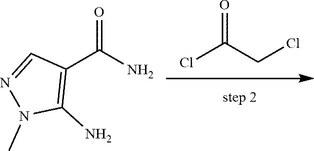

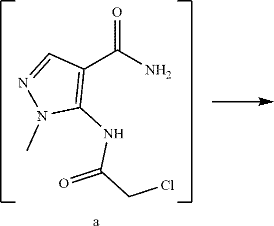

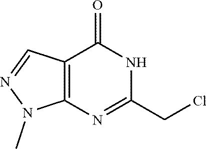

### Step 1

5-amino-1-methyl-1H-pyrazole-4-carbonitrile (2.00 g, 16.38 mmol, 1.0 eq.) was dissolved in sulphuric acid (1.2 M). The reaction mixture was stirred at room temperature for 2 h. The mixture was quenched by slowly pouring the mixture into cold water (50 mL) followed by a basification with ammonium hydroxide solution 25% w/w until pH = 8.0. The suspended solid was cooled down to 0° C. and stirred for 1 h. The solid was isolated by vacuum filtration and vacuum dried at 60° C. overnight to obtain 5-amino-1-methyl-1H-pyrazole-4-carboxamide as a yellow solid.

Yield: (2.0 g, 59%).

ES-MS [M+H] ⁺: 141.0, Rt = 0.920 min (Method-G).

### Step 2

5-amino-1-methyl-1H-pyrazole-4-carboxamide (0.10 g, 0.71 mmol, 1.0 eq.) was dissolved in anhydrous DMF (1.38 mL) and Et₃N (0.11 mL, 0.78 mmol, 1.1 eq.) and the mixture was cooled down to 0° C. in an ice bath. Then, 2-chloroacetyl chloride (60 µL, 0.78 mmol, 1.1 eq.) was added and the reaction mixture was stirred at 0° C. for 30 min and at room temperature for 2 more hours. After this time, only open intermediate a was formed. The reaction was quenched with 2N HCl and it was extracted with EtOAc (3 x). The combined organic layers were washed with sat. NaHCO₃ solution, dried over Na₂SO₄, filtered and evaporated to dryness. Since very low weight of intermediate a was isolated the pH of the aqueous phase was adjusted to 7.5 and it was extracted with DCM (3 x). The combined organic layers were dried over Na₂SO₄, filtered and evaporated to dryness to render a white solid that corresponded to the cyclized product. This crude was purified by flash column chromatography (DCM/DCM:MeOH (90:10, 0%-50%) to obtain the 6-(chloromethyl)-1-methyl-1,5-dihydro-4H-pyrazolo[3,4-d]pyrimidin-4-one as a white solid.

Yield: (0.035 g, 23%)

ES-MS [M+H] ⁺: 199.1, Rt = 3.602 min (Method-G).

¹H NMR (400 MHz, DMSO-d₆) δ 12.46 (s, 1 H), 7.95 (s, 1 H), 4.58 (s, 2 H), 3.90 (s, 3 H).

General Procedure F

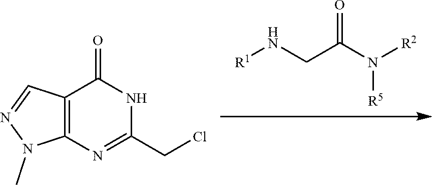

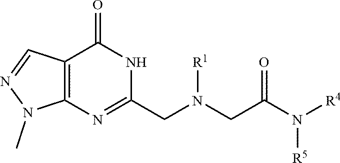

where R¹, R², and R⁵ are as defined above

To a stirred mixture of 6-(chloromethyl)-1-methyl-1,5-dihydro-4H-pyrazolo[3,4-d]pyrimidin-4-one (1.0 eq.) and any of the intermediates 33-43 (1.0 eq.) in ethanol (1.5 mL), DIPEA (1.0 eq.) was added. The mixture was heated up to 90° C. and allowed to stir for 16 h. The reaction was quenched with water (10 mL). The aqueous layer was extracted with DCM (x3). The organic layers were combined, dried over anhydrous sodium sulphate, filtered and concentrated to dryness. The resulting crude was purified by flash column chromatography (DCM/DCM:MeOH (90:10), 0%-50%) to obtain the corresponding product as a solid.

### Example 90

6-(((isoindolin-2-yl)-2-oxoethyl)(methyl)amino)methyl)-1-methyl-1,5-dihydro-4H-pyrazolo[3,4-d]pyrimidin-4-one

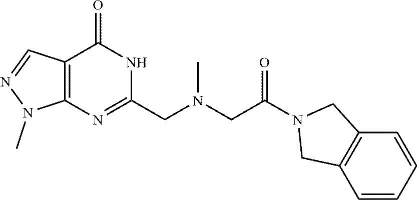 [SMILES: CN(CC(=O)N1Cc2ccccc2C1)Cc1nc2c(cnn2C)c(=O)[nH]1]

Yield: (0.033 g, 56%).

ES-MS [M+H] ⁺: 353.2, Rt = 15.220 min (Method-H).

¹H NMR (400 MHz, DMSO-d₆) δ 11.95 (s, 1 H), 8.01 (s, 1 H), 7.65 - 7.15 (m, 4 H), 4.84 (s, 2 H), 4.67 (s, 2 H), 3.86 (s, 3 H), 3.67 (s, 2 H), 3.58 (s, 2 H), 2.44 (s, 3 H).

### Example 91

6-(((indolin-1-yl)-2-oxoethyl)(methyl)amino)methyl)-1-methyl-1,5-dihydro-4H-pyrazolo[3,4-d]pyrimidin-4-one

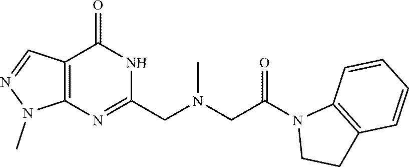 [SMILES: CN(CC(=O)N1CCc2ccccc21)Cc1nc2c(cnn2C)c(=O)[nH]1]

Yield: (0.047 g, 67%).

ES-MS [M+H] ⁺: 353.2, Rt = 15.654 min (Method-H).

¹H NMR (400 MHz, DMSO-d₆) δ 11.81 (s, 1 H), 8.07 (d, J = 8.0 Hz,1 H), 8.01 (d, J = 0.6 Hz, 1 H), 7.24 (d, J = 7.4 Hz, 1 H), 7.16 (t, J = 7.7 Hz, 1 H), 7.00 (td, J = 7.4, 1.1 Hz, 1 H), 4.09 (t, J = 8.5 Hz, 2 H), 3.86 (d, J = 0.6 Hz, 3 H), 3.74 (s, 2 H), 3.64 (s, 2 H), 3.15 (t, J = 8.4 Hz, 2 H), 2.46 (s, 3 H).

### Example 92

N-chlorophenyl)-N-methyl-2-(methyl((1-methyl-4-oxo-4,5-dihydro-1H-pyrazolo[3,4-d]pyrimidin-6-yl)methyl)amino)acetamide

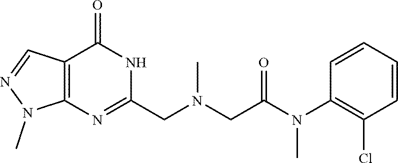 [SMILES: CN(CC(=O)N(C)c1ccccc1Cl)Cc1nc2c(cnn2C)c(=O)[nH]1]

This product required a second purification by semipreparative HPLC (Method-I).

Yield: (0.029 g, 35%).

ES-MS [M+H] ⁺: 375.0 / 377.1, Rt = 15.728 min (Method-H).

¹H NMR (400 MH_{z}, DMSO-d₆) δ 11.66 (s, 1 H), 8.00 (s, 1 H), 7.68 - 7.57 (m, 1 H), 7.57 - 7.46 (m, 1 H), 7.46 - 7.31 (m, 2 H), 3.83 (s, 3 H), 3.58 (d, J = 2.4 Hz, 2 H), 3.22 (d, J = 16.4 Hz, 1 H), 3.09 (s, 3 H), 3.01 (d, J = 16.4 Hz, 1 H), 2.33 (s, 3 H).

### Example 93

2-(N-ethyl-N-((4,5-dihydro-l-methyl-4-oxo-1H-pyrazolo[3,4-d]pylimidin-6-yl)methyl)amino)-N-(2,6-dichlorophenyl)-N-methylacetamide

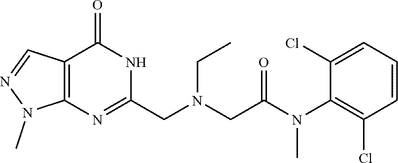 [SMILES: CCN(CC(=O)N(C)c1c(Cl)cccc1Cl)Cc1nc2c(cnn2C)c(=O)[nH]1]

Yield: (0.036 g, 61%).

ES-MS [M+H] ⁺: 423.2, Rt = 5.288 min (Method-J).

¹H NMR (400 MHz, DMSO-d₆) δ 11.68 (s, 1 H), 8.00 (s, 1 H), 7.73 - 7.54 (m, 2 H), 7.46 (dd, J = 8.6, 7.7 Hz, 1 H), 3.84 (s, 3 H), 3.67 (s, 2 H), 3.18 (s, 2 H), 3.07 (s, 3 H), 2.67 (q, J = 7.2 Hz, 2 H), 0.87 (t, J = 7.2 Hz, 3 H).

### Example 94

N-fluorophenyl)-N-methyl-2-(methyl((1-methyl-4-oxo-4,5-dihydro-1H-pyrazolo[3,4-d]pyrimidin-6-yl)methyl)amino)acetamide

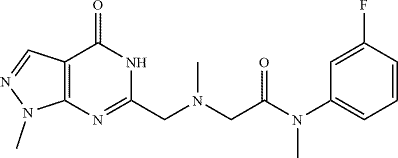 [SMILES: CN(CC(=O)N(C)c1cccc(F)c1)Cc1nc2c(cnn2C)c(=O)[nH]1]

Yield: (0.057 g, 65%).

ES-MS [M+H] ⁺: 359.2, Rt = 15.241 min (Method-H).

¹H NMR (400 MHz, DMSO-d₆) δ 11.70 (s, 1 H), 8.00 (s, 1 H), 7.46 (td, J = 8.2, 6.6 Hz, 1 H), 7.31 (d, J = 10.1 Hz, 1 H), 7.25 - 7.14 (m, 2 H), 3.85 (s, 3 H), 3.61 (s, 2 H), 3.32 (s, 2 H), 3.19 (s, 3 H), 2.34 (s, 3 H).

### Biological Assays

Compounds of the Disclosure are capable of stabilizing α-L-iduronidase thereby enhancing its activity.

### Differential Scanning Fluorimetry (DSF)

The capacity of Compounds of the Disclosure to stabilize α-L-iduronidase was assessed by differential scanning fluorimetry technique. The thermal denaturation of purified human native enzyme was monitored in the presence of the extrinsic fluorescent probe SYPRO Orange (Sigma-Aldrich, St. Louis, MO). The tested compounds were dissolved in 100% DMSO and diluted into the protein buffer to achieve final concentrations of 1% DMSO.

Recombinant human α-L-iduronidase protein (purchased from R&D) 12.5 µL of 1.5 µM in 100 mM Hepes 20 mM MgCl₂ pH 7 (protein final concentration 0.75 µM) with Sypro Orange 10X and 12.5 µl of the different compound solutions were dispensed into 96-well PCR-plates. The intensity of SYPRO Orange fluorescence after a systematic increase of temperature was monitored in the Roche LightCycler® 480 II device (Roche Diagnostics).

The capacity to stabilize α-L-iduronidase is denoted as follows:
ΔTm GALC > 1 is shown as A;
ΔTm GALC between 0.5 and 1 is shown as B;
ΔTm GALC between 0.1 and 0.5 is shown as C; and
ΔTm GALC less than 0.1 is shown as D.

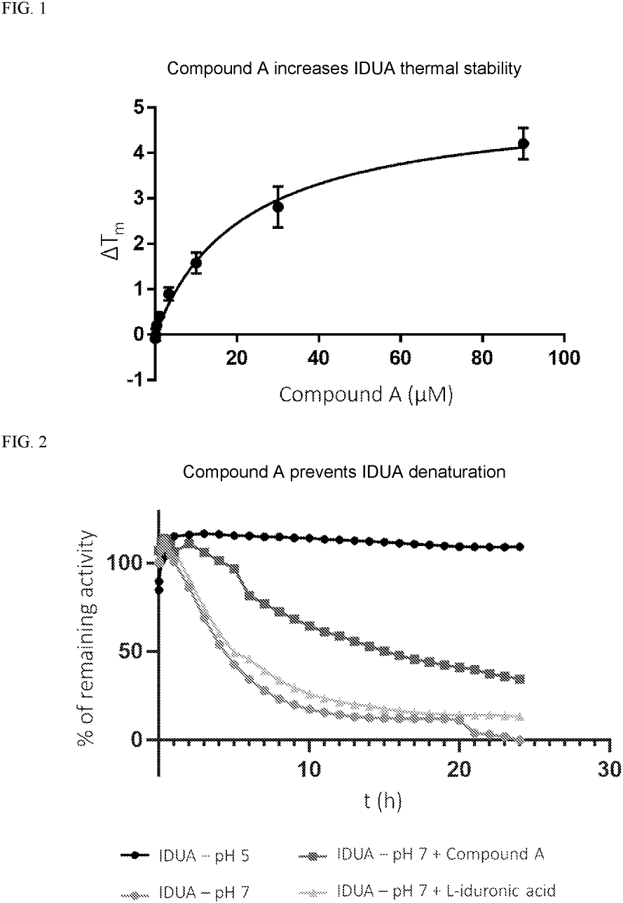

The thermal shift dose response curve for an exemplary Compound of the Disclosure, Compound A, is presented in FIG. 1. The results show that Compound A increases IDUA thermal stability. Compound A stabilizes recombinant human IDUA by up to 4° C. on a differential scanning fluorimetry (DSF) assay (K_{D} = 22µM).

Tables 1, 2, and 3 below provides DSF assay results for Examples 1-7, Examples 8-37, and Examples 38-94, respectively.

TABLE 1

DSF Assay results for synthetized Examples 1-7

| Example # | DSF Activity range |
|---|---|
| 1 | A |
| 2 | C |
| 3 | C |
| 4 | B |
| 5 | D |
| 6 | B |
| 7 | C |

TABLE 2

Assay results for commercially available Examples 8-37

| Example # | DSF Activity Range |
|---|---|
| 8 | A |
| 9 | A |
| 10 | C |
| 11 | B |
| 12 | B |
| 13 | D |
| 14 | A |
| 15 | D |
| 16 | B |
| 17 | B |
| 18 | A |
| 19 | C |
| 20 | B |
| 21 | B |
| 22 | C |
| 23 | B |
| 24 | B |
| 25 | B |
| 26 | B |
| 27 | B |
| 28 | B |
| 29 | B |
| 30 | B |
| 31 | C |
| 32 | D |
| 33 | C |
| 34 | D |
| 35 | B |
| 36 | B |
| 37 | B |

TABLE 3

DSF Assay results for synthetized Examples 38-94

| Example # | DSF Activity range |
|---|---|
| 38 | B |
| 39 | B |
| 40 | B |
| 41 | C |
| 42 | C |
| 43 | D |
| 44 | B |
| 45 | B |
| 46 | B |
| 47 | D |
| 48 | C |
| 49 | C |
| 50 | B |
| 51 | C |
| 52 | B |
| 53 | B |
| 54 | B |
| 55 | C |
| 56 | A |
| 57 | B |
| 58 | B |
| 59 | B |
| 60 | B |
| 61 | D |
| 62 | B |
| 63 | B |
| 64 | B |
| 65 | D |
| 66 | B |
| 67 | B |
| 68 | C |
| 69 | C |
| 70 | B |
| 71 | A |
| 72 | B |
| 73 | C |
| 74 | B |
| 75 | B |
| 76 | D |
| 77 | C |
| 78 | D |
| 79 | C |
| 80 | B |
| 81 | C |
| 82 | B |
| 83 | D |
| 84 | C |
| 85 | B |
| 86 | D |
| 87 | D |
| 88 | B |
| 89 | B |
| 90 | C |
| 91 | B |
| 92 | B |
| 93 | C |
| 94 | B |

### Denaturation Prevention

The capacity of Compounds of the Disclosure to stabilize α-L-iduronidase in neutral pH buffer was assessed using the denaturation prevention assay.

Briefly, recombinant human α-L-iduronidase protein (obtained in the Institute of Biotechnology and Biomedicine in the UAB, Bellaterra, Spain) was combined at a final concentration of 1 µM with 5X of SYPRO Orange and 30 µM of the corresponding compound in a final reaction volume of 25 µL in 100 mM Hepes 20 mM MgCl₂ pH 7 buffer. Reactions were incubated at 37° C. and SYPRO Orange fluorescence intensity was monitored at the indicated time points using Glomax® Discover Microplate Reader from Promega (Madison, WI, USA).

The result of an exemplary Compound of the Disclosure, Compound A, is presented in FIG. 2. The compound referred to as Compound A throughout this specification is the compound of Example 8 described above. The results show that Compound A prevents IDUA denaturation. Compound A (30 µM) slows down pH-induced denaturation of recombinant human IDUA. The reference compound L-iduronic acid (500 µM) shows a minimal effect. (T=37° C.).

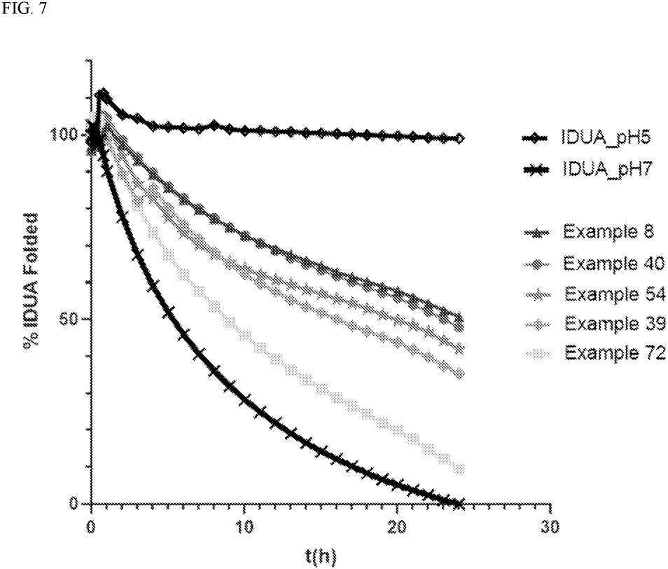

Compounds of Examples 8, 39, 40, 54, and 72 were also tested as described above, except that ALDURAZYME® was used instead of the recombinant human α-L-iduronidase protein (obtained in the Institute of Biotechnology and Biomedicine in the UAB, Bellaterra, Spain). The results are presented in FIG. 7. The results show that all the tested compounds prevent IDUA denaturation.

### IDUA Inhibition Assay

The ability of Compound of the Disclosure to inhibit IDUA was assessed by the assay described below.

Recombinant human α-L-iduronidase protein IDUA PR_06_WT_X (obtained in the Institute of Biotechnology and Biomedicine in the UAB, Bellaterra, Spain) was prepared at the desired concentration in PBS:

Recombinant Human IDUA. HEK293f. Ala26-Pro653 with a C-terminal 10_His tag. Protein buffer: 40 mM sodium acetate, 400 mM NaCl and 20% glycerol, pH=5. Produced by PPP IBB UAB.

The activity assay (3 replicates of each sample) included the following procedures:

In dark 96-well plates compatible with fluorometer:
1) 25 µL of Recombinant protein or PBS (in blank samples);
2) 1 µL CO (50X concentrate, made in an intermediate plate) or DMSO for the untreated samples;
3) 25 µL of 200 µM substrate solution (from a stock solution of 20 mM of substrate in DMSO), 4-Methylumbelliferyl alpha-L-iduronide (Glycosynth, 44076) in 0,4 M sodium formate, pH 3,5, and 0,2% triton;
4) Mix (shaker);
5) 37° C. 60 min;
6) Stop the reaction by adding 200 µL of 0,5 M Glycine / 0,3 M NaOH, pH=10; and
7) Read Fluorescence at Ex/Em: 340/460 nm.

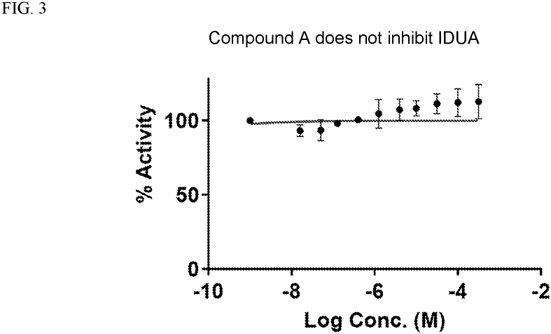

The result of an exemplary Compound of the Disclosure, Compound A, in the IDUA inhibition assay is presented in FIG. 3. The results show that Compound A does not inhibit human IDUA. Compound A is a silent allosteric modulator that does not affect the intrinsic enzymatic activity of recombinant human IDUA event high concentrations.

### Enzyme Enhancement Activity of Compounds of the Disclosure in Combination with α-L-iduronidase Protein

The effect of Compounds of the Disclosure in enhancing IDUA enzymatic activity can be tested as follows.

Patient-derived fibroblasts were seeded at 4×10⁴ cells per well in 12-well cell culture plates in Dulbecco’s Modified Eagle’s Media (DMEM) supplemented with 10% of fetal bovine serum (FBS), 1% penicillin/streptomycin (P/S) (Thermo Fisher Scientific, Waltham, MA, USA) and incubated at 37° C., 5% CO₂ overnight for cell attachment. Subsequently, cells were incubated in the absence or presence of the compounds and/or 1.25 nM recombinant human α-L-iduronidase protein (obtained in the Institute of Biotechnology and Biomedicine in the UAB) for 4 days. After incubation, cells were washed with phosphate-buffered saline (“PBS”) and detached using Trypsin-EDTA solution (Sigma Aldrich, St. Louis, MO, USA) to prepare cell pellets. The pellets were stored at -80° C. until activity assays were performed. α-L-iduronidase activity in cell lysates was measured by using 4-methylumbelliferyl alpha-L-iduronide substrate (Glycosynth, Warrington, UK). Briefly, lysates were resuspended in 25 µL of 0.9% NaCl containing 0.01% triton X-100 lysis buffer to promote membrane disruption. The cell suspension was sonicated and centrifuged to remove insoluble materials. Then, lysates were mixed with 25 µL of 200 µM 4-MU- alpha-L-iduronide substrate in 0.4 M sodium formate pH 3.5 0.2% triton X-100 buffer for 60 min at 37° C. The reaction was terminated by adding 200 µL of 0.5 M Glycine/0.3M NaOH buffer pH=10. The liberated 4-MU was measured on a Glomax® Discover Microplate Reader from Promega with excitation at 340 nm and emission at 460 nm. Protein quantification was determined using Pierce BCA Protein Assay Kit (Thermo Fisher Scientific, Waltham, MA, USA). Measurements were interpolated in a 4-MU standard curve and normalized by protein quantity.

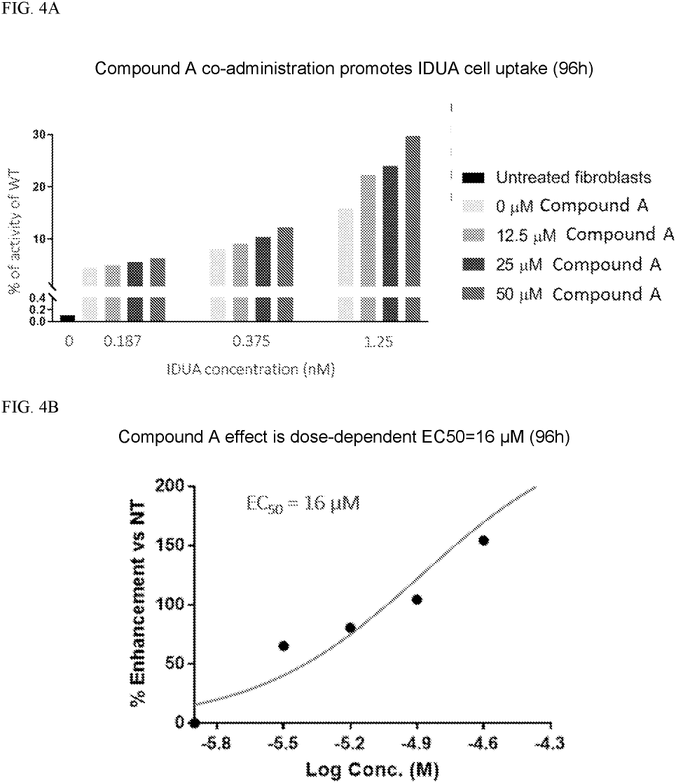

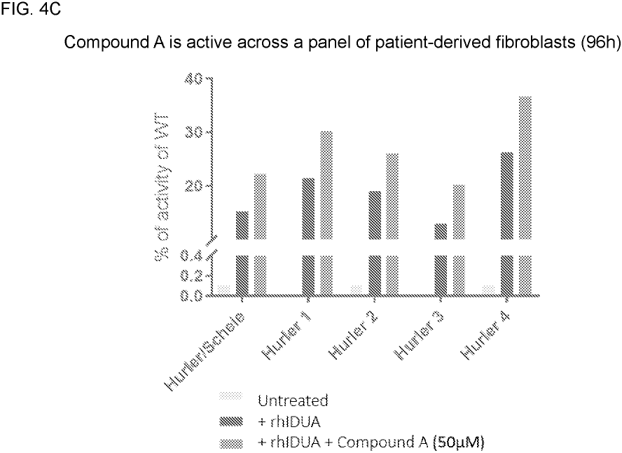

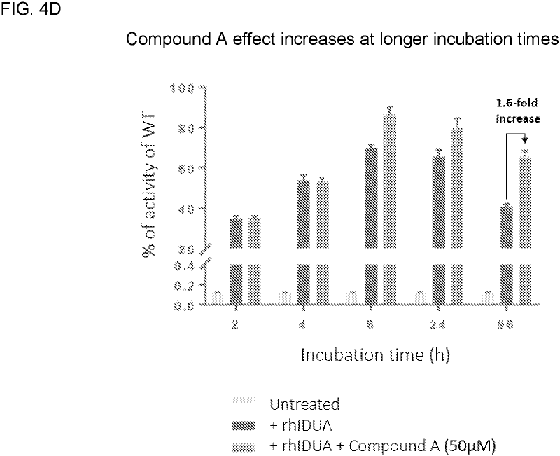

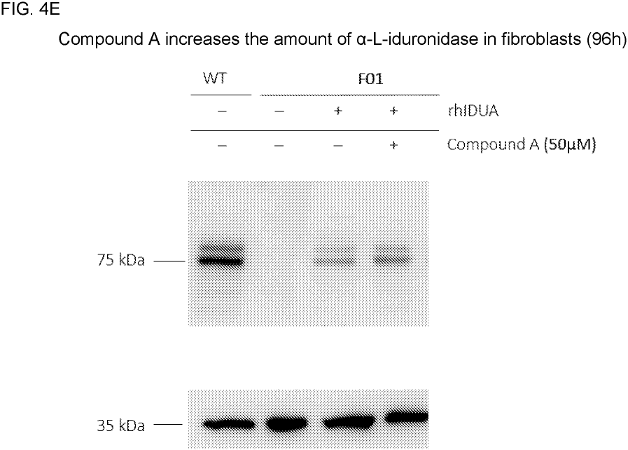

Results of an exemplary Compound of the Disclosure, Compound A, in cell-based assays are presented in FIGS. 4A-4E. Cell-based assays demonstrate the potential of Compound A as an ERT combination therapy. Compound A increases uptake of rhIDUA protein and its enzymatic activity under a wide range of conditions (rhIDUA concentration and cell-types). Benefits are particularly evident at longer incubation times (4 days). The results show that IDUA activity increases in a dose-dependent manner after the addition of rhIDUA to the culture medium in Hurler-Scheie fibroblasts (Basal activity: 0.23). Co-administration with Compound A shows a marked increase of IDUA cell uptake (vs. single agent) at two different concentrations and in dose-dependent manner. The cell-based EC₅₀ (16 µM) is in good agreement with the K_{D} by DSF (22 µM) (FIG. 4A). The results in FIG. 4B show that the effect of Compound A is dose dependent. FIG. 4C shows that Compound A is active across a panel of patient-derived fibroblasts (96h). FIG. 4D shows that the effect of Compound A in fibroblasts increases at longer incubation times. FIG. 4D shows that Compound A increases the amount of α-L-iduronidase in fibroblasts (96h).

### In Vivo Activity of an Exemplary Compound of the Disclosure (Compound A)

Assay goal:

Evaluation of IDUA activity in mouse bone joint cartilage samples obtained from BIO-PCY-19-025 study, where C57BL/6 male mice were administered with 1.2 mg/kg Aldurazyme (i.v. bolus administration) in the presence and absence of Compound A.

Groups Details:
G1: Vehicle 2 +Aldurazyme (1.2 mg/kg,i.v.,) QD×1(n=6) [vehicle 2:NMP (5%) + Solutol HS-15 (5%) + Normal saline (45%) + PEG-400 (45%)];
G2: Compound A (5 mg/kg, i.v.) + Aldurazyme (1.2 mg/kg, i.v.) QD x 1 (n=6);
G3: Compound A (10 mg/kg, i.v.) + Aldurazyme (1.2 mg/kg, i.v.) QD x 1 (n=6);
G4: Compound A (20 mg/kg, i.v.) + Aldurazyme (1.2 mg/kg, i.v.) QD x 1 (n=6); and
G5: Naïve Control (n=6).

IDUA assay in Plasma:

Plasma samples from mice were collected at indicated time points. Plasma samples were diluted in formate buffer and total protein was estimated using Bradford reagent. 25 µl of tissue homogenate and plasma samples were added to 25 µl of 0.18 mM of 4 MUI substrate and incubated at 37° C. for 30 min. Reaction was terminated using 200 µl of stop solution (0.3 M Glycine and 0.2 M sodium carbonate, pH 10.4). Plate was read at excitation 355 nm and emission 460 nm. 4-MU standard curve was obtained in duplicates from conc. range of 10 µM to 0.002 µM.

IDUA assay in Bone cartilage:

Bone joint cartilage samples from mice were collected at indicated time points. Tissues were homogenized in RIPA buffer, protein was extracted was diluted in water. Total protein was estimated using Bradford reagent. 25 µl of tissue homogenate and plasma samples were added to 25 µl of 0.18 mM of 4 MUI substrate and incubated at 37° C. for 30 min. Reaction was terminated using 200 µl of stop solution (0.3 M Glycine and 0.2 M sodium carbonate, pH 10.4). Plate was read at excitation 355 nm and emission 460 nm. 4-MU standard curve was obtained in duplicates from conc. range of 10 µM to 0.002 µM.

IDUA assay in Bone marrow:

Bone marrow samples from mice were collected at indicated time points. After repeated freeze thaw cycles for protein extraction, bone marrow samples were diluted in formate buffer (0.4 M, pH 3.5). Total protein was estimated using Bradford reagent. 25 µl of tissue homogenate and plasma samples were added to 25 µl of 0.18 mM of 4 MUI substrate and incubated at 37° C. for 30 min. Reaction was terminated using 200 µl of stop solution (0.3 M Glycine and 0.2 M sodium carbonate, pH 10.4). Plate was read at excitation 355 nm and emission 460 nm. 4-MU standard curve was obtained in duplicates from conc. range of 10 µM to 0.002 µM.

Plate reader:

TECAN Infinite M1000Pro.

Data Analysis:

IDUA activity was assessed from 4-MU standard curve. The 4 MU release from 4 MUI substrate reaction with incubation time for 30 min as µM values were converted into nmoles/ml and normalized to total protein content in each sample (mg/ml). Final IDUA enzyme activity was expressed as nmols/mg of protein/hr. Data was analyzed using GraphPad prism software with one way ANOVA followed by Dunnett’s comparison test. Data is represented as mean± SEM.

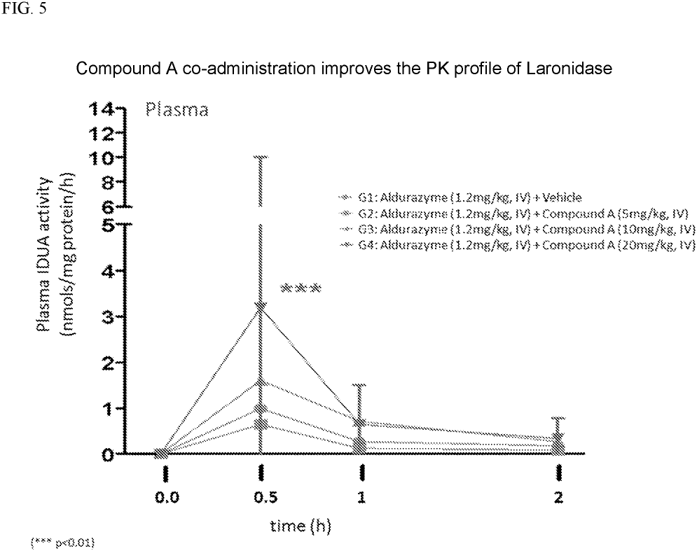

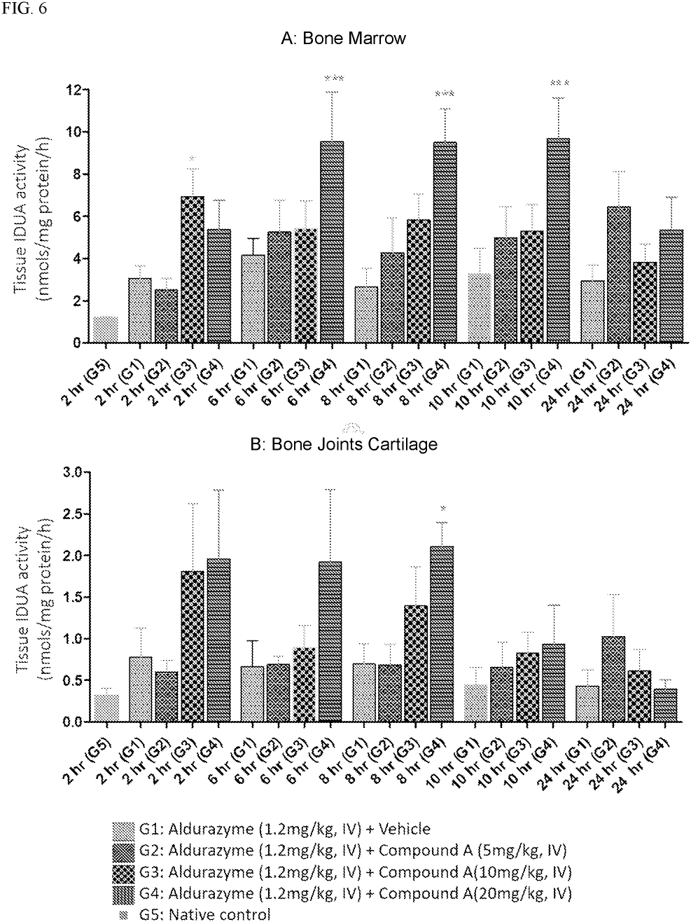

In vivo, Compound A improves the PK profile of Laronidase, increasing its plasma levels in a dose-dependent manner (see FIG. 5). It also increases IDUA enzymatic activity in a panel of tissues, including those that benefit the least from ERT (bone marrow, cartilages) (see FIG. 6). Compound A is safe at all doses tested (IV MTD 45 mg/kg).

The results show that co-administration of Laronidase with Compound A stabilizes the recombinant enzyme, increasing enzymatic activity levels in plasma, bone and cartilage in a dose-dependent manner. Bone and cartilage represent the most burning medical need due to poor ERT uptake. The benefit of combination therapy is particularly noticeable at longer times.

All publications cited in this specification are incorporated herein by reference. While the disclosure has been described with reference to particular embodiments, it will be appreciated that modifications can be made without departing from the spirit of the disclosure. Such modifications are intended to fall within the scope of the appended claims.

The disclosure also relates to the following particular embodiments designated as [1] for the first embodiment, [2] for the second embodiment, and so on:
[1] A method of treating or preventing a condition associated with the alteration of the activity of α-L-iduronidase in a patient, comprising administering to the patient in need thereof an effective amount of a compound of formula (I):

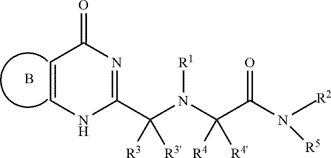

or a pharmaceutically acceptable salt or solvate thereof, optionally in combination with an effective amount of laronidase, wherein
   B is a fused benzene ring or a fused 5- or 6-membered heteroaromatic ring, wherein said benzene ring and said 5- or 6-membered heteroaromatic ring is optionally substituted;
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R³, R^{3'}, R⁴, and R^{4'} are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
   R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[2] A method of treating or preventing MPS1 in a patient, comprising administering to the patient in need thereof an effective amount of a compound of formula (I):

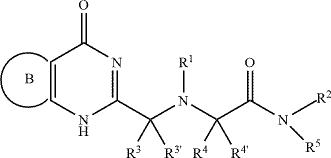

or a pharmaceutically acceptable salt or solvate thereof, optionally in combination with an effective amount of laronidase, wherein
   B is a fused benzene ring or a fused 5- or 6-membered heteroaromatic ring, wherein said benzene ring and said 5- or 6-membered heteroaromatic ring is optionally substituted;
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R³, R^{3'}, R⁴, and R^{4'} are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
   R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[3] The method of [1], wherein MPS1 is Hurler disease, Hurler-Scheie syndrome, or Scheie syndrome.
[4] A method of treating or preventing a heart valve disease, dysostosis multiplex, an eye disease, an ear disease, a respiratory obstruction or insufficiency, nerve compression, inflammatory arthritis, an amyloid related disorder, a disease condition associated with lipoprotein metabolism, solid cancers, infectious disease, an inflammatory disorder, or a developmental disorder in a patient, comprising administering to the patient in need thereof an effective amount of a compound of formula (I):

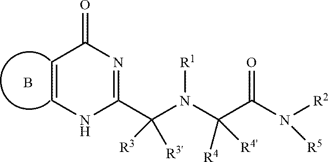

or a pharmaceutically acceptable salt or solvate thereof, optionally in combination with an effective amount of laronidase, wherein
   B is a fused benzene ring or a fused 5- or 6-membered heteroaromatic ring, wherein said benzene ring and said 5- or 6-membered heteroaromatic ring is optionally substituted; R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
   R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[5] The method of any one of [1]-[4], wherein B is a fused, optionally substituted benzene ring.
[6] The method of [5], wherein B is a fused, unsubstituted benzene ring.
[7] The method of [5], wherein B is a fused benzene ring substituted with one or more substituents selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[8] The method of any one of [1]-[4], wherein B is a fused, optionally substituted 5-membered heteroaromatic ring.
[9] The method of any one of [1]-[4] or [8], wherein B is a fused, unsubstituted 5-membered heteroaromatic ring.
[10] The method of any one of [1]-[4] or [8], wherein B is a fused 5-membered heteroaromatic ring substituted with 1 or 2 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[11] The method of any one of [1]-[4], wherein B is a fused, optionally substituted 6-membered heteroaromatic ring.
[12] The method of any one of [1]-[4] or [11], wherein B is a fused, unsubstituted 6-membered heteroaromatic ring.
[13] The method of any one of [1]-[4], [11], or [12], wherein B is a fused 6-membered heteroaromatic ring substituted with 1, 2 or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋ ₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[14] The method of any one of [1]-[4], wherein B is selected from the group consisting of

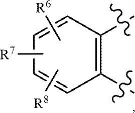

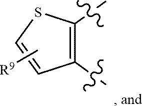

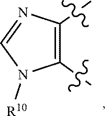

wherein R⁶, R⁷, R⁸, R⁹, and R¹⁰ are each independently selected from the group consisting of hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; wherein
   Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[15] The method of any one of [1]-[4] or [14], wherein B is selected from the group consisting of

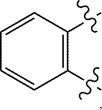

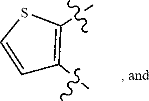

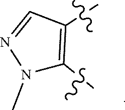

[16] The method of any one of [1]-[4], wherein B is

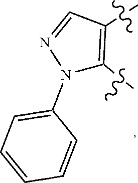

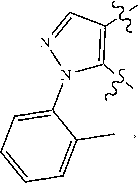

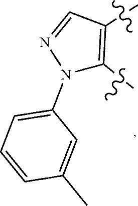

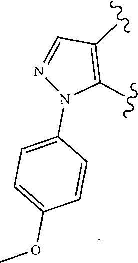

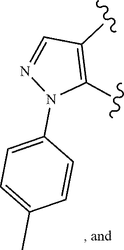

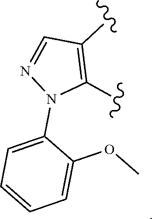

[17] The method of any one of [1]-[16], wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋ ₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
   R³, R^{3'}, R⁴, and R^{4'} are each independently selected from the group consisting of hydrogen, halogen, unsubstituted C₁₋₄ alkyl, and substituted C₁₋₄ alkyl.
[18] The method of any one of [1]-[17], wherein R³ and R⁴ are hydrogen, and R^{3'} and R⁴' are each independently selected from the group consisting of hydrogen, halogen, unsubstituted C₁₋₄ alkyl, and substituted C₁₋₄ alkyl.
[19] The method of any one of [1]-[18], wherein R³, R^{3'}, R⁴, and R^{4'} are each hydrogen.
[20] The method of any one of [1]-[16], wherein R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R^{3'}, R⁴, and R^{4'} are as defined in [1].
[21] The method of any one of [1]-[16], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are as defined in [1].
[22] The method of any one of [1]-[16], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are each hydrogen.
[23] The method of any one of [1]-[22], wherein R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋ ₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.
[24] The method of any one of [1]-[23], wherein R² is selected from the group consisting of

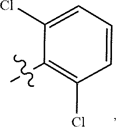

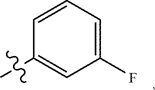

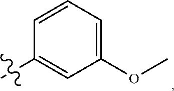

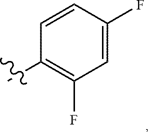

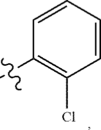

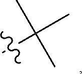

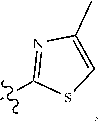

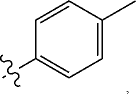

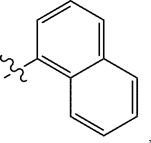

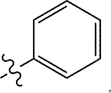

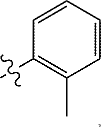

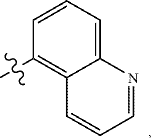

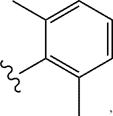

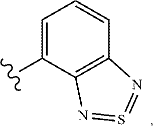

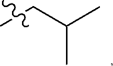

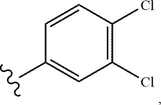

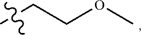

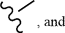

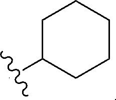

[25] The method of any one of [1]-[24], wherein R⁵ is unsubstituted C₁₋₆ alkyl.
[26] The method of any one of [1]-[22], wherein R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring.
[27] The method of any one of [1]-[22] or [26], wherein R² and R⁵ together with the nitrogen atom to which they are attached form a group

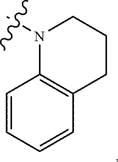

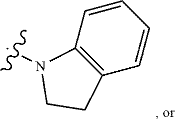

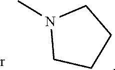

[28] The method of any one of [1]-[4], wherein the compound of formula (I) is the compound of formula (I′):

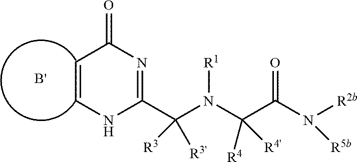

or a pharmaceutically acceptable salt or solvate thereof, wherein B′ is a fused ring selected from the group consisting of

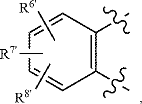

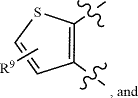

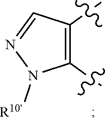

R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R⁴' are each independently selected from the group consisting of hydrogen, halogen, unsubstituted C₁₋₄ alkyl, and substituted C₁₋₄ alkyl; or
R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[29] The method of [28], wherein B′ is

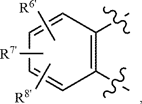

wherein R^{6'}, R^{7'}, and R^{8'} are as defined in [31].
[30] The method of [28], wherein B′ is

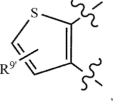

wherein R^{9'} is as defined in [31].
[31] The method of [28], wherein B′ is

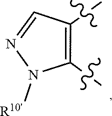

wherein R^{10'} is as defined in [31].
[32] The method of [28] or [31], wherein B′ is B3' selected from the group consisting of

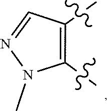

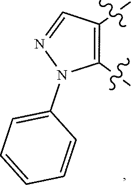

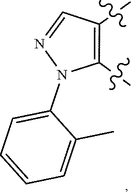

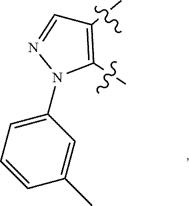

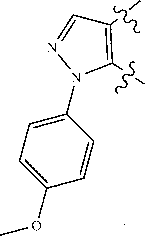

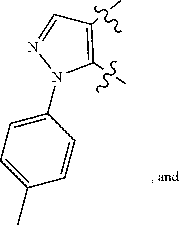

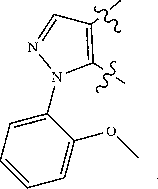

[33] The method of any one of [28]-[32], wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋ ₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and R³, R^{3'}, R⁴, and R⁴' are each independently selected from the group consisting of hydrogen, halogen, unsubstituted C₁₋₄ alkyl, and substituted C₁₋₄ alkyl.
[34] The method of any one of [28]-[33], wherein R³ and R⁴ are hydrogen, and R^{3'} and R⁴' are each independently selected from the group consisting of hydrogen, halogen, unsubstituted C₁₋₄ alkyl, and substituted C₁₋₄ alkyl.
[35] The method of any one of [28]-[33], wherein R³, R^{3'}, R⁴, and R^{4'} are each hydrogen.
[36] The method of any one of [28]-[32], wherein R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R^{3'}, R⁴, and R^{4'} are as defined in [28].
[37] The method of any one of [28]-[32], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are as defined in [28].
[38] The method of any one of [28]-[32], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are each hydrogen.
[39] The method of any one of [28]-[38], wherein R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; and
   R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.
[40] The method of any one of [28]-[39], wherein R^{2b} is selected from the group consisting of

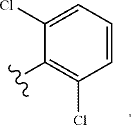

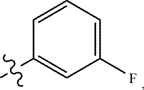

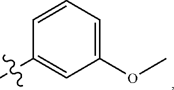

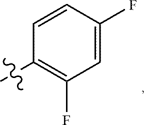

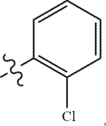

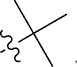

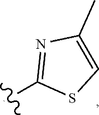

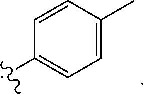

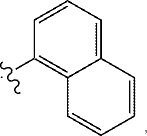

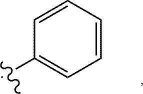

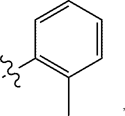

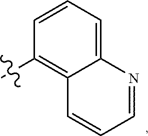

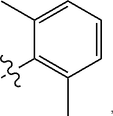

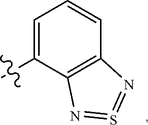

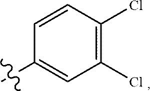

and

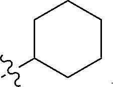

[41] The method of any one of [28]-[40], wherein R^{5b} is unsubstituted C₁₋₆ alkyl.
[42] The method of any one of [28]-[41], wherein R^{5b} is unsubstituted C₂-₆ alkyl.
[43] The method of any one of [1]-[42], wherein Ra is hydrogen or -C₁₋₄ alkyl optionally substituted by 1, 2 or 3 fluorine atoms.
[44] The method of any one of [1]-[42], wherein Ra -C₃₋₁₀ cycloalkyl or -(5- to 10-membered)-C₂₋₉ heterocyclyl, wherein cycloalkyl or heterocyclyl group is optionally substituted by 1, 2 or 3 fluorine atoms.
[45] The method of any one of [1]-[4], wherein the compound is selected from the group consisting of

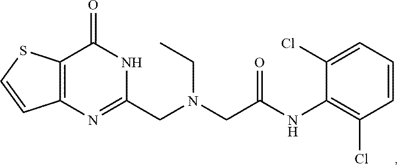

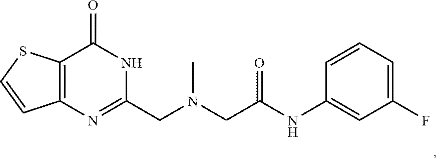

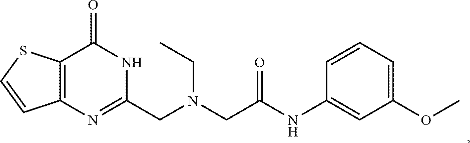

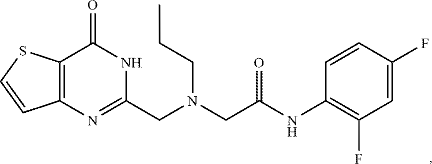

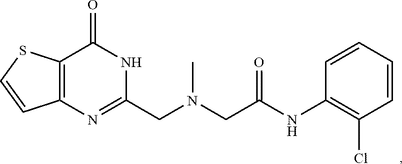

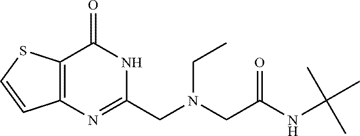 [SMILES: CCN(CC(=O)NC(C)(C)C)Cc1nc2ccsc2c(=O)[nH]1]

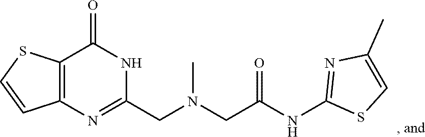

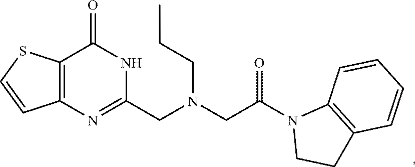

or a pharmaceutically acceptable salt or solvate thereof
[46] The method of any one of [1]-[4], wherein the compound is selected from the group consisting of

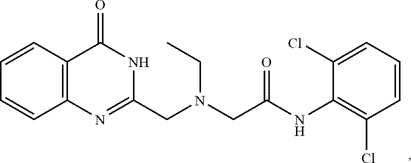

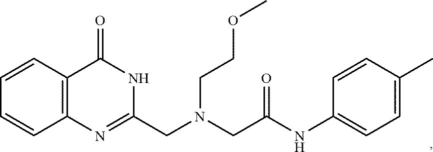

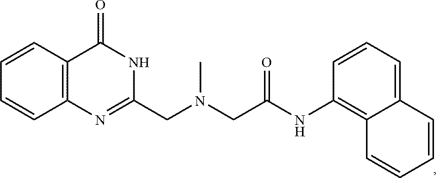

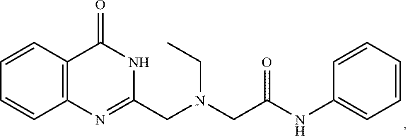

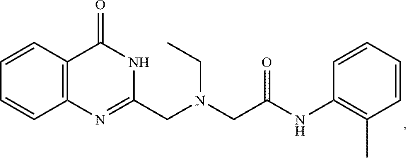

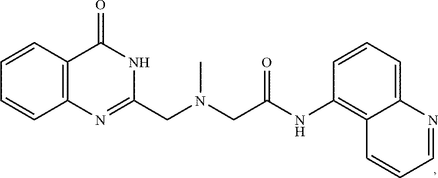

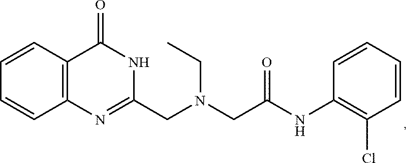

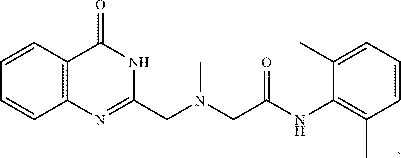

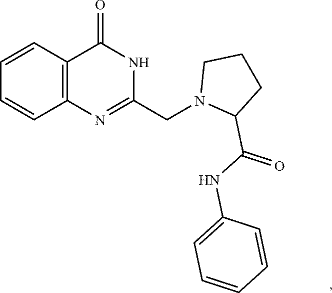

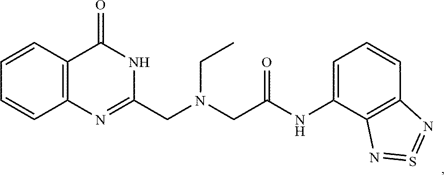

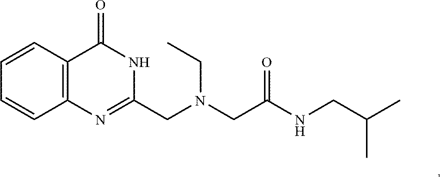

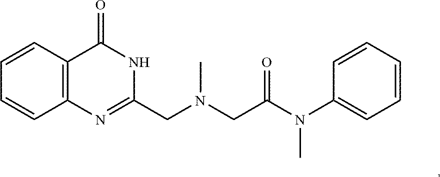

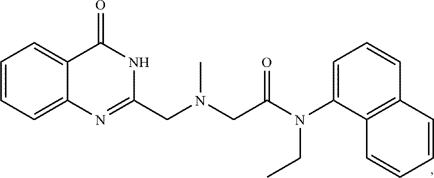

and

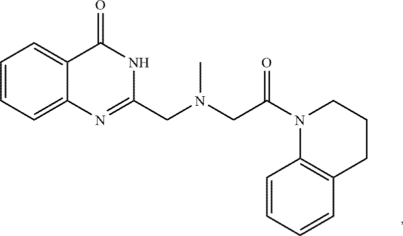

or a pharmaceutically acceptable salt or solvate thereof.
[47] The method of any one of claims [1]-[4], wherein the compound is selected from the group consisting of

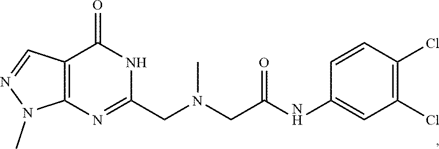

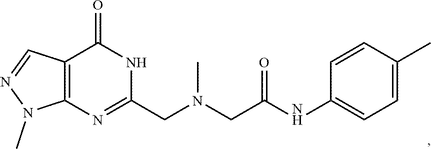

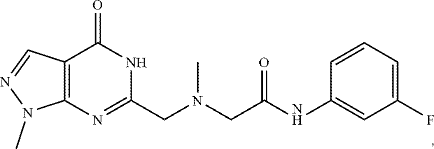

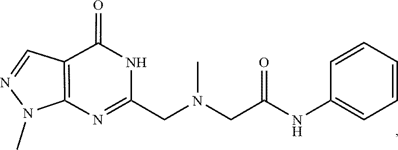

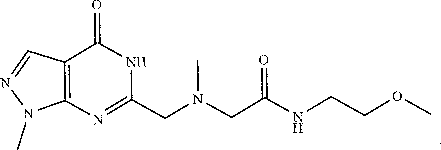

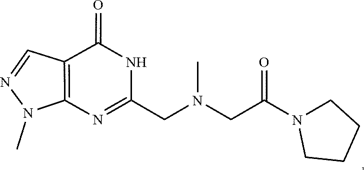

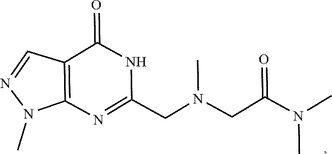

and

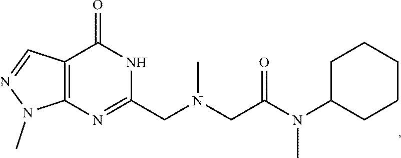

or a pharmaceutically acceptable salt or solvate thereof.
[48] The method of any one of [1]-[4] or [28], wherein the compound is selected from the group consisting of

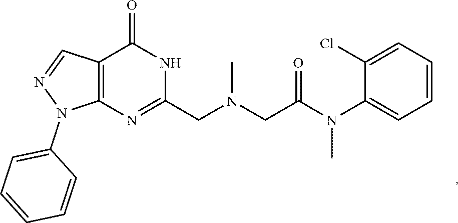

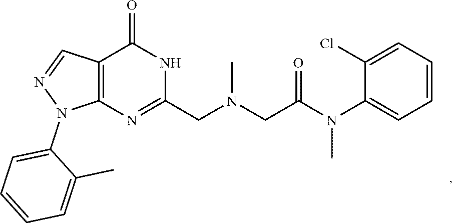

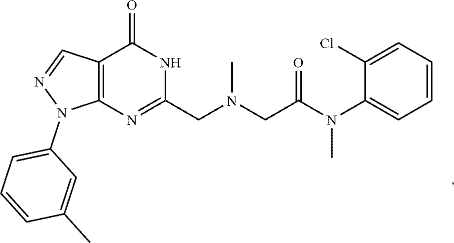

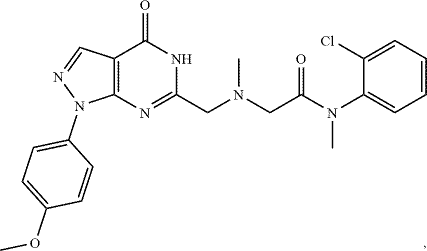

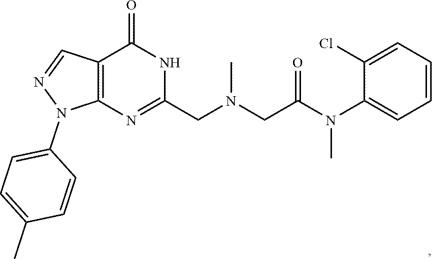

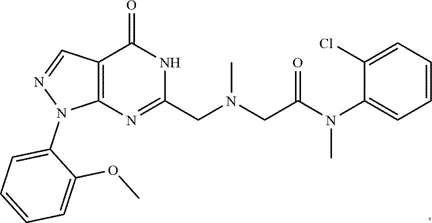

and

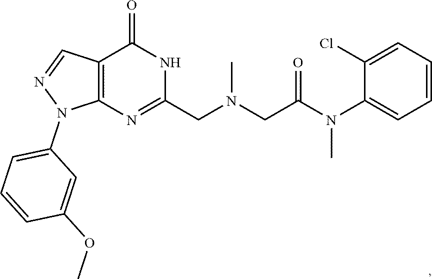

or a pharmaceutically acceptable salt or solvate thereof.
[49] The method of any one of [1]-[48], wherein said method does not include administering an effective amount of laronidase.
[50] The method of any one of [1]-[48], wherein said method comprises administering said compound of formula (I), or a pharmaceutically acceptable salt or solvate thereof, in combination with an effective amount of laronidase.
[51] The method of any one of [1]-[48] or [50], wherein said effective amount of a compound of formula (I), or a pharmaceutically acceptable salt or solvate thereof, and an effective amount of laronidase are administered simultaneously to the patient.
[52] The method of any one of [1]-[48] or [50], wherein said effective amount of a compound of formula (I), or a pharmaceutically acceptable salt or solvate thereof, and said effective amount of laronidase are administered to the patient sequentially.
[53] The method of any one of [50]-[52], wherein said effective amount of a compound of formula (I), or a pharmaceutically acceptable salt or solvate thereof, and said effective amount of laronidase are administered to the patient in separate pharmaceutical compositions.
[54] The method of any one of [50]-[52], wherein said effective amount of a compound of formula (I), or a pharmaceutically acceptable salt or solvate thereof, and said effective amount of laronidase are administered to the patient in a single pharmaceutical composition.
[55] A compound having the formula (I′):

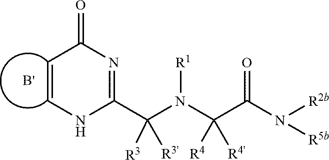

or a pharmaceutically acceptable salt or solvate thereof, wherein
   B′ is a fused ring selected from the group consisting of 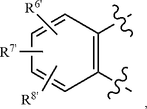 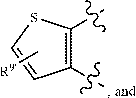 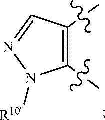
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, R^{7'}, and R^{8'} is other than hydrogen;
   R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
The compound of [55], wherein B′ is

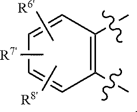

[57] The compound of [55], wherein B′ is

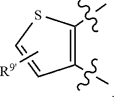

[58] The compound of [55], wherein B′ is

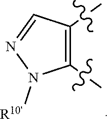

[59] The compound of [55] or [56], wherein B′ is B3' selected from the group consisting of

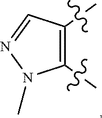

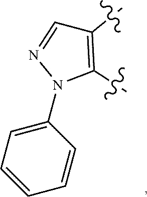

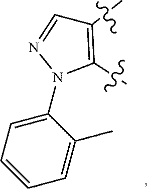

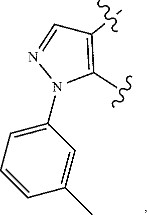

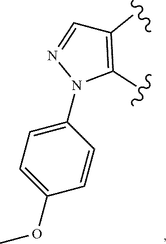

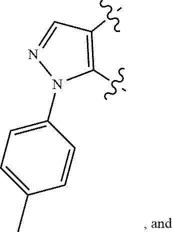

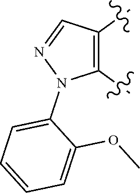

[60] The compound of any one of [55]-[59], wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
   R³, R^{3'}, R⁴, and R^{4'} are each independently selected from the group consisting of hydrogen, halogen, unsubstituted C₁₋₄ alkyl, and substituted C₁₋₄ alkyl.
[61] The compound of any one of [55]-[60], wherein R³ and R⁴ are hydrogen, and R³' and R⁴' are each independently selected from the group consisting of hydrogen, halogen, unsubstituted C₁₋₄ alkyl, and substituted C₁₋₄ alkyl.
[62] The compound of any one of [55]-[61], wherein R³, R^{3'}, R⁴, and R^{4'} are each hydrogen.
[63] The compound of any one of [55]-[60], wherein R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R^{3'}, R⁴, and R^{4'} are as defined in [54].
[64] The compound of any one of [55]-[60], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are as defined in [54].
[65] The compound of any one of [55]-[60], wherein R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O, and R³, R^{3'}, and R^{4'} are each hydrogen.
[66] The compound of any one of [55]-[65], wherein R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; and
   R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.
[67] The compound of any one of 55-66, wherein R^{2b} is selected from the group consisting of

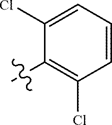

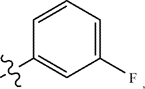

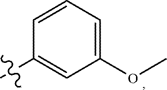

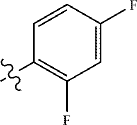

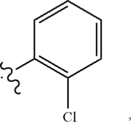

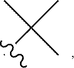

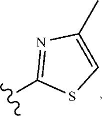

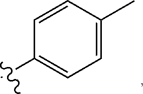

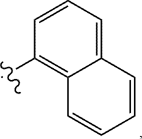

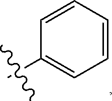

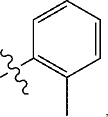

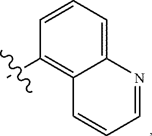

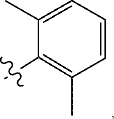

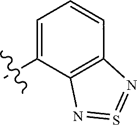

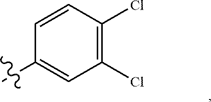

and

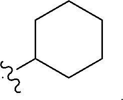

[68] The compound of any one of [55]-[67], wherein R^{5b} is unsubstituted C₁₋₆ alkyl.
[69] The compound of any one of [55]-[68], wherein R^{5b} is unsubstituted C₂₋₆ alkyl.
[70] The compound of any one of [55]-[69], wherein Ra is hydrogen or -C₁₋₄ alkyl optionally substituted by 1, 2 or 3 fluorine atoms.
[71] The compound of any one of [55]-[69], wherein Ra -C₃₋₁₀ cycloalkyl or -(5-to 10-membered)-C₂₋₉ heterocyclyl, wherein cycloalkyl or heterocyclyl group is optionally substituted by 1, 2 or 3 fluorine atoms.
[72] The compound of [55], wherein the compound is selected from the group consisting of

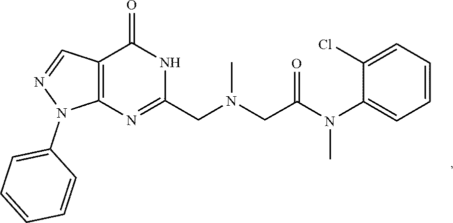

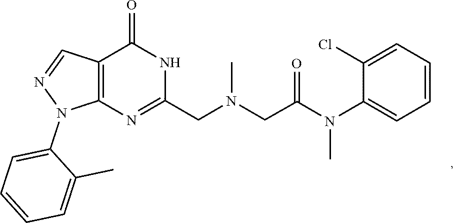

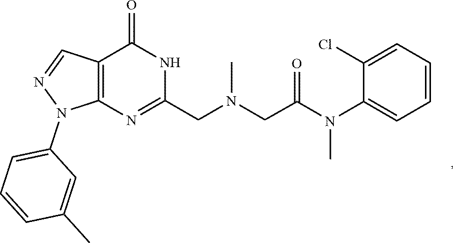

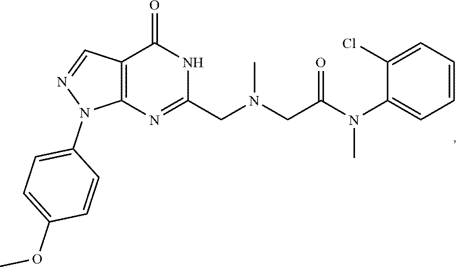

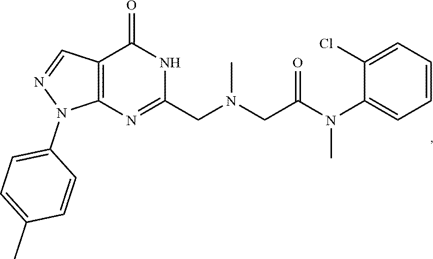

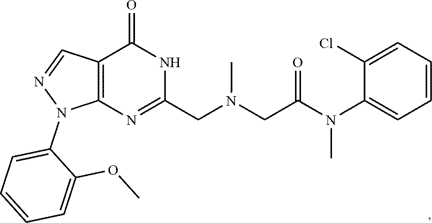

and

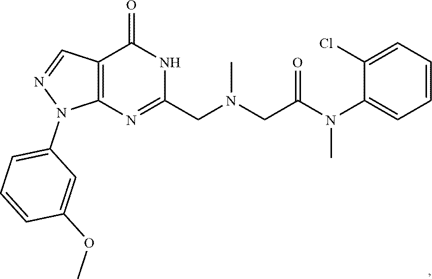

or a pharmaceutically acceptable salt or solvate thereof.
[73] A pharmaceutical composition, comprising an effective amount of a compound of formula (I), or a pharmaceutically acceptable salt or solvate thereof, and at least one pharmaceutically acceptable excipient, wherein the compound of formula (I) has the structure:

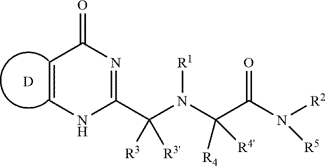

or a pharmaceutically acceptable salt or solvate thereof, wherein
   B is a fused benzene ring or a fused 5- or 6-membered heteroaromatic ring, wherein said benzene ring and said 5- or 6-membered heteroaromatic ring is optionally substituted;
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
   R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[74] The pharmaceutical composition of [73], wherein the compound is selected from the group consisting of

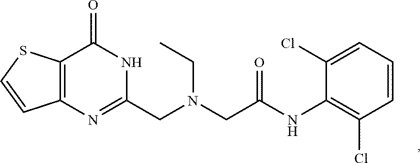

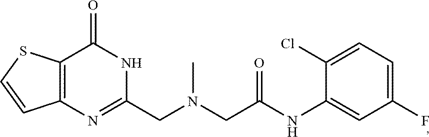

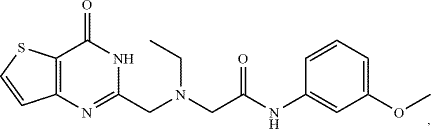

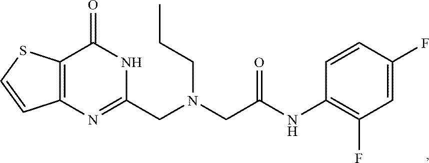

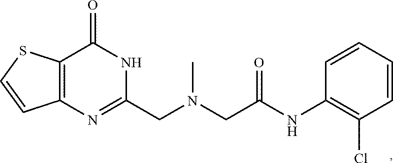

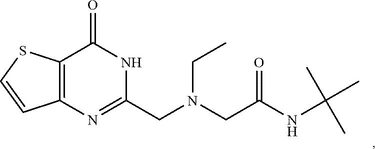

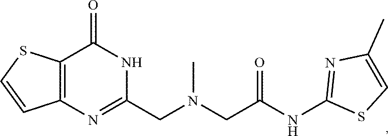

and

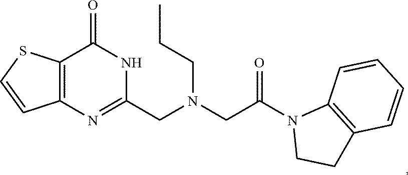

or a pharmaceutically acceptable salt or solvate thereof
[75] The pharmaceutical composition of [73], wherein the compound is selected from the group consisting of

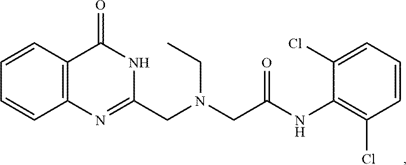

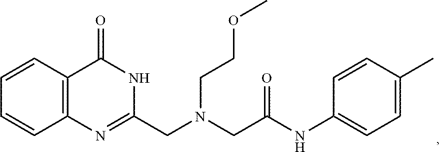

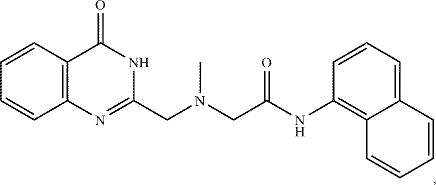

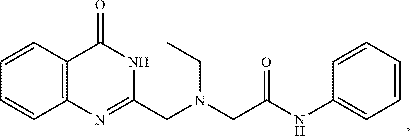

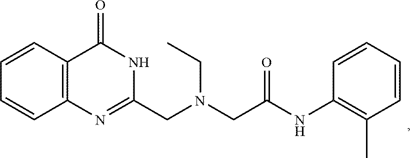

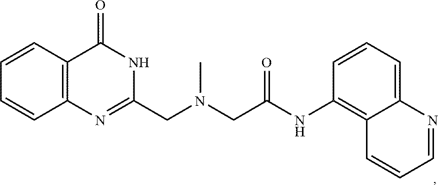

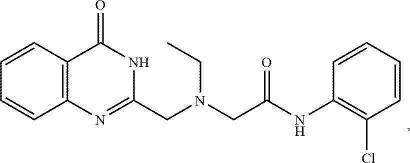

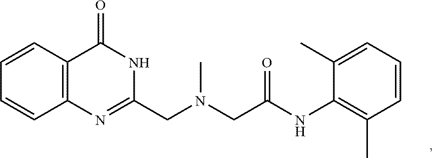

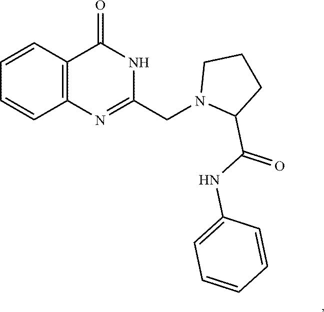

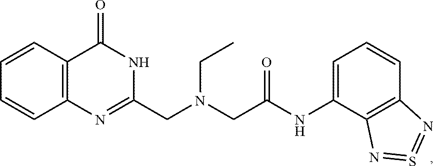

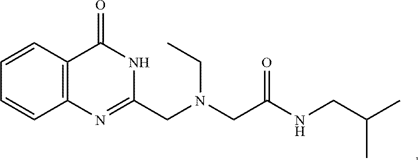

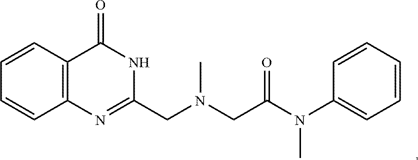

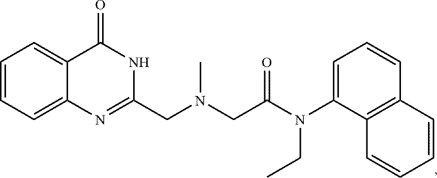

and

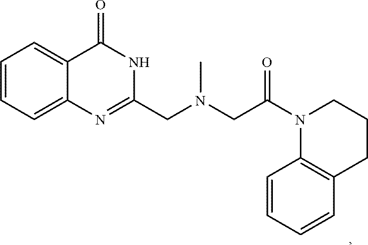

or a pharmaceutically acceptable salt or solvate thereof.
[76] The pharmaceutical composition of [73], wherein the compound is selected from the group consisting of

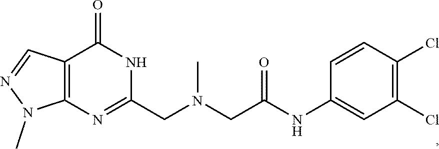

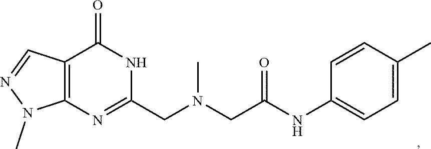

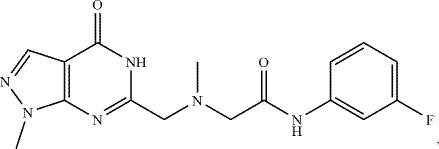

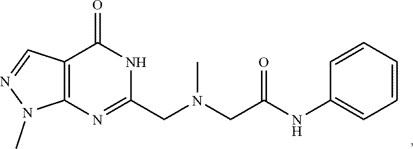

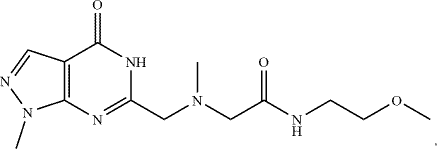

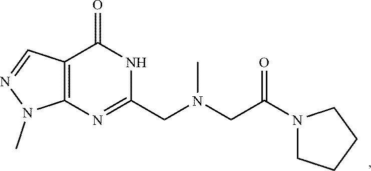

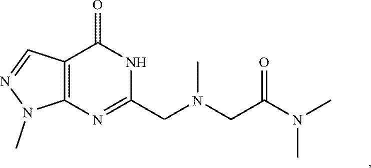

and

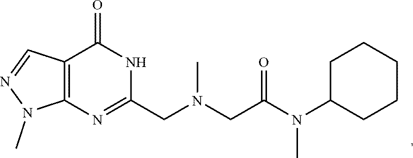

or a pharmaceutically acceptable salt or solvate thereof.
[77] The pharmaceutical composition of [73], wherein the compound of formula (I) is a compound of formula (I′) having the structure:

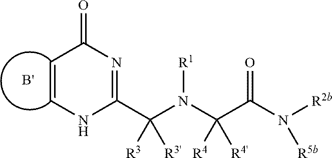

or a pharmaceutically acceptable salt or solvate thereof, wherein
   B′ is a fused ring selected from the group consisting of 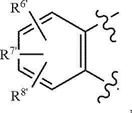 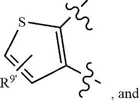 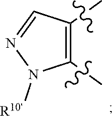
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, k^{7'}, and R^{8'} is other than hydrogen;
   R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[78] The pharmaceutical composition of [77], wherein the compound is selected from the group consisting of

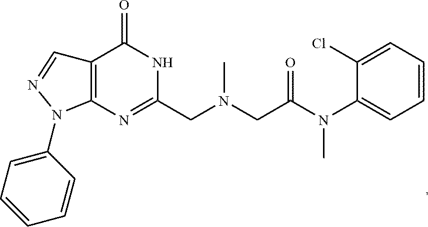

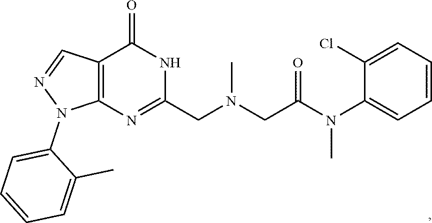

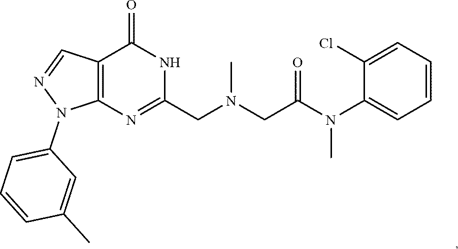

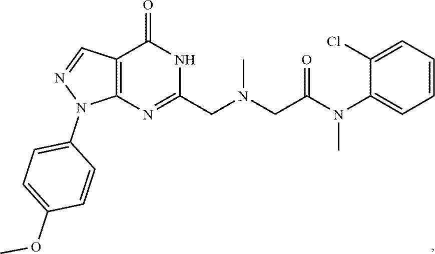

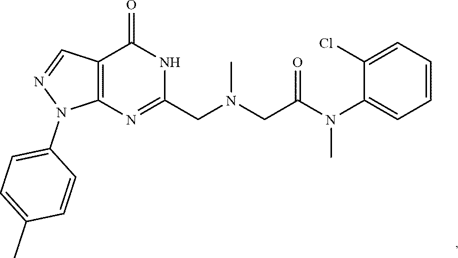

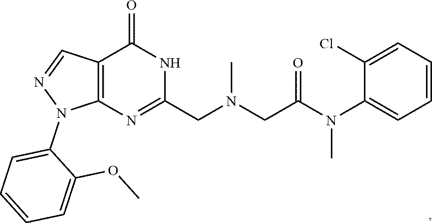

and

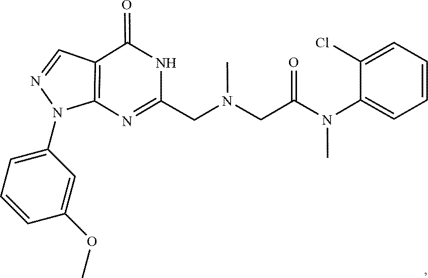

or a pharmaceutically acceptable salt or solvate thereof.
[79] A compound of formula (I):

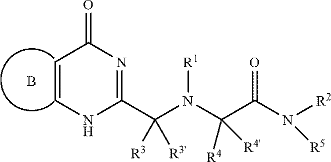

or a pharmaceutically acceptable salt or solvate thereof, for use as a medicament, optionally in a combination with laronidase, wherein
   B is a fused benzene ring or a fused 5- or 6-membered heteroaromatic ring, wherein said benzene ring and said 5- or 6-membered heteroaromatic ring is optionally substituted;
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
   R² and R⁵ together with the nitrogen atom to which they are attached form an optionally substituted 5- to 10-membered heterocyclic ring, wherein said heterocyclic ring optionally contains 1, 2, or 3 additional heteroatoms selected from the group consisting of N, S, or O, and wherein said heterocyclic ring is optionally fused to a phenyl ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[80] The compound for use according to [79], wherein the compound is selected from the group consisting of

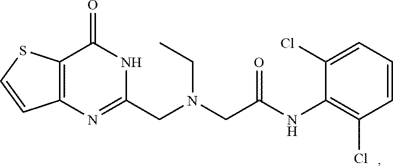

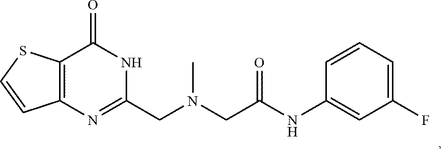

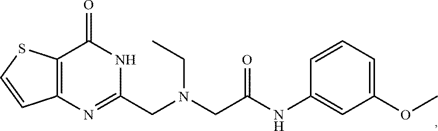

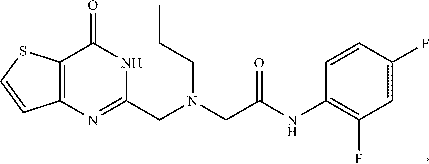

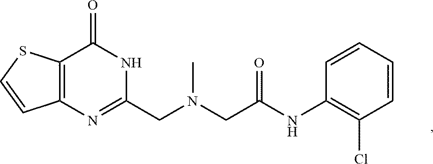

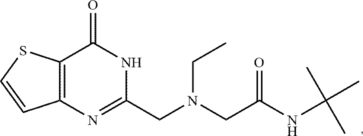

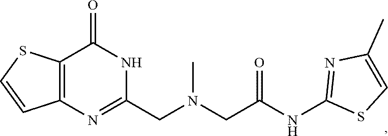

and

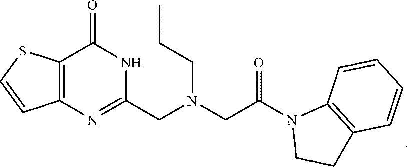

or a pharmaceutically acceptable salt or solvate thereof
[81] The compound for use according to [79], wherein the compound is selected from the group consisting of

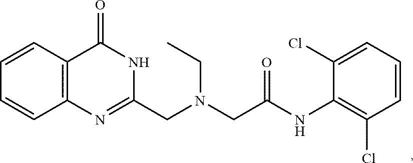

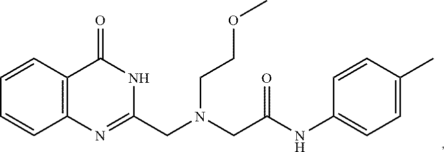

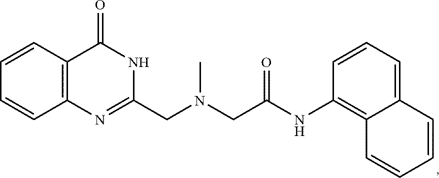

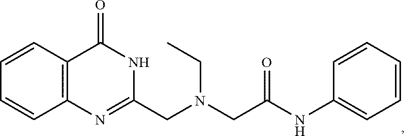

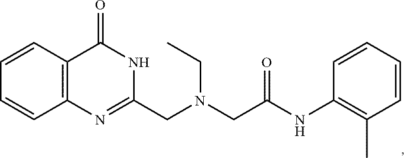

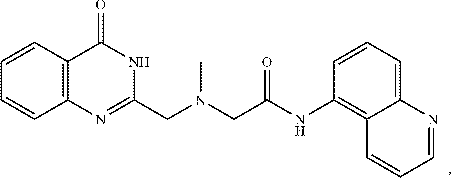

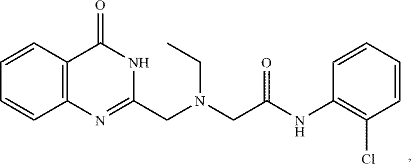

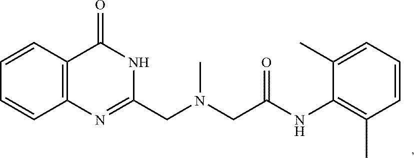

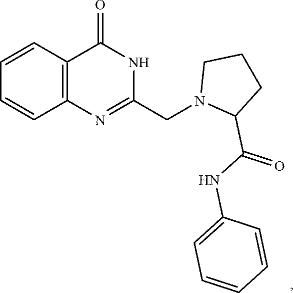

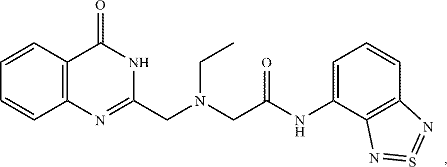

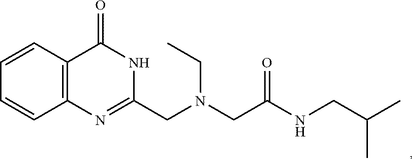

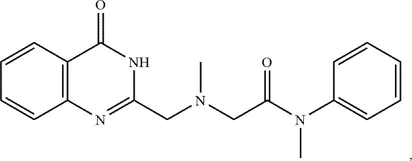

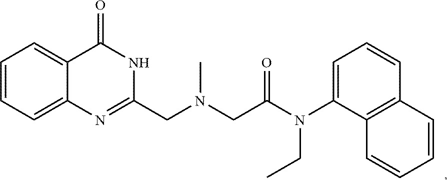

and

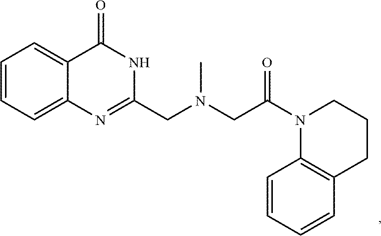

or a pharmaceutically acceptable salt or solvate thereof.
[82] The compound for use according to [79], wherein the compound is selected from the group consisting of

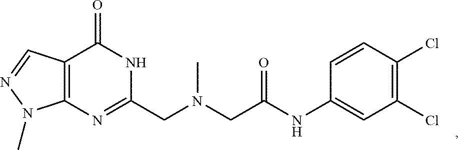

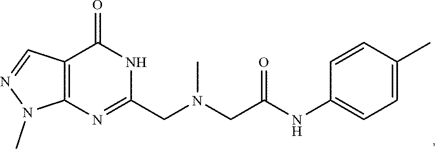

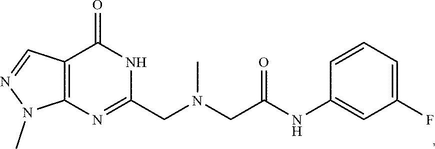

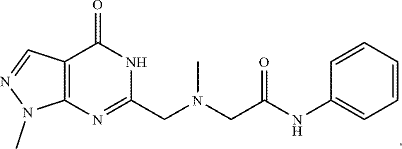

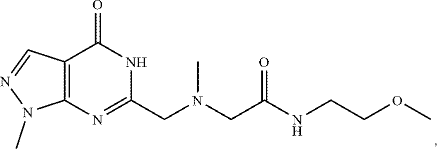

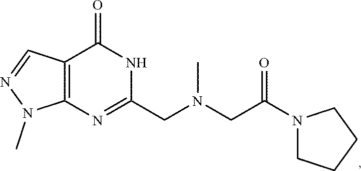

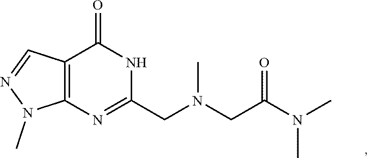

and

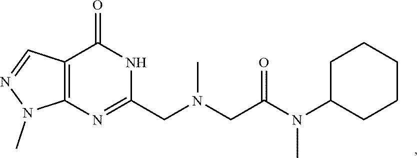

or a pharmaceutically acceptable salt or solvate thereof.
[83] The compound for use according to [79], wherein the compound of formula (I) is a compound of formula (I′) having the structure:

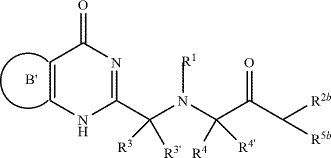

or a pharmaceutically acceptable salt or solvate thereof, wherein
   B′ is a fused ring selected from the group consisting of 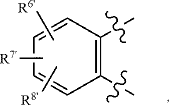 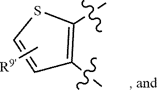 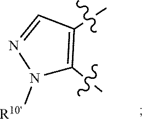
   R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{2b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
   R³, R^{3'}, R⁴, and R⁴' are each independently selected from the group consisting of hydrogen, halogen, unsubstituted C₁₋₄ alkyl, and substituted C₁₋₄ alkyl; or
   R¹ and R³ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O; or
   R¹ and R⁴ together with the nitrogen and carbon atoms to which they are attached form an optionally substituted 5- to 10-membered heterocyclic ring, wherein said heterocyclic ring optionally contains 1, 2, or 3 additional heteroatoms selected from the group consisting of N, S, or O;
   R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
   R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R^{6'}, R^{7'}, and R^{8'} is other than hydrogen;
   R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁-₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
   each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.
[84] The compound for use according to [83], wherein the compound is selected from the group consisting of

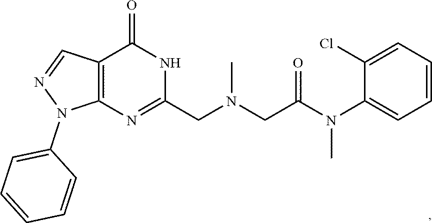

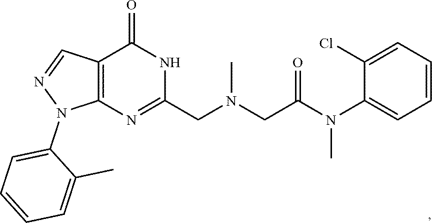

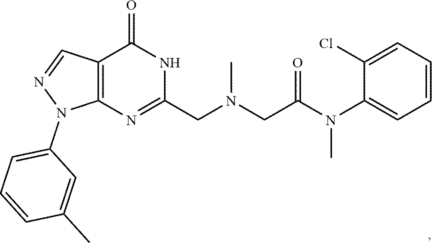

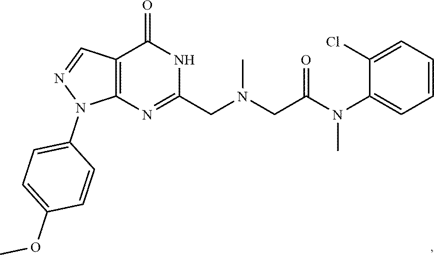

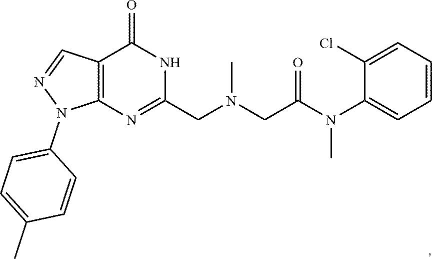

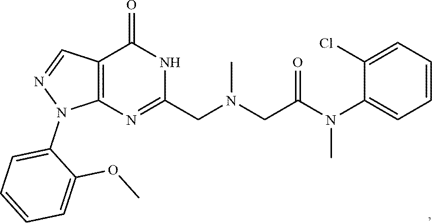

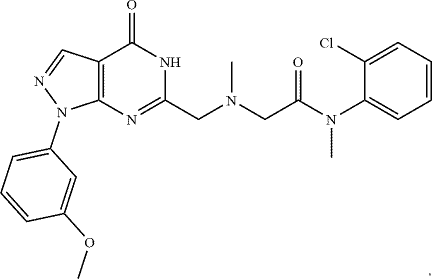

or a pharmaceutically acceptable salt or solvate thereof.
[85] The compound for use according to any one of [79]-[84], wherein the medicament is for use in the treatment or prevention of a condition associated with the alteration of the activity of α-L-iduronidase.
[86] The compound for use according to [85], wherein the condition is MPS 1.
[87] The compound for use according to [85], wherein the conditions is selected from the group consisting of a heart valve disease, dysostosis multiplex, an eye disease, an ear disease, a respiratory obstruction or insufficiency, nerve compression, inflammatory arthritis, an amyloid related disorder, a disease condition associated with lipoprotein metabolism, solid cancers, infectious disease, an inflammatory disorder, and a developmental disorder.

What is claimed is:

1. A method of treating or preventing a condition associated with the alteration of the activity of α-L-iduronidase in a patient, comprising administering to the patient in need thereof an effective amount of a compound of formula (I): 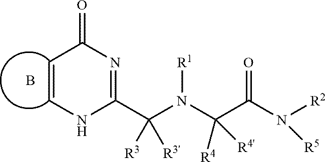
or a pharmaceutically acceptable salt or solvate thereof, optionally in combination with an effective amount of α-L-iduronidase or an analog or variant thereof, wherein
B is a fused benzene ring or a fused 5- or 6-membered heteroaromatic ring, wherein said benzene ring and said 5- or 6-membered heteroaromatic ring is optionally substituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂-₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃-₁₀ cycloalkyl, -C₁₋₄ alkyl-C₃-₁₀ cycloalkyl, -C₆₋₁₀ aryl, -C₁₋₄ alkyl-C₆₋₁₀ aryl, (5- to 10-membered)-C₁₋₉ heteroaryl, -C₁-₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl; orR¹ and R² together with the nitrogen and carbon atoms to which they are attached form an optionally substituted 5- to 10-membered heterocyclic ring, wherein 1, 2, or of the carbon atoms of said heterocyclic ring are optionally replaced by a heteroatom selected from the group consisting of N, S, and O;
R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by aheteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

2. A method of treating or preventing MPS 1 in a patient, comprising administering to the patient in need thereof an effective amount of a compound of formula (I): 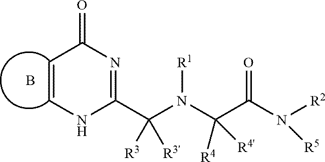
or a pharmaceutically acceptable salt or solvate thereof, optionally in combination with an effective amount of α-L-iduronidase or an analog or variant thereof, wherein
B is a fused benzene ring or a fused 5- or 6-membered heteroaromatic ring, wherein said benzene ring and said 5- or 6-membered heteroaromatic ring is optionally substituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂-₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R³', R⁴, and R⁴' are each independently selected from the group consisting of hydrogen, halogen, unsubstituted C₁₋₄ alkyl, and substituted C₁₋₄ alkyl;
R⁵ is selected from the group consisting of hydrogen, C₁-₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by aheteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

3. The method of claim 2, wherein MPS1 is Hurler disease, Hurler-Scheie syndrome, or Scheie syndrome.

4. A method of treating or preventing a heart valve disease, dysostosis multiplex, an eye disease, an ear disease, a respiratory obstruction or insufficiency, nerve compression, inflammatory arthritis, an amyloid related disorder, a disease condition associated with lipoprotein metabolism, solid cancers, infectious disease, an inflammatory disorder, or a developmental disorder in a patient, comprising administering to the patient in need thereof an effective amount of a compound of formula (I): 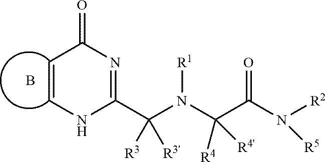
or a pharmaceutically acceptable salt or solvate thereof, optionally in combination with an effective amount of α-L-iduronidase or an analog or variant thereof, wherein
B is a fused benzene ring or a fused 5- or 6-membered heteroaromatic ring, wherein said benzene ring and said 5- or 6-membered heteroaromatic ring is optionally substituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂-₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃-₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R³', R⁴, and R⁴' are each independently selected from the group consisting of hydrogen, halogen, unsubstituted C₁₋₄ alkyl, and substituted C₁₋₄ alkyl;
R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by aheteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

5. The method of any one of claims 1-4, wherein B is a fused, optionally substituted benzene ring.

6. The method of any one of claims 1-5, wherein B is a fused, unsubstituted benzene ring.

7. The method of any one of claims 1-5, wherein B is a fused benzene ring substituted with one or more substituents selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

8. The method of any one of claims 1-4, wherein B is a fused, optionally substituted 5-membered heteroaromatic ring.

9. The method of any one of claims 1-4 or 8, wherein B is a fused, unsubstituted 5-membered heteroaromatic ring.

10. The method of any one of claims 1-4 or 8, wherein B is a fused 5-membered heteroaromatic ring substituted with 1 or 2 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

11. The method of any one of claims 1-4, wherein B is a fused, optionally substituted 6-membered heteroaromatic ring.

12. The method of any one of claims 1-4 or 11, wherein B is a fused, unsubstituted 6-membered heteroaromatic ring.

13. The method of any one of claims 1-4, 11, or 12, wherein B is a fused 6-membered heteroaromatic ring substituted with 1, 2 or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, wherein Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

14. The method of any one of claims 1-4, wherein B is selected from the group consisting of 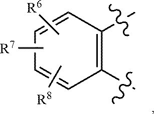 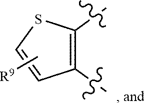 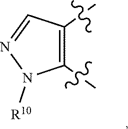
wherein R⁶, R⁷, R⁸, R⁹, and R¹⁰ are each independently selected from the group consisting of hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; wherein
Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

15. The method of any one of claims 1-4 or 14, wherein B is selected from the group consisting of 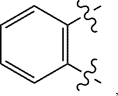 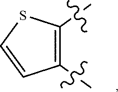
and 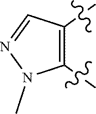
.

16. The method of any one of claims 1-4, wherein B is 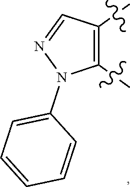 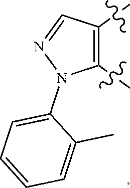 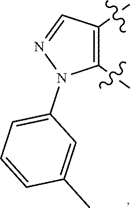 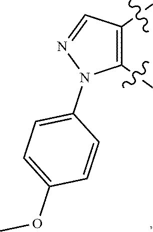 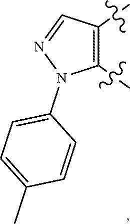
and 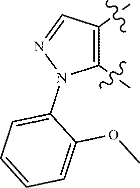
.

17. The method of any one of claims 1-16, wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5-to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R³, R^{3'}, R⁴, and R^{4'} are each independently selected from the group consisting of hydrogen, halogen, unsubstituted C₁₋₄ alkyl, and substituted C₁₋₄ alkyl.

18. The method of any one of claims 1-17, wherein R³ and R⁴ are hydrogen, and R³' and R⁴' are each independently selected from the group consisting of hydrogen, halogen, unsubstituted C₁₋₄ alkyl, and substituted C₁₋₄ alkyl.

19. The method of any one of claims 1-18, wherein R³, R^{3'}, R⁴, and R^{4'} are each hydrogen.

20. The method of any one of claims 1-16, wherein R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', R⁴, and R⁴' are as defined in claim 1.

21. The method of any one of claims 1-16, wherein R¹ and R³ together with the nitrogen and carbon atoms to which they are attached form an optionally substituted 5- to 10-membered heterocyclic ring, wherein 1, 2, or 3 carbon atoms of said heterocyclic ring are optionally replaced by a heteroatom selected from the group consisting of N, S, and O, and R^{3'}, R⁴, and R^{4'} are as defined in claim 1.

22. The method of any one of claims 1-16, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are as defined on claim 1.

23. The method of any one of claims 1-16 or 22, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are each hydrogen.

24. The method of any one of claims 1-23, wherein R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5-to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring.

25. The method of any one of claims 1-24, wherein R² is selected from the group consisting of 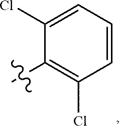 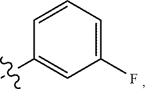 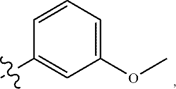 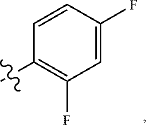 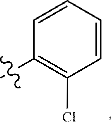 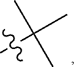 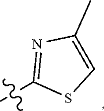 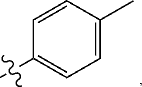 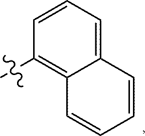 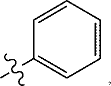 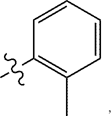 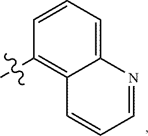 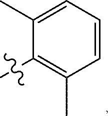 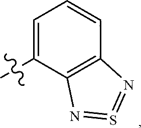 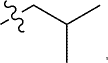 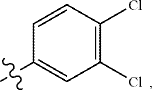 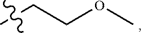 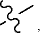
and 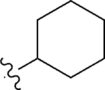
.

26. The method of any one of claims 1-25, wherein R⁵ is unsubstituted C₁₋₆ alkyl.

27. The method of any one of claims 1-23, wherein R² and R⁵ together with the nitrogen atom to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and wherein said heterocyclic ring is optionally fused to a phenyl ring.

28. The method of any one of claims 1-23 or 27, wherein R² and R⁵ together with the nitrogen atom to which they are attached form a group 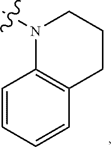 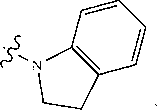 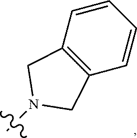
or 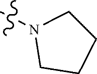
.

29. The method of any one of claims 1-4, wherein the compound of formula (I) is the compound of formula (I′): 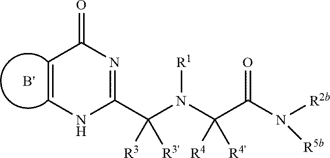
or a pharmaceutically acceptable salt or solvate thereof, wherein
B′ is a fused ring selected from the group consisting of 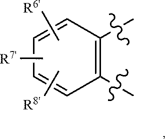 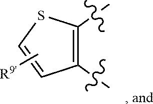 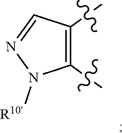
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring; or
R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋ ₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring;
R⁶', R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R⁶', R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

30. The method of claim 29, wherein B′ is 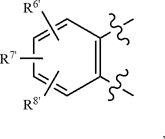
wherein R⁶', R^{7'}, and R^{8'} are as defined in claim 29.

31. The method of claim 29, wherein B′ is 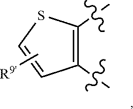
wherein R^{9'} is as defined in claim 29.

32. The method of claim 29, wherein B′ is 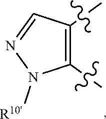
wherein R^{10'} is as defined in claim 29.

33. The method of claim 29 or 32, wherein B′ is B3' selected from the group consisting of 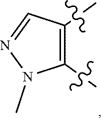 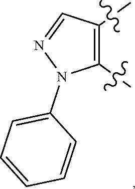 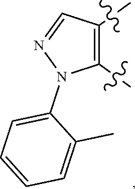 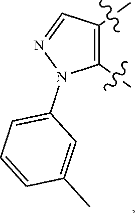 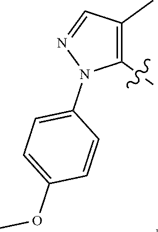 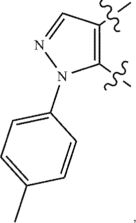 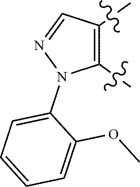
.

34. The method of any one of claims 29-33, wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R³, R^{3'}, R⁴, and R⁴' are each independently selected from the group consisting of hydrogen, halogen, unsubstituted C₁₋₄ alkyl, and substituted C₁₋₄ alkyl.

35. The method of any one of claims 29-34, wherein R³ and R⁴ are hydrogen, and R^{3'} and R^{4'} are each independently selected from the group consisting of hydrogen, halogen, unsubstituted C₁₋₄ alkyl, and substituted C₁₋₄ alkyl.

36. The method of any one of claims 29-34, wherein R³, R^{3'}, R⁴, and R^{4'} are each hydrogen.

37. The method of any one of claims 29-33, wherein R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R^{3'}, R⁴, and R^{4'} are as defined in claim 29.

38. The method of any one of claims 29-33, wherein R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{3'}, R⁴, and R⁴' are as defined in claim 29.

39. The method of any one of claims 29-33, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are as defined in claim 29.

40. The method of any one of claims 29-33 or 39, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are each hydrogen.

41. The method of any one of claims 29-36, wherein R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; and
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring.

42. The method of any one of claims 29-36 or 41, wherein R^{2b} is selected from the group consisting of 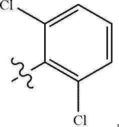 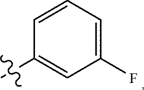 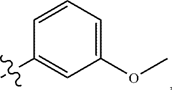 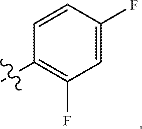 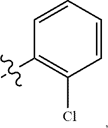 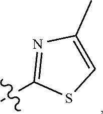 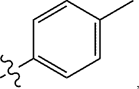 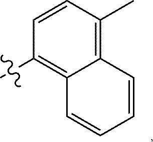 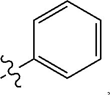 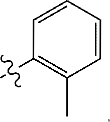 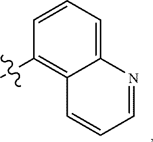 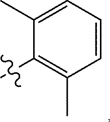 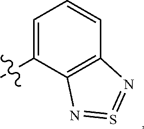
and 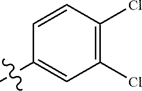
.

43. The method of any one of claims 29-36, 41, or 42, wherein R^{5b} is unsubstituted C₁₋₆ alkyl.

44. The method of any one of claims 29-36 or 41-43, wherein R^{5b} is unsubstituted C₂-₆ alkyl.

45. The method of any one of claims 29-36, wherein R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring.

46. The method of any one of claims 1-45, wherein Ra is hydrogen or -C₁₋₄ alkyl optionally substituted by 1, 2 or 3 fluorine atoms.

47. The method of any one of claims 1-45, wherein Ra -C₃₋₁₀ cycloalkyl or -(5- to 10-membered)-C₂₋₉ heterocyclyl, wherein cycloalkyl or heterocyclyl group is optionally substituted by 1, 2 or 3 fluorine atoms.

48. The method of any one of claims 1-4, wherein the compound is selected from the group consisting of 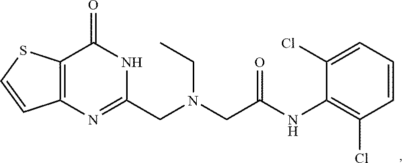 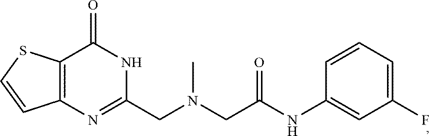 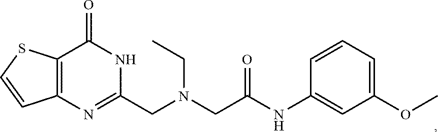 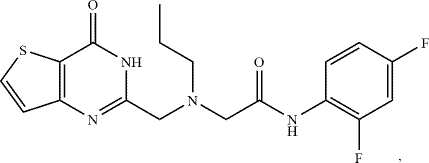 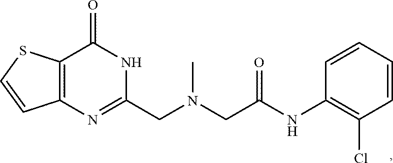 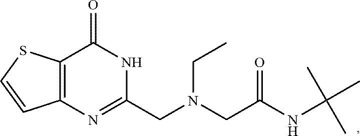 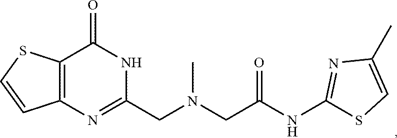
and 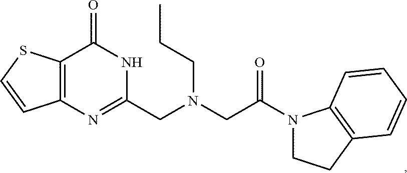
or a pharmaceutically acceptable salt or solvate thereof.

49. The method of any one of claims 1-4, wherein the compound is selected from the group consisting of 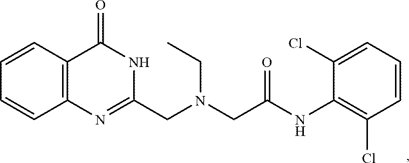 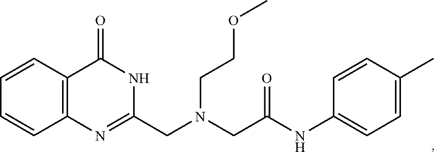 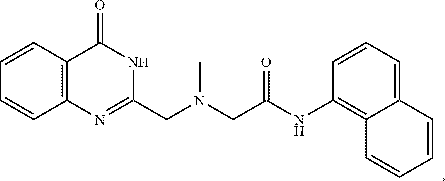 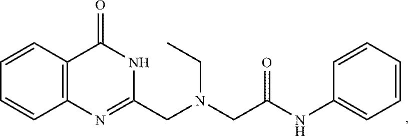 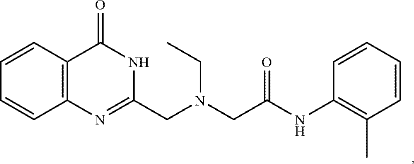 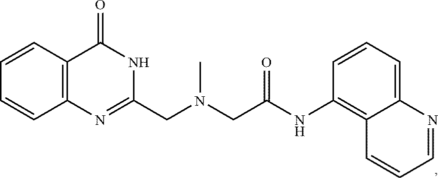 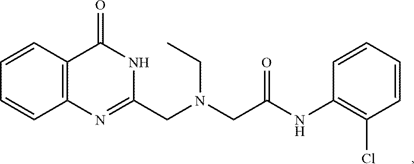 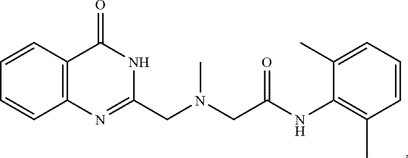 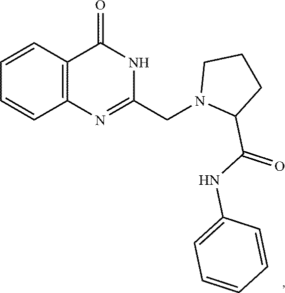 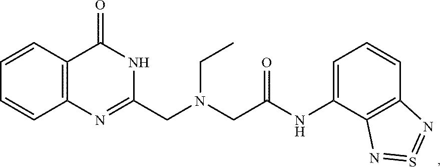 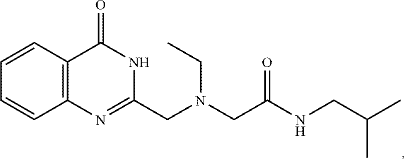 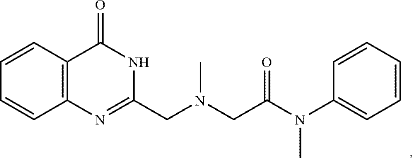 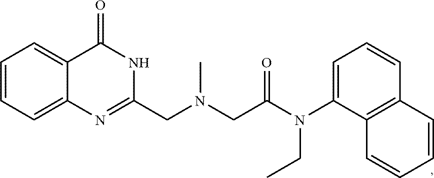
and 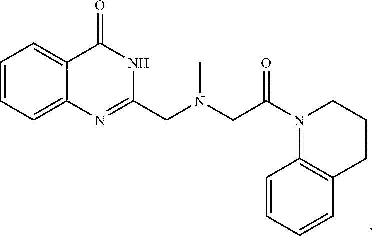
or a pharmaceutically acceptable salt or solvate thereof.

50. The method of any one of claims 1-4, wherein the compound is selected from the group consisting of 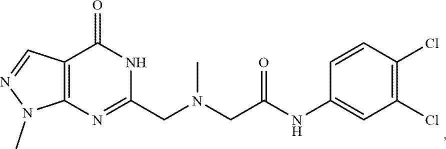 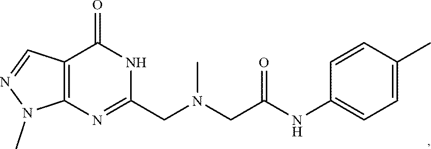 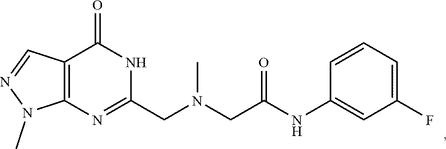 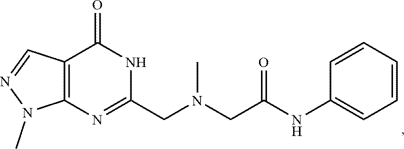 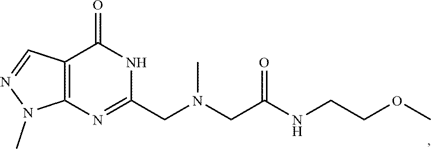 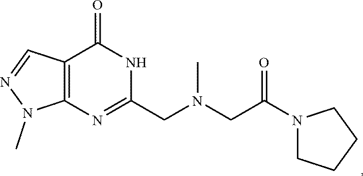 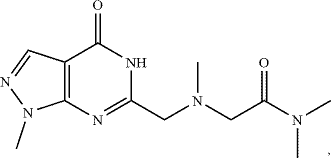
and 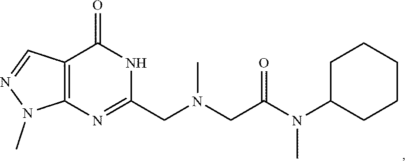
or a pharmaceutically acceptable salt or solvate thereof.

51. The method of any one of claims 1-4 or 29, wherein the compound is selected from the group consisting of 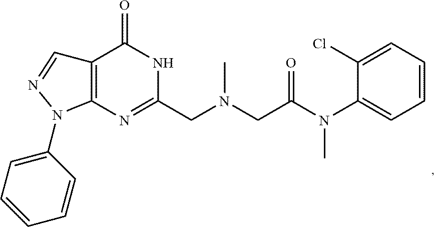 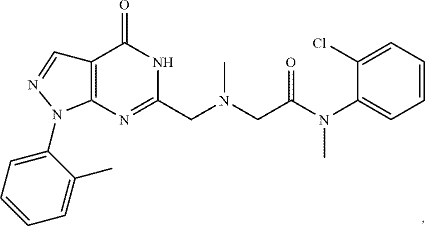 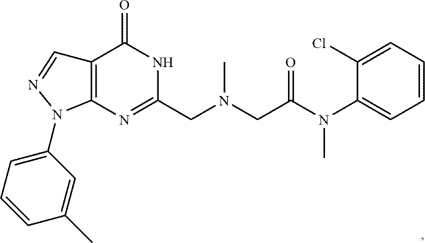 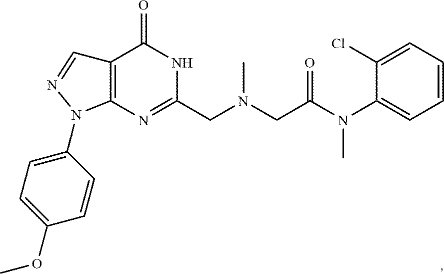 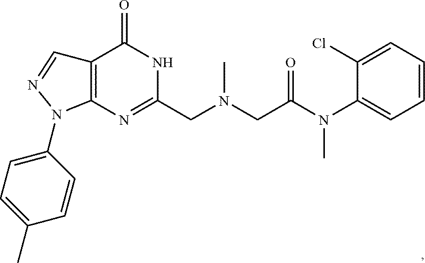 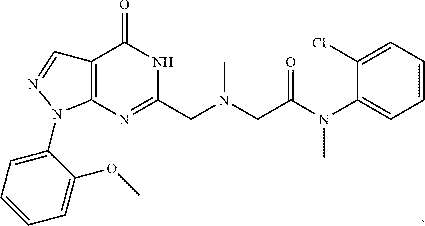
and 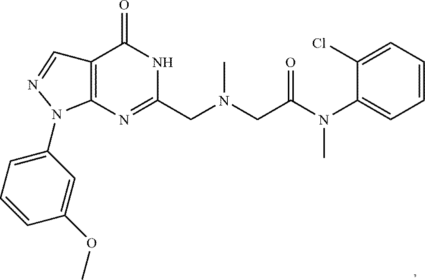
or a pharmaceutically acceptable salt or solvate thereof.

52. The method of any one of claims 1-4 or 29, wherein the compound is selected from the group consisting of 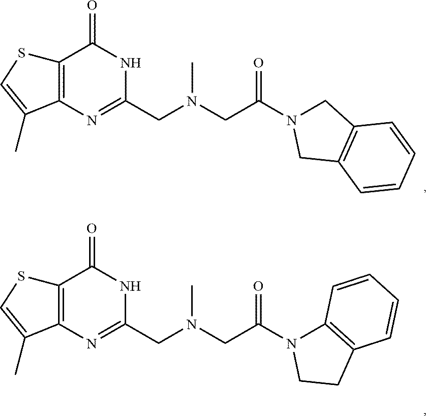 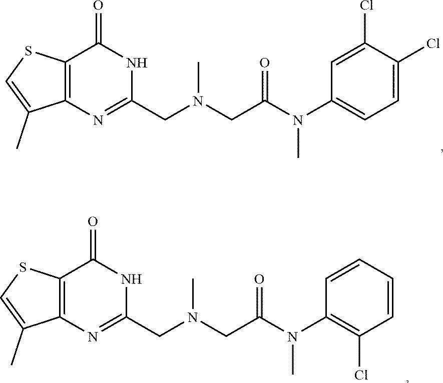 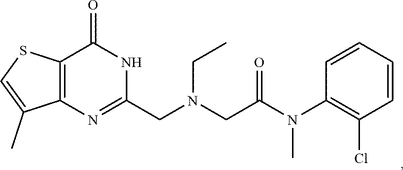 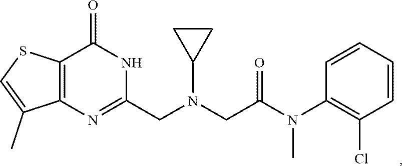 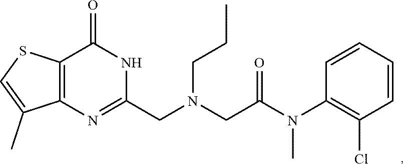 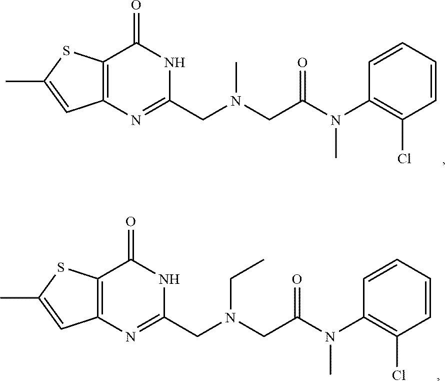 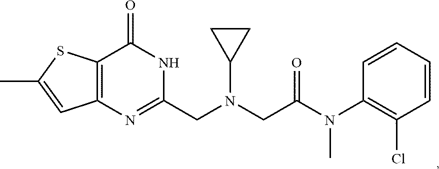 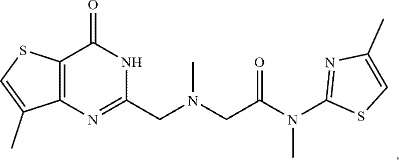 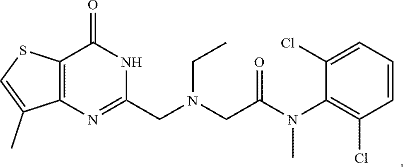 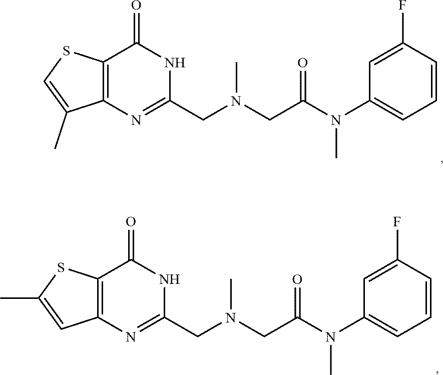 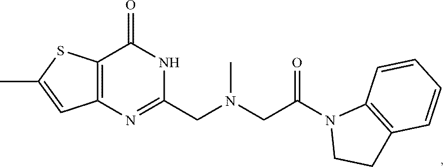 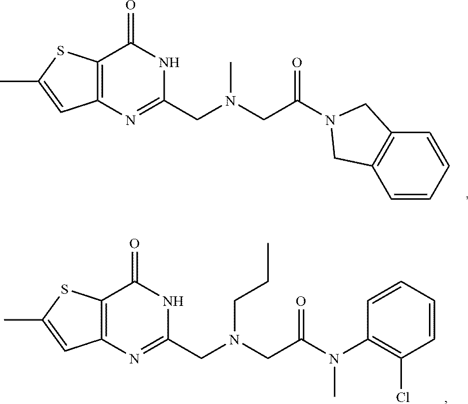 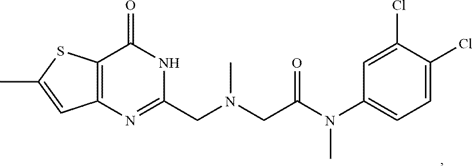 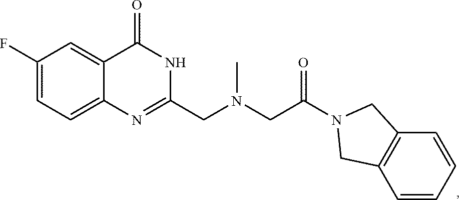 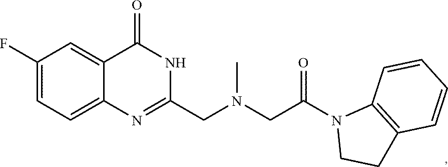 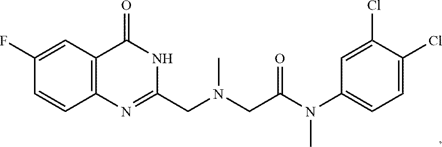 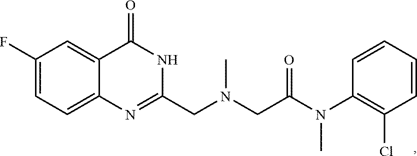 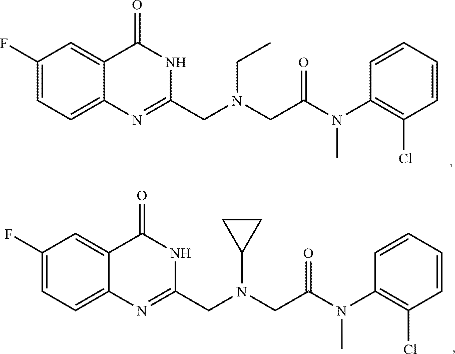 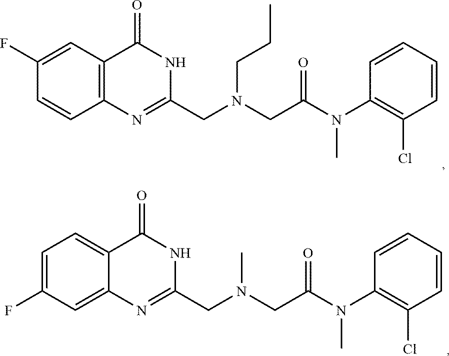 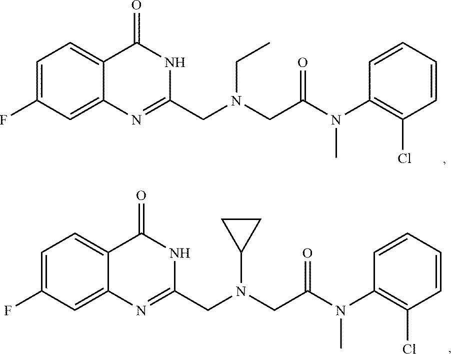 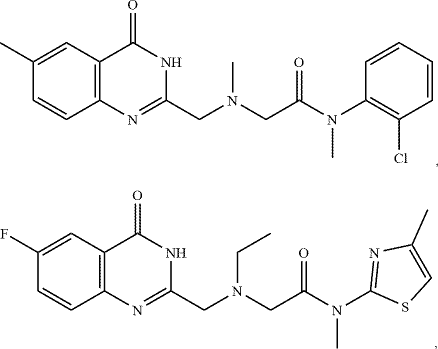 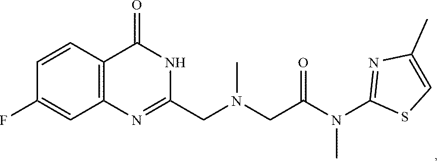 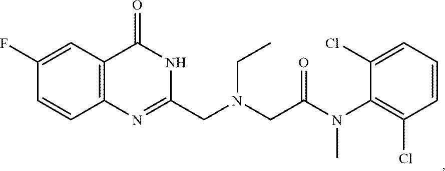 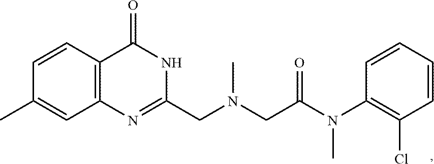 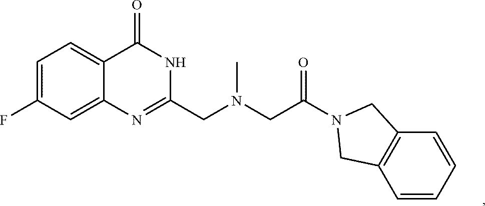 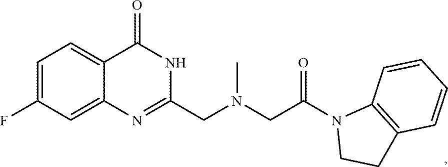 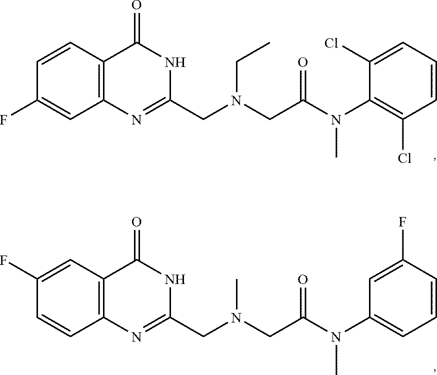 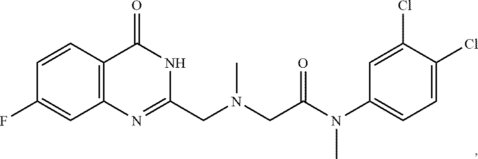 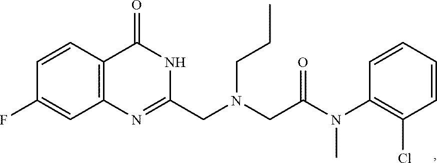 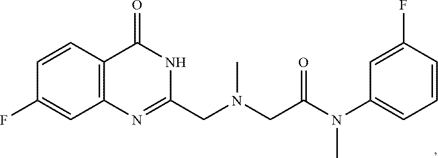 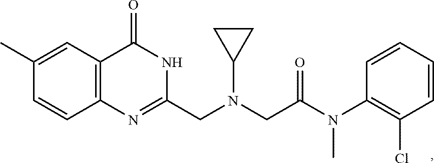 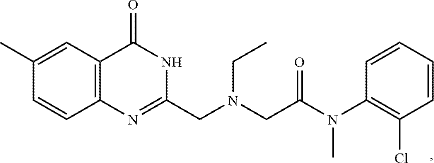 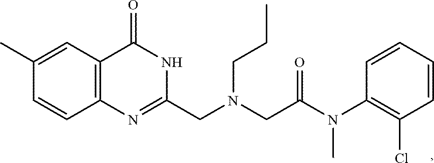 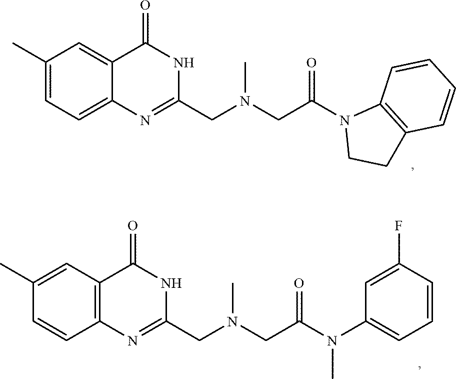 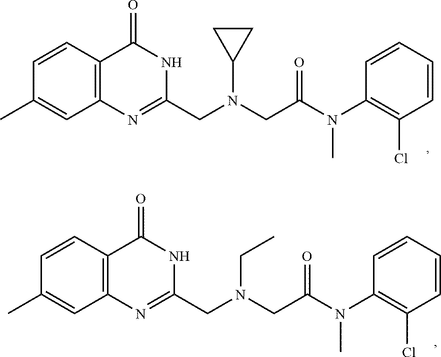 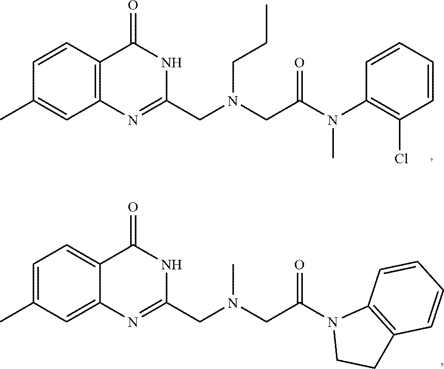 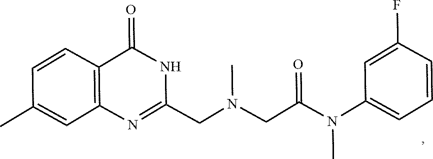 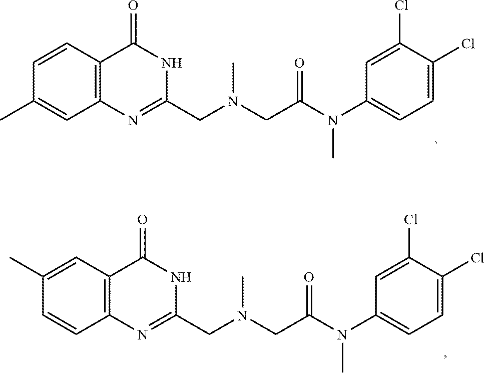 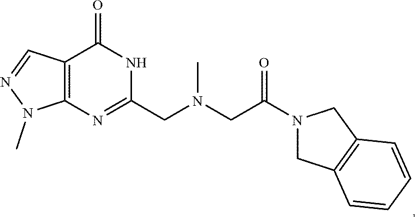 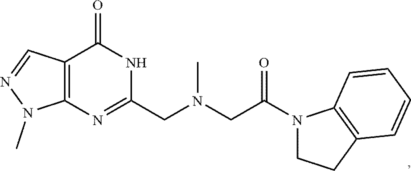 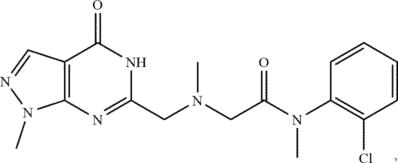 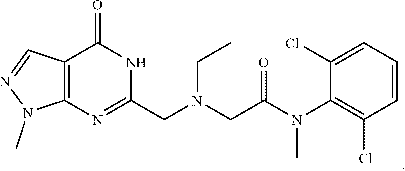
and 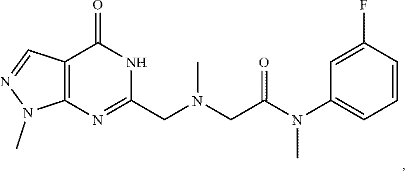
or a pharmaceutically acceptable salt or solvate thereof.

53. The method of any one of claims 1-52, wherein said method does not include administering an effective amount of α-L-iduronidase or an analog thereof.

54. The method of any one of claims 1-53, wherein said α-L-iduronidase or an analog thereof comprises laronidase.

55. The method of any one of claims 1-53, wherein said method does not include administering an effective amount of laronidase.

56. The method of any one of claims 1-52 or 54, wherein said method comprises administering said compound of formula (I), or a pharmaceutically acceptable salt or solvate thereof, in combination with an effective amount of laronidase.

57. The method of any one of claims 1-52, 54, or 56, wherein said effective amount of a compound of formula (I), or a pharmaceutically acceptable salt or solvate thereof, and an effective amount of laronidase are administered simultaneously to the patient.

58. The method of any one of claims 1-52, 54, or 56, wherein said effective amount of a compound of formula (I), or a pharmaceutically acceptable salt or solvate thereof, and said effective amount of laronidase are administered to the patient sequentially.

59. The method of any one of claims 56-58, wherein said effective amount of a compound of formula (I), or a pharmaceutically acceptable salt or solvate thereof, and said effective amount of laronidase are administered to the patient in separate pharmaceutical compositions.

60. The method of any one of claims 56-58, wherein said effective amount of a compound of formula (I), or a pharmaceutically acceptable salt or solvate thereof, and said effective amount of laronidase are administered to the patient in a single pharmaceutical composition.

61. A compound having the formula (I′): 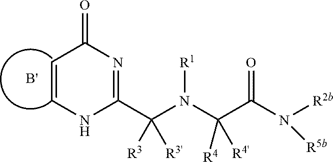
or a pharmaceutically acceptable salt or solvate thereof, wherein
B′ is a fused ring selected from the group consisting of 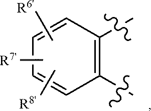 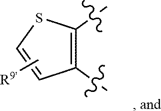 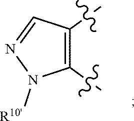
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring; or
R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{5b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋ ₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring; provided that when B′ is B1' or B3' then the heterocyclic ring is:
1) substituted by at least one substituent selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋₁₀ aryl, -(5- to 10-membered)-C₁₋₉ heteroaryl, -(5- to 10-membered)-C₂₋₉ heterocyclyl, and -C₃₋₁₀ cycloalkyl, wherein Ra is as defined above, or
2) fused to a phenyl ring;
R⁶', R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R⁶', R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms;
provided that the compound is not 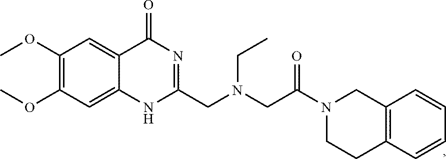 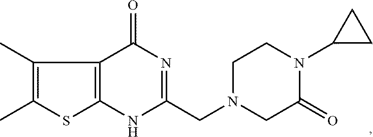 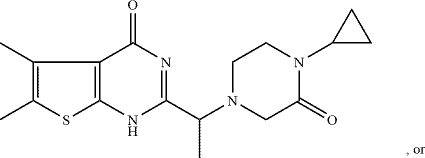 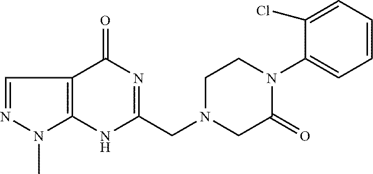 [SMILES: Cn1ncc2c(=O)nc(CN3CCN(c4ccccc4Cl)C(=O)C3)[nH]c21]
.

62. The compound of claim 61, wherein B′ is 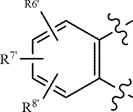
.

63. The compound of claim 61, wherein B′ is 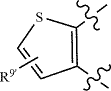
.

64. The compound of claim 61, wherein B′ is 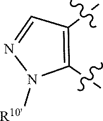
.

65. The compound of claim 61 or 64, wherein B′ is B3' selected from the group consisting of 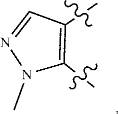 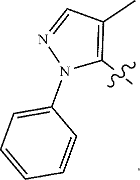 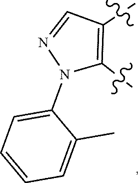 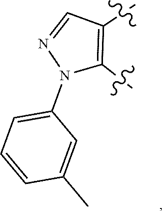 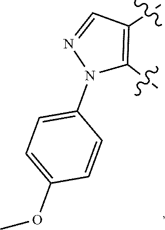 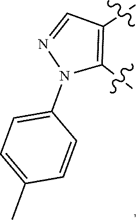
and 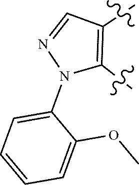
.

66. The compound of any one of claims 61-65, wherein R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; and
R³, R^{3'}, R⁴, and R^{4'} are each independently selected from the group consisting of hydrogen, halogen, unsubstituted C₁₋₄ alkyl, and substituted C₁₋₄ alkyl.

67. The compound of any one of claims 61-66, wherein R³ and R⁴ are hydrogen, and R^{3'} and R^{4'} are each independently selected from the group consisting of hydrogen, halogen, unsubstituted C₁₋₄ alkyl, and substituted C₁₋₄ alkyl.

68. The compound of any one of claims 61-67, wherein R³, R^{3'}, R⁴, and R^{4'} are each hydrogen.

69. The compound of any one of claims 61-68, wherein R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R^{5b} is selected from the group consisting of -C₃₋₁₀ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring, provided that the compound is not 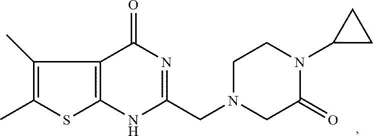 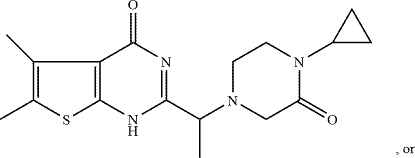 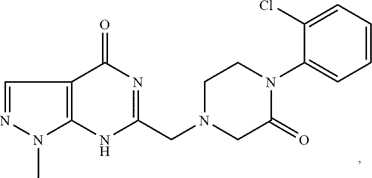
.

70. The compound of any one of claims 61-66, wherein R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³', R⁴, and R⁴' are as defined in claim 61.

71. The compound of any one of claims 61-66, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are as defined in claim 61.

72. The compound of any one of claims 61-66 or 71, wherein R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O, and R³, R³', and R⁴' are each hydrogen.

73. The compound of any one of claims 61-68, wherein R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring; and
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring.

74. The compound of any one of claims 61-68 or 73, wherein R^{2b} is selected from the group consisting of 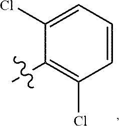 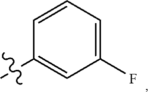 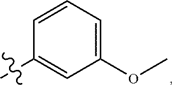 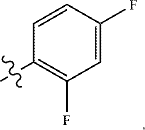 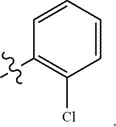 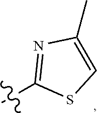 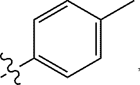 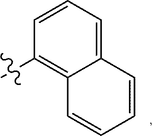 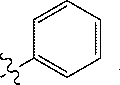 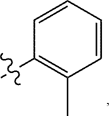 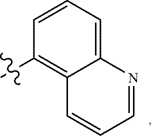 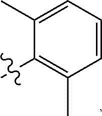 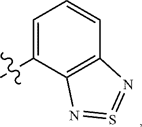
and 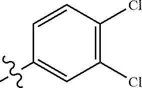
.

75. The compound of any one of claims 61-68, 73, or 74, wherein R^{5b} is unsubstituted C₁₋₆ alkyl.

76. The compound of any one of claims 61-68 or 73-75, wherein R^{5b} is unsubstituted C₂-₆ alkyl.

77. The compound of any one of claims 61-76, wherein Ra is hydrogen or -C₁₋₄ alkyl optionally substituted by 1, 2 or 3 fluorine atoms.

78. The compound of any one of claims 61-76, wherein Ra -C₃₋₁₀ cycloalkyl or -(5- to 10-membered)-C₂₋₉ heterocyclyl, wherein cycloalkyl or heterocyclyl group is optionally substituted by 1, 2 or 3 fluorine atoms.

79. The compound of claims 61, wherein the compound is selected from the group consisting of 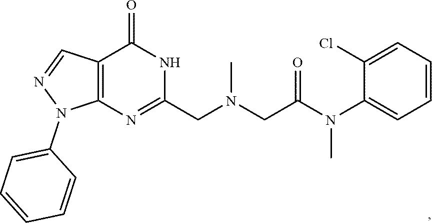 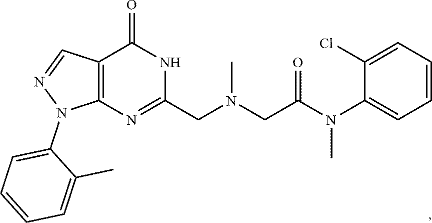 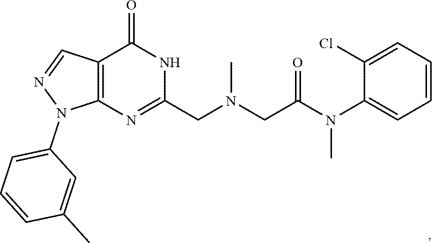 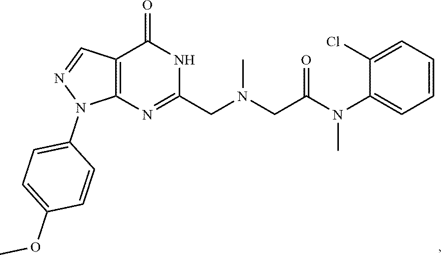 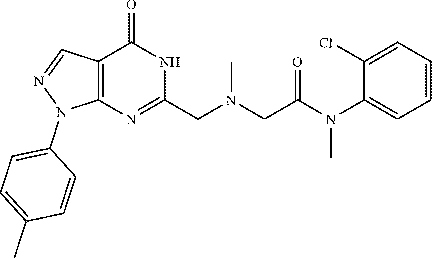 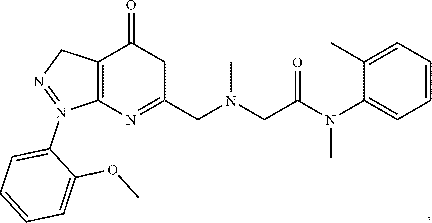 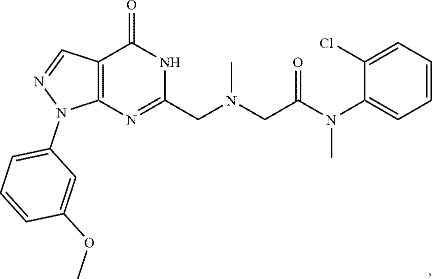
or a pharmaceutically acceptable salt or solvate thereof.

80. The compound of claim 61, wherein the compound is selected from the group consisting of 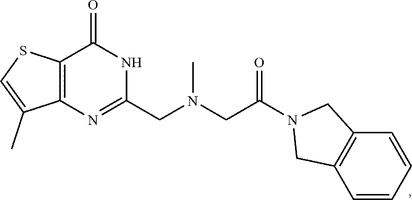 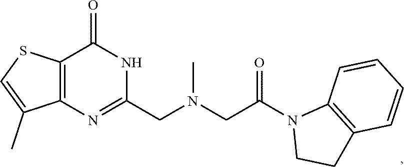 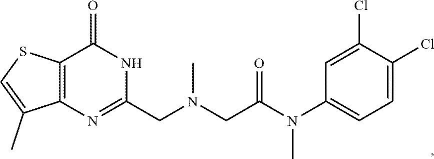 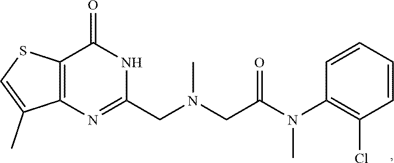 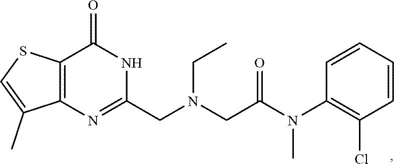 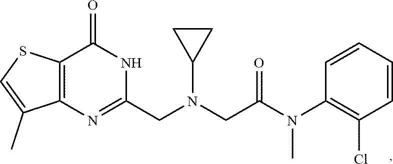 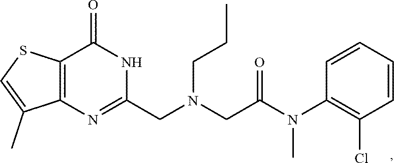 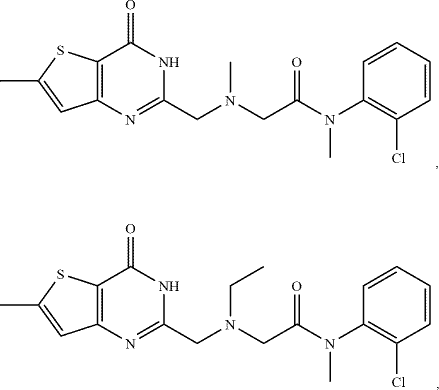 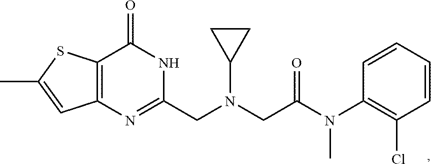 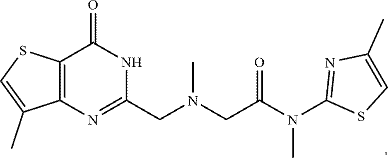 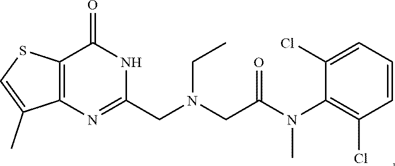 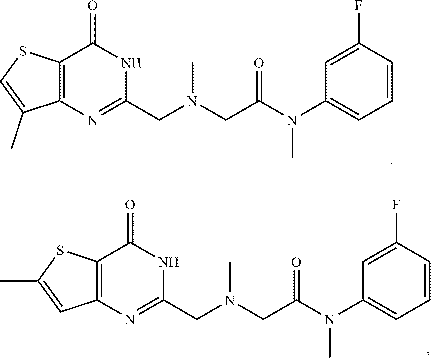 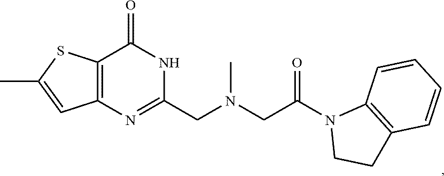 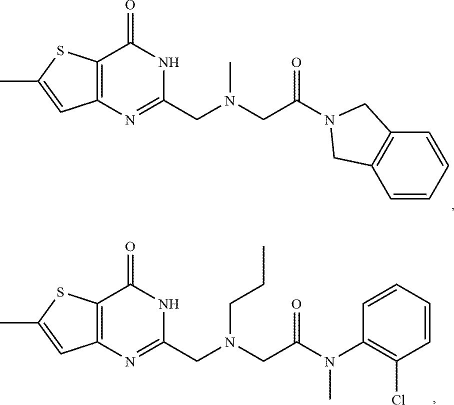 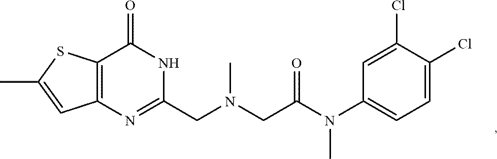 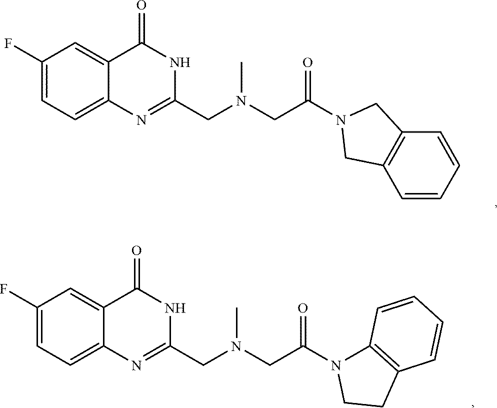 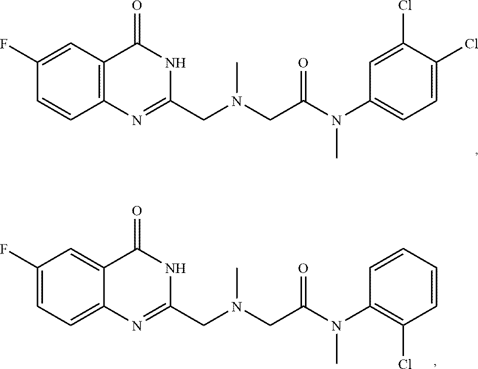 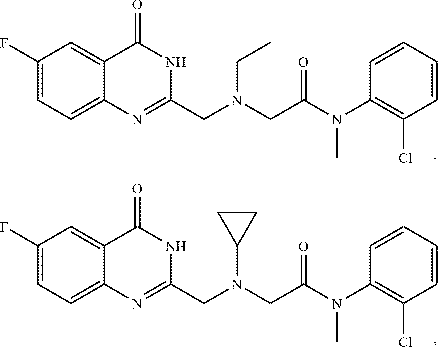 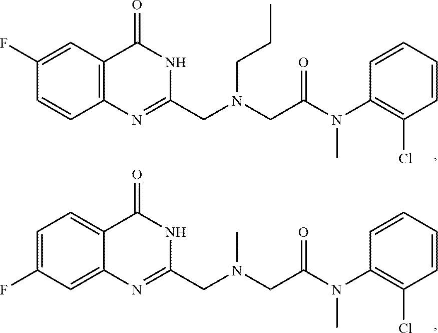 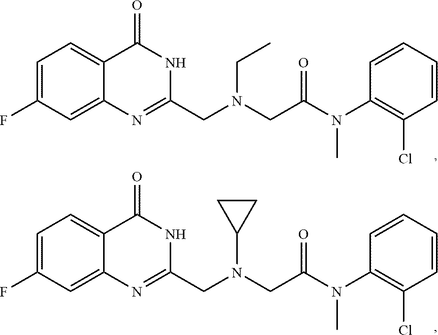 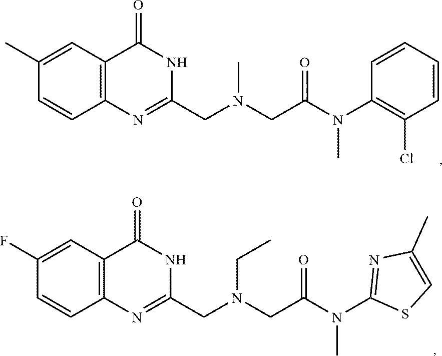 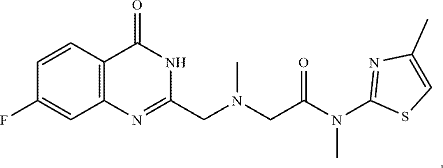 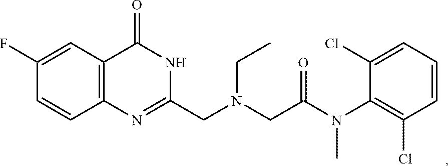 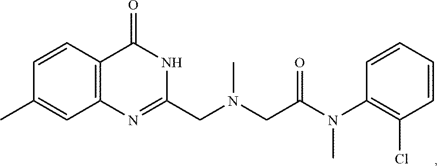 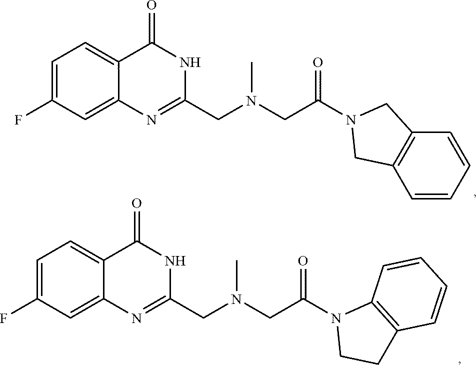 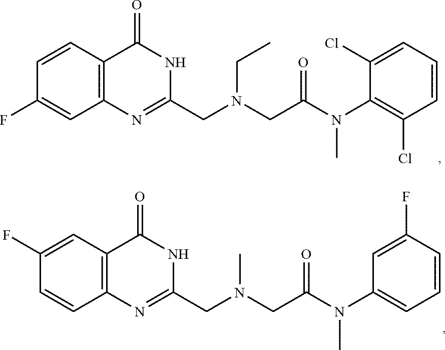 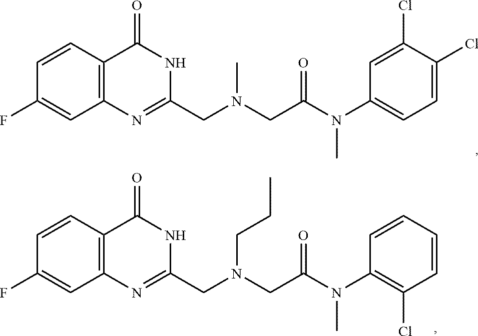 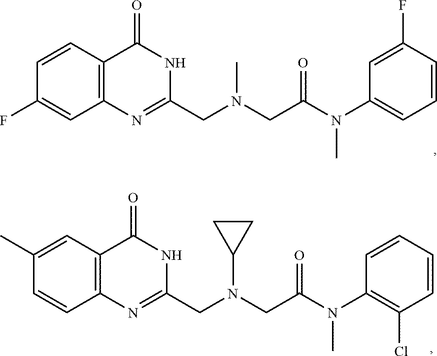 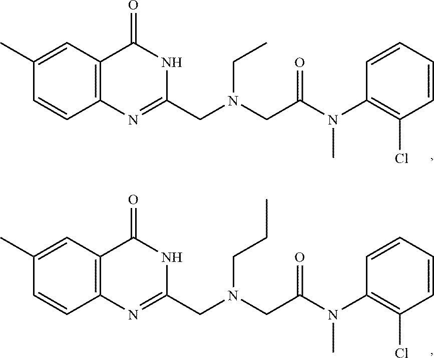 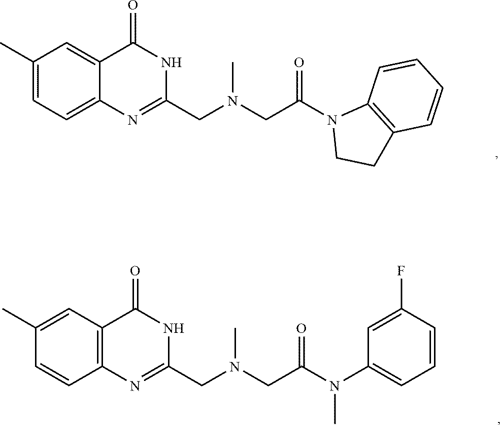 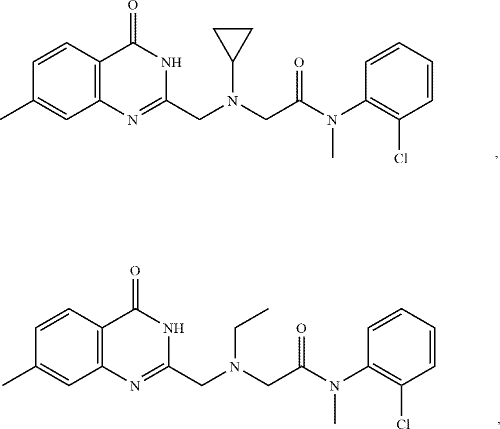 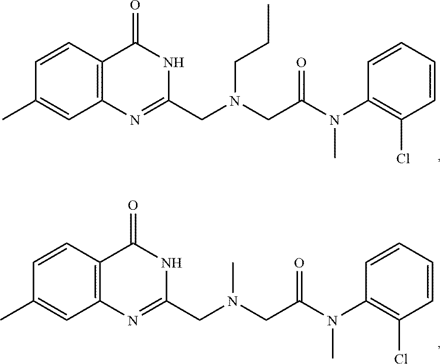 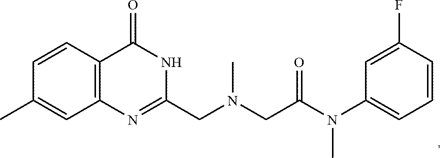 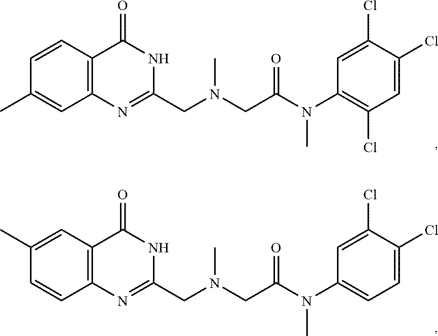 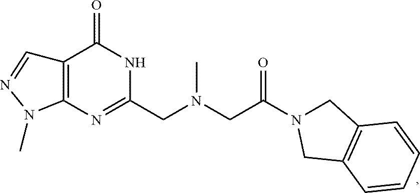 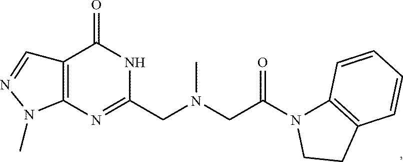 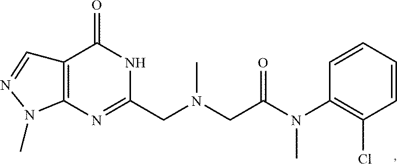 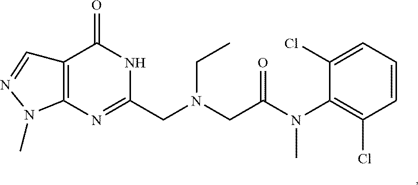
and 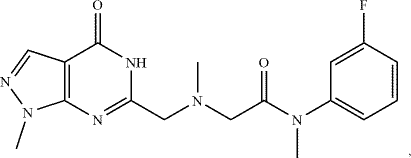
or a pharmaceutically acceptable salt or solvate thereof.

81. A pharmaceutical composition, comprising an effective amount of a compound of formula (I), or a pharmaceutically acceptable salt or solvate thereof, and at least one pharmaceutically acceptable excipient, wherein the compound of formula (I) has the structure: 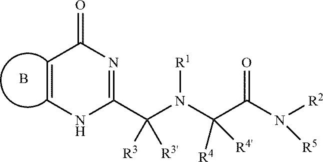
or a pharmaceutically acceptable salt or solvate thereof, wherein
B is a fused benzene ring or a fused 5- or 6-membered heteroaromatic ring, wherein said benzene ring and said 5- or 6-membered heteroaromatic ring is optionally substituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R³', R⁴, and R⁴' are each independently selected from the group consisting of hydrogen, halogen, unsubstituted C₁₋₄ alkyl, and substituted C₁₋₄ alkyl; R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by aheteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

82. The pharmaceutical composition of claim 81, wherein the compound is selected from the group consisting of 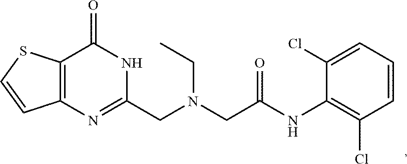 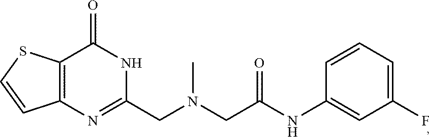 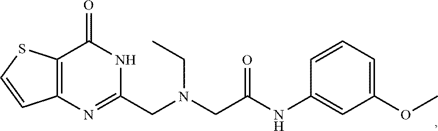 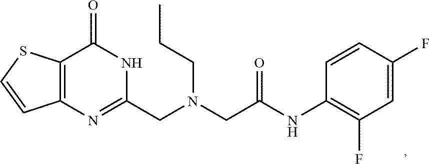 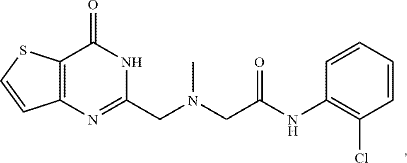 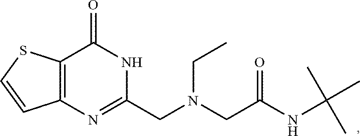 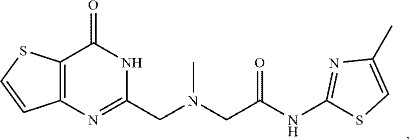
and 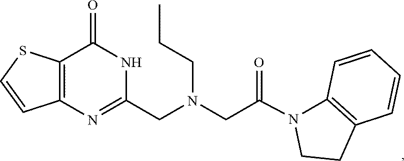
or a pharmaceutically acceptable salt or solvate thereof.

83. The pharmaceutical composition of claim 81, wherein the compound is selected from the group consisting of 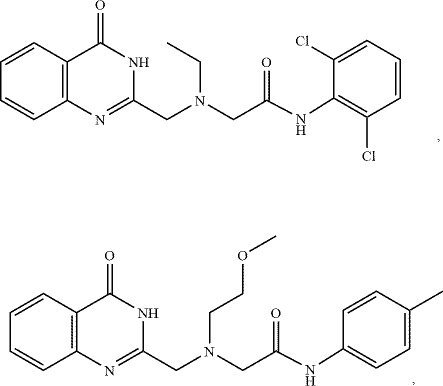 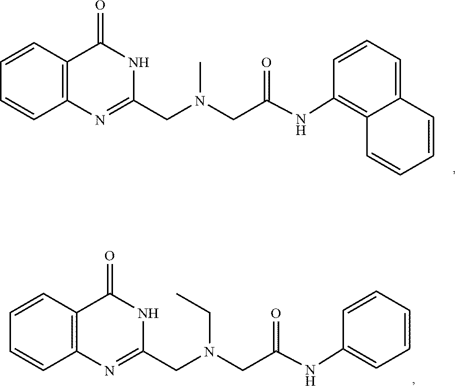 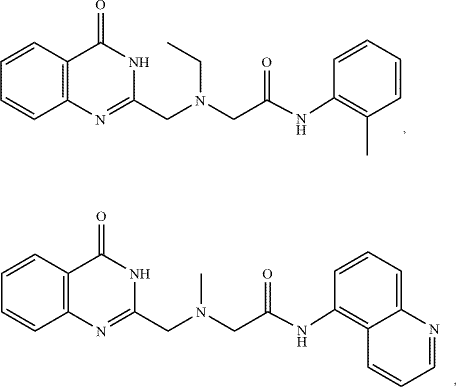 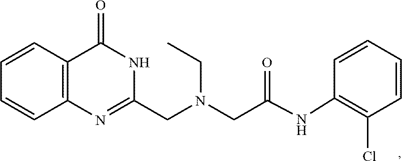 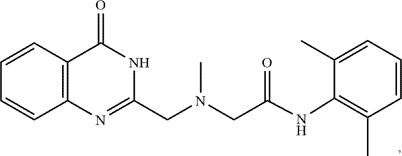 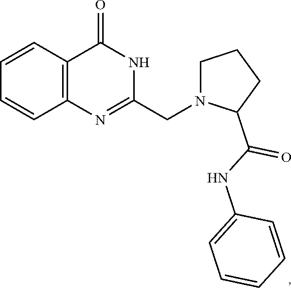 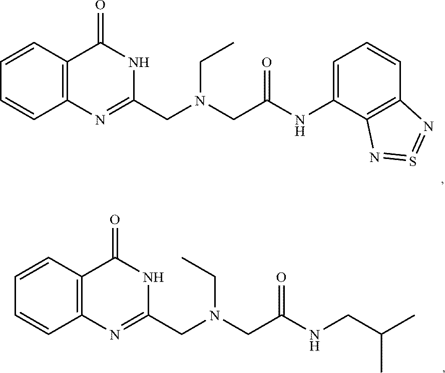 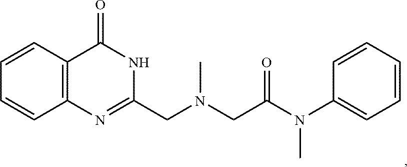 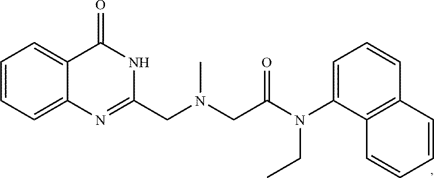 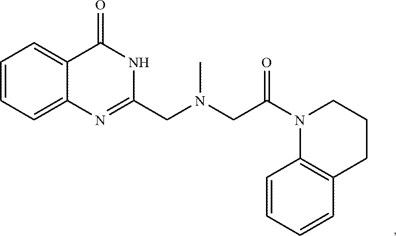
or a pharmaceutically acceptable salt or solvate thereof.

84. The pharmaceutical composition of claim 81, wherein the compound is selected from the group consisting of 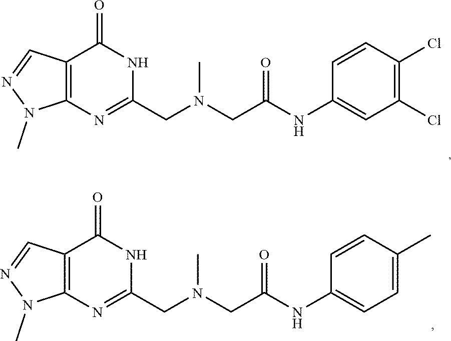 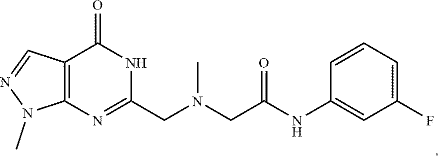 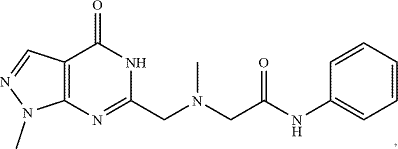 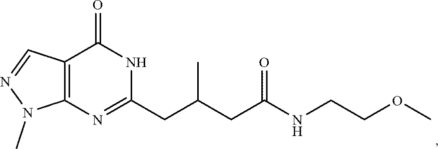 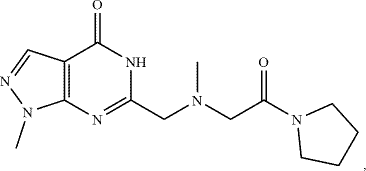 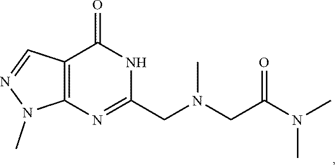
and 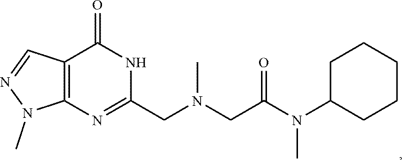
or a pharmaceutically acceptable salt or solvate thereof.

85. The pharmaceutical composition of claim 81, wherein the compound of formula (I) is a compound of formula (I′) having the structure: 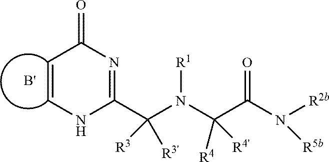
or a pharmaceutically acceptable salt or solvate thereof, wherein
B′ is a fused ring selected from the group consisting of 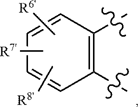 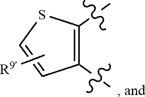 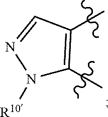
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring; or
R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋ ₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring;
R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R⁶', R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

86. The pharmaceutical composition of claim 85, wherein the compound is selected from the group consisting of 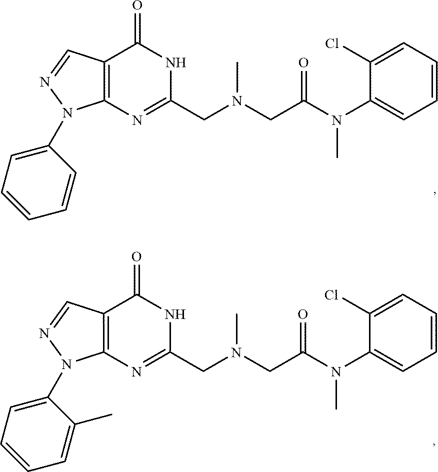 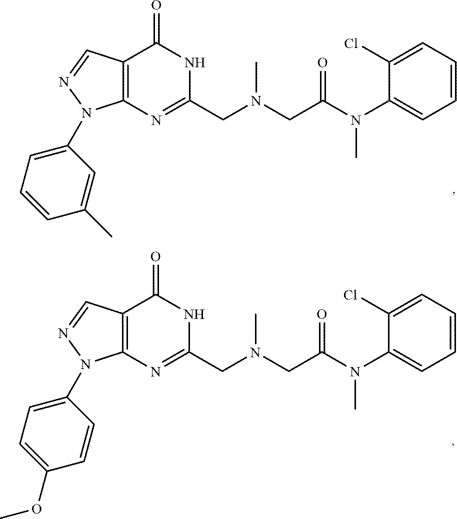 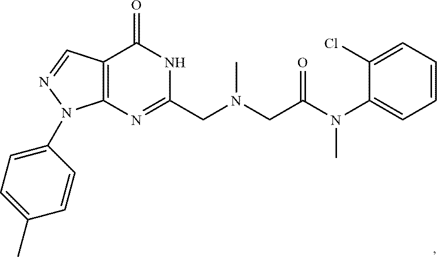 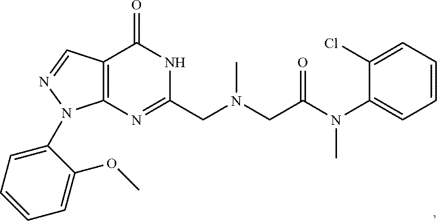
and 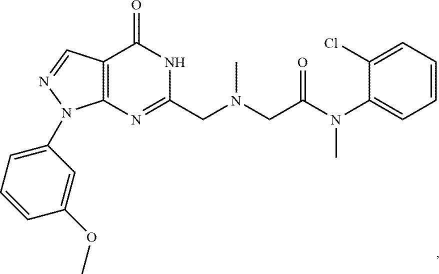
or a pharmaceutically acceptable salt or solvate thereof.

87. The pharmaceutical composition of claim 85, wherein the compound is selected from the group consisting of 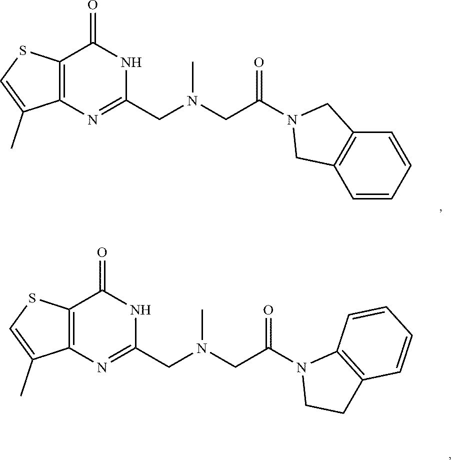 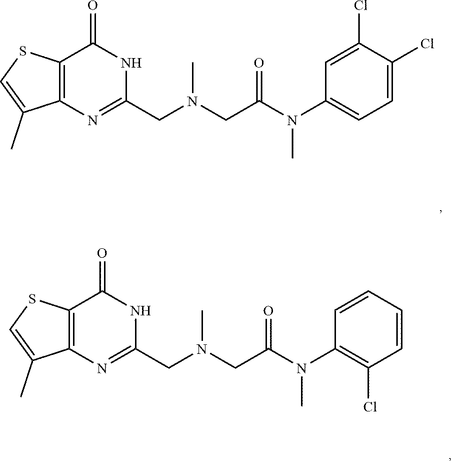 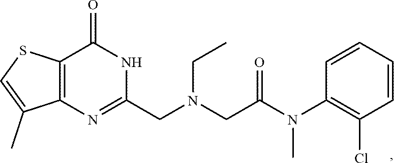 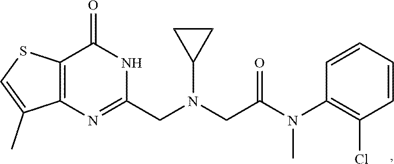 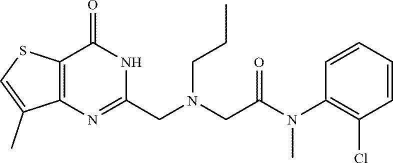 [SMILES: CCCN(CC(=O)N(C)c1ccccc1Cl)Cc1nc2c(C)csc2c(=O)[nH]1] 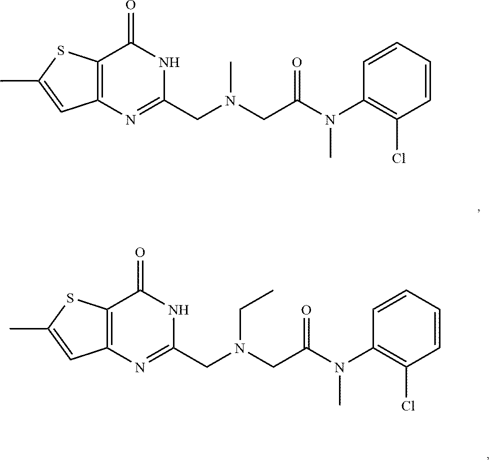 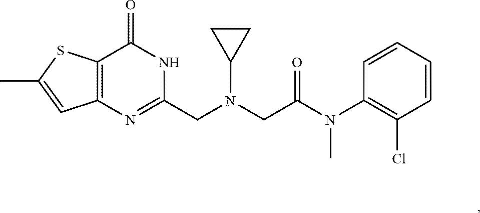 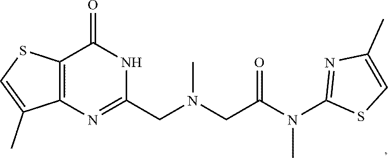 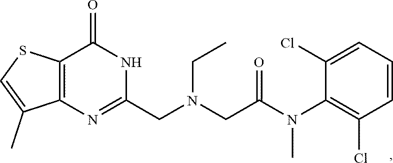 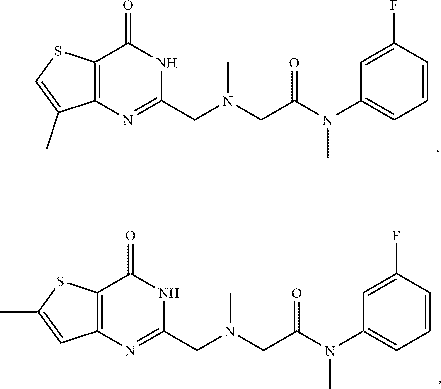 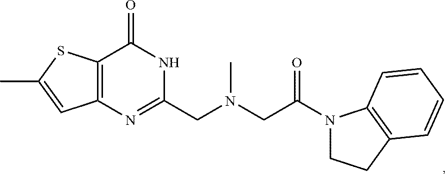 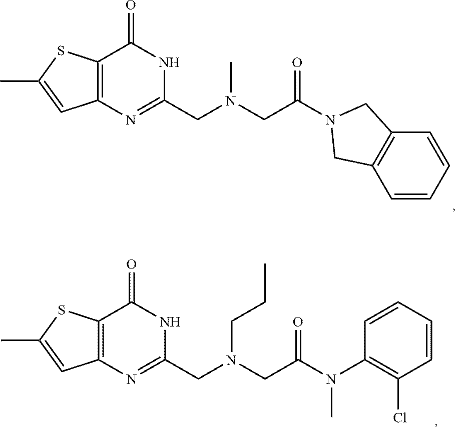 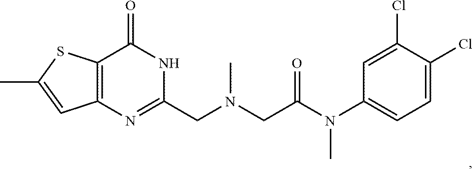 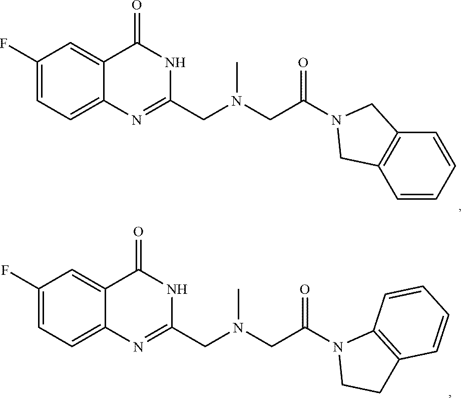 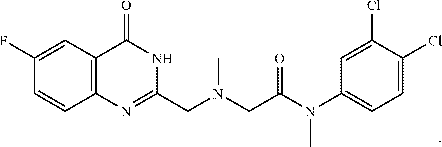 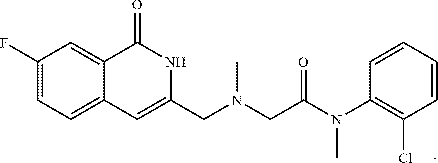 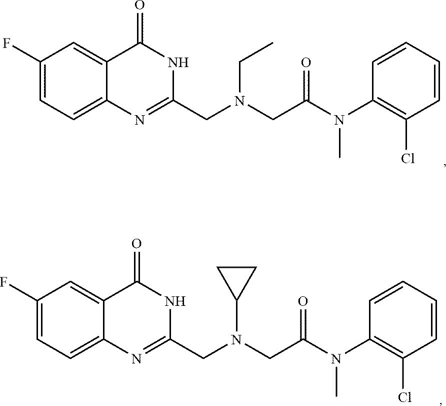 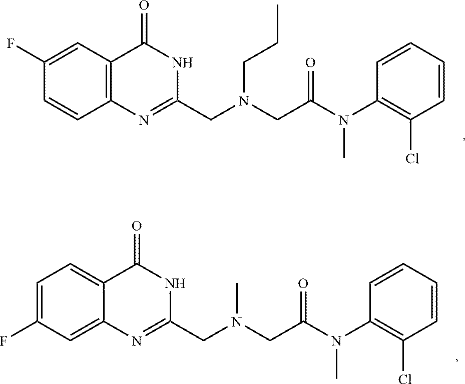 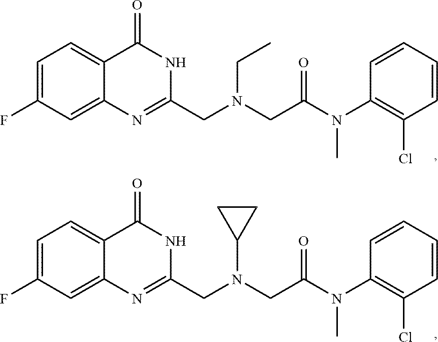 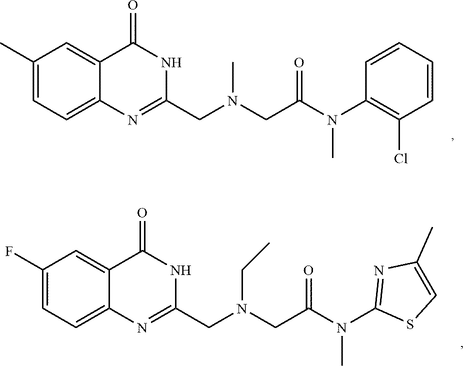 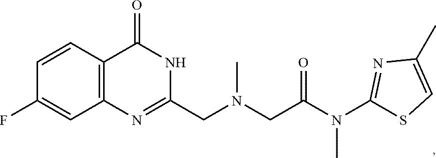 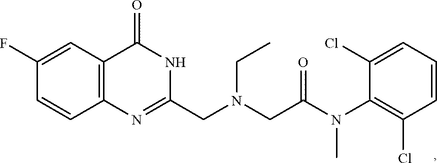 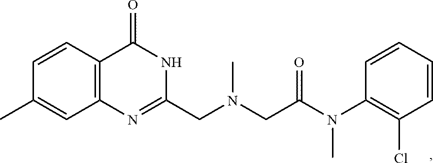 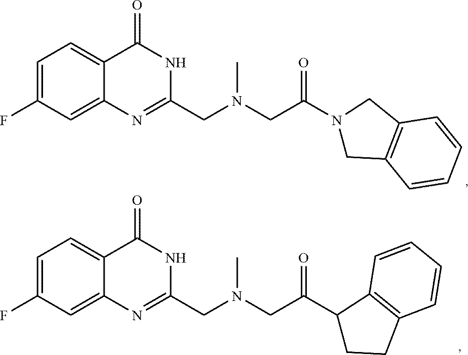 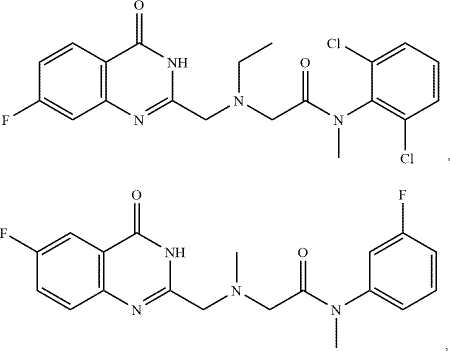 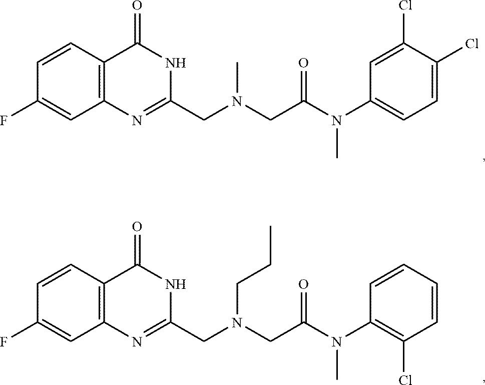 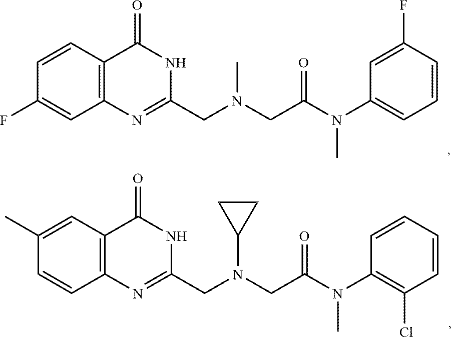 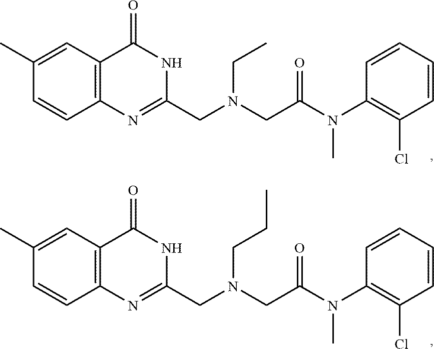 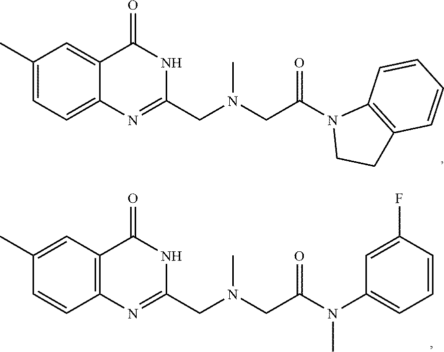 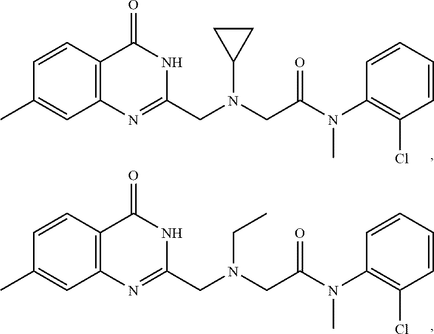 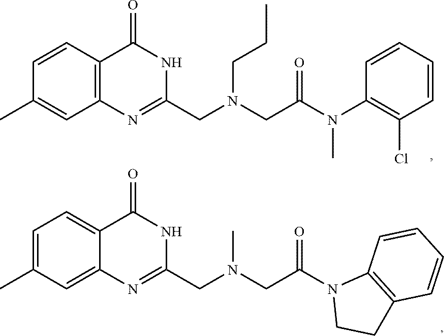 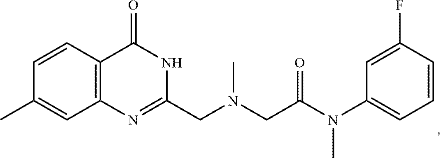 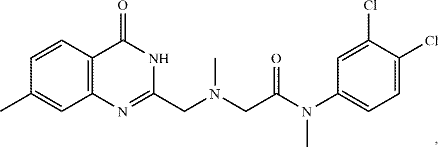 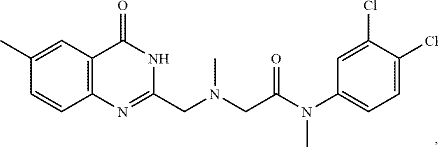 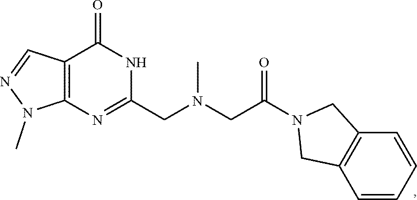 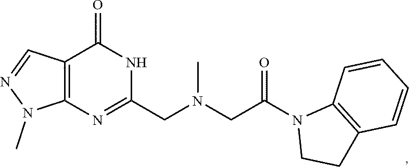 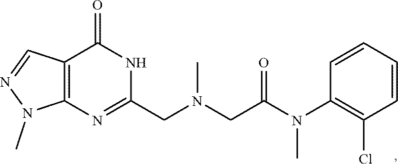 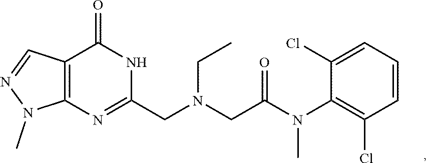
and 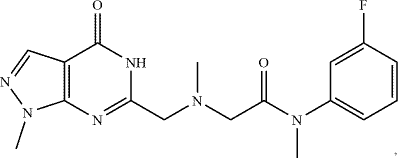
, or a pharmaceutically acceptable salt or solvate thereof.

88. A compound of formula (I): 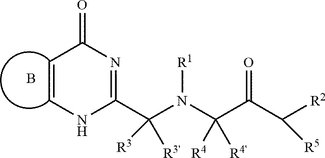
or a pharmaceutically acceptable salt or solvate thereof, for use as a medicament, optionally in a combination with α-L-iduronidase or an analog or variant thereof, wherein
B is a fused benzene ring or a fused 5- or 6-membered heteroaromatic ring, wherein said benzene ring and said 5- or 6-membered heteroaromatic ring is optionally sub stituted;
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R² is selected from the group consisting of C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R³, R³', R⁴, and R⁴' are each independently selected from the group consisting of hydrogen, halogen, unsubstituted C₁₋₄ alkyl, and substituted C₁₋₄ alkyl;
R⁵ is selected from the group consisting of hydrogen, C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring; or
R¹ and R² together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R² and R⁵ together with the nitrogen atom to which they are attached form an optionally substituted 5- to 10-membered heterocyclic ring, wherein 1, 2, or 3 of the carbon atoms of said heterocyclic ring are optionally replaced by aheteroatom selected from the group consisting of N, S, and O, and wherein said heterocyclic ring is optionally fused to a phenyl ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

89. The compound for use according to claim 88, wherein the compound is selected from the group consisting of 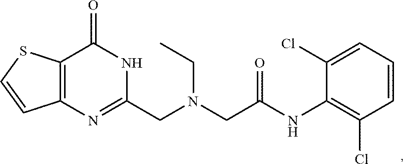 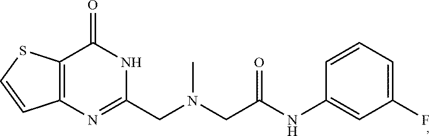 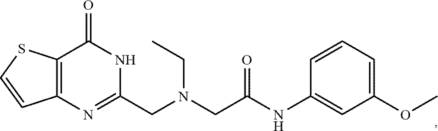 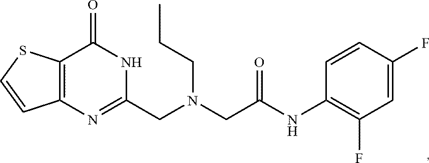 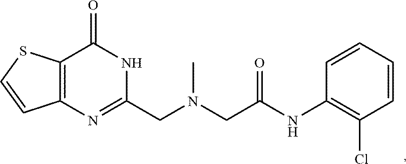 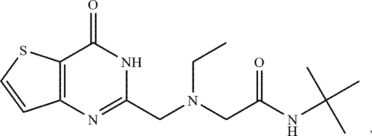 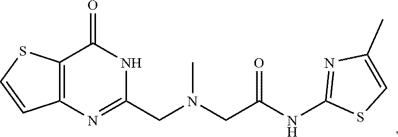
and 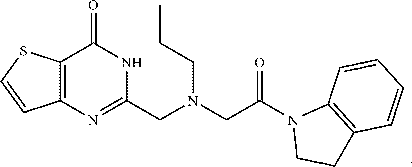
or a pharmaceutically acceptable salt or solvate thereof.

90. The compound for use according to claim 88, wherein the compound is selected from the group consisting of 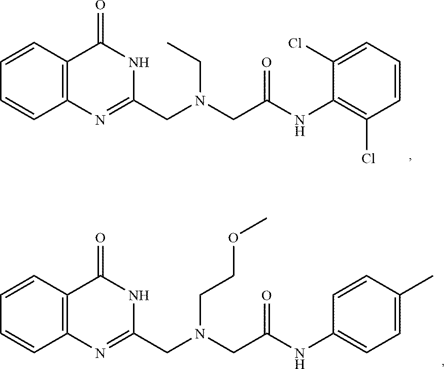 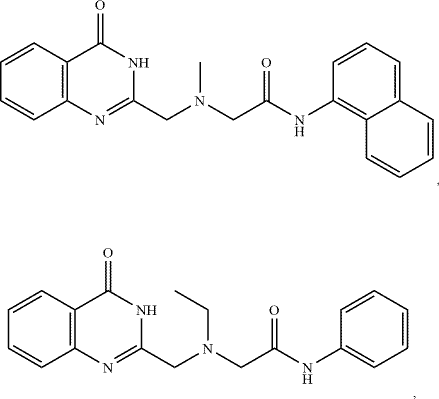 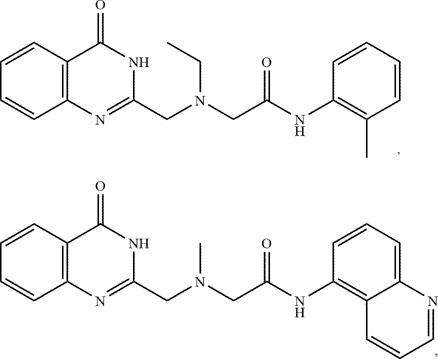 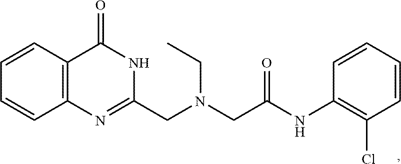 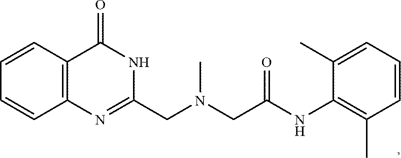 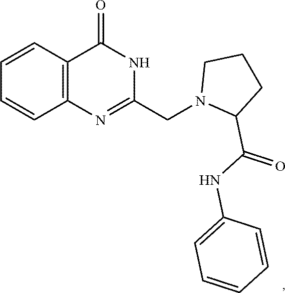 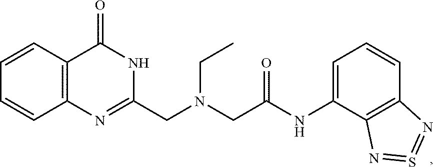 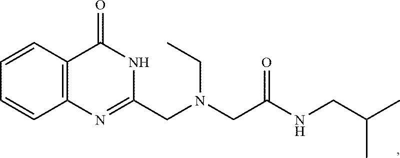 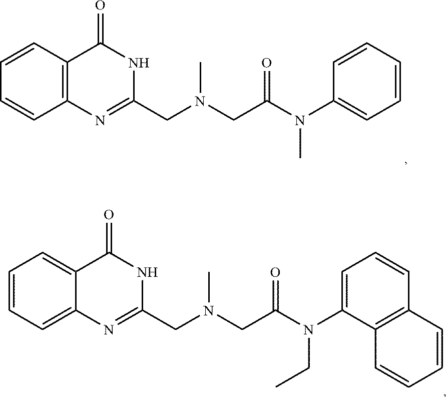
and 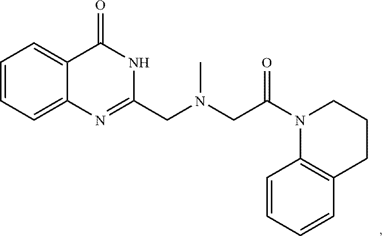
or a pharmaceutically acceptable salt or solvate thereof.

91. The compound for use according to claim 88, wherein the compound is selected from the group consisting of 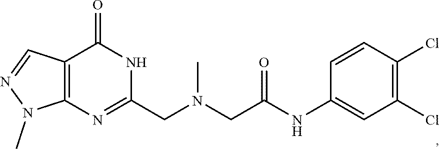 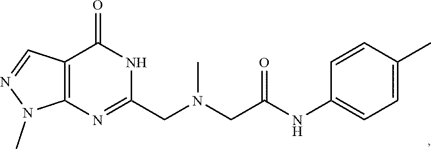 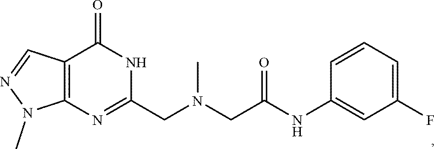 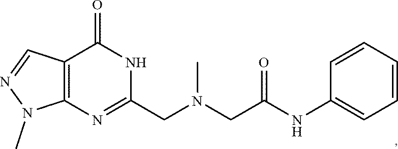 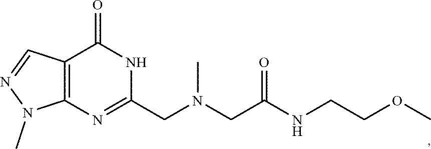 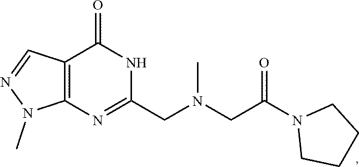 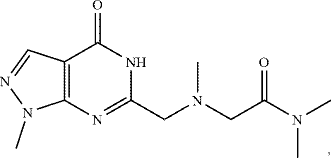
and 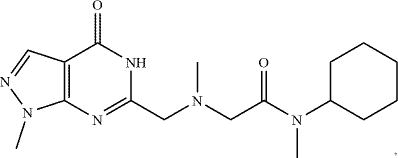
or a pharmaceutically acceptable salt or solvate thereof.

92. The compound for use according to claim 88, wherein the compound of formula (I) is a compound of formula (I′) having the structure: 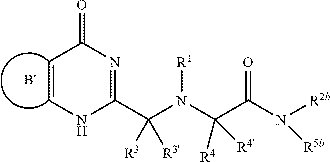
or a pharmaceutically acceptable salt or solvate thereof, wherein
B′ is a fused ring selected from the group consisting of 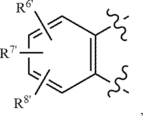 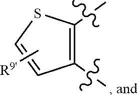 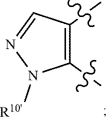
R¹ is selected from the group consisting of -C₁₋₄ alkyl, -C₃₋₁₀ cycloalkyl, -C₁₋₄ alkyl-C₃₋₁₀ cycloalkyl, -C₆₋₁₀ aryl, -C₁₋₄ alkyl-C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, alkylaryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5-to 10-membered)-C₂₋₉ heterocyclyl; and wherein said cycloalkyl, alkylcycloalkyl, aryl, alkylaryl, heteroaryl, alkylheteroaryl, heterocyclyl and alkylheterocyclyl is optionally fused to a further (second) ring;
R^{2b} is selected from the group consisting of -C₃₋₅ cycloalkyl, -C₆₋₁₀ aryl, (5- to 10-membered)-C₁₋₉ heteroaryl, and (5- to 10-membered)-C₂₋₉ heterocyclyl, wherein said cycloalkyl, aryl, heteroaryl, and heterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, aryl, heteroaryl, and heterocyclyl is optionally fused to a further (second) ring;
R³, R^{3'}, R⁴, and R^{4'} are each independently selected from the group consisting of hydrogen, halogen, unsubstituted C₁₋₄ alkyl, and substituted C₁₋₄ alkyl;
R^{5b} is selected from the group consisting of C₁₋₄ alkyl, -C₃₋₁₀ cycloalkyl, -C₁₋₄ alkyl-C₃₋₁₀ cycloalkyl, -C₆₋₁₀ aryl, (5- to 10-membered)-C₁₋₉ heteroaryl, -C₁₋₄ alkyl-(5- to 10-membered)-C₁₋₉ heteroaryl, (5- to 10-membered)-C₂₋₉ heterocyclyl, and -C₁₋₄ alkyl-(5- to 10-membered)-C₂₋₉ heterocyclyl, wherein said alkyl, cycloalkyl, alkylcycloalkyl, aryl, heteroaryl, alkylheteroaryl, heterocyclyl and alkylheterocyclyl groups are optionally substituted with 1, 2 or 3 substituents each independently selected from the group consisting of halogen, hydroxy, -CN, -ORa, -SRa, -N(Ra)₂, -C₁₋₄ alkyl optionally substituted with 1, 2, or 3 halogen atoms, optionally substituted C₆₋₁₀ aryl, optionally substituted (5- to 10-membered)-C₁₋₉ heteroaryl, and (5- to 10-membered)-C₂₋₉ heterocyclyl; and wherein said cycloalkyl, alkylcycloalkyl, aryl, heteroaryl, alkylheteroaryl, heterocyclyl and alkylheterocyclyl is optionally fused to a further (second) ring; or
R^{2b} and R^{5b} together with the nitrogen atom to which they are attached form a 5- or 6-membered heterocyclic ring, optionally substituted with 1, 2, or 3 substituents each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, -C₆₋ ₁₀ aryl, -(5- to 10-membered)-C₁₋₉ heteroaryl, -(5- to 10-membered)-C₂₋₉ heterocyclyl, and -C₃₋₁₀ cycloalkyl, wherein 1, 2, or 3 of the carbon atoms of said heterocyclic ring are optionally replaced by a heteroatom selected from the group consisting of N, S, and O, and wherein said heterocyclic ring is optionally fused to a phenyl ring; or
R¹ and R^{2b} together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R³ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O; or
R¹ and R⁴ together with the nitrogen and carbon atoms to which they are attached form an optionally substituted 5- to 10-membered heterocyclic ring, wherein 1, 2, or 3 of the carbon atoms of said heterocyclic ring are optionally replaced by a heteroatom selected from the group consisting of N, S, and O;
R^{6'}, R^{7'}, and R^{8'} are each independently selected from the group consisting of wherein hydrogen, halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring, provided that at least one of R⁶', R^{7'}, and R^{8'} is other than hydrogen;
R^{9'} and R^{10'} are each independently selected from the group consisting of halogen, hydroxy, CN, -ORa, -SRa, -N(Ra)₂, (=O), -C₁₋₄ alkyl optionally substituted with 1, 2, or 3 substituents each independently selected from the group consisting of halogen, CN, -ORa, and -N(Ra)₂, optionally substituted -C₆₋₁₀ aryl, optionally substituted -(5- to 10-membered)-C₁₋₉ heteroaryl, -(5- to 10-membered)-C₂₋₉ heterocyclyl, and -C₃₋₁₀ cycloalkyl; and wherein said aryl, heteroaryl, and heterocyclyl is optionally fused to a further (second) ring; and
each Ra is independently hydrogen, -C₁₋₄ alkyl, -C₃₋₁₀ cycloalkyl, or -(5- to 10-membered)-C₂₋₉ heterocyclyl, wherein said alkyl, cycloalkyl or heterocyclyl group is optionally substituted by 1, 2 or 3 fluorine atoms.

93. The compound for use according to claim 92, wherein the compound is selected from the group consisting of 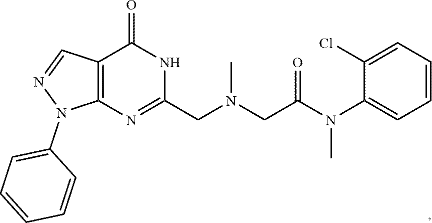 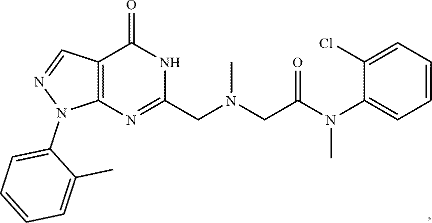 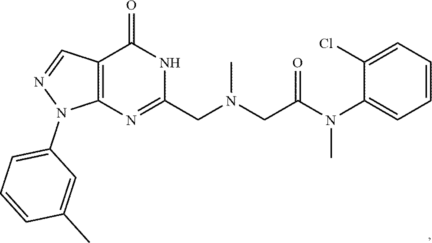 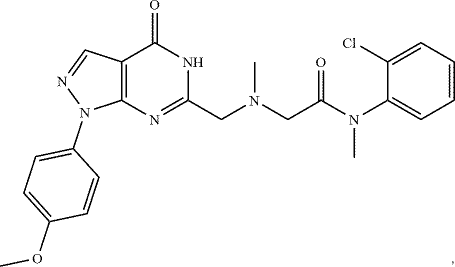 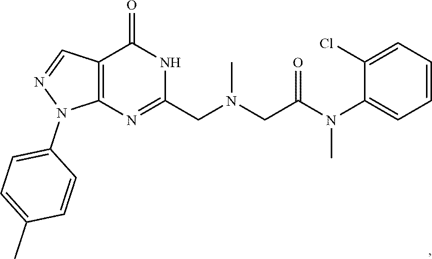 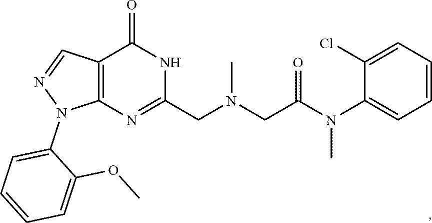
and 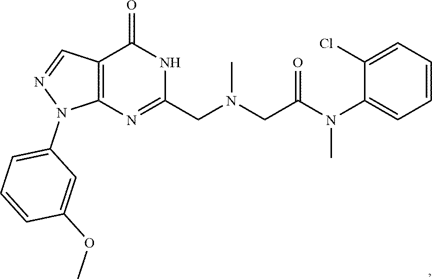
or a pharmaceutically acceptable salt or solvate thereof.

94. The compound for use according to claim 92, wherein the compound is selected from the group consisting of 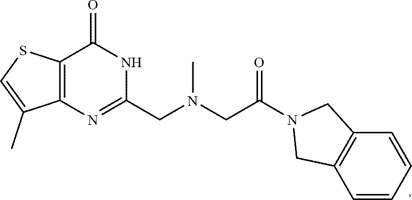 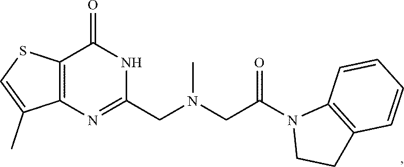 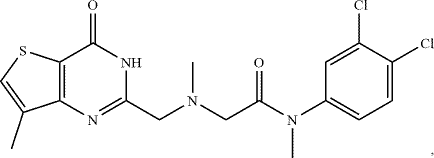 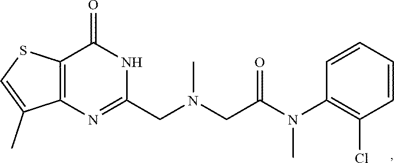 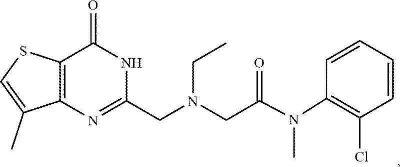 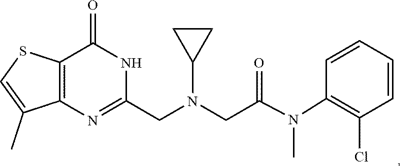 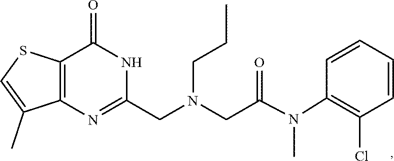 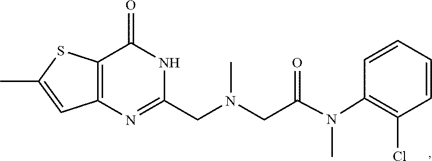 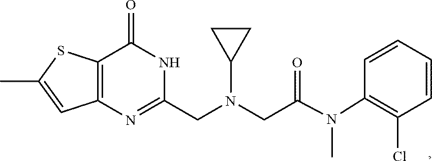 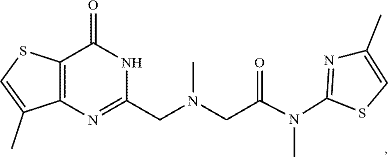 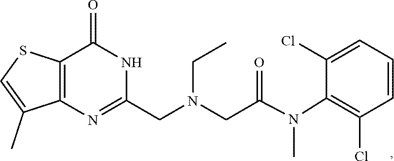 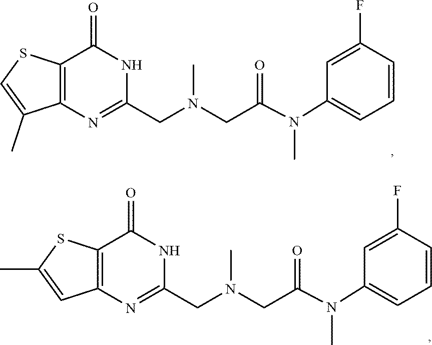 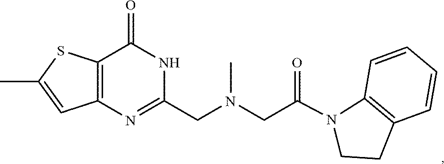 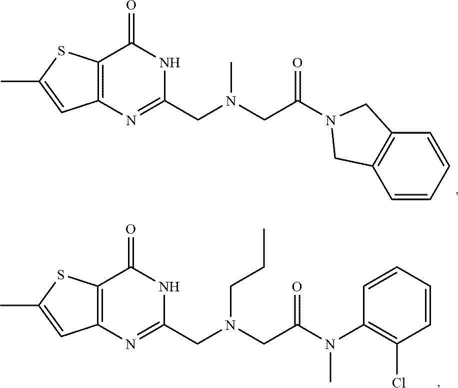 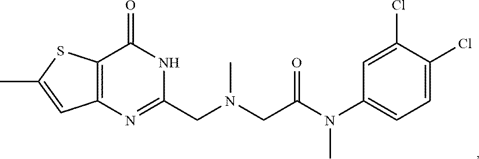 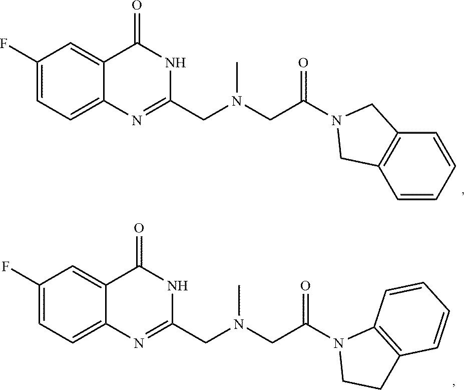 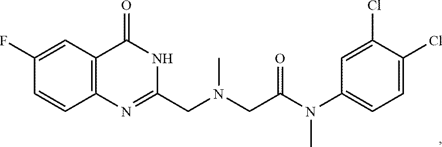 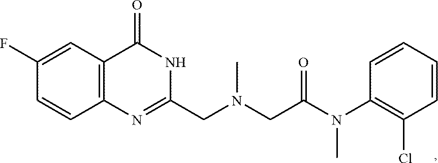 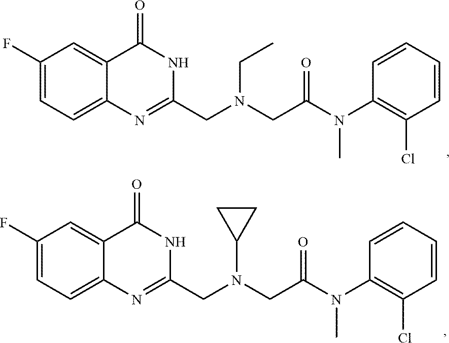 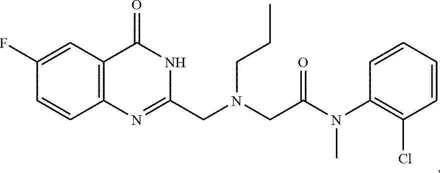 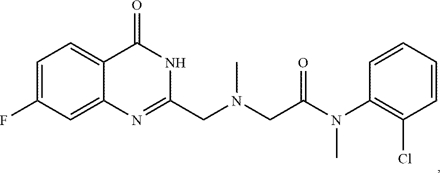 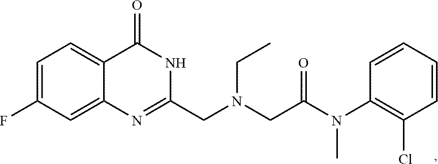 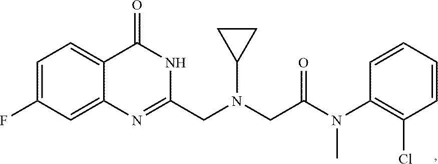 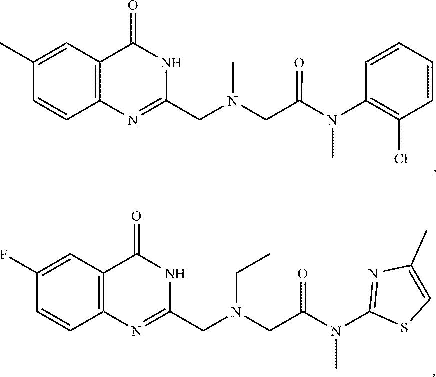 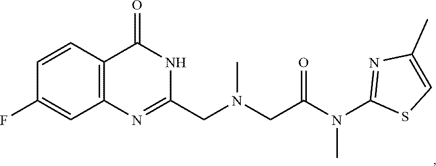 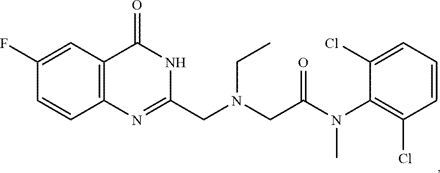 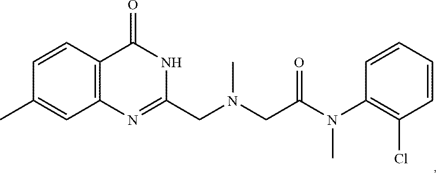 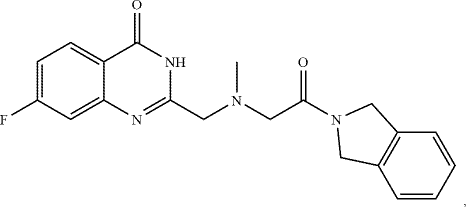 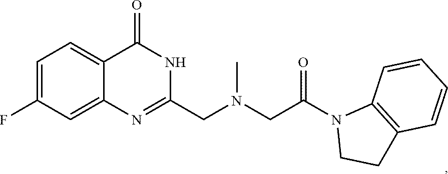 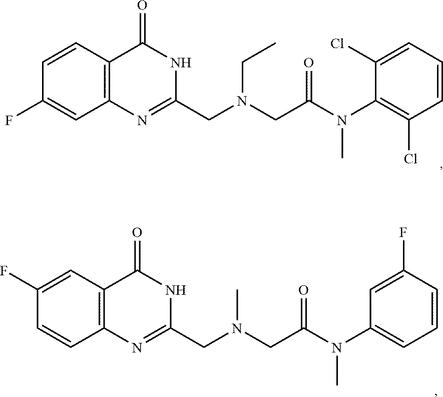 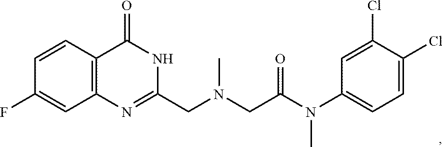 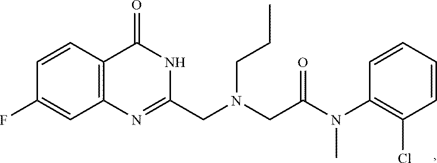 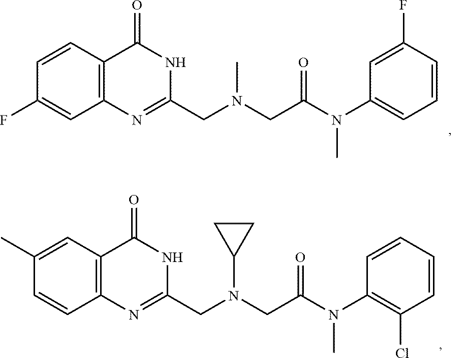 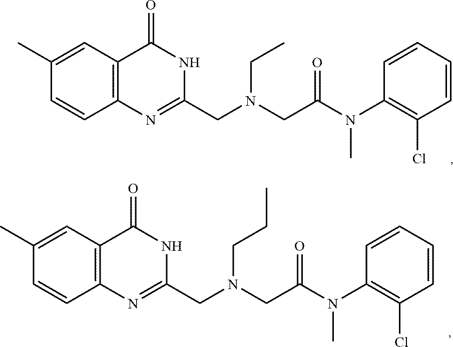 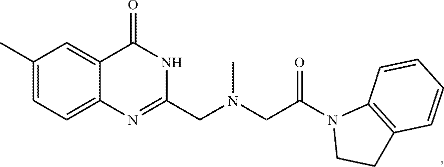 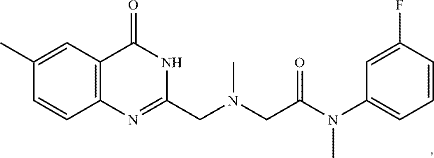 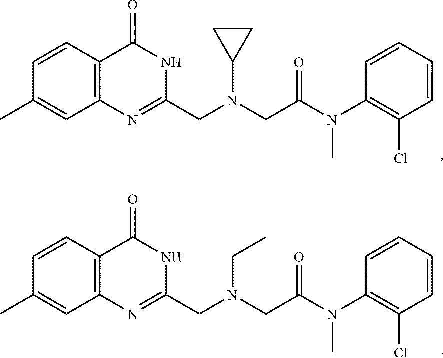 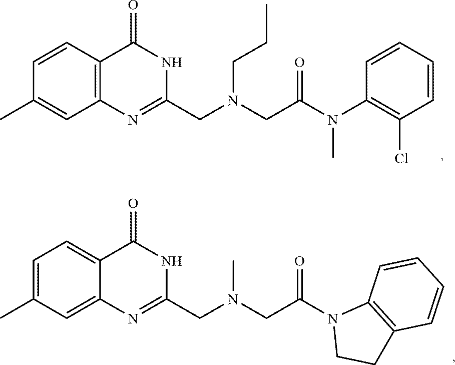 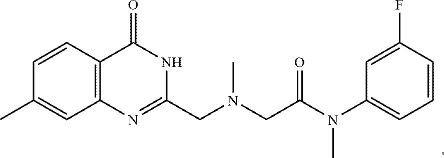 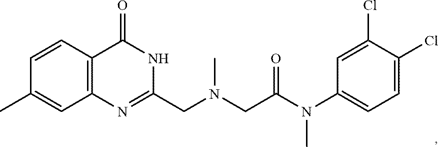 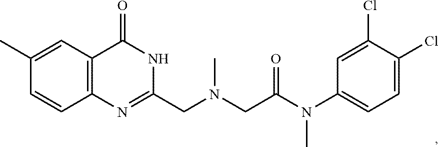 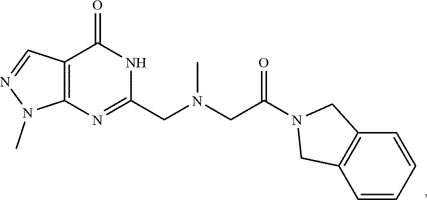 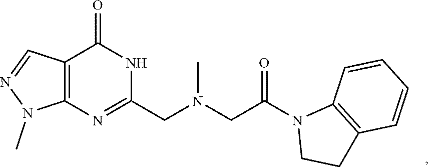 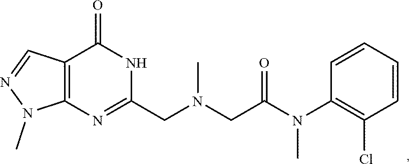 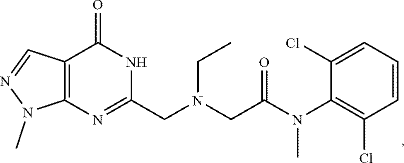
and 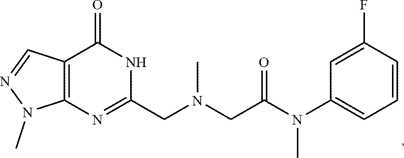
or a pharmaceutically acceptable salt or solvate thereof.

95. The compound for use according to any one of claims 88-94, wherein said α-L-iduronidase or an analog or variant thereof comprises laronidase.

96. The compound for use according to any one of claims 88-95, wherein the medicament is for use in the treatment or prevention of a condition associated with the alteration of the activity of α-L-iduronidase.

97. The compound for use according to claim 96, wherein the condition is MPS 1.

98. The compound for use according to claim 97, wherein MPS1 is Hurler disease, Hurler-Scheie syndrome, or Scheie syndrome.

99. The compound for use according to claim 96, wherein the conditions is selected from the group consisting of a heart valve disease, dysostosis multiplex, an eye disease, an ear disease, a respiratory obstruction or insufficiency, nerve compression, inflammatory arthritis, an amyloid related disorder, a disease condition associated with lipoprotein metabolism, solid cancers, infectious disease, an inflammatory disorder, and a developmental disorder.
